SEQUENCE LISTING (1 sequence)

SEQ ID NO: 1 - protein, 13 aa, synthetic construct
  SITE 13: note = lysine-CPQ2 chromophore-amide
ESATLQSGLRKAK

(19) United States
(12) Patent Application Publication
(10) Pub. No.: US 2024/0228535 A1
(43) Pub. Date: Jul. 11, 2024

(54) PROTEASE INHIBITORS FOR TREATING OR PREVENTING CORONAVIRUS INFECTION

(71) Applicant: Merck Sharp & Dohme LLC, Rahway, NJ (US)

(72) Inventors: John J. Acton, III, Cranford, NJ (US); Mayuri Gupta, Union, NJ (US); Michael J. Kelly, III, Paoli, PA (US); Franca-Maria Klingler, Cambridge (GB); Mark Eric Layton, Harleysville, PA (US); John A. McCauley, Maple Glen, PA (US); Gregori J. Morriello, Randolph, NJ (US); Christopher Charles Nawrat, Union, NJ (US); Craig A. Parish, Tenafly, NJ (US); Anthony J. Roecker, Harleysville, PA (US); Manuel de Lera Ruiz, Perkasie, PA (US); Jing Su, Scotch Plains, NJ (US); Valerie W. Shurtleff, Philadelphia, PA (US); Quang T. Truong, Morganville, NJ (US)

(73) Assignee: Merck Sharp & Dohme LLC, Rahway, NJ (US)

(21) Appl. No.: 18/513,791

(22) Filed: Nov. 20, 2023

Related U.S. Application Data
(60) Provisional application No. 63/485,677, filed on Feb. 17, 2023.
(60) Provisional application No. 63/427,330, filed on Nov. 22, 2022.

(51) Int. Cl. C07K 5/097; A61K 45/06

(52) U.S. Cl. CPC C07K 5/0821; A61K 45/06; A61K 38/00

(57) ABSTRACT

The present invention provides a compound of Formula I

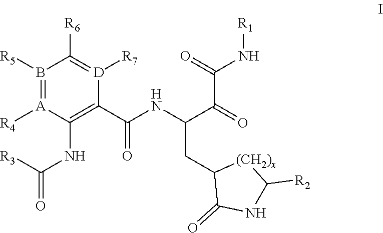

wherein R₁, R₂, R₃, R₄, R₅, R₆, R₇, and subscripts x and n are as described herein and pharmaceutical compositions comprising one or more said compounds, and methods for using said compounds for the treatment, inhibition, or amelioration of one or more disease states that could benefit from inhibition of a coronavirus, including SARS-CoV, MERS-CoV and SARS-CoV-2. The compounds of this invention could further be used in combination with other therapeutically effective agents, including but not limited to, other drugs useful for the treatment of coronavirus infection. The invention furthermore relates to processes for preparing compounds of Formula I, and pharmaceutical compositions which comprise compounds of Formula I and pharmaceutically acceptable salts thereof.

### CROSS REFERENCE TO RELATED APPLICATIONS

The present application claims the benefit of U.S. Provisional Application Nos. 63/485,677, filed Feb. 17, 2023, and 63/427,330, filed Nov. 22, 2022, hereby incorporated by reference in its entirety.

### REFERENCE TO SEQUENCE LISTING SUBMITTED ELECTRONICALLY

The sequence listing of the present application is submitted electronically via EFS-Web as an ASCII formatted sequence listing with a file name 25613-SEQLIST-11OCT2023.xml, creation date of Oct. 11, 2023, and a size of 3 KB.

### FIELD OF THE INVENTION

The present invention relates to certain protease inhibitors, pharmaceutical compositions comprising such inhibitors, and methods for using said compounds for the treatment, inhibition or amelioration of one or more disease states that could benefit from inhibition of a coronavirus, including SARS-CoV, MERS-CoV and SARS-CoV-2.

### BACKGROUND OF THE INVENTION

Coronaviruses (CoVs) are large, enveloped, positive-stranded, RNA viruses that comprise the Coronavirinae subfamily in the Nirovirales order. CoVs are further classified into four genera: alpha coronavirus, beta coronavirus, gamma coronavirus and delta coronavirus. Alpha and beta CoVs infect humans and other mammals, whereas the gamma and delta CoVs infect only animals (e.g., birds, sea mammals, pigs). CoV infection can result in a wide range of acute to chronic diseases of the respiratory, enteric and central nervous systems (Fields Virology Emerging Viruses Vol. 1. 2021. pp. 410-412).

To date, seven different coronaviruses that cause disease in humans have been identified: HCoV-229E, HCoV-NL63, HCoV-OC43, HCoV-HKU1, severe acute respiratory syndrome coronavirus (SARS-CoV), Middle East respiratory syndrome coronavirus (MERS-CoV) and, most recently SARS-CoV-2. HCoV-229E, HCoV-NL63, HCoV-OC43 and HCoV-HKU1 circulate on a yearly basis and cause mild symptoms similar to a common cold (Forni D, Cagliani R, Clerici M, and Sironi M. 2017. Trends in Microbiology, January 2017, Vol. 25, No. 1. 35-48). SARS-CoV, MERS-CoV and SARS-CoV-2 however, which have emerged in three zoonotic CoV transmission events over the last 21 years, are associated with mild to severe symptoms of respiratory infection such as fever, cough, dyspnea, pneumonia and acute respiratory distress syndrome that can ultimately lead to death.

The SARS-CoV epidemic in 2002 to 2003 was contained, but it resulted in 8,000 SARS-CoV infections and more than 800 deaths (Fields Virology Emerging Viruses Vol. 1. 2021. pp. 438). Camel-human zoonotic transmission of MERS-CoV occurred in Saudi Arabia in 2012. Although human to human transmission has been documented, most de novo infections occur as a result of camel-human interactions, and outbreaks are generally localized to the Arabian Peninsula (Zaki A M, van Zaki A M, van Boheemen S, Bestebroer T M, Osterhaus A, Fouchier R A M. 2012 N Engl J Med 367:1814-1820). The fatality rate of MERS infection is about 36% (www.who.int/csr/don/16-october-2014-mers/en/). SARS-CoV-2, the pandemic strain causal of COVID-19, is of bat origin, and transmission from bat to humans may have occurred directly or via an unknown intermediate host animal (Lu R, Zhao X, Li J, et al. 2020. Lancet; 395(10224):565-574). SARS-CoV-2 is now a pandemic CoV and has resulted, as of December 2021, in a worldwide health and economic crisis with global deaths exceeding 5 million (JHU CSSE COVID-19 Data github.com/CSSEGISandData/COVID-19). These three well-characterized zoonotic events, and the likelihood of future spillover events with novel CoVs, underscores the need for broad-spectrum CoV antiviral therapies that will be active against both existing CoVs, such as MERS-CoV and SARS-CoV-2, and also CoVs that may emerge in the future.

CoV particles consist of a cell-derived lipid membrane containing structural proteins spike (S), membrane (M), envelope (E), and nucleocapsid (N) (Fields Virology Emerging Viruses Vol. 1 2021 pp. 416-417). The virion also contains a large (25-32 kb) non-segmented positive-sense single-strand viral RNA genome that, similar to cellular mRNAs, is 5′-capped, contains 5′ and 3′ untranslated regions (UTRs) and a 3′ polyadenylated tail. All CoV viral genomes contain six basic common genes: two long open reading frames (1a and 1b) that encode two polypeptides that constitute the non-structural proteins (nsps) that form the multiprotein replicase-transcription complex (RTC) and four open reading frames for the structural proteins S, M, E and N that make up the virion. Depending on the CoV, one to eight additional genes, called accessory genes, can be encoded in the genome. The genomic organization amongst all CoVs is conserved and invariant across different genera such that the gene sequence is always 1a, 1b, S, M, E and N.

CoV replication is initiated through binding of the S protein to a specific cell surface receptor. SARS-CoV and SARS-CoV-2, for example, engage the angiotensin converting enzyme 2 (ACE-2) on cells of the upper respiratory tract (Lu R, Zhao X, Li J, et al. 2020. Lancet; 395(10224):565-574). Viral attachment leads to either viral endocytosis followed by fusion of the viral and endosome membranes, or direct fusion of the viral and cellular plasma members at the cell surface, to release virions into the cytoplasm. After entry, the viral genomic RNA is uncoated and serves as a template for cap-dependent translation of Orf 1a and Orf 1b to produce the viral polypeptides pp1a and pp1ab (Fung S, Liu D, 2019. Annu. Rev. Microbiol. 73: 529-57). Cleavage of the viral polypeptides to yield the individual replisome proteins is carried out by the viral papain-like protease (PLPro or nsp3) and 3CL main protease (Mpro or nsp5). The nsps form double-membraned vesicles and assemble to form RTCs responsible for genome replication, sub-genomic RNA (sgRNA) synthesis and transcription of the sgRNAs. The sgRNA serve as templates from which the mRNAs encoding for the structural and accessory proteins are translated. Assembly of new viral particles occurs in the endoplasmic reticulum—golgi intermediate complex and mature particles are released through secretory vesicles.

Vaccines for prevention of COVID-19 have been developed using the S protein of SARS-CoV-2 as an antigen to elicit a protective immune response (Kryikidis et al., npj Vaccines 28 (2021) 6:28). Vaccines based on mRNA/lipid nanoparticle and replication-defective adenoviruses vectored platforms have both been demonstrated to be highly effective for prevention of serious illness. However, there is limited data on the effectiveness of these vaccines for transmission of SARS-CoV-2. A liability of using the S protein for vaccine development is that the amino acid sequence is highly variable, enabling the SARS-CoV-2 to adapt to immune pressure (Chen R E et al. Nature Medicine. Mar. 4, 2021). Multiple independent spike mutations have been detected, even in the absence of vaccine selective pressure, and some variants will likely lead to reduced efficacy in vaccine clinical trials conducted where those variants are circulating.

Given the limitations of the current vaccines and the potential for zoonotic emergence of new pandemic strains, there is an urgent need for broad-spectrum anti-coronaviral treatment and prophylactic regimens. An anti-coronavirus intervention with efficacy against SARS-CoV, SARS-CoV-2, and the more distantly related MERS-CoV would be expected to have broad-spectrum activity against both SARS-CoV-2 and future CoVs that may emerge through zoonotic events.

### SUMMARY OF THE INVENTION

The present invention provides compounds of Formula I:

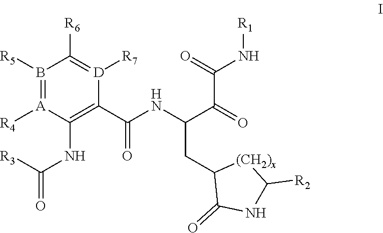

and pharmaceutically acceptable salts thereof. The compounds of Formula I are protease inhibitors, and as such may be useful in the treatment, inhibition, or amelioration of one or more disease states that could benefit from inhibition of a coronavirus, including SARS-CoV, MERS-CoV and SARS-CoV-2. Thus, the present invention also provides a method for prophylaxis or treatment of a coronavirus infection (e.g., a SARS-CoV, a SARS-CoV-2 or a MERS-CoV infection), comprising administering an effective amount of the compound of any of the compounds of Formula I disclosed herein or a pharmaceutically acceptable salt thereof to a patient in need thereof.

The compounds of this invention could further be used in combination with other therapeutically effective agents (one or more additional therapeutic agents), including but not limited to, other drugs useful for the treatment of coronavirus infection. Such additional therapeutic agents could include molnupiravir, pomotrelvir, ensitrelvir, nirmatrelvir, and ritonavir. The invention furthermore relates to processes for preparing compounds of Formula I, and pharmaceutical compositions which comprise compounds of Formula I and pharmaceutically acceptable salts thereof.

### DETAILED DESCRIPTION OF THE INVENTION

In one aspect, the present invention a compound of Formula I:

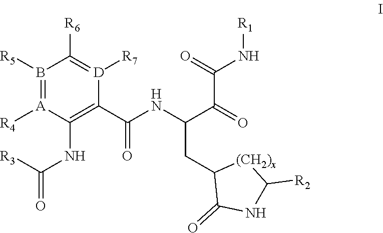

or a pharmaceutically acceptable salt thereof, wherein:
R₁ is H; (C₃-C₆)cycloalkyl; (C₁-C₆)alkyl; (C₁-C₆)alkyl-OH; phenyl; (C₁-C₆)alkyl-phenyl; (C₄-C₆)heterocycloalkyl containing 1 to 3 heteroatom(s) independently selected from N, O, or S; (C₁-C₆)alkyl-(C₄-C₆)heterocycloalkyl containing 1 to 3 heteroatom(s) independently selected from N, O, or S; (C₁-C₆)alkyl-(C₅-C₆)heteroaryl containing 1 to 3 heteroatom(s) independently selected from N, O, or S; (C₅-C₆)heteroaryl containing 1 to 3 heteroatom(s) independently selected from N, O, or S; R₂ is H; (C₁-C₆)alkyl; (C₃-C₆)cycloalkyl; or CF₃; R₃ is (C₁-C₆)alkyl; (C₁-C₆)alkyl-CF₃; (C₁-C₆)alkyl-OH; (C₁-C₆)alkyl-O—CH₃; (C₁-C₆)alkyl-O—CF₃; (C₁-C₆)alkyl-O—(C₃-C₁₀)cycloalkyl optionally substituted by an OH, —(C₁-C₆)alkyl, CF₃, —(C₁-C₆)alkyl-CF₃, or up to 3 halogen; (CF₂)-phenyl; (C₁-C₆)alkyl-phenyl; (C₃-C₁₀)cycloalkyl optionally substituted by an OH, —(C₁-C₆)alkyl, CF₃, —(C₁-C₆)alkyl-CF₃, or up to 3 halogen; (C₁-C₆)alkyl-(C₃-C₁₀)cycloalkyl optionally substituted by a OH, —(C₁-C₆)alkyl, CF₃, —(C₁-C₆)alkyl-CF₃, or up to 3 halogen; (C₄-C₁₀)heterocycle including up to 3 heteroatoms independently selected from N, O, and S and optionally substituted by a carbonyl, OH, —(C₁-C₆)alkyl, CF₃, —(C₁-C₆)alkyl-CF₃, or up to 3 halogen; (C₁-C₆)alkyl-(C₄-C₁₀)heterocycle including up to 3 heteroatoms independently selected from N, O, and S and optionally substituted by a carbonyl, OH, —(C₁-C₆)alkyl, CF₃, —(C₁-C₆)alkyl-CF₃, or up to 3 halogen; (C₆-C₁₀)aryl optionally substituted by an OH, —(C₁-C₆)alkyl, CF₃, —(C₁-C₆)alkyl-CF₃, or up to 3 halogen; (C₁-C₆)alkyl-(C₆-C₁₀)aryl optionally substituted by an OH, —(C₁-C₆)alkyl, CF₃, —(C₁-C₆)alkyl-CF₃, or up to 3 halogen; (C₅-C₁₀)heteroaryl including up to 4 heteroatoms independently selected from N, O, and S and optionally substituted by an OH, CN, CHF₂, —(C₁-C₆)alkyl, CF₃, —(C₁-C₆)alkyl-CF₃, —(C₃-C₆)cycloalkyl, O—(C₁-C₆)alkyl, O—CF₃, O—(C₃-C₆)cycloalkyl, or up to 3 halogen; (C₁-C₆)alkyl-(C₅-C₁₀)heteroaryl including up to 4 heteroatoms independently selected from N, O, and S and optionally substituted by an OH, CN, CHF₂, (C₃-C₆)cycloalkyl, O—(C₃-C₆)cycloalkyl, —(C₁-C₆)alkyl, O—(C₁-C₆)alkyl, CF₃, O—CF₃, —(C₁-C₆)alkyl-CF₃, or up to 3 halogen; —O—(C₁-C₆)alkyl; —O—(C₁-C₆)alkyl-(C₃-C₆)cycloalkyl optionally substituted by an OH, CF₃, —(C₁-C₆)alkyl, —(C₁-C₆)alkyl-CF₃, or up to 3 halogen; —O—(C₃-C₆)cycloalkyl optionally substituted by an OH, CF₃, —(C₁-C₆)alkyl, —(C₁-C₆)alkyl-CF₃, or up to 3 halogen; CHF₂; CF₃; (C₁-C₆)alkyl-N(CH₃)—C(O)—O—CH₃; or N(R₈)₂;
R₄ is H; F; Cl; or (C₁-C₆)alkyl;
R₅ is H; F; Cl; or (C₁-C₆)alkyl;
R₆ is H; F; Cl; CN; CF₃; O—CHF₂; O—CF₃; —(C₁-C₆)alkyl-CF₃; CHF₂; CF₃; (C₁-C₆)alkyl; (C₃-C₆)cycloalkyl; —O—(C₃-C₆)cycloalkyl; or O—(C₁-C₆)alkyl;
R₇ is H; F; Cl; or (C₁-C₆)alkyl;
R₈ is independently H; (C₁-C₆)alkyl; (C₃-C₁₀)cycloalkyl optionally substituted by an OH, —(C₁-C₆)alkyl, CF₃, —(C₁-C₆)alkyl-CF₃, or up to 3 halogen; (C₁-C₆)alkyl-(C₃-C₁₀)cycloalkyl optionally substituted by an OH, —(C₁-C₆)alkyl, CF₃, —(C₁-C₆)alkyl-CF₃, or up to 3 halogen; (C₄-C₁₀)heterocycle including up to 3 heteroatoms selected from N, O, and S and optionally substituted by an OH, —(C₁-C₆)alkyl, CF₃, —(C₁-C₆)alkyl-CF₃, or up to 3 halogen; (C₁-C₆)alkyl-(C₄-C₁₀)heterocycle including up to 3 heteroatoms selected from N, O, and S and optionally substituted by an OH, —(C₁-C₆)alkyl, CF₃, —(C₁-C₆)alkyl-CF₃, or up to 3 halogen; (C₆-C₁₀)aryl optionally substituted by an OH, —(C₁-C₆)alkyl, CF₃, —(C₁-C₆)alkyl-CF₃, or up to 3 halogen; (C₁-C₆)alkyl-(C₆-C₁₀)aryl optionally substituted by an OH, —(C₁-C₆)alkyl, CF₃, —(C₁-C₆)alkyl-CF₃, or up to 3 halogen; (C₅-C₁₀)heteroaryl including up to 3 heteroatoms selected from N, O, and S and optionally substituted by an OH, —(C₁-C₆)alkyl, CF₃, —(C₁-C₆)alkyl-CF₃, or up to 3 halogen; (C₁-C₆)alkyl-(C₅-C₁₀)heteroaryl including up to 3 heteroatoms selected from N, O, and S and optionally substituted by an OH, —(C₁-C₆)alkyl, CF₃, —(C₁-C₆)alkyl-CF₃, or up to 3 halogen;
A is C or N;
B is C or N;
D is C or N, provided that only 1 of A, B, or D can be N and that if A, B, or D is N then the respective R₄, R₅, or R₇ is absent; and
x is 1 or 2.

In some embodiments of the present invention, R₁ is H, (C₃-C₆)cycloalkyl, or (C₁-C₆)alkyl. In specific embodiments, R₁ is H, CH₃ or cyclopropyl.

In certain embodiments of the present invention, R₂ is CF₃, CH₃ or H.

In some embodiments of the present invention, the group R₃ is CH₃,

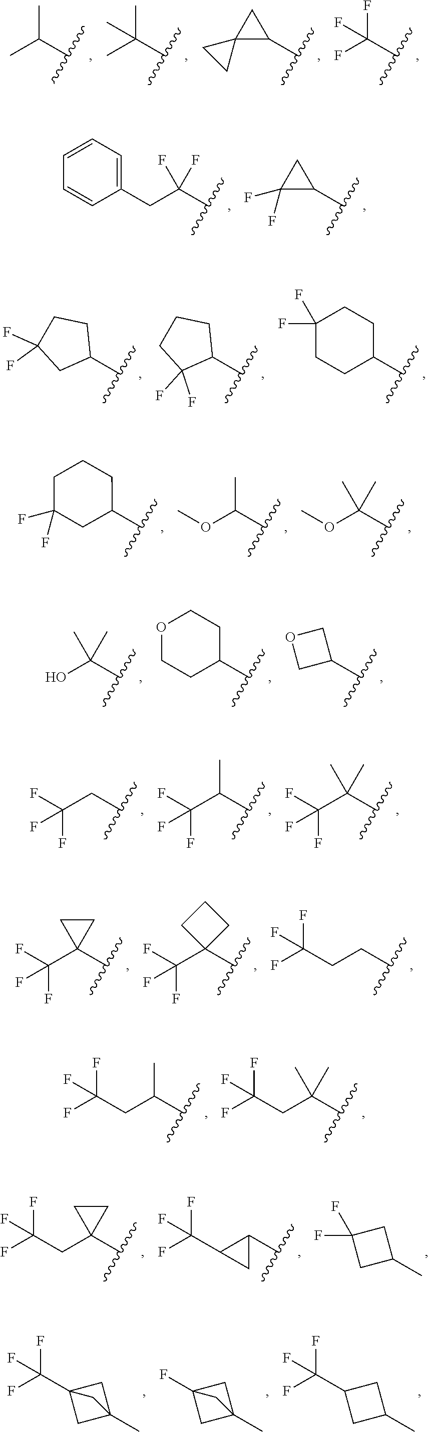

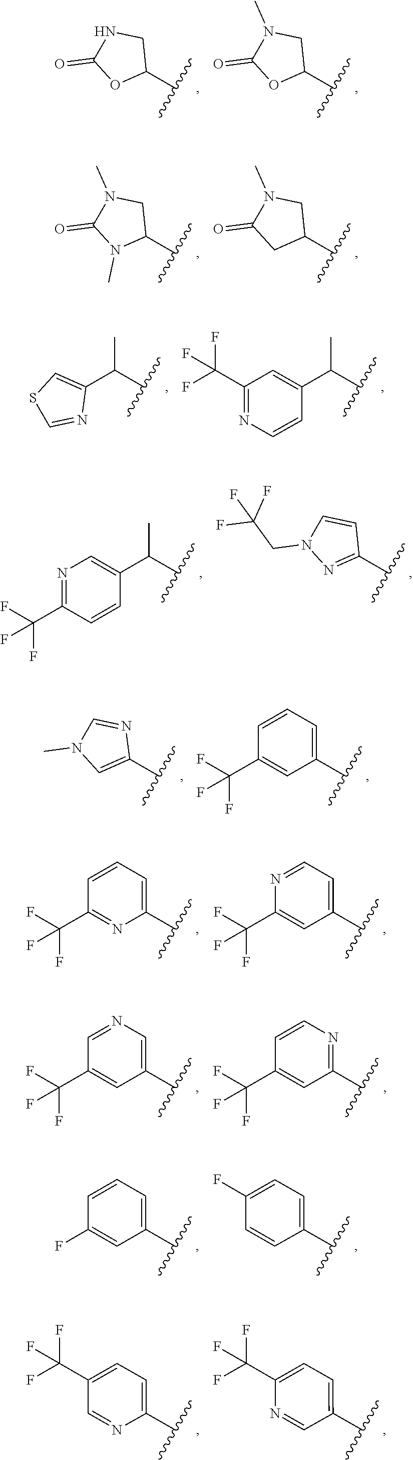

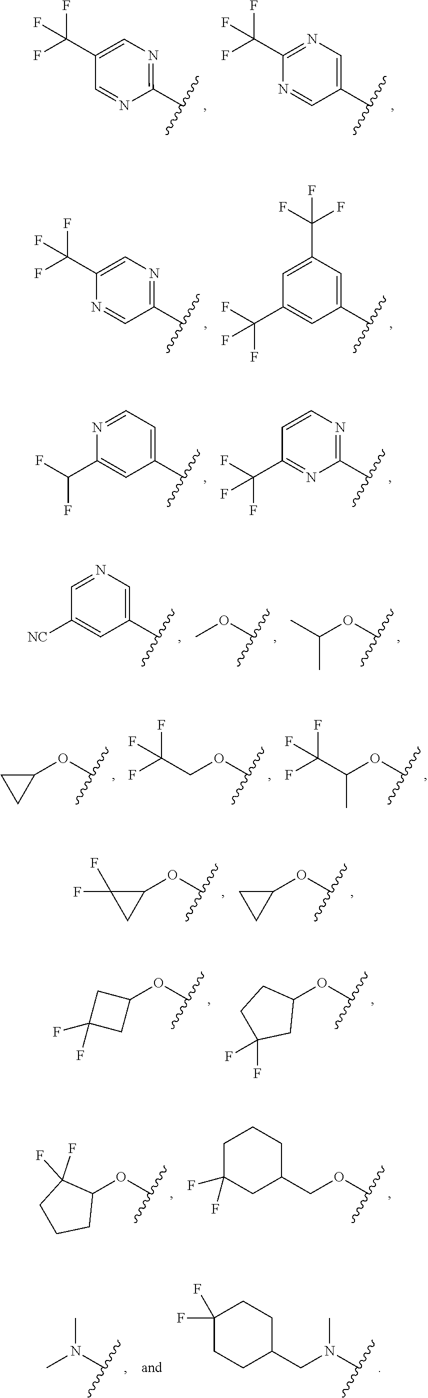

In certain embodiments of the present invention, R₄ is H, F, or Cl.

In some embodiments of the present invention, R₅ is H, F, or Cl.

In some embodiments of the present invention, R₆ is F, Cl, CHF₂, or CN.

In certain embodiments of the present invention, R₇ is H, F, or Cl.

In certain embodiments of the present invention, one of A, B or D is N.

In other embodiments of the present invention, A, B and D are all C.

In certain embodiments of the present invention, x is 1.

Reference to the specific classes and subclasses set forth above is meant to include all combinations of particular and preferred groups unless stated otherwise.

Specific embodiments of the present invention include, but are not limited to, the compounds disclosed in Examples 1 to 100, or pharmaceutically acceptable salts thereof.

Other specific embodiments include compounds enumerated below or pharmaceutically acceptable salts thereof:
5-chloro-N-[(1S)-3-(cyclopropylamino)-1-[[(3S,5R)-5-methyl-2-oxo-pyrrolidin-3-yl]methyl]-2,3-dioxo-propyl]-2-[[1-(trifluoromethyl)cyclopropanecarbonyl]amino]benzamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-2-(trifluoromethyl)pyridine-4-carboxamide;
5-chloro-N-[((1S)-3-(cyclopropylamino)-1-[[(3S,5R)-5-methyl-2-oxo-pyrrolidin-3-yl]methyl]-2,3-dioxo-propyl]-2-(4,4,4-trifluorobutanoylamino)benzamide;
2-[[3,5-bis(trifluoromethyl)benzoyl]amino]-5-cyano-N-[(1S)-3-(methylamino)-1-[[(3S,5R)-5-methyl-2-oxo-pyrrolidin-3-yl]methyl]-2,3-dioxo-propyl]pyridine-3-carboxamide;
5-chloro-N-[((1S)-3-(cyclopropylamino)-1-[[(3S,5R)-5-methyl-2-oxo-pyrrolidin-3-yl]methyl]-2,3-dioxo-propyl]-2-[[3-(trifluoromethyl)bicyclo[1.1.1]pentane-1-carbonyl]amino]pyridine-3-carboxamide;
isopropyl N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]carbamate;
5-chloro-N-[((1S)-3-(cyclopropylamino)-1-[[(3S,5R)-5-methyl-2-oxo-pyrrolidin-3-yl]methyl]-2,3-dioxo-propyl]-2-[(2,2-difluoro-3-phenyl-propanoyl)amino]benzamide;
5-chloro-N-[((1S)-3-(cyclopropylamino)-1-[[(3S,5R)-5-methyl-2-oxo-pyrrolidin-3-yl]methyl]-2,3-dioxo-propyl]-2-[[(1R)-2,2-difluorocyclopropanecarbonyl]amino]benzamide;
methyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
N-[(1S)-3-(cyclopropylamino)-2,3-dioxo-1-[[(3S)-2-oxopyrrolidin-3-yl]methyl]propyl]-5-fluoro-2-[[1-(trifluoromethyl)cyclopropanecarbonyl]amino]benzamide;
N-[4-cyano-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-3-fluoro-bicyclo[1.1.1]pentane-1-carboxamide;
5-chloro-2-[(2,2-difluorocyclopropanecarbonyl)amino]-N-[(1S)-3-(methylamino)-1-[[(3S,5R)-5-methyl-2-oxo-pyrrolidin-3-yl]methyl]-2,3-dioxo-propyl]benzamide;
5-chloro-N-[((1S)-3-(cyclopropylamino)-1-[[(3S,5R)-5-methyl-2-oxo-pyrrolidin-3-yl]methyl]-2,3-dioxo-propyl]-2-[(3,3,3-trifluoro-2,2-dimethyl-propanoyl)amino]benzamide;
5-chloro-2-[(3,3-difluorocyclopentanecarbonyl)amino]-N-[(1S)-3-(methylamino)-1-[[(3S,5R)-5-methyl-2-oxo-pyrrolidin-3-yl]methyl]-2,3-dioxo-propyl]benzamide;
N-[4-cyano-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-3-(trifluoromethyl)bicyclo[1.1.1]pentane-1-carboxamide;
5-(difluoromethoxy)-N-[(1S)-3-(methylamino)-1-[[(3S,5R)-5-methyl-2-oxo-pyrrolidin-3-yl]methyl]-2,3-dioxo-propyl]-2-[[3-(trifluoromethyl)benzoyl]amino]pyridine-3-carboxamide;
5-chloro-N-[(1S)-3-(methylamino)-1-[[(3S,5R)-5-methyl-2-oxo-pyrrolidin-3-yl]methyl]-2,3-dioxo-propyl]-2-[(3,3,3-trifluoro-2-methyl-propanoyl)amino]benzamide;
N-[(1S)-3-(cyclopropylamino)-1-[[(3S,5R)-5-methyl-2-oxo-pyrrolidin-3-yl]methyl]-2,3-dioxo-propyl]-5-(difluoromethoxy)-2-[[3-(trifluoromethyl)benzoyl]amino]benzamide;
5-chloro-2-[(3,3-difluorocyclobutanecarbonyl)amino]-N-[(1S)-3-(methylamino)-1-[[(3S,5R)-5-methyl-2-oxo-pyrrolidin-3-yl]methyl]-2,3-dioxo-propyl]benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[[1-(trifluoromethyl)cyclopropanecarbonyl]amino]pyridine-3-carboxamide;
N-[(1S)-3-(cyclopropylamino)-1-[[(3S,5R)-5-methyl-2-oxo-pyrrolidin-3-yl]methyl]-2,3-dioxo-propyl]-2-[(3,3-difluorocyclobutanecarbonyl)amino]-5-fluoro-pyridine-3-carboxamide;
5-chloro-N-[(1S)-3-(methylamino)-1-[[(3S,5R)-5-methyl-2-oxo-pyrrolidin-3-yl]methyl]-2,3-dioxo-propyl]-2-[(3,3,3-trifluoro-2-methyl-propanoyl)amino]benzamide;
N-[(1S)-3-(cyclopropylamino)-1-[[(3S,5R)-5-methyl-2-oxo-pyrrolidin-3-yl]methyl]-2,3-dioxo-propyl]-5-fluoro-2-[[1-(trifluoromethyl)cyclopropanecarbonyl]amino]benzamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-6-(trifluoromethyl)pyridine-2-carboxamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[(3-fluorobicyclo[1.1.1]pentane-1-carbonyl)amino]pyridine-3-carboxamide;
5-fluoro-N-[(1S)-3-(methylamino)-1-[[(3S,5R)-5-methyl-2-oxo-pyrrolidin-3-yl]methyl]-2,3-dioxo-propyl]-2-[[3-(trifluoromethyl)benzoyl]amino]pyridine-3-carboxamide;
5-chloro-N-[(1S)-3-(cyclopropylamino)-2,3-dioxo-1-[[(3S)-2-oxopyrrolidin-3-yl]methyl]propyl]-2-(4,4,4-trifluorobutanoylamino)benzamide;
N-[4-chloro-2-[[(1S)-3-(cyclopropylamino)-2,3-dioxo-1-[[(3S)-2-oxopyrrolidin-3-yl]methyl]propyl]carbamoyl]phenyl]-3-fluoro-bicyclo[1.1.1]pentane-1-carboxamide;
isopropyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
5-cyano-N-[(1S)-3-(cyclopropylamino)-1-[[(3S,5R)-5-methyl-2-oxo-pyrrolidin-3-yl]methyl]-2,3-dioxo-propyl]-2-[(3-fluorobenzoyl)amino]benzamide;
5-chloro-N-[(1S)-3-(cyclopropylamino)-1-[[(3S,5R)-5-methyl-2-oxo-pyrrolidin-3-yl]methyl]-2,3-dioxo-propyl]-4-fluoro-2-[[1-(trifluoromethyl)cyclopropanecarbonyl]amino]benzamide;
5-chloro-N-[((1S)-3-(methylamino)-1-[[(3S,5R)-5-methyl-2-oxo-pyrrolidin-3-yl]methyl]-2,3-dioxo-propyl]-2-[[1-(trifluoromethyl)cyclopropanecarbonyl]amino]benzamide;
5-chloro-N-[((1S)-3-(cyclopropylamino)-1-[[(3S,5R)-5-methyl-2-oxo-pyrrolidin-3-yl]methyl]-2,3-dioxo-propyl]-2-[[1-(trifluoromethyl)cyclobutanecarbonyl]amino]benzamide;
5-chloro-N-[(1S)-3-(cyclopropylamino)-2,3-dioxo-1-[[(3S)-2-oxopyrrolidin-3-yl]methyl]propyl]-2-[[1-(2,2,2-trifluoroethyl)cyclopropanecarbonyl]amino]benzamide;
N-[4-chloro-2-[[(1S)-3-(cyclopropylamino)-2,3-dioxo-1-[[(3S)-2-oxopyrrolidin-3-yl]methyl]propyl]carbamoyl]phenyl]-3-fluoro-bicyclo[1.1.1]pentane-1-carboxamide;
5-chloro-N-[(1S)-3-(cyclopropylamino)-2,3-dioxo-1-[[(3S)-2-oxopyrrolidin-3-yl]methyl]propyl]-2-(3,3,3-trifluoropropanoylamino)benzamide;
5-chloro-N-[((1S)-3-(cyclopropylamino)-1-[[(3S,5R)-5-methyl-2-oxo-pyrrolidin-3-yl]methyl]-2,3-dioxo-propyl]-2-[(4,4-difluorocyclohexanecarbonyl)amino]pyridine-3-carboxamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-6-(trifluoromethyl)pyridine-2-carboxamide;
5-chloro-2-[(3,3-difluorocyclobutanecarbonyl)amino]-N-[(1S)-3-(methylamino)-2,3-dioxo-1-[[(3R,5S)-2-oxo-5-(trifluoromethyl)pyrrolidin-3-yl]methyl]propyl]benzamide;
5-chloro-N-[((1S)-3-(cyclopropylamino)-1-[[(3S,5R)-5-methyl-2-oxo-pyrrolidin-3-yl]methyl]-2,3-dioxo-propyl]-2-(3,3,3-trifluoropropanoylamino)benzamide;
5-chloro-N-[(1S)-3-(methylamino)-2,3-dioxo-1-[[(3R,5S)-2-oxo-5-(trifluoromethyl)pyrrolidin-3-yl]methyl]propyl]-2-[[3-(trifluoromethyl)benzoyl]amino]pyridine-3-carboxamide;
N-[(1S)-3-(cyclopropylamino)-1-[[(3S,5R)-5-methyl-2-oxo-pyrrolidin-3-yl]methyl]-2,3-dioxo-propyl]-5-(difluoromethyl)-2-[[3-(trifluoromethyl)benzoyl]amino]benzamide;
5-chloro-N-[(1S)-3-(methylamino)-1-[[(3S,5R)-5-methyl-2-oxo-pyrrolidin-3-yl]methyl]-2,3-dioxo-propyl]-2-[[3-(trifluoromethyl)benzoyl]amino]benzamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-3-(trifluoromethyl)bicyclo[1.1.1]pentane-1-carboxamide;
N-[4-chloro-2-[[(1S)-3-(methylamino)-2,3-dioxo-1-[[(3S)-2-oxopyrrolidin-3-yl]methyl]propyl]carbamoyl]phenyl]-3-fluoro-bicyclo[1.1.1]pentane-1-carboxamide;
N-[(1S)-3-amino-1-[[(3S,5R)-5-methyl-2-oxo-pyrrolidin-3-yl]methyl]-2,3-dioxo-propyl]-5-chloro-2-[[3-(trifluoromethyl)benzoyl]amino]benzamide;
N-[2-[[(1S)-3-(cyclopropylamino)-1-[[(3S,5R)-5-methyl-2-oxo-pyrrolidin-3-yl]methyl]-2,3-dioxo-propyl]carbamoyl]-4,5-difluoro-phenyl]-2-(trifluoromethyl)pyridine-4-carboxamide;
5-chloro-2-[(3-fluorobicyclo[1.1.1]pentane-1-carbonyl)amino]-N-[(1S)-3-(methylamino)-1-[[(3S,5R)-5-methyl-2-oxo-pyrrolidin-3-yl]methyl]-2,3-dioxo-propyl]pyridine-3-carboxamide;
5-chloro-N-[(1S)-3-(cyclopropylamino)-2,3-dioxo-1-[[(3S)-2-oxopyrrolidin-3-yl]methyl]propyl]-2-[(2,2,2-trifluoroacetyl)amino]benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-(4,4,4-trifluorobutanoylamino)pyridine-3-carboxamide;
5-chloro-N-[(1S)-3-(cyclopropylamino)-1-[[(3S,5R)-5-methyl-2-oxo-pyrrolidin-3-yl]methyl]-2,3-dioxo-propyl]-4-fluoro-2-(4,4,4-trifluorobutanoylamino)benzamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-3-fluoro-bicyclo[1.1.1]pentane-1-carboxamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-5-(trifluoromethyl)pyridine-2-carboxamide;
5-chloro-N-[(1S)-3-(cyclopropylamino)-2,3-dioxo-1-[[(3S)-2-oxopyrrolidin-3-yl]methyl]propyl]-4-fluoro-2-(4,4,4-trifluorobutanoylamino)benzamide;
5-chloro-2-[(4-fluorobenzoyl)amino]-N-[(1S)-3-(methylamino)-1-[[(3S,5R)-5-methyl-2-oxo-pyrrolidin-3-yl]methyl]-2,3-dioxo-propyl]pyridine-3-carboxamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[(3-fluorobenzoyl)amino]pyridine-3-carboxamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[(3,3-difluorocyclobutanecarbonyl)amino]benzamide;
5-chloro-2-[(2,2-difluorocyclopropanecarbonyl)amino]-N-[(1S)-3-(methylamino)-1-[[(3S,5R)-5-methyl-2-oxo-pyrrolidin-3-yl]methyl]-2,3-dioxo-propyl]benzamide;
5-cyano-N-[(1S)-3-(cyclopropylamino)-1-[[(3S,5R)-5-methyl-2-oxo-pyrrolidin-3-yl]methyl]-2,3-dioxo-propyl]-2-[[3-(trifluoromethyl)benzoyl]amino]benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[[1-(2,2,2-trifluoroethyl)cyclopropanecarbonyl]amino]benzamide;
5-chloro-N-[(1S)-3-(cyclopropylamino)-2,3-dioxo-1-[[(3S)-2-oxopyrrolidin-3-yl]methyl]propyl]-2-[(3,3-difluorocyclobutanecarbonyl)amino]benzamide;
N-[(1S)-3-(cyclopropylamino)-1-[[(3S,5R)-5-methyl-2-oxo-pyrrolidin-3-yl]methyl]-2,3-dioxo-propyl]-5-(trifluoromethyl)-2-[[3-(trifluoromethyl)benzoyl]amino]benzamide;
5-chloro-2-[(3,3-difluorocyclopentanecarbonyl)amino]-N-[(1S)-3-(methylamino)-1-[[(3S,5R)-5-methyl-2-oxo-pyrrolidin-3-yl]methyl]-2,3-dioxo-propyl]benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[[3-(trifluoromethyl)benzoyl]amino]benzamide;
N-[(1S)-3-(cyclopropylamino)-1-[[(3S,5R)-5-methyl-2-oxo-pyrrolidin-3-yl]methyl]-2,3-dioxo-propyl]-5-fluoro-2-[[3-(trifluoromethyl)benzoyl]amino]pyridine-3-carboxamide;
5-chloro-N-[(1S)-3-(cyclopropylamino)-2,3-dioxo-1-[[(3R,5S)-2-oxo-5-(trifluoromethyl)pyrrolidin-3-yl]methyl]propyl]-2-(4,4,4-trifluorobutanoylamino)benzamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-3-(trifluoromethyl)bicyclo[1.1.1]pentane-1-carboxamide;
5-(difluoromethyl)-N-[(1S)-3-(methylamino)-1-[[(3S,5R)-5-methyl-2-oxo-pyrrolidin-3-yl]methyl]-2,3-dioxo-propyl]-2-[[3-(trifluoromethyl)benzoyl]amino]benzamide;
N-[4-chloro-2-[[(1S)-3-(cyclopropylamino)-2,3-dioxo-1-[[(3S)-2-oxopyrrolidin-3-yl]methyl]propyl]carbamoyl]phenyl]-5-(trifluoromethyl)pyridine-3-carboxamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-4-(trifluoromethyl)pyridine-2-carboxamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[(3-fluorobenzoyl)amino]benzamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-2-(trifluoromethyl)pyridine-4-carboxamide;
N-[4-chloro-2-[[(1S)-3-(cyclopropylamino)-2,3-dioxo-1-[[(3S)-2-oxopyrrolidin-3-yl]methyl]propyl]carbamoyl]phenyl]-2-(trifluoromethyl)pyridine-4-carboxamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-6-(trifluoromethyl)pyridine-3-carboxamide;
N-[2-[[(1S)-3-(cyclopropylamino)-1-[[(3S,5R)-5-methyl-2-oxo-pyrrolidin-3-yl]methyl]-2,3-dioxo-propyl]carbamoyl]-4-fluoro-phenyl]-2-(trifluoromethyl)pyridine-4-carboxamide;
N-[(1S)-3-(cyclopropylamino)-1-[[(3S,5R)-5-methyl-2-oxo-pyrrolidin-3-yl]methyl]-2,3-dioxo-propyl]-5-fluoro-2-[[3-(trifluoromethyl)benzoyl]amino]benzamide;
N-[4-chloro-2-[[(1S)-3-(cyclopropylamino)-2,3-dioxo-1-[[(3S)-2-oxopyrrolidin-3-yl]methyl]propyl]carbamoyl]-5-fluoro-phenyl]-5-(trifluoromethyl)pyridine-3-carboxamide;
N-[2,4-dichloro-6-[[(1S)-3-(cyclopropylamino)-1-[[(3S,5R)-5-methyl-2-oxo-pyrrolidin-3-yl]methyl]-2,3-dioxo-propyl]carbamoyl]phenyl]-3-fluoro-bicyclo[1.1.1]pentane-1-carboxamide;
5-chloro-N-[(1S)-3-(methylamino)-2,3-dioxo-1-[[(3S)-2-oxopyrrolidin-3-yl]methyl]propyl]-2-[[1-(trifluoromethyl)cyclopropanecarbonyl]amino]benzamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]spiro[2.2]pentane-2-carboxamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]spiro[2.2]pentane-2-carboxamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[[2-(trifluoromethyl)cyclopropanecarbonyl]amino]benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[[2-(trifluoromethyl)cyclopropanecarbonyl]amino]benzamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-4-(trifluoromethyl)pyridine-2-carboxamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]-5-fluoro-phenyl]-2-(trifluoromethyl)pyridine-4-carboxamide;
5-cyano-N-[3-(methylamino)-2,3-dioxo-1-[[(5S)-2-oxo-5-(trifluoromethyl)pyrrolidin-3-yl]methyl]propyl]-2-(4,4,4-trifluorobutanoylamino)benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-(2-thiazol-4-ylpropanoylamino)benzamide;
N-[4-chloro-2-[[(1S)-3-(methylamino)-2,3-dioxo-1-[[(3S)-2-oxopyrrolidin-3-yl]methyl]propyl]carbamoyl]phenyl]-2-(trifluoromethyl)pyridine-4-carboxamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-5-(trifluoromethyl)pyridine-3-carboxamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-2-(difluoromethyl)pyridine-4-carboxamide;
5-chloro-N-[(1S)-3-(cyclopropylamino)-1-[[(3S,5R)-5-methyl-2-oxo-pyrrolidin-3-yl]methyl]-2,3-dioxo-propyl]-2-(2-thiazol-4-ylpropanoylamino)benzamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-5-(trifluoromethyl)pyridine-3-carboxamide;
N-[(1S)-3-(methylamino)-2,3-dioxo-1-[[(3S)-2-oxopyrrolidin-3-yl]methyl]propyl]-5-(trifluoromethyl)-2-[[1-(trifluoromethyl)cyclopropanecarbonyl]amino]benzamide;
isopropyl N-[4-chloro-2-[[(1S)-3-(cyclopropylamino)-2,3-dioxo-1-[[(3S)-2-oxopyrrolidin-3-yl]methyl]propyl]carbamoyl]phenyl]carbamate;
5-chloro-N-[(1S)-3-(cyclopropylamino)-1-[[(3S,5R)-5-methyl-2-oxo-pyrrolidin-3-yl]methyl]-2,3-dioxo-propyl]-2-[[2-(trifluoromethyl)cyclopropanecarbonyl]amino]benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[[2-(trifluoromethyl)cyclopropanecarbonyl]amino]benzamide;
N-[2-[[(1S)-3-(cyclopropylamino)-1-[[(3S,5R)-5-methyl-2-oxo-pyrrolidin-3-yl]methyl]-2,3-dioxo-propyl]carbamoyl]-4,5-difluoro-phenyl]-5-(trifluoromethyl)pyridine-3-carboxamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-1-(2,2,2-trifluoroethyl)pyrazole-3-carboxamide;
methyl N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]carbamate;
cyclopropyl (4-chloro-2-(((S)-4-(methylamino)-3,4-dioxo-1-((S)-2-oxopyrrolidin-3-yl)butan-2-yl)carbamoyl)phenyl)carbamate;
cyclopropyl (4-chloro-2-(((S)-1-((3S,5R)-5-methyl-2-oxopyrrolidin-3-yl)-4-(methylamino)-3,4-dioxobutan-2-yl)carbamoyl)phenyl)carbamate;
5-chloro-N-[(1S)-3-(cyclopropylamino)-2,3-dioxo-1-[[(3S)-2-oxopyrrolidin-3-yl]methyl]propyl]-2-[[1-(trifluoromethyl)cyclopropanecarbonyl]amino]benzamide;
methyl N-[2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
methyl N-[4-fluoro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
methyl N-[4-chloro-2-[[(1S)-3-(methylamino)-2,3-dioxo-1-[[(3S)-2-oxopyrrolidin-3-yl]methyl]propyl]carbamoyl]phenyl]carbamate;
methyl N-[2-[[(1S)-3-amino-1-[[(3S,5R)-5-methyl-2-oxo-pyrrolidin-3-yl]methyl]-2,3-dioxo-propyl]carbamoyl]-4-chloro-phenyl]carbamate;
trideuteriomethyl N-[4-chloro-2-[[(1S)-3-(methylamino)-2,3-dioxo-1-[[(3S)-2-oxopyrrolidin-3-yl]methyl]propyl]carbamoyl]phenyl]carbamate;
methyl N-[4,5-difluoro-2-[[(1S)-3-(methylamino)-2,3-dioxo-1-[[(3S)-2-oxopyrrolidin-3-yl]methyl]propyl]carbamoyl]phenyl]carbamate;
5-chloro-2-(cyclopropanecarbonylamino)-N-[(1S)-3-(methylamino)-2,3-dioxo-1-[[(3S)-2-oxopyrrolidin-3-yl]methyl]propyl]benzamide;
ethyl N-[4-chloro-2-[[(1S)-3-(methylamino)-2,3-dioxo-1-[[(3S)-2-oxopyrrolidin-3-yl]methyl]propyl]carbamoyl]phenyl]carbamate;
trideuteriomethyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
methyl N-[6-chloro-2-[[(1S)-3-(methylamino)-1-[[(3S,5R)-5-methyl-2-oxo-pyrrolidin-3-yl]methyl]-2,3-dioxo-propyl]carbamoyl]-3-pyridyl]carbamate;
5-chloro-N-[(1S)-1-[[(3S,5R)-5-methyl-2-oxo-pyrrolidin-3-yl]methyl]-2,3-dioxo-3-(4-pyridylmethylamino)propyl]-2-(4,4,4-trifluorobutanoylamino)benzamide;
5-chloro-N-[(1S)-1-[[(3S,5R)-5-methyl-2-oxo-pyrrolidin-3-yl]methyl]-2,3-dioxo-3-(1H-1,2,4-triazol-3-ylmethylamino)propyl]-2-(4,4,4-trifluorobutanoylamino)benzamide;
5-chloro-N-[(1S)-3-(2-hydroxyethylamino)-1-[[(3S,5R)-5-methyl-2-oxo-pyrrolidin-3-yl]methyl]-2,3-dioxo-propyl]-2-(4,4,4-trifluorobutanoylamino)benzamide;
methyl N-[6-chloro-4-[[(1S)-3-(methylamino)-1-[[(3S,5R)-5-methyl-2-oxo-pyrrolidin-3-yl]methyl]-2,3-dioxo-propyl]carbamoyl]-3-pyridyl]carbamate;
methyl N-[5-chloro-3-[[(1S)-3-(methylamino)-1-[[(3S,5R)-5-methyl-2-oxo-pyrrolidin-3-yl]methyl]-2,3-dioxo-propyl]carbamoyl]-2-pyridyl]carbamate;
methyl N-[4,5-difluoro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
cyclopropyl N-[4-fluoro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
5-chloro-2-(cyclopropanecarbonylamino)-N-[(1S)-3-(methylamino)-1-[[(3S,5R)-5-methyl-2-oxo-pyrrolidin-3-yl]methyl]-2,3-dioxo-propyl]benzamide;
methyl N-[4-chloro-2-[[(1S)-3-(cyclopropylamino)-2,3-dioxo-1-[[(3S)-2-oxopyrrolidin-3-yl]methyl]propyl]carbamoyl]phenyl]carbamate;
trideuteriomethyl N-[4-chloro-2-[[(1S)-3-(cyclopropylamino)-2,3-dioxo-1-[[(3S)-2-oxopyrrolidin-3-yl]methyl]propyl]carbamoyl]phenyl]carbamate;
5-chloro-N-[(1S)-3-(methylamino)-1-[[(3S,5R)-5-methyl-2-oxo-pyrrolidin-3-yl]methyl]-2,3-dioxo-propyl]-2-(2-methylpropanoylamino)benzamide;
5-chloro-2-(2,2-dimethylpropanoylamino)-N-[(1S)-3-(methylamino)-2,3-dioxo-1-[[(3S)-2-oxopyrrolidin-3-yl]methyl]propyl]benzamide;
cyclopropyl N-[4,5-difluoro-2-[[(1S)-3-(methylamino)-2,3-dioxo-1-[[(3S)-2-oxopyrrolidin-3-yl]methyl]propyl]carbamoyl]phenyl]carbamate;
isopropyl N-[4-chloro-2-[[(1S)-3-(methylamino)-2,3-dioxo-1-[[(3S)-2-oxopyrrolidin-3-yl]methyl]propyl]carbamoyl]phenyl]carbamate;
ethyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
methyl N-[4-(difluoromethyl)-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
cyclopropyl N-[4-cyano-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
methyl N-[4-chloro-5-fluoro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
methyl N-[4-chloro-2-fluoro-6-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
methyl N-[5-chloro-4-fluoro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]oxetane-3-carboxamide;
cyclopropylmethyl N-[4-chloro-2-[[(1S)-3-(methylamino)-2,3-dioxo-1-[[(3S)-2-oxopyrrolidin-3-yl]methyl]propyl]carbamoyl]phenyl]carbamate;
cyclobutyl N-[4-chloro-2-[[(1S)-3-(methylamino)-2,3-dioxo-1-[[(3S)-2-oxopyrrolidin-3-yl]methyl]propyl]carbamoyl]phenyl]carbamate;
(1-methylcyclopropyl)N-[4-chloro-2-[[(1S)-3-(methylamino)-2,3-dioxo-1-[[(3S)-2-oxopyrrolidin-3-yl]methyl]propyl]carbamoyl]phenyl]carbamate;
ethyl N-[4-chloro-2-[[(1S)-3-(cyclopropylamino)-2,3-dioxo-1-[[(3S)-2-oxopyrrolidin-3-yl]methyl]propyl]carbamoyl]phenyl]carbamate;
cyclopropyl N-[5-chloro-3-[[(1S)-3-(methylamino)-1-[[(3S,5R)-5-methyl-2-oxo-pyrrolidin-3-yl]methyl]-2,3-dioxo-propyl]carbamoyl]-2-pyridyl]carbamate;
cyclopropyl N-[4,5-difluoro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
tert-butyl N-[4-chloro-2-[[(1S)-3-(methylamino)-2,3-dioxo-1-[[(3S)-2-oxopyrrolidin-3-yl]methyl]propyl]carbamoyl]phenyl]carbamate;
5-chloro-2-[[(1R)-2,2-difluorocyclopropanecarbonyl]amino]-N-[(1S)-3-(methylamino)-2,3-dioxo-1-[[(3S)-2-oxopyrrolidin-3-yl]methyl]propyl]benzamide;
methyl N-[4,5-dichloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
5-chloro-2-(cyclopropanecarbonylamino)-N-[(1S)-3-(cyclopropylamino)-1-[[(3S,5R)-5-methyl-2-oxo-pyrrolidin-3-yl]methyl]-2,3-dioxo-propyl]benzamide;
cyclopropyl N-[4-chloro-2-[[(1S)-3-(cyclopropylamino)-2,3-dioxo-1-[[(3S)-2-oxopyrrolidin-3-yl]methyl]propyl]carbamoyl]phenyl]carbamate;
5-chloro-N-[(1S)-3-(cyclopropylamino)-1-[[(3S,5R)-5-methyl-2-oxo-pyrrolidin-3-yl]methyl]-2,3-dioxo-propyl]-2-(2-methylpropanoylamino)benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-(dimethylcarbamoylamino)benzamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-1-methyl-azetidine-3-carboxamide;
cyclopropyl N-[2-[[(1S)-3-(cyclopropylamino)-2,3-dioxo-1-[[(3S)-2-oxopyrrolidin-3-yl]methyl]propyl]carbamoyl]-4,5-difluoro-phenyl]carbamate;
cyclobutyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
cyclopropylmethyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
tert-butyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
cyclopropyl N-[4-chloro-5-fluoro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
cyclopropyl N-[5-chloro-4-fluoro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
(2,2-difluorocyclopropyl)N-[4-chloro-2-[[(1S)-3-(methylamino)-2,3-dioxo-1-[[(3S)-2-oxopyrrolidin-3-yl]methyl]propyl]carbamoyl]phenyl]carbamate;
(2,2-difluorocyclopropyl)N-[4-chloro-2-[[(1S)-3-(methylamino)-2,3-dioxo-1-[[(3S)-2-oxopyrrolidin-3-yl]methyl]propyl]carbamoyl]phenyl]carbamate;
N-[(1S)-3-amino-1-[[(3S,5R)-5-methyl-2-oxo-pyrrolidin-3-yl]methyl]-2,3-dioxo-propyl]-5-chloro-2-(4,4,4-trifluorobutanoylamino)benzamide;
5-chloro-N-[((1S)-3-(methylamino)-2,3-dioxo-1-[[(3S)-2-oxopyrrolidin-3-yl]methyl]propyl]-2-(4,4,4-trifluorobutanoylamino)benzamide;
cyclopropyl N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]carbamate;
cyclobutyl N-[4-chloro-2-[[(1S)-3-(cyclopropylamino)-2,3-dioxo-1-[[(3S)-2-oxopyrrolidin-3-yl]methyl]propyl]carbamoyl]phenyl]carbamate;
cyclopropylmethyl N-[4-chloro-2-[[(1S)-3-(cyclopropylamino)-2,3-dioxo-1-[[(3S)-2-oxopyrrolidin-3-yl]methyl]propyl]carbamoyl]phenyl]carbamate;
2,2,2-trifluoroethyl N-[4-chloro-2-[[(1S)-3-(methylamino)-2,3-dioxo-1-[[(3S)-2-oxopyrrolidin-3-yl]methyl]propyl]carbamoyl]phenyl]carbamate;
5-chloro-N-[(1S)-3-(cyclopropylamino)-1-[[(3S,5R)-5-methyl-2-oxo-pyrrolidin-3-yl]methyl]-2,3-dioxo-propyl]-2-[[(2R)-2-methoxypropanoyl]amino]benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[(2-hydroxy-2-methyl-propanoyl)amino]benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[[(2S)-2-methoxypropanoyl]amino]benzamide;
cyclopentyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
5-chloro-N-[(1S)-3-(cyclopropylamino)-2,3-dioxo-1-[[(3S)-2-oxopyrrolidin-3-yl]methyl]propyl]-2-[[(1R)-2,2-difluorocyclopropanecarbonyl]amino]benzamide;
cyclopropyl N-[2-[[(1S)-3-(methylamino)-1-[[(3S,5R)-5-methyl-2-oxo-pyrrolidin-3-yl]methyl]-2,3-dioxo-propyl]carbamoyl]-4-(trifluoromethyl)phenyl]carbamate;
cyclopropyl N-[4,5-dichloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
(2,2-difluorocyclopropyl)N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
(2,2-difluorocyclopropyl)N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
(3,3-difluorocyclobutyl)N-[4-chloro-2-[[(1S)-3-(methylamino)-2,3-dioxo-1-[[(3S)-2-oxopyrrolidin-3-yl]methyl]propyl]carbamoyl]phenyl]carbamate;
benzyl N-[4-chloro-2-[[(1S)-3-(methylamino)-2,3-dioxo-1-[[(3S)-2-oxopyrrolidin-3-yl]methyl]propyl]carbamoyl]phenyl]carbamate;
5-chloro-N-[(1S)-3-(methylamino)-2,3-dioxo-1-[[(3S)-2-oxopyrrolidin-3-yl]methyl]propyl]-2-[[2-(trifluoromethyl)cyclopropanecarbonyl]amino]benzamide;
5-chloro-N-[(1S)-3-(methylamino)-2,3-dioxo-1-[[(3S)-2-oxopyrrolidin-3-yl]methyl]propyl]-2-[[2-(trifluoromethyl)cyclopropanecarbonyl]amino]benzamide;
5-chloro-N-[((1S)-3-(cyclopropylamino)-1-[[(3S,5R)-5-methyl-2-oxo-pyrrolidin-3-yl]methyl]-2,3-dioxo-propyl]-2-[(2,2,2-trifluoroacetyl)amino]benzamide;
5-chloro-N-[((1S)-3-(methylamino)-1-[[(3S,5R)-5-methyl-2-oxo-pyrrolidin-3-yl]methyl]-2,3-dioxo-propyl]-2-(4,4,4-trifluorobutanoylamino)benzamide;
5-chloro-N-[(1S)-3-(methylamino)-2,3-dioxo-1-[[(3S)-2-oxopyrrolidin-3-yl]methyl]propyl]-2-(2-thiazol-4-ylpropanoylamino)benzamide;
(2,2,2-trifluoro-1-methyl-ethyl)N-[4-chloro-2-[[(1S)-3-(methylamino)-2,3-dioxo-1-[[(3S)-2-oxopyrrolidin-3-yl]methyl]propyl]carbamoyl]phenyl]carbamate;
2,2,2-trifluoroethyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
(5S)—N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-3-methyl-2-oxo-oxazolidine-5-carboxamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-5-cyano-pyridine-3-carboxamide;
5-chloro-2-[(2,2-difluorocyclopentanecarbonyl)amino]-N-[(1S)-3-(methylamino)-1-[[(3S,5R)-5-methyl-2-oxo-pyrrolidin-3-yl]methyl]-2,3-dioxo-propyl]benzamide;
5-chloro-2-[(2,2-difluorocyclopentanecarbonyl)amino]-N-[(1S)-3-(methylamino)-1-[[(3S,5R)-5-methyl-2-oxo-pyrrolidin-3-yl]methyl]-2,3-dioxo-propyl]benzamide;
(2,2-difluorocyclopropyl)methyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
(2,2-difluorocyclopropyl)methyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
(3,3-difluorocyclobutyl)N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
5-chloro-N-[(1S)-3-(methylamino)-1-[[(3S,5R)-5-methyl-2-oxo-pyrrolidin-3-yl]methyl]-2,3-dioxo-propyl]-2-[[2-(trifluoromethyl)cyclopropanecarbonyl]amino]benzamide;
5-chloro-N-[(1S)-3-(methylamino)-1-[[(3S,5R)-5-methyl-2-oxo-pyrrolidin-3-yl]methyl]-2,3-dioxo-propyl]-2-[[2-(trifluoromethyl)cyclopropanecarbonyl]amino]benzamide;
2,2,2-trifluoroethyl N-[4-chloro-2-[[(1S)-3-(cyclopropylamino)-2,3-dioxo-1-[[(3S)-2-oxopyrrolidin-3-yl]methyl]propyl]carbamoyl]phenyl]carbamate;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]tetrahydropyran-4-carboxamide;
(5S)—N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-2-oxo-oxazolidine-5-carboxamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]morpholine-4-carboxamide;
5-chloro-N-[(1S)-3-(methylamino)-1-[[(3S,5R)-5-methyl-2-oxo-pyrrolidin-3-yl]methyl]-2,3-dioxo-propyl]-2-(2-thiazol-4-ylpropanoylamino)benzamide;
N-[4-fluoro-2-[[(1S)-3-(methylamino)-2,3-dioxo-1-[[(3S)-2-oxopyrrolidin-3-yl]methyl]propyl]carbamoyl]phenyl]-2-(trifluoromethyl)pyridine-4-carboxamide;
5-chloro-2-[(4,4-difluorocyclohexanecarbonyl)amino]-N-[(1S)-3-(methylamino)-1-[[(3S,5R)-5-methyl-2-oxo-pyrrolidin-3-yl]methyl]-2,3-dioxo-propyl]benzamide;
5-chloro-2-[(3,3-difluorocyclohexanecarbonyl)amino]-N-[(1S)-3-(methylamino)-1-[[(3S,5R)-5-methyl-2-oxo-pyrrolidin-3-yl]methyl]-2,3-dioxo-propyl]benzamide;
5-chloro-2-[(3,3-difluorocyclohexanecarbonyl)amino]-N-[(1S)-3-(methylamino)-1-[[(3S,5R)-5-methyl-2-oxo-pyrrolidin-3-yl]methyl]-2,3-dioxo-propyl]benzamide;
(3,3-difluorocyclopentyl)N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
(3,3-difluorocyclopentyl)N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
5-chloro-N-[(1S)-3-(methylamino)-1-[[(3S,5R)-5-methyl-2-oxo-pyrrolidin-3-yl]methyl]-2,3-dioxo-propyl]-2-[[1-(2,2,2-trifluoroethyl)cyclopropanecarbonyl]amino]benzamide;
5-(difluoromethyl)-N-[(1S)-3-(methylamino)-1-[[(3S,5R)-5-methyl-2-oxo-pyrrolidin-3-yl]methyl]-2,3-dioxo-propyl]-2-[[1-(trifluoromethyl)cyclopropanecarbonyl]amino]benzamide;
(5S)—N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-3-methyl-2-oxo-oxazolidine-5-carboxamide;
(5R)—N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-3-methyl-2-oxo-oxazolidine-5-carboxamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]-6-fluoro-phenyl]-3-fluoro-bicyclo[1.1.1]pentane-1-carboxamide;
N-[4-fluoro-2-[[(1S)-3-(methylamino)-1-[[(3S,5R)-5-methyl-2-oxo-pyrrolidin-3-yl]methyl]-2,3-dioxo-propyl]carbamoyl]phenyl]-2-(trifluoromethyl)pyridine-4-carboxamide;
N-[2-[[(1S)-3-amino-1-[[(3S,5R)-5-methyl-2-oxo-pyrrolidin-3-yl]methyl]-2,3-dioxo-propyl]carbamoyl]-4-chloro-phenyl]-2-(trifluoromethyl)pyridine-4-carboxamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[[(2R)-4,4,4-trifluoro-2-methyl-butanoyl]amino]benzamide;
(2,2,2-trifluoro-1-methyl-ethyl)N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]carbamate;
(2,2,2-trifluoro-1-methyl-ethyl)N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]carbamate;
(4S)—N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-1,3-dimethyl-2-oxo-imidazolidine-4-carboxamide;
5-chloro-N-[(1S)-3-(cyclopropylamino)-1-[[(3S,5R)-5-methyl-2-oxo-pyrrolidin-3-yl]methyl]-2,3-dioxo-propyl]-3-fluoro-2-(4,4,4-trifluorobutanoylamino)benzamide;
5-chloro-4-fluoro-N-[(1S)-3-(methylamino)-1-[[(3S,5R)-5-methyl-2-oxo-pyrrolidin-3-yl]methyl]-2,3-dioxo-propyl]-2-[[1-(2,2,2-trifluoroethyl)cyclopropanecarbonyl]amino]benzamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-2-(trifluoromethyl)pyrimidine-5-carboxamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-4-(trifluoromethyl)pyrimidine-2-carboxamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-5-(trifluoromethyl)pyrimidine-2-carboxamide;
(3,3-difluorocyclohexyl)methyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
(3,3-difluorocyclohexyl)methyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
N-[(1S)-3-(cyclopropylamino)-1-[[(3S,5R)-5-methyl-2-oxo-pyrrolidin-3-yl]methyl]-2,3-dioxo-propyl]-5-(difluoromethyl)-2-[[1-(trifluoromethyl)cyclopropanecarbonyl]amino]benzamide;
methyl N-[(1S)-1-[[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamoyl]-2,2-dimethyl-propyl]-N-methyl-carbamate;
N-[4-(difluoromethyl)-2-[[(1S)-3-(methylamino)-1-[[(3S,5R)-5-methyl-2-oxo-pyrrolidin-3-yl]methyl]-2,3-dioxo-propyl]carbamoyl]phenyl]-2-(trifluoromethyl)pyridine-4-carboxamide;
N-[4-chloro-5-fluoro-2-[[(1S)-3-(methylamino)-1-[[(3S,5R)-5-methyl-2-oxo-pyrrolidin-3-yl]methyl]-2,3-dioxo-propyl]carbamoyl]phenyl]-6-(trifluoromethyl)pyridine-3-carboxamide;
N-[4-chloro-2-fluoro-6-[[(1S)-3-(methylamino)-1-[[(3S,5R)-5-methyl-2-oxo-pyrrolidin-3-yl]methyl]-2,3-dioxo-propyl]carbamoyl]phenyl]-2-(trifluoromethyl)pyridine-4-carboxamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-6-(trifluoromethyl)pyridine-3-carboxamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-5-(trifluoromethyl)pyrazine-2-carboxamide;
5-chloro-N-[(1S)-3-(methylamino)-1-[[(3S,5R)-5-methyl-2-oxo-pyrrolidin-3-yl]methyl]-2,3-dioxo-propyl]-2-[2-[2-(trifluoromethyl)-4-pyridyl]propanoylamino]benzamide;
5-chloro-N-[(1S)-3-(methylamino)-1-[[(3S,5R)-5-methyl-2-oxo-pyrrolidin-3-yl]methyl]-2,3-dioxo-propyl]-2-[2-[2-(trifluoromethyl)-4-pyridyl]propanoylamino]benzamide;
5-chloro-N-[(1S)-3-(methylamino)-1-[[(3S,5R)-5-methyl-2-oxo-pyrrolidin-3-yl]methyl]-2,3-dioxo-propyl]-2-[2-[6-(trifluoromethyl)-3-pyridyl]propanoylamino]benzamide;
5-chloro-N-[(1S)-3-(methylamino)-1-[[(3S,5R)-5-methyl-2-oxo-pyrrolidin-3-yl]methyl]-2,3-dioxo-propyl]-2-[2-[6-(trifluoromethyl)-3-pyridyl]propanoylamino]benzamide;
N-[2,4-dichloro-6-[[(1S)-3-(methylamino)-1-[[(3S,5R)-5-methyl-2-oxo-pyrrolidin-3-yl]methyl]-2,3-dioxo-propyl]carbamoyl]phenyl]-2-(trifluoromethyl)pyridine-4-carboxamide;
N-[2-[[(1S)-3-(cyclopropylamino)-1-[[(3S,5R)-5-methyl-2-oxo-pyrrolidin-3-yl]methyl]-2,3-dioxo-propyl]carbamoyl]-4-(difluoromethyl)phenyl]-2-(trifluoromethyl)pyridine-4-carboxamide;
N-[2-[[(1S)-3-(cyclopropylamino)-1-[[(3S,5R)-5-methyl-2-oxo-pyrrolidin-3-yl]methyl]-2,3-dioxo-propyl]carbamoyl]-3,4,5-trifluoro-phenyl]-2-(trifluoromethyl)pyridine-4-carboxamide;
N-[4-chloro-2-[[(1S)-3-(cyclopropylamino)-2,3-dioxo-1-[[(3S)-2-oxo-3-piperidyl]methyl]propyl]carbamoyl]phenyl]-2-(trifluoromethyl)pyridine-4-carboxamide; and
5-chloro-N-[(1S)-3-(cyclopropylamino)-2,3-dioxo-1-[[(3S)-2-oxo-3-piperidyl]methyl]propyl]-2-(4,4,4-trifluorobutanoylamino)benzamide.

In certain embodiments of the present invention the compound of formula I is selected form the group consisting of:

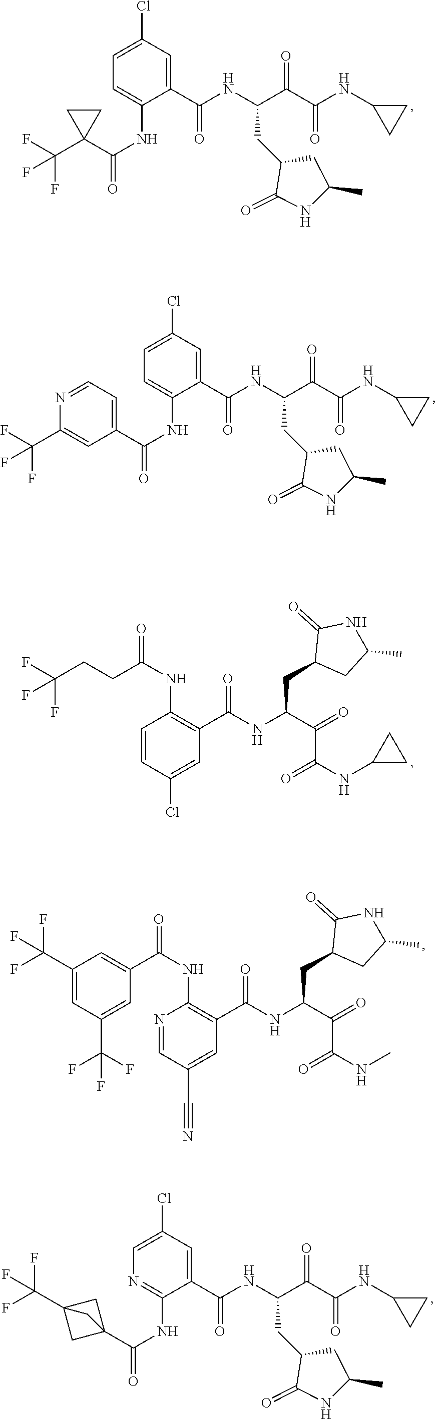 [SMILES: C[C@@H]1C[C@@H](C[C@H](NC(=O)c2cc(Cl)ccc2NC(=O)C2(C(F)(F)F)CC2)C(=O)C(=O)NC2CC2)C(=O)N1 ; C[C@@H]1C[C@@H](C[C@H](NC(=O)c2cc(Cl)ccc2NC(=O)c2ccnc(C(F)(F)F)c2)C(=O)C(=O)NC2CC2)C(=O)N1 ; C[C@@H]1C[C@@H](C[C@H](NC(=O)c2cc(Cl)ccc2NC(=O)CCC(F)(F)F)C(=O)C(=O)NC2CC2)C(=O)N1 ; CNC(=O)C(=O)[C@H](C[C@@H]1C[C@@H](C)NC1=O)NC(=O)c1cc(C#N)cnc1NC(=O)c1cc(C(F)(F)F)cc(C(F)(F)F)c1 ; C[C@@H]1C[C@@H](C[C@H](NC(=O)c2cc(Cl)cnc2NC(=O)C23CC(C(F)(F)F)(C2)C3)C(=O)C(=O)NC2CC2)C(=O)N1]

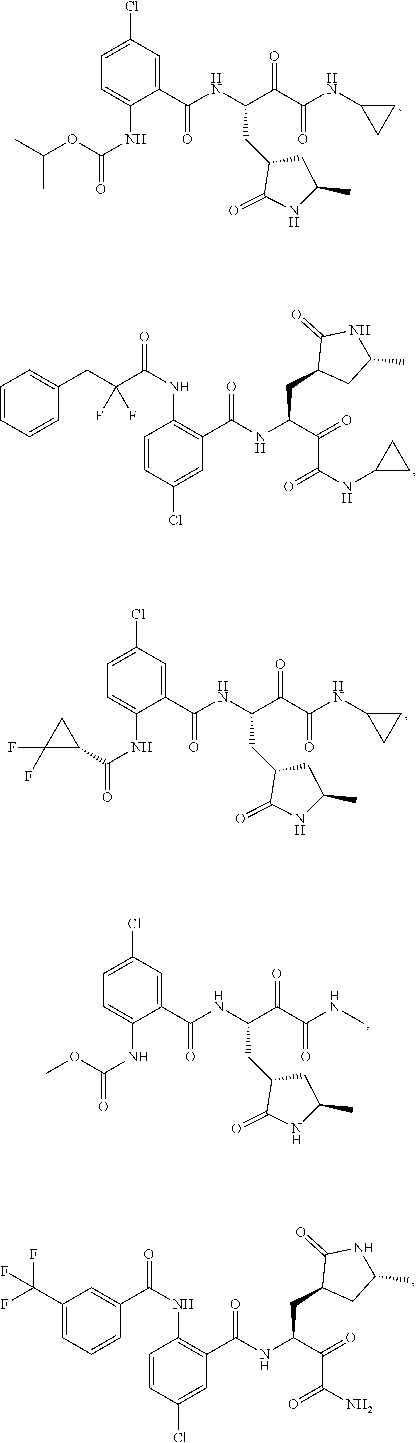

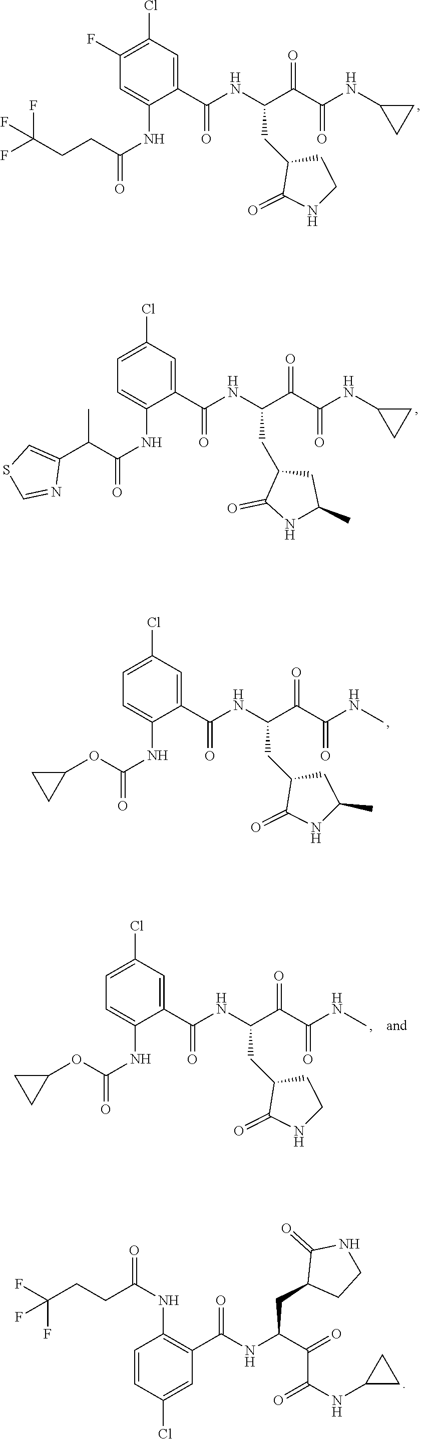 [SMILES: O=C(CCC(F)(F)F)Nc1cc(F)c(Cl)cc1C(=O)N[C@@H](C[C@@H]1CCNC1=O)C(=O)C(=O)NC1CC1 ; CC(C(=O)Nc1ccc(Cl)cc1C(=O)N[C@@H](C[C@@H]1C[C@@H](C)NC1=O)C(=O)C(=O)NC1CC1)c1cscn1 ; CNC(=O)C(=O)[C@H](C[C@@H]1C[C@@H](C)NC1=O)NC(=O)c1cc(Cl)ccc1NC(=O)OC1CC1 ; CNC(=O)C(=O)[C@H](C[C@@H]1CCNC1=O)NC(=O)c1cc(Cl)ccc1NC(=O)OC1CC1 ; O=C(CCC(F)(F)F)Nc1ccc(Cl)cc1C(=O)N[C@@H](C[C@@H]1CCNC1=O)C(=O)C(=O)NC1CC1]

Also included within the scope of the present invention is a pharmaceutical composition which is comprised of a compound of Formula I as described above or a pharmaceutically acceptable salt thereof, and a pharmaceutically acceptable carrier. The pharmaceutical composition can be, for example, in the form of an orally administered tablet or capsule. The invention is also contemplated to encompass a pharmaceutical composition which is comprised of a pharmaceutically acceptable carrier and any of the compounds specifically disclosed in the present application, including pharmaceutically acceptable salts thereof. These and other aspects of the invention will be apparent from the teachings contained herein.

The invention also includes compositions for inhibiting protease in a coronavirus, treating a disease caused by a coronavirus, treating coronavirus infection and preventing coronavirus infection, in a mammal, comprising a compound of the invention in a pharmaceutically acceptable carrier. These compositions may optionally include other antiviral agents. The compositions can be added to blood, blood products, or mammalian organs in order to effect the desired inhibitions.

The invention further includes methods for prophylaxis or treatment of a coronavirus infection by administering compounds of formula I. Such coronavirus infections include a SARS-CoV, SARS-CoV-2 or MERS-CoV infection

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, ascorbate, adipate, alginate, aspirate, benzenesulfonate, benzoate, bicarbonate, bisulfate, bitartrate, borate, bromide, butyrate, camphorate, camphorsulfonate, camsylate, carbonate, chloride, clavulanate, citrate, cyclopentane propionate, diethylacetic, digluconate, dihydrochloride, dodecylsulfanate, edetate, edisylate, estolate, esylate, ethanesulfonate, formic, fumarate, gluceptate, glucoheptanoate, gluconate, glutamate, glycerophosphate, glycollylarsanilate, hemisulfate, heptanoate, hexanoate, hexylresorcinate, hydrabamine, hydrobromide, hydrochloride, 2-hydroxyethanesulfonate, hydroxynaphthoate, iodide, isonicotinic, isothionate, lactate, lactobionate, laurate, malate, maleate, mandelate, mesylate, methylbromide, methylnitrate, methylsulfate, methanesulfonate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, phosphate/diphosphate, pimelic, phenylpropionic, polygalacturonate, propionate, salicylate, stearate, sulfate, subacetate, succinate, tannate, tartrate, teoclate, thiocyanate, tosylate, triethiodide, trifluoroacetate, undeconate, valerate and the like. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Also included are the ammonium, calcium, magnesium, potassium, and sodium salts. Salts derived from pharmaceutically acceptable organic non-toxic bases include salts of primary, secondary, and tertiary amines, cyclic amines, dicyclohexyl amines and basic ion-exchange resins, such as arginine, betaine, caffeine, choline, N,N-dibenzylethylenediamine, diethylamine, 2-diethylaminoethanol, 2-dimethylaminoethanol, ethanolamine, ethylamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. Also, included are the basic nitrogen-containing groups that may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

These salts can be obtained by known methods, for example, by mixing a compound of the present invention with an equivalent amount and a solution containing a desired acid, base, or the like, and then collecting the desired salt by filtering the salt or distilling off the solvent. The compounds of the present invention and salts thereof may form solvates with a solvent such as water, ethanol, or glycerol. The compounds of the present invention may form an acid addition salt and a salt with a base at the same time according to the type of substituent of the side chain.

If the compounds of Formula I simultaneously contain acidic and basic groups in the molecule the invention also includes, in addition to the salt forms mentioned, inner salts or betaines (zwitterions).

The present invention encompasses all stereoisomeric forms of the compounds of Formula I. Unless a specific stereochemistry is indicated, the present invention is meant to comprehend all such isomeric forms of these compounds. Centers of asymmetry that are present in the compounds of Formula I can all independently of one another have (R) configuration or (S) configuration. When bonds to the chiral carbon are depicted as straight lines in the structural Formulas of the invention, it is understood that both the (R) and (S) configurations of the chiral carbon, and hence both each individual enantiomer and mixtures thereof, are embraced within the Formula. When a particular configuration is depicted, that enantiomer (either (R) or (S), at that center) is intended. Similarly, when a compound name is recited without a chiral designation for a chiral carbon, it is understood that both the (R) and (S) configurations of the chiral carbon, and hence individual enantiomers and mixtures thereof, are embraced by the name. The production of specific stereoisomers or mixtures thereof may be identified in the Examples where such stereoisomers or mixtures were obtained, but this in no way limits the inclusion of all stereoisomers and mixtures thereof from being within the scope of this invention.

Unless a specific enantiomer or diastereomer is indicated, the invention includes all possible enantiomers and diastereomers and mixtures of two or more stereoisomers, for example mixtures of enantiomers and/or diastereomers, in all ratios. Thus, enantiomers are a subject of the invention in enantiomerically pure form, both as levorotatory and as dextrorotatory antipodes, in the form of racemates and in the form of mixtures of the two enantiomers in all ratios. In the case of a cis/trans isomerism the invention includes both the cis form and the trans form as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound of Formula I or it can be done on a final racemic product. Absolute stereochemistry may be determined by X-ray crystallography of crystalline products or crystalline intermediates which are derivatized, if necessary, with a reagent containing a stereogenic center of known configuration. Where compounds of this invention are capable of tautomerization, all individual tautomers as well as mixtures thereof are included in the scope of this invention. The present invention includes all such isomers, as well as salts, solvates (including hydrates) and solvated salts of such racemates, enantiomers, diastereomers and tautomers and mixtures thereof.

In the compounds of the invention, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the specifically and generically described compounds. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds can be prepared without undue experimentation by conventional techniques well known to those skilled in the art or by processes analogous to those described in the general process schemes and examples herein using appropriate isotopically-enriched reagents and/or intermediates.

When any variable occurs more than one time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is bicyclic, it is intended that the bond be attached to any of the suitable atoms on either ring of the bicyclic moiety.

It is understood that one or more silicon (Si) atoms can be incorporated into the compounds of the instant invention in place of one or more carbon atoms by one of ordinary skill in the art to provide compounds that are chemically stable and that can be readily synthesized by techniques known in the art from readily available starting materials. Carbon and silicon differ in their covalent radius leading to differences in bond distance and the steric arrangement when comparing analogous C-element and Si-element bonds. These differences lead to subtle changes in the size and shape of silicon-containing compounds when compared to carbon. One of ordinary skill in the art would understand that size and shape differences can lead to subtle or dramatic changes in potency, solubility, lack of off-target activity, packaging properties, and so on. (Diass, J. O. et al. Organometallics (2006) 5:1188-1198; Showell, G. A. et al. Bioorganic & Medicinal Chemistry Letters (2006) 16:2555-2558).

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted” (with one or more substituents) should be understood as meaning that the group in question is either unsubstituted or may be substituted with one or more substituents.

Furthermore, compounds of the present invention may exist in amorphous form and/or one or more crystalline forms, and as such all amorphous and crystalline forms and mixtures thereof of the compounds of Formula I are intended to be included within the scope of the present invention. In addition, some of the compounds of the instant invention may form solvates with water (i.e., a hydrate) or common organic solvents. Such solvates and hydrates, particularly the pharmaceutically acceptable solvates and hydrates, of the instant compounds are likewise encompassed within the scope of this invention, along with un-solvated and anhydrous forms.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as O-acetyl, O-pivaloyl, O-benzoyl, and O-aminoacyl, can be employed. Included are those esters and acyl groups known in the art for modifying the solubility or hydrolysis characteristics for use as sustained-release or prodrug formulations.

Any pharmaceutically acceptable pro-drug modification of a compound of this invention which results in conversion in vivo to a compound within the scope of this invention is also within the scope of this invention. For example, esters can optionally be made by esterification of an available carboxylic acid group or by formation of an ester on an available hydroxy group in a compound. Similarly, labile amides can be made. Pharmaceutically acceptable esters or amides of the compounds of this invention may be prepared to act as pro-drugs which can be hydrolyzed back to an acid (or —COO— depending on the pH of the fluid or tissue where conversion takes place) or hydroxy form particularly in vivo and as such are encompassed within the scope of this invention. Examples of pharmaceutically acceptable pro-drug modifications include, but are not limited to, —C₁-C₆ alkyl esters and —C₁-C₆ substituted with phenyl esters.

Accordingly, the compounds within the generic structural formulas, embodiments and specific compounds described and claimed herein encompass salts, all possible stereoisomers and tautomers, physical forms (e.g., amorphous and crystalline forms), solvate and hydrate forms thereof and any combination of these forms, as well as the salts thereof, pro-drug forms thereof, and salts of pro-drug forms thereof, where such forms are possible unless specified otherwise.

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “-O-alkyl,” etc.

As used herein, and throughout this disclosure, the following terms, unless otherwise indicated, shall be understood to have the following meanings:

A “subject” is a human or non-human mammal. In one embodiment, a subject is a human. In another embodiment, a subject is a primate. In another embodiment, a subject is a monkey. In another embodiment, a subject is a chimpanzee. In still another embodiment, a subject is a rhesus monkey.

As used herein, the terms “treatment” and “treating” refer to all processes in which there may be a slowing, interrupting, arresting, controlling, or stopping of the progression of a disease or disorder described herein. The terms do not necessarily indicate a total elimination of all disease or disorder symptoms.

The terms “preventing,” or “prophylaxis,” as used herein, refers to reducing the likelihood of contracting disease or disorder described herein, or reducing the severity of a disease or disorder described herein.

The term “alkyl,” as used herein, refers to an aliphatic hydrocarbon group having one of its hydrogen atoms replaced with a bond. An alkyl group may be straight or branched and contain from about 1 to about 20 carbon atoms. In one embodiment, an alkyl group contains from about 1 to about 12 carbon atoms. In different embodiments, an alkyl group contains from 1 to 6 carbon atoms (C₁-C₆ alkyl) or from about 1 to about 4 carbon atoms (C₁-C₄ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. In one embodiment, an alkyl group is linear. In another embodiment, an alkyl group is branched. Unless otherwise indicated, an alkyl group is unsubstituted.

The term “fluoroalkyl,” as used herein refers to an alkyl group as defined above, wherein one or more of the alkyl group's hydrogen atoms has been replaced with a fluorine. In one embodiment, a fluoroalkyl group has from 1 to 6 carbon atoms. In another embodiment, a haloalkyl group is substituted with from 1 to 3 F atoms. Non-limiting examples of fluoroalkyl groups include —CH₂F, —CHF₂, —CF₃, and —CH₂CF₃. The term “C₁-C₆ fluoroalkyl” refers to a fluoroalkyl group having from 1 to 6 carbon atoms.

The term “halo,” as used herein, means —F, —Cl, —Br or —I.

The term “cycloalkyl” means a monocyclic or bicyclic saturated aliphatic hydrocarbon group having the specified number of carbon atoms. For example, “cycloalkyl” includes cyclopropyl, cyclobutyl, cyclopentyl, cyclohexyl, and so on. Bicyclic cycloalkyl ring systems include fused ring systems, where two rings share two atoms, spiro ring systems, where two rings share one atom, and bridged systems.

The term “aryl”, as used herein, represents a stable bicyclic or tricyclic ring system of up to 10 atoms in each ring, wherein at least one ring is aromatic, and all of the ring atoms are carbon. Bicyclic and tricyclic ring systems include fused ring systems, where two rings share two atoms, and spiro ring systems, where two rings share one atom.

The term “heteroaryl”, as used herein, represents a stable monocyclic or bicyclic ring system of up to 10 atoms in each ring, wherein at least one ring is aromatic, and at least one ring contains from 1 to 4 heteroatoms selected from the group consisting of O, N and S. Bicyclic heteroaryl ring systems include fused ring systems, where two rings share two atoms, and spiro ring systems, where two rings share one atom. Heteroaryl groups within the scope of this definition include but are not limited to: azaindolyl, benzoimidazolyl, benzisoxazolyl, benzofuranyl, benzofurazanyl, benzopyrazolyl, benzotriazolyl, benzothiophenyl, benzoxazolyl, carbazolyl, carbolinyl, cinnolinyl, dihydroindenyl, furanyl, indolinyl, indolyl, indolazinyl, indazolyl, isobenzofuranyl, isoindolyl, isoquinolyl, isothiazolyl, isoxazolyl, naphthalenyl, naphthpyridinyl, oxadiazolyl, oxazolyl, oxazoline, isoxazoline, pyranyl, pyrazinyl, pyrazolyl, pyrazolopyrimidinyl, pyridazinyl, pyridopyridinyl, pyridyl, pyrimidinyl, pyrrolyl, quinazolinyl, quinolyl, quinoxalinyl, tetrazolyl, tetrazolopyridyl, thiadiazolyl, thiazolyl, thienyl, triazolyl, dihydrobenzoimidazolyl, dihydrobenzofuranyl, dihydrobenzothiophenyl, dihydrobenzoxazolyl, dihydroindolyl, dihydroquinolinyl, dihydrobenzodioxinyl, dihydropyrazoloxazinyl, dihydropyrazolyothiazinedioxidyl, methylenedioxybenzene, benzothiazolyl, benzothienyl, quinolinyl, isoquinolinyl, oxazolyl, tetra-hydroquinoline and 3-oxo-3,4dihydro-2N-benzo[b][1,4]thiazine. If the heteroaryl contains nitrogen atoms, it is understood that the corresponding N-oxides thereof are also encompassed by this definition.

The term “heterocycloalkyl”, “heterocycle,” or “heterocyclyl” as used herein is intended to mean a stable nonaromatic monocyclic or bicyclic ring system of up to 10 atoms in each ring, unless otherwise specified, containing from 1 to 4 heteroatoms selected from the group consisting of O, N, S, SO, or SO₂. In some embodiments, heterocycloalkyl are saturated. Bicyclic heterocyclic ring systems include fused ring systems, where two rings share two atoms, and spiro ring systems, where two rings share one atom. “Heterocycloalkyl” therefore includes, but is not limited to the following: azaspirononanyl, azaspirooctanyl, azetidinyl, dioxanyl, oxadiazaspirodecenyl, oxaspirooctanyl, oxazolidinonyl, piperazinyl, piperidinyl, pyrrolidinyl, morpholinyl, thiomorpholinyl, tetrahydrofuranyl, tetrahydropyranyl, dihydropiperidinyl, tetrahydrothiophenyl and the like. If the heterocycle contains a nitrogen, it is understood that the corresponding N-oxides thereof are also encompassed by this definition.

“Celite®” (Fluka) diatomite is diatomaceous earth and can be referred to as “celite”.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al., Protective Groups in Organic Synthesis (1991), Wiley, New York.

When any substituent or variable (e.g., R₂) occurs more than one time in any constituent or in Formula I, its definition on each occurrence is independent of its definition at every other occurrence, unless otherwise indicated.

As used herein, the term “composition” is intended to encompass a product comprising the specified ingredients in the specified amounts, as well as any product which results from combination of the specified ingredients in the specified amounts.

The invention also relates to medicaments containing at least one compound of the Formula I and/or of a pharmaceutically acceptable salt of the compound of the Formula I and/or an optionally stereoisomeric form of the compound of the Formula I or a pharmaceutically acceptable salt of the stereoisomeric form of the compound of Formula I, together with a pharmaceutically suitable and pharmaceutically acceptable vehicle, additive and/or other active substances and auxiliaries.

The term “patient” used herein is taken to mean mammals such as primates, humans, sheep, horses, cattle, pigs, dogs, cats, rats, and mice.

The term “coronavirus” includes HCoV-229E, HCoV-NL63, HCoV-OC43, HCoV-HKU1, severe acute respiratory syndrome coronavirus (SARS-CoV), Middle East respiratory syndrome coronavirus (MERS-CoV) and SARS-CoV-2.

The medicaments according to the invention can be administered by oral, inhalative, rectal or transdermal administration or by subcutaneous, intraarticular, intraperitoneal or intravenous injection. Oral administration is preferred. Coating of stents with compounds of the Formula (I) and other surfaces which come into contact with blood in the body is possible.

The invention also relates to a process for the production of a medicament, which comprises bringing at least one compound of the Formula (I) into a suitable administration form using a pharmaceutically suitable and pharmaceutically acceptable carrier and optionally further suitable active substances, additives or auxiliaries.

Suitable solid or galenical preparation forms are, for example, granules, powders, coated tablets, tablets, (micro)capsules, suppositories, syrups, juices, suspensions, emulsions, drops or injectable solutions and preparations having prolonged release of active substance, in whose preparation customary excipients such as vehicles, disintegrants, binders, coating agents, swelling agents, glidants or lubricants, flavorings, sweeteners and solubilizers are used. Frequently used auxiliaries which may be mentioned are magnesium carbonate, titanium dioxide, lactose, mannitol and other sugars, talc, lactose, gelatin, starch, cellulose and its derivatives, animal and plant oils such as cod liver oil, sunflower, peanut or sesame oil, polyethylene glycol and solvents such as, for example, sterile water and mono- or polyhydric alcohols such as glycerol.

The dosage regimen utilizing the protease inhibitors of the instant invention is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Oral dosages of the protease inhibitors, when used for the indicated effects, will range between about 0.01 mg per kg of body weight per day (mg/kg/day) to about 30 mg/kg/day, for instance, 0.01-20 mg/kg/day, 0.01-15 mg/kg/day, 0.01-10 mg/kg/day or 0.01-5 mg/kg/day (unless specified otherwise, amounts of active ingredients are on free base basis). For example, an 80 kg patient would receive between about 0.8 mg/day and 2.4 g/day, e.g., 0.8-1600 mg/day, 0.8-1200 mg/day, 0.8-800 mg/kg/day, or 0.8-400 mg/day. A suitably prepared medicament for once a day administration would thus contain between 0.8 mg and 2.4 g, between 0.8 mg and 1600 mg, between 0.8 mg and 1200 mg, between 0.8 mg and 800 mg, or between 0.8 and 400 mg, e.g., 1 mg, 4 mg, 8 mg, 10 mg, 20 mg, 40 mg, 80 mg, 160 mg, 200 mg, 300 mg, or 400 mg. Advantageously, the protease inhibitors may be administered in divided doses of two, three, or four times daily. For administration twice a day, a suitably prepared medicament would contain between 0.4 mg and 1.2 g, between 0.4 mg and 800 mg, between 0.4 mg and 600 mg, between 0.4 mg and 400 mg, or between 0.4 and 200 mg, e.g., 0.5 mg, 2 mg, 4 mg, 5 mg, 10 mg, 20 mg, 40 mg, 80 mg, 100 mg, 150 mg, or 200 mg.

Intravenously, the patient would receive the active ingredient in quantities sufficient to deliver between 0.01-15 mg/kg/day, e.g., 0.01-7.5 mg/kg/day or 0.1-5 mg/kg/day. Such quantities may be administered in a number of suitable ways, e.g., large volumes of low concentrations of active ingredient during one extended period of time or several times a day, low volumes of high concentrations of active ingredient during a short period of time, e.g., once a day. Glucuronic acid, L-lactic acid, acetic acid, citric acid or any pharmaceutically acceptable acid/conjugate base with reasonable buffering capacity in the pH range acceptable for intravenous administration may be used as buffers. The choice of appropriate buffer and pH of a formulation, depending on solubility of the drug to be administered, is readily determined by a person having ordinary skill in the art.

Compounds of Formula I can be administered both as a monotherapy and in combination with additional therapeutic agents (also referred to herein as “second therapeutic agents”), including other antivirals or treatments of coronavirus infection.

The protease inhibitors of the instant invention can also be co-administered with suitable antivirals, including, but not limited to, agents that inhibit the replication of viruses such as nucleoside polymerase inhibitors, agents that induce viral error catastrophe protease inhibitors, eEF1A inhibitors, androgen receptor antagonists, dihydroorotate dehydrogenase (DHODH) inhibitors, sphingosine kinase inhibitors, MEK inhibitors, antimalarials, CCR5 inhibitors, PIKfyve kinase inhibitors, serine protease inhibitors and glycosylation inhibitors. In a class of the invention, the protease inhibitors of the instant invention can be co-administered with a nucleoside polymerase inhibitor, a protease inhibitor, or a combination thereof. Skilled practitioners will acknowledge that such antivirals in some cases may be co-administered as prodrugs.

Polymerase inhibitors that can be co-administered with the protease inhibitors of the instant invention include, but are not limited to, clevudine, remdesivir (VEKLURY), favipiravir (AVIGAN) and AT-527.

Protease inhibitors that can be co-administered with the protease inhibitors of the instant invention include, but are not limited to, camostat mesylate, upamostat, SLV213, PF-0083523, CDI-45205, ALG-097111, GC-376 and TJC-0642.

Agents that induce viral error catastrophe that can be co-administered with the protease inhibitors of the invention include molnupiravir and nirmatrelvir.

eEF1A inhibitors that can be co-administered with the protease inhibitors of the instant invention include, but are not limited to, plitidepsin.

Androgen receptor antagonists that can be co-administered with the protease inhibitors of the instant invention include, but are not limited to, proxalutamide.

Dihydroorotate dehydrogenase (DHODH) inhibitors that can be co-administered with the protease inhibitors of the instant invention include, but are not limited to, PTC299 and brequinlar.

Sphingosine kinase inhibitors that can be co-administered with the protease inhibitors of the instant invention include, but are not limited to, opaganib.

MEK inhibitors that can be co-administered with the protease inhibitors of the instant invention include, but are not limited to, ATR-002.

Antimalarials that can be co-administered with the protease inhibitors of the instant invention include, but are not limited to, tafenoquine (ARAKODA).

CCR5 inhibitors that can be co-administered with the protease inhibitors of the instant invention include, but are not limited to, maraviroc and vicriviroc.

PIKfyve kinase inhibitors that can be co-administered with the protease inhibitors of the instant invention include, but are not limited to, Apilimod.

Serine protease inhibitors that can be co-administered with the protease inhibitors of the instant invention include, but are not limited to, nafamostat mesylate.

Glycosylation inhibitors that can be co-administered with the protease inhibitors of the instant invention include, but are not limited to, WP1122.

Alternatively or additionally, one or more additional pharmacologically active agents may be administered in combination with a compound of the invention. The additional active agent (or agents) is intended to mean a pharmaceutically active agent (or agents) that is active in the body, including pro-drugs that convert to pharmaceutically active form after administration, which is different from the compound of the invention, and also includes free-acid, free-base and pharmaceutically acceptable salts of said additional active agents when such forms are sold commercially or are otherwise chemically possible. Generally, any suitable additional active agent or agents, including but not limited to polymerase nucleoside inhibitors, protease inhibitors, agents that induce viral error catastrophe, eEF1A inhibitors, androgen receptor antagonists, dihydroorotate dehydrogenase (DHODH) inhibitors, sphingosine kinase inhibitors, MEK inhibitors, antimalarials, CCR5 inhibitors, PIKfyve kinase inhibitors, serine protease inhibitors and glycosylation inhibitors can be used in any combination with the compound of the invention in a single dosage formulation (a fixed dose drug combination), or may be administered to the patient in one or more separate dosage formulations which allows for concurrent or sequential administration of the active agents (co-administration of the separate active agents).

Typical doses of the protease inhibitors of the invention in combination with other suitable polymerase nucleoside inhibitors, protease inhibitors, agents that induce viral error catastrophe, eEF1A inhibitors, androgen receptor antagonists, dihydroorotate dehydrogenase (DHODH) inhibitors, sphingosine kinase inhibitors, MEK inhibitors, antimalarials, CCR5 inhibitors, PIKfyve kinase inhibitors, serine protease inhibitors and glycosylation inhibitors may be the same as those doses of the protease inhibitors administered without coadministration of additional polymerase nucleoside inhibitors, protease inhibitors, agents that induce viral error catastrophe, eEF1A inhibitors, androgen receptor antagonists, Dihydroorotate dehydrogenase (DHODH) inhibitors, sphingosine kinase inhibitors, MEK inhibitors, antimalarials, CCR5 inhibitors, PIKfyve kinase inhibitors, serine protease inhibitors and glycosylation inhibitors, or may be substantially less that those doses of protease inhibitors administered without coadministration of polymerase nucleoside inhibitors, protease inhibitors, agents that induce viral catastrophe, eEF1A inhibitors, androgen receptor antagonists, dihydroorotate dehydrogenase (DHODH) inhibitors, sphingosine kinase inhibitors, MEK inhibitors, antimalarials, CCR5 inhibitors, PIKfyve kinase inhibitors, serine protease inhibitors and glycosylation inhibitors depending on a patient's therapeutic needs.

The compounds are administered to a mammal in a therapeutically effective amount. By “therapeutically effective amount” it is meant an amount of a compound of the present invention that, when administered alone or in combination with an additional therapeutic agent to a mammal, is effective to treat (i.e., prevent, inhibit or ameliorate) the viral condition or treat the progression of the disease in a host.

The compounds of the invention are preferably administered alone to a mammal in a therapeutically effective amount. However, the compounds of the invention can also be administered in combination with an additional therapeutic agent, as defined below, to a mammal in a therapeutically effective amount. When administered in a combination, the combination of compounds is preferably, but not necessarily, a synergistic combination. Synergy, as described for example by Chou and Talalay, Adv. Enzyme Regul. 1984, 22, 27-55, occurs when the effect (in this case, inhibition of the desired target) of the compounds when administered in combination is greater than the additive effect of each of the compounds when administered individually as a single agent. In general, a synergistic effect is most clearly demonstrated at suboptimal concentrations of the compounds. Synergy can be in terms of lower cytotoxicity, increased anticoagulant effect, or some other beneficial effect of the combination compared with the individual components.

By “administered in combination” or “combination therapy” it is meant that the compound of the present invention and one or more additional therapeutic agents are administered concurrently to the mammal being treated. When administered in combination each component may be administered at the same time or sequentially in any order at different points in time. Thus, each component may be administered separately but sufficiently closely in time so as to provide the desired therapeutic effect.

The present invention is not limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the relevant art and are intended to fall within the scope of the appended claims.

### General Procedures

Starting materials and intermediates were purchased or were prepared using known procedures described in the chemical synthetic literature or as otherwise described. The preparation of the various starting materials used herein is well within the skill of a person versed in the art. Routes applied to the synthesis of compounds of Formula I are described in the following schemes. In some cases, the sequence of reaction steps may be varied to facilitate reactions or to avoid unwanted reaction products. In some cases, the final product may be further modified, for example, by manipulation of substituents. These manipulations may include, but are not limited to, reduction, oxidation, alkylation, acylation, and hydrolysis reactions which are commonly known to those skilled in the art. Because the schemes are an illustration, the invention should not be construed as being limited by the chemical reactions and conditions expressed. The examples described below are provided so that the invention might be more fully understood. These examples are illustrative only and should not be construed as limiting the invention in any way

It is understood that a chiral center in a compound may exist in the S or R absolute configuration, or as a mixture of both. Within a molecule, each bond drawn as a straight line from a chiral center includes both the R and S stereoisomers as well as mixtures thereof. An asterisk denotes a stereocenter in a single configuration, either R or S. Absolute stereochemistry of separate stereoisomers in the examples and intermediates are not determined unless stated otherwise in an example or explicitly in the nomenclature.

Substituent numbering as shown in the schemes does not necessarily correlate to that used in the claims and often, for clarity, a single substituent is shown attached to the compound where multiple substituents are allowed under the definitions hereinabove. Reactions used to generate the compounds of this invention are carried out by employing reactions as shown in the schemes and examples herein, in addition to other standard manipulations such as ester hydrolysis, cleavage of protecting groups, etc., as may be known in the literature or exemplified in the experimental procedures.

Reactions sensitive to moisture or air were performed under nitrogen or argon using anhydrous solvents and reagents. The progress of reactions was determined by either liquid chromatography-mass spectrometry (LCMS) or analytical thin layer chromatography (TLC) usually performed with Merck KGaA glass-backed TLC plates, silica gel 60 F₂₅₄.

Analytical LCMS was commonly performed on a Waters SQD single quadrupole mass spectrometer with electrospray ionization in positive ion detection mode (mass range set at 150-900 daltons, data collected in centroid mode and scan time set to 0.2 seconds) and a Waters Acquity UPLC system (binary solvent manager, sample manager, and TUV). The column used was a Waters Acquity BEH C18 1×50 mm, 1.7 μm, heated to 50° C. The mobile phases used were modified with either acidic or basic additives. The acidic mobile phase consisted of 0.1% trifluoroacetic acid in water for Solvent A and 100% acetonitrile for Solvent B. A two-minute run was established at a flow rate of 0.3 ml/min with Initial conditions of 95% Solvent A and ramping up to 99% Solvent B at 1.60 minutes and holding at 99% Solvent B for 0.40 minutes. The injection volume was 0.5 μL using partial loop needle overfill injection mode. The TUV monitored wavelength 215 or 254 nm with a sampling rate of 20 points/second, normal filter constant and absorbance data mode. The basic mobile phase consisted of 0.1% ammonium hydroxide in water for solvent A and 100% Acetonitrile for solvent B. A two-minute run was established at a flow rate of 0.3 ml/min with initial conditions of 99% Solvent A and ramping up to 99% Solvent B at 1.90 minutes and holding at 99% Solvent B for 0.10 minutes. A five-minute run was established at a flow rate of 0.3 ml/min with initial conditions of 95% Solvent A and ramping up to 99% Solvent B at 4.90 minutes and holding at 99% Solvent B for 0.10 minutes. For both methods, the injection volume was 5.0 μL using Partial Loop Needle Overfill Injection mode. The TUV monitored wavelength 215 nm with a sampling rate of 20 points/second, normal filter constant and absorbance data mode. Alternatively, a commonly used system consisted of a Waters ZQ™ platform with electrospray ionization in positive ion detection mode with an Agilent 1100 series HPLC with autosampler. The column was commonly a Waters Xterra MS C18, 3.0×50 mm, 5 μm or a Waters Acquity UPLC® BEH C18 1.0×50 mm, 1.7 μm. The flow rate was 1 mL/min, and the injection volume was 10 μL. UV detection was in the range 210-400 nm. The mobile phase consisted of solvent A (water plus 0.05% TFA) and solvent B (MeCN plus 0.05% TFA) with a gradient of 100% solvent A for 0.7 min changing to 100% solvent B over 3.75 min, maintained for 1.1 min, then reverting to 100% solvent A over 0.2 min.

Preparative reverse-phase chromatography was generally carried out on a Teledyne ISCO ACCQPrep HP125 or HP150 apparatus equipped with UV and ELSD detectors. The UV detector typically monitored wavelengths of 215 and 254 nm. The column was commonly one of the following: Waters XBridge Prep C18 OBD 5 μm 30×150 mm, Waters XBridge Prep C18 OBD 5 μm 30×250 mm, Waters XBridge Prep C18 OBD 5 μm 50×250 mm, Waters SunFire Prep C18 OBD 5 μm 30×150 mm, Waters SunFire Prep C18 OBD 10 m 30×150 mm, Waters SunFire Prep C18 OBD 5 μm 50×250 mm, Waters SunFire Prep C18 OBD 10 μm 50×250 mm, or Phenomenex Luna Prep C18 5 μm 50×250 mm. The mobile phases consisted of mixtures of 0.1% TFA in acetonitrile with 0.1% TFA in water or mixtures of 100% acetonitrile with 5 mM (NH₄)HCO₃. Alternatively, a commonly used system was a Waters Chromatography Workstation configured with an LCMS system consisting of: Waters ZQ™ single quad MS system with Electrospray Ionization, Waters 2525 Gradient Pump, Waters 2767 Injector/Collector, Waters 996 PDA Detector. MS conditions were: 150-750 amu, positive electrospray, collection triggered by MS. Columns used were commonly a Waters SunFire C18 5 μm 30×150 mm, a Boston Green ODS 5 μm 150×30 mm, or a YMC-Actus Triart C18 5 μm 150×30 mm column. The mobile phases consisted of mixtures of acetonitrile (10-100%) in water containing 0.1% TFA. Flow rates were maintained at 50 mL/min, and the UV detection range was 210-400 nm. An additional preparative HPLC system used was a Gilson Workstation consisting of: Gilson GX-281 Injector/Collector, Gilson UV/VIS-155 Detector, Gilson 333 and 334 Pumps, and either a Phenomenex Gemini-NX C18 5 μm 50×250 mm column, a Waters XBridge Prep C18 OBD 5 μm 30×250 mm, or a Welch Xtimate C18 5 μm 150×25 mm. The mobile phases consisted of mixtures of acetonitrile (0-75%) in water containing 5 mM (NH₄)HCO₃. Flow rates were maintained at 50 mL/min for the Waters XBridge column, 90 mL/min for the Phenomenex Gemini column, and 25 mL/min for the Welch Xtimate column. The UV detection range was 210-400 nm. Mobile phase gradients were optimized for the individual compounds.

Flash chromatography was usually performed using an ISCO CombiFlash Rf apparatus, a Biotage® Flash Chromatography apparatus (Dyax Corp.), or an ISCO CombiFlash® Companion XL apparatus on silica gel (60 Å pore size) in pre-packed RediSep Rf, RediSep Rf Gold, or SepaFlash columns. Mobile phases generally consisted of mixtures of hexanes or dichloromethane with EtOAc, 3:1 EtOAc:EtOH, or MeOH. Mobile phase gradients were optimized for the individual compounds.

Chiral chromatography was commonly performed by supercritical fluid chromatography with a column chosen from one of the following: Daicel CHIRALPAK AD-H 2 x 25 cm, Daicel CHIRALPAK AD-H 3×25 cm, YMC Chiral ART Cellulose-SC, Lux Cellulose-2 5 μm 30×250 mm, or Exsil Chiral-NR 8 μm 30×250 mm. Mobile phases consisted of mixtures of CO₂ with methanol, ethanol, isopropanol+0.1% diethylamine, isopropanol+0.1% NH₄OH, or 1:1 isopropanol:hexanes+0.1% 2 M NH₃/MeOH. Mobile phase gradients were optimized for the individual compounds. Pressure was typically maintained at 100 bar, and flow rates ranged from 50-200 mL/min. UV monitoring was generally carried out at 220 or 205 nM.

¹H NMR data were typically acquired using a Bruker NEO 500 MHz NMR spectrometer equipped with a room temperature 5 mm BBF iProbe, a Bruker Avance NEO 400 MHz NMR spectrometer equipped with a Bruker PI HR-BBO400S1-BBF/H/D-5.0-Z SP probe, or a Bruker Avance III 500 MHz NMR spectrometer equipped with a Bruker 5 mm PABBO probe. Chemical shift values are reported in delta (δ) units, parts per million (ppm). Chemical shifts for ¹H NMR spectra are given relative to signals for residual non-deuterated solvent (CDCl₃ referenced at δ 7.26 ppm; DMSO-d₆ referenced at δ 2.50 ppm and CD₃OD referenced at δ 3.31 ppm). Multiplets are reported by the following abbreviations: s=singlet, d=doublet, t=triplet, q=quartet, dd=doublet of doublets, m=multiplet or overlap of nonequivalent resonances. Coupling constants (J) are reported in Hertz (Hz). When compounds appear as mixtures of rotamers by NMR, spectral data corresponding to the major species observed in solution are reported.

### Abbreviations

ACN is acetonitrile; AOP is tris(dimethylamino)(3H-1,2,3-triazolo[4,5-b]pyridin-3-yloxy)phosphorus hexafluorophosphate; aq. is aqueous; Bn is benzyl; Boc is tert-butoxycarbonyl; Burgess Reagent is methyl N-(triethylammoniumsulfonyl)carbamate; Cbz is benzyloxycarbonyl; CDI is 1,1′-carbonyldiimidazole; DCM is dichloromethane; DIBAL or DIBAL-H is diisobutylaluminium hydride; DIEA or DIPEA is N,N-diisopropylethylamine; DMA is dimethylacetamide, DMF is N,N-dimethylformamide; DMP is Dess-Martin periodinane; DMSO is dimethyl sulfoxide; EDC or EDCI is 1-ethyl-3-(3-dimethylaminopropyl)carbodiimide; ELSD is evaporative light scattering detector; Et is ethyl; EtOAc is ethyl acetate; HATU is (1-[Bis(dimethylamino)methylene]-1H-1,2,3-triazolo[4,5-b]pyridinium 3-oxide hexafluorophosphate; HOBt is hydroxybenzotriazole; HPLC is high-pressure liquid chromatography; LAH is lithium aluminum hydride; LCMS is liquid chromatography-mass spectrometry; LiHMDS or LHMDS=Lithium bis(trimethylsilyl)amide; LRMS is low resolution mass spectrometry; mCPBA is meta-chloroperoxybenzoic acid; Me is methyl; MeCN is acetonitrile; MeOH is methanol; MTBE is methyl tert-butyl ether; OAc is acetate; OMe is methoxy; Na2CO3 is sodium carbonate; NaHCO₃ is sodium bicarbonate; Na₂SO₄ is anhydrous sodium sulfate; NH₄HCO₃ is ammonium bicarbonate; NaOH is sodium hydroxide; NMP is N-methyl-2-pyrrolidone; NMR is nuclear magnetic resonance; Pd is Palladium; Pd/C is palladium on carbon; PE is petroleum ether; Ph is phenyl; POCl₃ is phosphorus oxychloride; Rochelle's salt is Sodium Potassium Tartrate; RP HPLC is reverse phase high pressure liquid chromatography; RT or rt is room temperature; sat. is saturated; SFC is supercritical fluid chromatography; TEMPO is (2,2,6,6-tetramethylpiperidin-1-yl)oxy; ^{t}Bu is tert-butyl; TBS is tert-butyldimethylsilyl; TEA is triethylamine; TFA is trifluoroacetic acid; THF is tetrahydrofuran; TMS is trimethylsilyl; UV is ultraviolet.

As illustrated in Scheme A, in general, compounds of the invention can be prepared by acylation of an appropriately functionalized amine A-1 to provide compounds of formula A-2. Esters A-2 can be hydrolyzed to yield acids of formula A-3, which can be coupled with amines of formula INT-1 to afford products of formula A-4. Hydroxyamides A-4 can be oxidized to afford ketoamides of formula A-5. In some embodiments, stereoisomers may be separated during the course of the synthesis. Amines of type A-1, acylating agents, and amines of type INT-1 are commercially available or may be synthesized from appropriate intermediates.

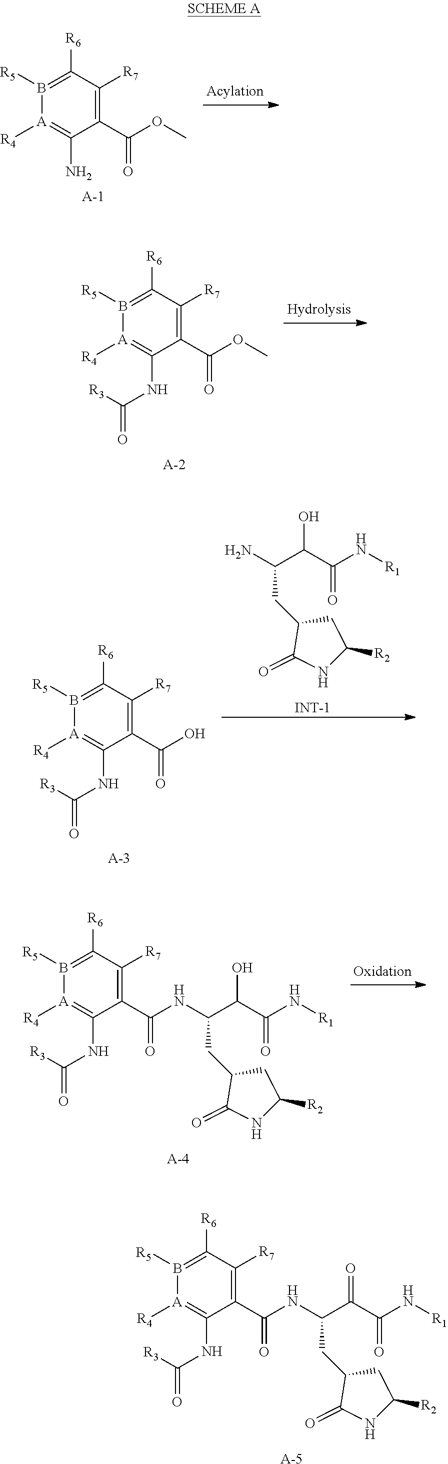

As illustrated in Scheme B, in general, compounds of the invention can be prepared by acylation of an appropriately functionalized amine B-1 to provide compounds of formula B-2, which can be coupled with amines of formula INT-1 to afford products of formula B-3. Hydroxyamides B-3 can be oxidized to afford ketoamides of formula B-4. In some embodiments, stereoisomers may be separated during the course of the synthesis. Amines of type B-1, acylating agents, and amines of type INT-1 are commercially available or may be synthesized from appropriate intermediates.

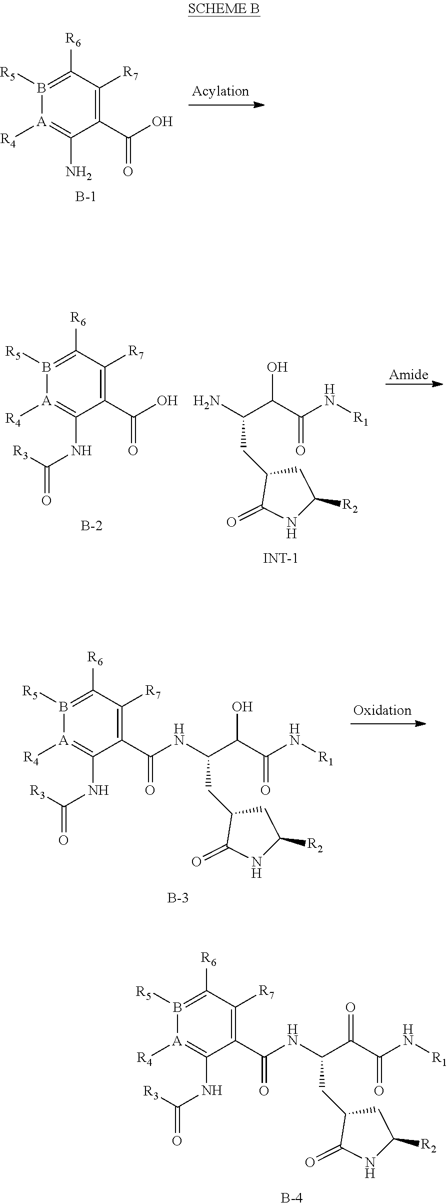

As illustrated in Scheme C, in general, compounds of the invention can be prepared by amidation of an appropriate aryl-halide/heteroaryl-halide C-1 (X=Cl or Br) with appropriate primary amide coupling partner to provide compounds of formula C-2. Esters C-2 can be hydrolyzed to yield acids of formula C-3 which can be coupled with amines of formula INT-1 to afford products of formula C-4. Hydroxyamides C-4 can be oxidized to afford ketoamides of formula C-5. In some embodiments, stereoisomers may be separated during the course of the synthesis. Aryl-halides/heteroaryl-halides of type C-1, primary amide coupling partners, and amines of type INT-1 are commercially available or may be synthesized from appropriate intermediates.

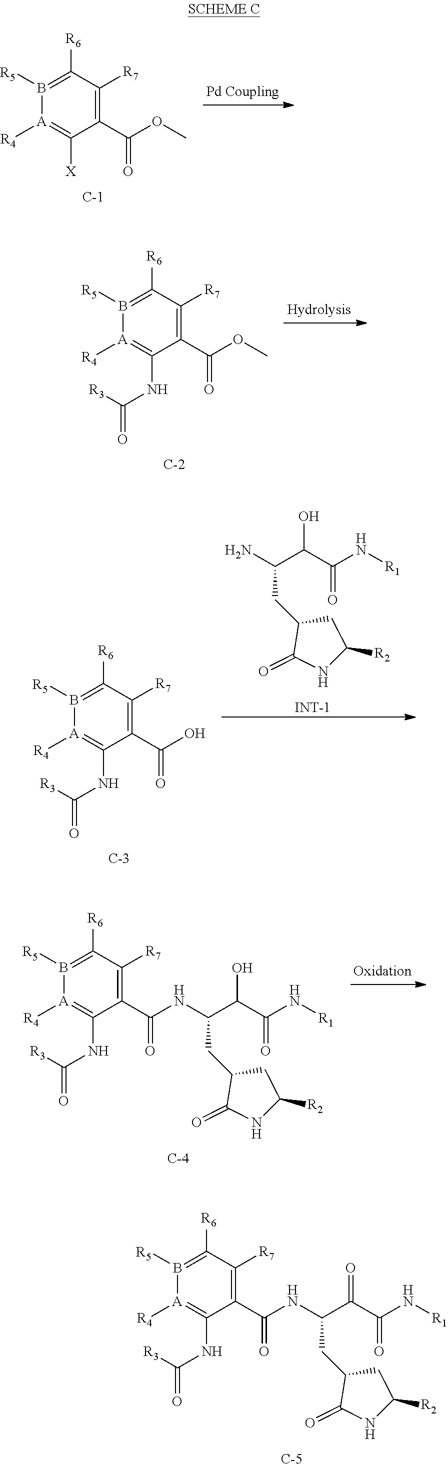

### Intermediate 1

### methyl (S)-2-(((benzyloxy)carbonyl)amino)-3-((3S,5R)-5-methyl-2-oxopyrrolidin-3-yl)propanoate

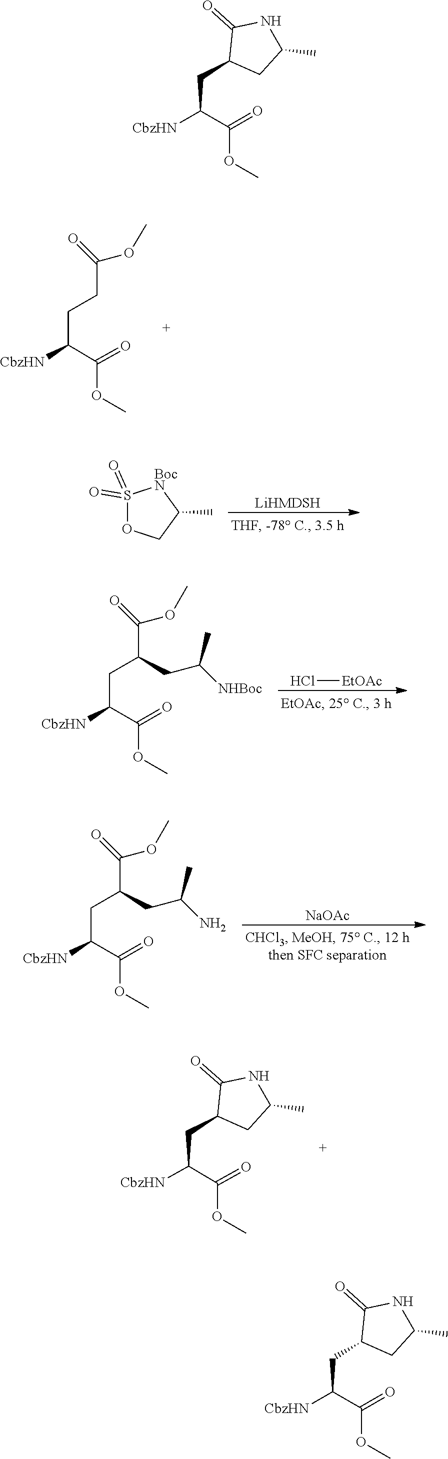

### Step 1: dimethyl (2S,4S)-2-(((benzyloxy)carbonyl)amino)-4-((R)-2-((tert-butoxycarbonyl)amino)propyl)pentanedioate

To a mixture of dimethyl ((benzyloxy)carbonyl)-L-glutamate (4 g, 12.93 mmol) in THE (26 mL) was added LiHMDS (28.4 mL, 28.4 mmol, 1 M in THF) at −78° C. under an atmosphere of nitrogen. The reaction mixture was stirred at −78° C. for 1.5 h. Then tert-butyl (R)-4-methyl-2,2-dioxo-[1,2,3]oxathiazolidine-3-carboxylate (4.60 g, 19.40 mmol) was added to the mixture at −78° C. The reaction was stirred at −78° C. for 2 h. TLC showed most of the starting material was consumed. The reaction mixture was quenched with precooled MeOH (2 mL) at −78° C. and stirred at −78° C. for 10 min. The resulting mixture was quenched with acetic acid in THE (1.8 mL acetic acid/12 mL THF) then stirred at −78° C. for another 10 min. The mixture was allowed to warm up to 0° C., and then brine (50 mL) was added and the mixture was warmed to room temperature then extracted with EtOAc (2×60 mL). The combined organic extracts were dried over Na₂SO₄, filtered and concentrated. The resulting residue was purified using silica gel chromatography eluting with 20% EtOAc/PE. The desired fractions were concentrated to give the title compound.

### Step 2: dimethyl (2S,4S)-2-((R)-2-aminopropyl)-4-(((benzyloxy)carbonyl)amino)pentanedioate

To a solution of dimethyl (2S,4S)-2-(((benzyloxy)carbonyl)amino)-4-((R)-2-((tert-butoxycarbonyl)amino)propyl)pentanedioate (3 g, 6.43 mmol) in EtOAc (10 mL) was added 4M HCl in EtOAc (20 mL). The reaction mixture was stirred at 25° C. for 1 h. LC/MS showed the starting material was consumed. The reaction mixture was then concentrated to give the HCl salt of the title compound which was used without further purification. LRMS m/z: (M+H)⁺ calculated 367.1; found 367.3.

### Step 3: methyl (S)-2-(((benzyloxy)carbonyl)amino)-3-((3S,5R)-5-methyl-2-oxopyrrolidin-3-yl)propanoate

To a solution of dimethyl (2S,4S)-2-((R)-2-aminopropyl)-4-(((benzyloxy)carbonyl)amino)pentanedioate HCl salt (2.3 g, 5.71 mmol) in MeOH (24 mL) and CHCl3 (2 mL) was added sodium acetate (2.342 g, 28.5 mmol). The reaction mixture was then stirred at 75° C. for 12 h. TLC showed the starting material was consumed. Then the reaction was quenched with water (50 mL). The resulting suspension was extracted with EtOAc (2×50 mL). The combined organic extracts were dried over Na₂SO₄, filtered and concentrated. The resulting residue was purified using silica gel chromatography eluting with 75% EtOAc/PE. The desired fractions were concentrated to give the title compound. LRMS m/z: (M+H)⁺ calculated 335.1; found 335.2.

¹H NMR (400 MHz, CDCl₃) δ=7.37-7.31 (m, 5H), 5.96-5.95 (br d, J=7.80 Hz, 1H), 5.83 (br d, J=7.80 Hz, 1H), 5.12 (s, 2H), 4.41-4.35 (m, 1H), 3.76 (s, 4H), 2.59-2.56 (dq, J=5.00, 8.80 Hz, 1H), 2.12-2.08 (m, 1H), 2.06-2.05 (m, 2H), 2.03-1.88 (m, 1H), 1.22-1.19 (d, J=6.40 Hz, 3H).

### Intermediate 2

### benzyl ((S)-1-((3S,5R)-5-methyl-2-oxopyrrolidin-3-yl)-3-oxopropan-2-yl)carbamate

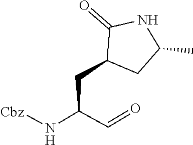 [SMILES: C[C@@H]1C[C@@H](C[C@@H](C=O)NC(=O)OCc2ccccc2)C(=O)N1]

### Intermediate 2 Scheme

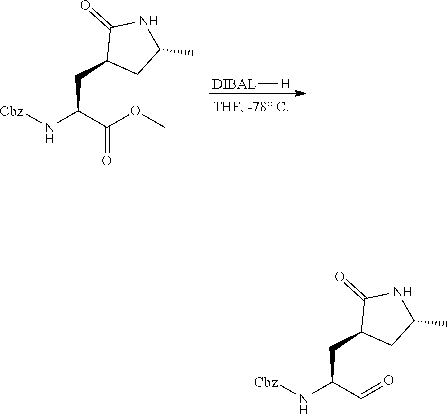

### Step 1: benzyl ((S)-1-((3S,5R)-5-methyl-2-oxopyrrolidin-3-yl)-3-oxopropan-2-yl)carbamate

To a solution of methyl (S)-2-(((benzyloxy)carbonyl)amino)-3-((3S,5R)-5-methyl-2-oxopyrrolidin-3-yl)propanoate (2 g, 5.98 mmol) in THF (30 mL) was added DIBAL-H (23.93 mL, 23.93 mmol) (1 M in toluene) at −78° C. under an atmosphere of nitrogen. The reaction mixture was stirred at −78° C. for 1 h. LC/MS showed the starting material was consumed and the desired product was formed. MeOH (10 mL) was added to the mixture at −78° C. and then the mixture was allowed to warm to room temperature and stirred for 15 min at 25° C. The reaction mixture was then quenched with saturated Rochelle's salt (20 mL), then added EtOAc (20 mL). Stirred for 30 minutes at room temperature then the mixture was extracted with EtOAc (3×20 mL), the combined organic phases were washed with brine (2×20 mL). The organic layer was then dried over Na₂SO₄, filtered and concentrated to give the title compound which was used for the next step without further purification. LRMS m/z: (M+H)⁺ calculated 305.1; found 305.1. ¹H NMR (400 MHz, CHLOROFORM-d) δ 9.55 (s, 1H), 7.40-7.28 (m, 5H), 6.60 (br d, J=6.2 Hz, 1H), 6.16 (br s, 1H), 5.13 (s, 2H), 4.24 (br d, J=5.6 Hz, 1H), 3.86-3.67 (m, 1H), 2.66-2.51 (m, 1H), 2.04-1.90 (m, 4H), 1.19 (br d, J=6.3 Hz, 3H).

### Intermediate 3

### (3S)-3-amino-2-hydroxy-N-methyl-4-((3R,5R)-5-methyl-2-oxopyrrolidin-3-yl)butanamide

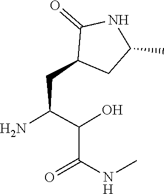 [SMILES: CNC(=O)C(O)[C@@H](N)C[C@@H]1C[C@@H](C)NC1=O]

### Intermediate 3 Scheme

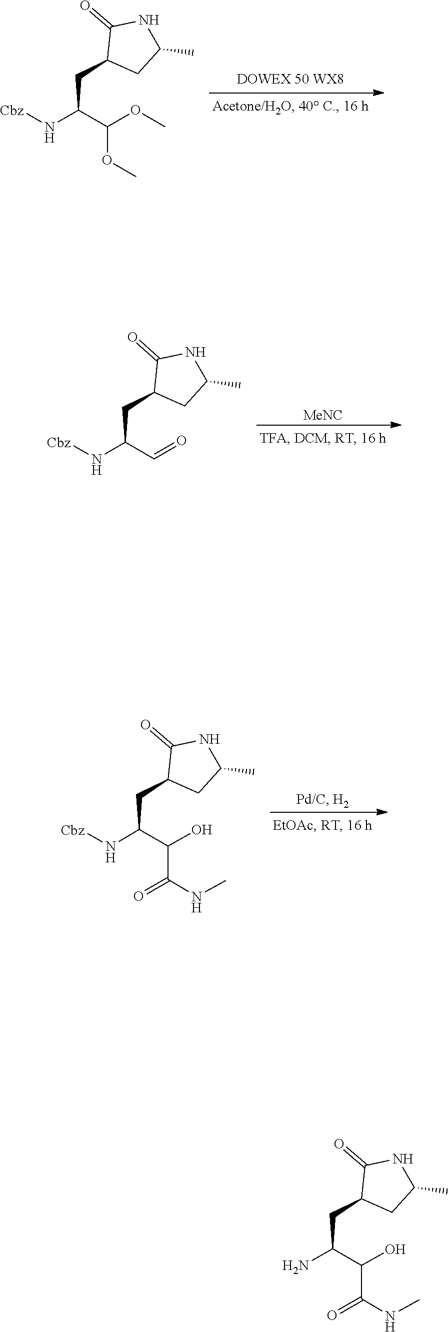 [SMILES: "MeNC" ; COC(OC)[C@H](C[C@@H]1C[C@@H](C)NC1=O)NC(=O)OCc1ccccc1 ; C[C@@H]1C[C@@H](C[C@@H](C=O)NC(=O)OCc2ccccc2)C(=O)N1 ; CNC(=O)C(O)[C@H](C[C@@H]1C[C@@H](C)NC1=O)NC(=O)OCc1ccccc1 ; "Pd/C, H2" ; CNC(=O)C(O)[C@@H](N)C[C@@H]1C[C@@H](C)NC1=O]

Step 1: benzyl ((S)-1-((3S,5R)-5-methyl-2-oxopyrrolidin-3-yl)-3-oxopropan-2-yl)carbamate To a solution of benzyl ((S)-1,1-dimethoxy-3-((3S,5R)-5-methyl-2-oxopyrrolidin-3-yl)propan-2-. yl)carbamate (2 g, 5.71 mmol) in Acetone (20 mL) and Water (20 mL) was added the DOWEX(R) 50WX8 (H) resin (20 g, 5.71 mmol). The resulting mixture was stirred at 40° C. for 16 h. LC/MS showed starting material was consumed and desired product was formed. The resin was then filtered off and the filter cake was washed with 1:1 acetone/water (4×30 mL). The resulting residue was partially concentrated under a stream of nitrogen until the acetone was removed, then the aqueous layer was lyophilized to give the title compound which was used for next step without further purification. LRMS m/z: (M+H)⁺ calculated 305.1; found 305.1. ¹H NMR (400 MHz, CHLOROFORM-d) δ 9.55 (s, 1H), 7.40-7.28 (m, 5H), 6.60 (br d, J=6.2 Hz, 1H), 6.16 (br s, 1H), 5.13 (s, 2H), 4.24 (br d, J=5.6 Hz, 1H), 3.86-3.67 (m, 1H), 2.66-2.51 (m, 1H), 2.04-1.90 (m, 4H), 1.19 (br d, J=6.3 Hz, 3H).

### Step 2: benzyl ((2S)-3-hydroxy-1-((3S,5R)-5-methyl-2-oxopyrrolidin-3-yl)-4-(methylamino)-4-oxobutan-2-yl)carbamate

To a mixture of benzyl ((S)-1-((3S,5R)-5-methyl-2-oxopyrrolidin-3-yl)-3-oxopropan-2-yl)carbamate (1.7 g, 5.59 mmol) and isocyanomethane (0.333 mL, 6.14 mmol) in DCM (30 mL) was added TFA (0.764 g, 6.70 mmol) at 0° C. The reaction mixture was stirred for 16 h at 25° C. LC/MS showed the starting material was consumed and desired MS was found. Water (30 mL) was added, then the mixture was extracted with DCM (3×20 mL). The organic layer was washed with brine (20 mL), then dried over MgSO4, filtered and concentrated. The resulting residue was purified by flash silica gel chromatography (ISCO®; 20 g SepaFlash® Silica Flash Column, eluent of 10% MeOH/DCM gradient @ 24 mL/min). The desired fractions were concentrated, then re-purified by prep-HPLC (Column: Boston Green ODS 150*30 mm*5 m, Condition_(water (TFA)-ACN Begin B 60 End B 80 Gradient Time(min) 10 100% B Hold Time (min) 2, Flow Rate (mL/min) 25). The desired fractions were concentrated to give the title compound. LRMS m/z: (M+H)⁺ calculated 364.1; found 364.2.

¹H NMR (500 MHz, CHLOROFORM-d) δ 7.37-7.28 (m, 5H), 6.89 (br s, 1H), 5.76 (br s, 1H), 5.06 (br s, 2H), 4.24 (br d, J=8.1 Hz, 2H), 3.83-3.61 (m, 1H), 2.79 (br d, J=4.4 Hz, 3H), 2.70-2.50 (m, 1H), 2.03-1.79 (m, 4H), 1.19 (br d, J=5.3 Hz, 3H).

### Step 3: (3S)-3-amino-2-hydroxy-N-methyl-4-((3R,5R)-5-methyl-2-oxopyrrolidin-3-yl)butanamide

To a solution of benzyl ((2S)-3-hydroxy-1-((3S,5R)-5-methyl-2-oxopyrrolidin-3-yl)-4-(methylamino)-4-oxobutan-2-yl)carbamate (800 mg, 2.201 mmol) in EtOAc (15 mL) was added 10% Pd on carbon (234 mg, 0.220 mmol). The mixture was degassed and backfilled with hydrogen (3×). The reaction mixture was then stirred under hydrogen atmosphere at 25° C. for 16 h. LC/MS showed that the starting material was consumed and desired MS was observed. The catalyst was filtered off and the filtrate was concentrated to give the title compound which was used without further purification. LRMS m/z: (M+H)⁺ calculated 230.1; found 230.1. ¹H NMR (500 MHz, METHANOL-d4) δ 4.24-4.14 (m, 1H), 3.86-3.70 (m, 1H), 2.90-2.81 (m, 1H), 2.79 (br d, J=4.4 Hz, 3H), 2.03 (br dd, J=1.4, 2.4 Hz, 3H), 1.91-1.52 (m, 2H), 1.22 (s, 3H).

### Intermediate 4

### (3S)-3-amino-2-hydroxy-N-methyl-4-((S)-2-oxopyrrolidin-3-yl)butanamide

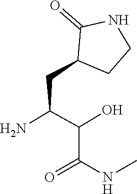

### Intermediate 4 Scheme

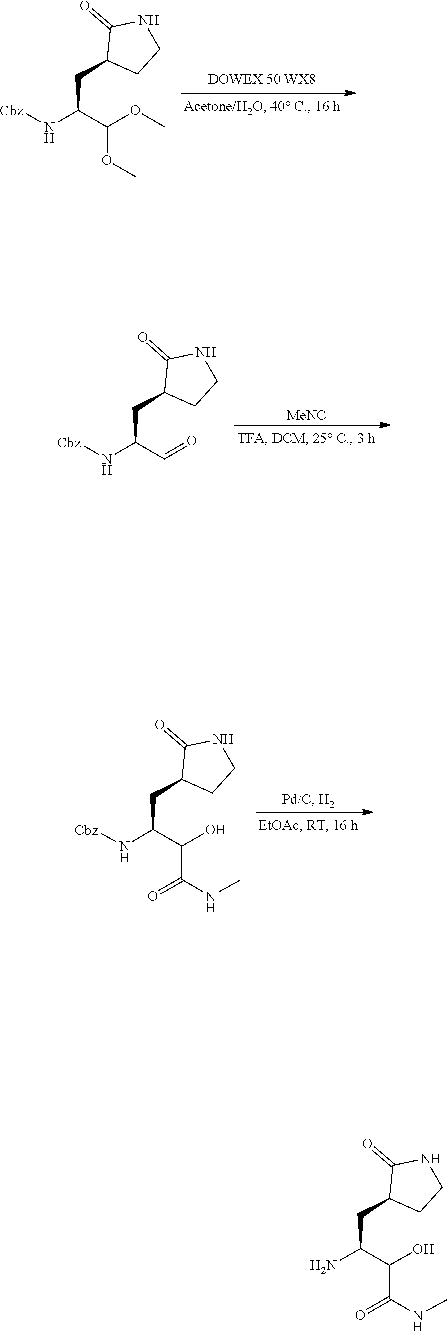 [SMILES: "MeNC" ; COC(OC)[C@H](C[C@@H]1CCNC1=O)NC(=O)OCc1ccccc1 ; O=C[C@H](C[C@@H]1CCNC1=O)NC(=O)OCc1ccccc1 ; CNC(=O)C(O)[C@H](C[C@@H]1CCNC1=O)NC(=O)OCc1ccccc1 ; "Pd/C, H2" ; CNC(=O)C(O)[C@@H](N)C[C@@H]1CCNC1=O]

### Step 1: benzyl ((S)-1-oxo-3-((S)-2-oxopyrrolidin-3-yl)propan-2-yl)carbamate

To a solution of benzyl ((S)-1,1-dimethoxy-3-((S)-2-oxopyrrolidin-3-yl)propan-2-yl)carbamate (5 g, 14.86 mmol) in Acetone (25 mL) and Water (25 mL) was added the DOWEX(R) 50WX8 (H) resin (50 g). The reaction mixture was then stirred at 40° C. for 16 h. LC/MS showed starting material was consumed and desired product was observed. The reaction mixture was filtered and the filter cake was washed with EtOAc (3×30 mL). The filtrate was concentrated to remove the organic solvent and the aqueous was extracted with EtOAc (3×30 mL). The combined organic phases were dried over anhydrous Na₂SO₄, filtered and concentrated under reduced pressure to give the title compound which was used without further purification. LRMS m/z: (M+H)⁺ calculated 291.1; found 291.1.

### Step 2: benzyl ((2S)-3-hydroxy-4-(methylamino)-4-oxo-1-((S)-2-oxopyrrolidin-3-yl)butan-2-yl)carbamate

To a mixture of benzyl ((S)-1-oxo-3-((S)-2-oxopyrrolidin-3-yl)propan-2-yl)carbamate (3 g, 10.33 mmol) in DCM (40 mL), was added TFA (0.921 mL, 12.40 mmol), followed by isocyanomethane (0.6 mL, 11.06 mmol). The reaction mixture was stirred for 3 h at 25° C. LC/MS showed the desired mass was observed. The reaction mixture was then filtered and the filtrate was concentrated under reduced pressure. The residue was dissolved in THF (2 mL) and basified with saturated Na2CO3 to pH=10 then stirred for 16 h. The mixture was extracted with EtOAc (3×10 mL). The combined organics were dried over Na₂SO₄, filtered and concentrated. The resulting residue was purified by RP-HPLC (Column: Boston Green ODS 150*30 mm*5 m; Condition: water (0.01% TFA)-ACN Begin B 17 End B 37 Gradient Time (min) 10,100% B Hold Time 2; Flow Rate (mL/min): 25). The desired fractions were concentrated to give the title compound. LRMS m/z: (M+H)⁺ calculated 350.1; found 350.2.

¹H NMR (400 MHz, METHANOL-d4) δ 7.38-7.22 (m, 5H), 5.07-4.88 (m, 2H), 4.18-3.90 (m, 2H), 3.34-3.31 (m, 2H), 3.25-3.17 (m, 1H), 2.72-2.65 (m, 3H), 2.41-2.20 (m, 1H), 2.13-1.99 (m, 1H), 1.84-1.64 (m, 1H), 1.47-1.03 (m, 1H).

### Step 3: (3S)-3-amino-2-hydroxy-N-methyl-4-((S)-2-oxopyrrolidin-3-yl)butanamide

To a solution of benzyl ((2S)-3-hydroxy-4-(methylamino)-4-oxo-1-((S)-2-oxopyrrolidin-3-yl)butan-2-yl)carbamate (4.5 g, 12.88 mmol) in EtOAc (2 mL) was added 10% Pd on carbon (1.371 g). The reaction mixture was degassed and backfilled with hydrogen (3×). The resulting mixture was stirred under hydrogen atmosphere at 25° C. for 16 h. LC/MS showed that the starting material was consumed and desired mass was observed. The catalyst was filtered off and the filtrate was concentrated to give the title compound which was used without further purification. LRMS m/z: (M+H)⁺ calculated 216.1; found 216.1.

¹H NMR (400 MHz, METHANOL-d4) δ 4.08-3.89 (m, 1H), 3.34-3.32 (m, 1H), 3.16-3.05 (m, 1H), 2.82-2.73 (m, 3H), 2.69-2.58 (m, 1H), 2.43-2.29 (m, 1H), 1.96-1.65 (m, 2H), 1.47-118 (m, 1H).

### Intermediate 5

### (3S)-3-amino-N-cyclopropyl-2-hydroxy-4-((3R,5R)-5-methyl-2-oxopyrrolidin-3-yl)butanamide

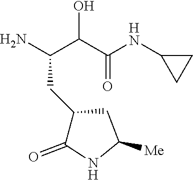 [SMILES: C[C@@H]1C[C@@H](C[C@H](N)C(O)C(=O)NC2CC2)C(=O)N1]

### Intermediate 5 Scheme

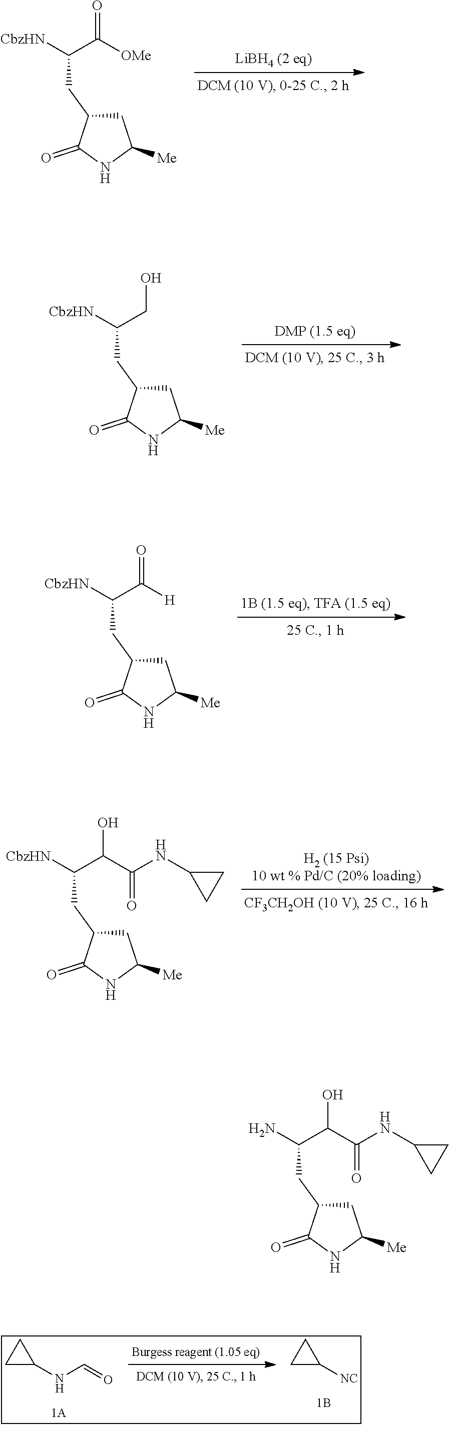

### Step 1: benzyl ((S)-1-hydroxy-3-((3S,5R)-5-methyl-2-oxopyrrolidin-3-yl)propan-2-yl)carbamate

To a 1 L three neck flask was added methyl (S)-2-(((benzyloxy)carbonyl)amino)-3-((3S,5R)-5-methyl-2-oxopyrrolidin-3-yl)propanoate (50 g, 150 mmol) at 25° C. with magnetic stirrer under an atmosphere of nitrogen. Then DCM (500 mL, 10 V) was added, followed by stirring for 10 mins until solubilized. LiBH₄ (6.6 g, 300 mol) was then added into the solution in portions at 0° C. over 15 min. The reaction mixture was then warmed to 25° C. and stirred under an atmosphere of nitrogen, followed by LC/MS. After 2 hrs the starting material was consumed and desired product mass was observed. The reaction was then diluted with 10 wt % aq. NH₄Cl (400 mL) at 25° C. The reaction mixture was then stirred for 20 minutes, then organic layer separated. The aqueous phase was extracted with DCM (2×100 mL). The combined organic layers were dried over Na₂SO₄, filtered and concentrated. The resulting residue was purified by silica gel chromatography eluting with DCM/MeOH from 100/1 to 5/1. The desired fractions were concentrated to give the title compound. LRMS m/z: (M+H)⁺ calculated 307.1; found 307.1. 1H NMR (400 MHz, DMSO-d6) δ: 7.65 (s, 1H), 7.34 (s, 5H), 7.07 (d, J=8.9 Hz, 1H), 5.18-4.90 (m, 2H), 4.67 (t, J=5.7 Hz, 1H), 3.63-3.42 (m, 2H), 3.40-3.20 (m, 4H), 2.37-2.22 (m, 1H), 1.88-1.60 (m, 3H), 1.45-1.29 (m, 1H), 1.04 (d, J=6.3 Hz, 3H).

### Step 2a: benzyl ((S)-1-((3S,5R)-5-methyl-2-oxopyrrolidin-3-yl)-3-oxopropan-2-yl)carbamate

To a 500 mL three-neck flask was added benzyl ((S)-1-hydroxy-3-((3S,5R)-5-methyl-2-oxopyrrolidin-3-yl)propan-2-yl)carbamate (20 g, 65 mmol) at 25° C. with magnetic stirrer under an atmosphere of nitrogen. DCM (200 mL) was then added into the flask and stirred for 10 minutes until solubilized. The reaction mixture was then cooled to 0° C. Then DMP (42 g, 98 mmol) was added to the solution in portions over 20 minutes while keeping the temperature between 0-10° C. The reaction mixture was then warmed to 25° C. with stirring, followed by LC/MS. After 3 hours the reaction was filtered, then the filter cake was washed with DCM (50 mL) to give the title compound which was used without purification. LRMS m/z: (M+H)⁺ calculated 305.1; found 305.1.

### Step 2b: Isocyanocyclopropane

To a to a 250 mL three-neck flask was added N-cyclopropylformamide (0.4 g, 122 mmol) followed by anhydrous DCM (100 mL) at 25° C. with magnetic stirrer under an atmosphere of nitrogen. Burgess Reagent (29 g, 122 mmol) was then added into the solution in portions at 25° C. with stirring, followed by LC/MS. After 1 hr 1H NMR showed ˜20% remaining starting material and ˜80% desired product. The solution in DCM (100 mL) was used directly into the next step without workup or purification.

### Step 3: benzyl ((2S)-4-(cyclopropylamino)-3-hydroxy-1-((3S,5R)-5-methyl-2-oxopyrrolidin-3-yl)-4-oxobutan-2-yl)carbamate

To a flask containing crude reaction mixture from Step 2a containing benzyl ((S)-1-((3S,5R)-5-methyl-2-oxopyrrolidin-3-yl)-3-oxopropan-2-yl)carbamate (65 mmol; theoretical yield) in DCM (250 mL) was added TFA (11.2 g, 98 mmol) by dropwise addition into the organic layer at 25° C. Then the crude solution of isocyanocyclopropane (122 mmol theoretical yield) was added from Step 2b dropwise into the above solution over 20 minutes while maintaining the temperature between 25-30° C., followed by LC/MS. After 1 hour at 25° C., the reaction mixture was quenched with 10 wt % aq. Na₂CO₃, until the pH=7. The organic layer was then separated and the aqueous phase was extracted with DCM (3×80 mL). The combined organic layers were dried over Na₂SO₄, filtered and concentrated. The resulting residue was then purified by Prep-HPLC (water(NH₄HCO₃)-ACN). The desired fractions were then concentrated to give the title compound. LRMS m/z: (M+H)⁺ calculated 390.2; found 390.2.

¹H NMR (400 MHz, MeOD) δ: 7.61-7.19 (m, 5H), 5.19-5.04 (m, 2H), 4.20-3.94 (m, 2H), 3.83-3.61 (m, 1H), 2.82-2.56 (m, 1H), 2.55-2.34 (m, 1H), 2.23-1.81 (m, 3H), 1.56-0.97 (m, 4H), 0.81-0.65 (m, 2H), 0.63-0.39 (m, 2H).

### Step 4: (3S)-3-amino-N-cyclopropyl-2-hydroxy-4-((3R,5R)-5-methyl-2-oxopyrrolidin-3-yl)butanamide

To a 500 mL three neck flask with magnetic stirrer was added benzyl ((2S)-4-(cyclopropylamino)-3-hydroxy-1-((3S,5R)-5-methyl-2-oxopyrrolidin-3-yl)-4-oxobutan-2-yl)carbamate (5.5 g, 14.1 mmol), 10 wt % Pd on carbon (20% loading) and CF₃CH₂OH (55 mL). The reaction mixture was then purged with hydrogen/vacuum and purged with hydrogen (3×) then stirred at 25° C. under hydrogen atmosphere (hydrogen balloon), followed by LC/MS. After 16 hours the reaction mixture was filtered. The filter cake was then washed with MeOH (50 mL). The combined organic layers were concentrated. The resulting residue was purified by prep-HPLC (eluting with water(NH₄HCO₃)-ACN). The desired fractions were concentrated to give the title compound. LRMS m/z: (M+H)⁺ calculated 256.1; found 256.3.

¹H NMR (400 MHz, MeOD) δ: 4.08 (d, J=3.7 Hz, 1H), 3.95 (d, J=3.4 Hz, 1H), 3.82-3.64 (m, 1H), 3.26-3.15 (m, 1H), 2.86-2.55 (m, 2H), 2.11-1.74 (m, 3H), 1.65-1.51 (m, 1H), 1.63-1.10 (m, 1H), 0.85-0.68 (m, 2H), 0.63-0.41 (m, 2H).

### Intermediate 6

### (3S)-3-amino-N-cyclopropyl-2-hydroxy-4-((S)-2-oxopyrrolidin-3-yl)butanamide

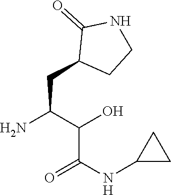 [SMILES: N[C@@H](C[C@@H]1CCNC1=O)C(O)C(=O)NC1CC1]

### Intermediate 6 Scheme

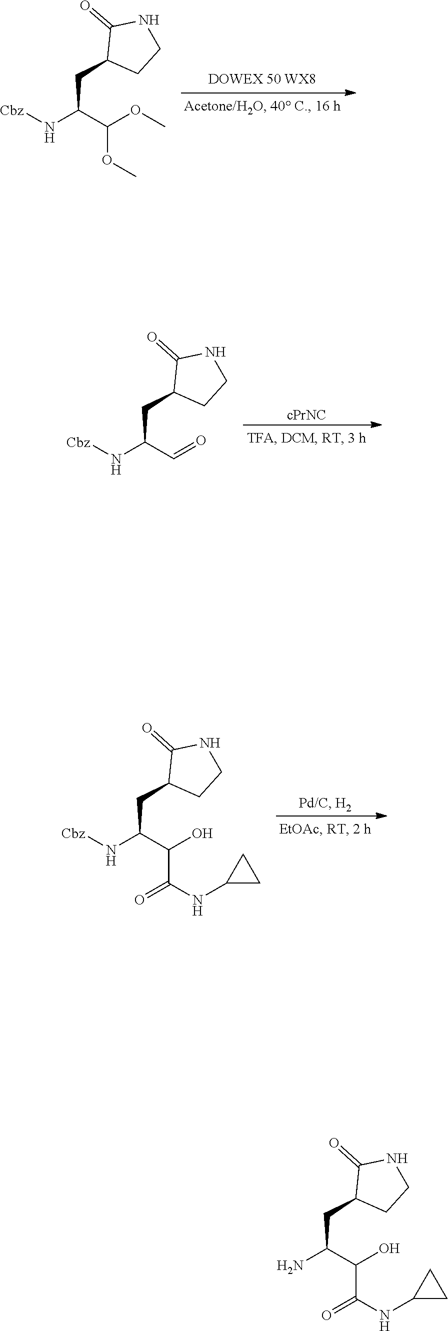 [SMILES: COC(OC)[C@H](C[C@@H]1CCNC1=O)NC(=O)OCc1ccccc1 ; O=C[C@H](C[C@@H]1CCNC1=O)NC(=O)OCc1ccccc1 ; "cPrNC" ; O=C(N[C@@H](C[C@@H]1CCNC1=O)C(O)C(=O)NC1CC1)OCc1ccccc1 ; "Pd/C, H2" ; N[C@@H](C[C@@H]1CCNC1=O)C(O)C(=O)NC1CC1]

### Step 1: benzyl ((S)-1-oxo-3-((S)-2-oxopyrrolidin-3-yl)propan-2-yl)carbamate

To a solution of benzyl ((S)-1,1-dimethoxy-3-((S)-2-oxopyrrolidin-3-yl)propan-2-yl)carbamate (5 g, 14.86 mmol) in Acetone (25 mL) and Water (25 mL) was added the DOWEX(R) 50WX8 (H) resin (50 g, 14.86 mmol). The resulting reaction mixture was stirred at 40° C., followed by LC/MS. After 16 hours the reaction mixture was filtered and the resin was washed with EtOAc (3×30 mL). The filtrate was concentrated to remove the organic solvent and the aqueous layer was extracted with EtOAc (3×30 mL). The combined organic phases were dried over anhydrous Na₂SO₄, filtered and concentrated to give the title compound which was used directly for next step. LRMS m/z: (M+H)+ calculated 291.1; found 291.1.

¹H NMR (400 MHz, CHLOROFORM-d) δ 9.57 (s, 1H), 7.45-7.28 (m, 5H), 6.64 (br d, J=6.0 Hz, 1H), 6.10 (br s, 1H), 5.24-5.04 (m, 2H), 4.30-4.19 (m, 1H), 3.44-3.21 (m, 2H), 2.59-2.26 (m, 2H), 2.04-1.76 (m, 3H).

### Step 2: benzyl ((2S)-4-(cyclopropylamino)-3-hydroxy-4-oxo-1-((S)-2-oxopyrrolidin-3-yl)butan-2-yl)carbamate

To a mixture of benzyl ((S)-1-oxo-3-((S)-2-oxopyrrolidin-3-yl)propan-2-yl)carbamate (2 g, 6.89 mmol) in DCM (30 mL) was added TFA (0.614 mL, 8.27 mmol) and isocyanocyclopropane (0.761 mL, 10.33 mmol). The reaction mixture was stirred at 25° C., followed by LC/MS. After 3 hours the reaction mixture was concentrated and the resulting residue was treated with NaHCO₃ (1.157 g, 13.78 mmol), MeOH (18 mL) and Water (12 mL). The resulting mixture was stirred for 2 hours. LC/MS showed that the reaction was complete. Then the mixture was concentrated and the residue was dissolved in water (10 mL) and EtOAc (20 mL). The organic layer was separated and the aqueous was re-extracted with EtOAc (3×10 mL) and the combined organic layers were washed with brine (10 mL), dried over anhydrous Na₂SO₄, filtered and concentrated. The resulting residue was purified by flash silica gel chromatography (ISCO®; 20 g SepaFlash® Silica Flash Column, eluent of 25% ethyl acetate/pet. ether gradient @ 50 mL/min). The desired fractions were concentrated to give the title compound. LRMS m/z: (M+H)+ calculated 376.1; found 376.1.

### Step 3: (3 S)-3-amino-N-cyclopropyl-2-hydroxy-4-((S)-2-oxopyrrolidin-3-yl)butanamide

To a solution of benzyl ((2S)-4-(cyclopropylamino)-3-hydroxy-4-oxo-1-((S)-2-oxopyrrolidin-3-yl)butan-2-yl)carbamate (600 mg, 1.598 mmol) in EtOAc (20 mL) was added 10% Pd on carbon (170 mg) under nitrogen atmosphere. The reaction mixture was degassed and backfilled with hydrogen/vacuum (3×). The resulting mixture was stirred under hydrogen (balloon) at 25° C., followed by LC/MS. After 1 hour the reaction mixture was filtered, then the filtrate was concentrated to give the title compound. LRMS m/z: (M+H)+ calculated 242.1; found 242.1.

### EXAMPLES

### Example 1

### 5-chloro-N—((S)-4-(cyclopropylamino)-1-((3S,5R)-5-methyl-2-oxopyrrolidin-3-yl)-3,4 dioxobutan-2-yl)-2-(1-(trifluoromethyl)cyclopropane-1-carboxamido)benzamide

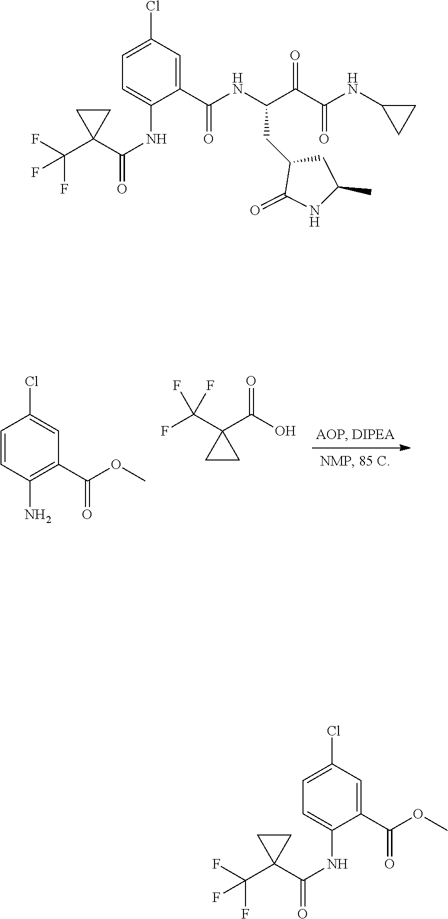 [SMILES: C[C@@H]1C[C@@H](C[C@H](NC(=O)c2cc(Cl)ccc2NC(=O)C2(C(F)(F)F)CC2)C(=O)C(=O)NC2CC2)C(=O)N1 ; COC(=O)c1cc(Cl)ccc1N ; O=C(O)C1(C(F)(F)F)CC1 ; COC(=O)c1cc(Cl)ccc1NC(=O)C1(C(F)(F)F)CC1]

### Step 1: methyl 5-chloro-2-(1-(trifluoromethyl)cyclopropane-1-carboxamido)benzoate

To a vial containing methyl 2-amino-5-chlorobenzoate (304 mg, 1.638 mmol) and 1-(trifluoromethyl)cyclopropane-1-carboxylic acid (415 mg, 2.69 mmol) was added 7-azabenzotriazol-1-yloxytris(dimethylamino)phosphonium hexafluorophosphate (1.16 g, 2.62 mmol) followed by NMP (4 mL) and finally DIPEA (450 μL, 2.58 mmol). The reaction mixture was then capped and heated immediately to 85° C. in the hood, followed by LC/MS. After 1 night at 85° C., the reaction mixture was diluted with 200 μL MeOH, then purified (without workup) by reverse phase chromatography (10-100% MeCN/H2O; 0.1% TFA modifier; 30 min gradient; Waters 50×250 mm Sunfire 5 micron C18 column; Flow=118.1 mL/min). The desired fractions were free based; suspended in EtOAc, washed with saturated NaHCO₃, then water, then brine. The organic layer was then dried over Na₂SO₄, filtered and concentrated, then dissolved in DCM/MeOH and concentrated to give the title compound. LRMS m/z: (M+H)⁺ calculated 322.6; found 322.0.

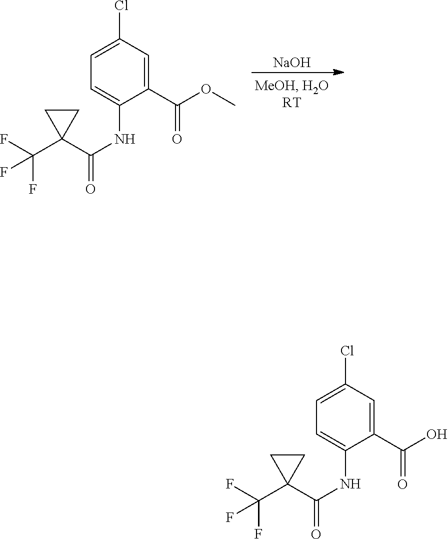 [SMILES: COC(=O)c1cc(Cl)ccc1NC(=O)C1(C(F)(F)F)CC1 ; "NaOH" ; O=C(O)c1cc(Cl)ccc1NC(=O)C1(C(F)(F)F)CC1]

### Step 2: 5-chloro-2-(1-(trifluoromethyl)cyclopropane-1-carboxamido)benzoic acid

To a flask containing methyl 5-chloro-2-(1-(trifluoromethyl)cyclopropane-1-carboxamido)benzoate (150 mg, 0.466 mmol) was added MeOH (4 mL) then water (2 mL) and finally 5N sodium hydroxide (350 μL, 1.750 mmol). The reaction mixture was then capped and stirred at room temperature, followed by LC/MS. After −30 min at room temperature the reaction mixture was diluted/acidified with 1N HCl, then suspended in EtOAc, washed with 1N HCl, then brine. The organic layer was then dried over Na₂SO₄, filtered and concentrated to give the title compound which was used without further purification. LRMS m/z: (M+H)⁺ calculated 308.6; found 308.1.

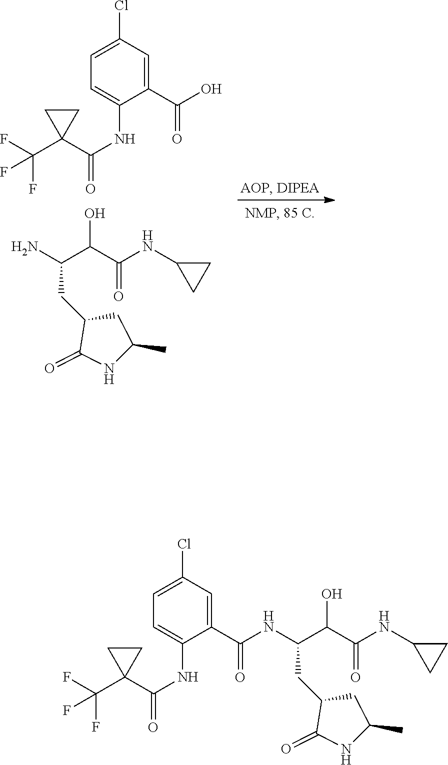

### Step 3: 5-chloro-N-((2S)-4-(cyclopropylamino)-3-hydroxy-1-((3S,5R)-5-methyl-2-oxopyrrolidin-3-yl)-4-oxobutan-2-yl)-2-(1-(trifluoromethyl)cyclopropane-1 carboxamido)benzamide

To a vial containing 5-chloro-2-(1-(trifluoromethyl)cyclopropane-1-carboxamido)benzoic acid (67 mg, 0.218 mmol) and (3S)-3-amino-N-cyclopropyl-2-hydroxy-4-((3R,5R)-5-methyl-2-oxopyrrolidin-3-yl)butanamide (74 mg, 0.290 mmol) was added 7-azabenzotriazol-1-yloxytris(dimethylamino)phosphonium hexafluorophosphate (162 mg, 0.365 mmol) followed by NMP (1.5 mL) and finally DIPEA (105 μL, 0.601 mmol). The reaction mixture was then capped and heated immediately to 85° C. in the hood, followed by LC/MS. After 1 night at 85° C., the reaction mixture was diluted with 200 μL MeOH, then purified (without workup) by reverse phase chromatography (5-60% MeCN/H2O; 0.1% TFA modifier; 20 min gradient; Waters 30×150 mm Sunfire 5 micron C18 column; Flow=42.5 mL/min). The desired fractions were diluted with MeOH, concentrated, and then dissolved in DCM/MeOH and concentrated to give the title compound. LRMS m/z: (M+H)⁺ calculated 545.9; found 545.2.

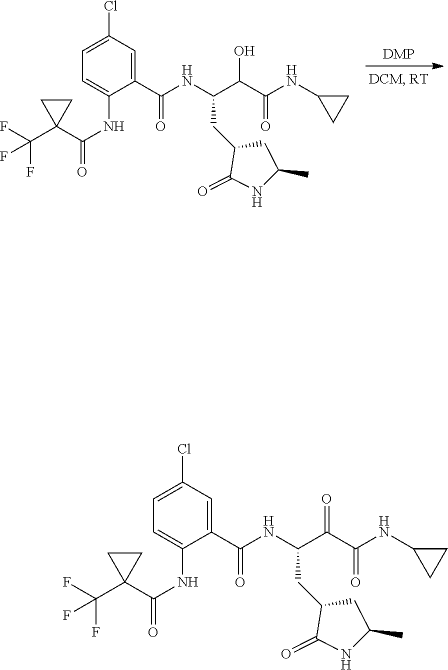

### Step 4: 5-chloro-N—((S)-4-(cyclopropylamino)-1-((3S,5R)-5-methyl-2-oxopyrrolidin-3-yl)-3,4 dioxobutan-2-yl)-2-(1-(trifluoromethyl)cyclopropane-1-carboxamido)benzamide

To a vial containing 5-chloro-N-((2S)-4-(cyclopropylamino)-3-hydroxy-1-((3S,5R)-5-methyl-2-oxopyrrolidin-3-yl)-4-oxobutan-2-yl)-2-(1-(trifluoromethyl)cyclopropane-1-carboxamido)benzamide (100 mg, 0.184 mmol) was added Dess-Martin Periodinane (138 mg, 0.325 mmol) and sodium bicarbonate (76 mg, 0.905 mmol) followed by DCM (5 mL). The reaction mixture was then capped and stirred at room temperature, followed by LC/MS. After 30 min the reaction mixture was quenched/diluted with 4 mL of saturated sodium thiosulfate followed by 1 mL water, then diluted with 10 mL EtOAc. Stirred for 10 minutes then suspended in EtOAc, washed with saturated sodium thiosulfate, then water, then brine. The organic layer was then dried over Na₂SO₄, filtered and concentrated. The resulting residue was purified by silica gel chromatography (30-100% EtOAc/Hex; 14 CV; 40 g ISCO). The desired fractions were concentrated to give the title compound. LRMS m/z: (M+H)⁺ calculated 543.9; found 543.1. ¹H NMR (500 MHz, DMSO-d6) δ 9.65 (d, J=6.6 Hz, 1H), 8.81 (d, J=5.1 Hz, 1H), 8.36 (d, J=9.0 Hz, 1H), 7.97-7.89 (m, 2H), 7.63 (dd, J=9.0, 2.5 Hz, 1H), 5.23-5.16 (m, 1H), 3.69-3.57 (m, 1H), 2.80-2.73 (m, 1H), 2.68-2.59 (m, 1H), 2.03-1.94 (m, 2H), 1.91-1.84 (m, 1H), 1.78-1.71 (m, 1H), 1.47-1.42 (m, 2H), 1.41-1.36 (m, 2H), 1.09 (d, J=6.4 Hz, 3H), 0.69-0.66 (m, 2H), 0.61-0.56 (m, 2H).

### Example 2

### N-(4-chloro-2-(((S)-4-(cyclopropylamino)-1-((3S,5R)-5-methyl-2-oxopyrrolidin-3-yl)-3,4-dioxobutan-2-yl)carbamoyl)phenyl)-2-(trifluoromethyl)isonicotinamide

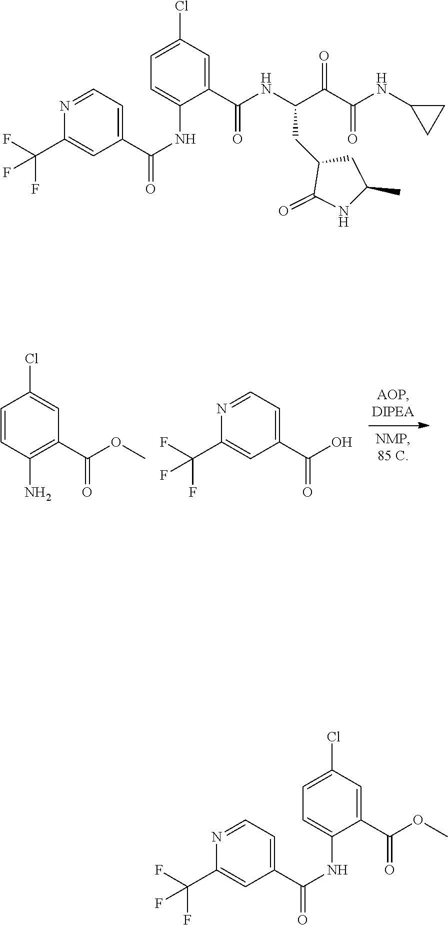

### Step 1: methyl 5-chloro-2-(2-(trifluoromethyl)isonicotinamido)benzoate

To a vial containing methyl 2-amino-5-chlorobenzoate (354 mg, 1.907 mmol) and 2-(trifluoromethyl)isonicotinic acid (507 mg, 2.65 mmol) was added 7-azabenzotriazol-1-yloxytris(dimethylamino)phosphonium hexafluorophosphate (2.13 g, 4.81 mmol) followed by NMP (6 mL) and finally DIPEA (850 μL, 4.87 mmol). The reaction mixture was then capped and heated immediately to 85° C. in the hood, followed by LC/MS. After 1 night at 85° C., the reaction mixture was diluted with 200 μL MeOH, then purified (without workup) by reverse phase chromatography (25-100% MeCN/H2O; 0.1% TFA modifier; 30 min gradient; Waters 50×250 mm Sunfire 5 micron C18 column; Flow=118.1 mL/min). The desired fractions were concentrated, then dissolved in DCM/MeOH and concentrated to give the title compound. LRMS m/z: (M+H)⁺ calculated 359.7; found 359.1.

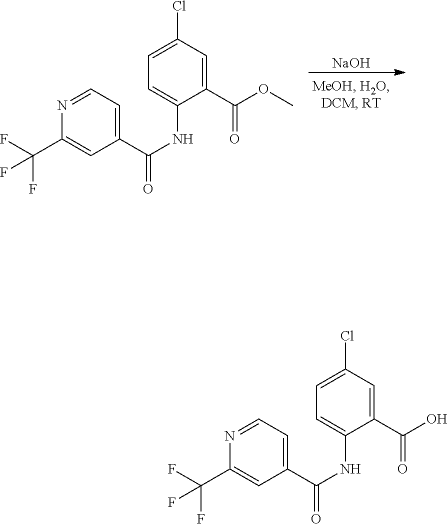 [SMILES: COC(=O)c1cc(Cl)ccc1NC(=O)c1ccnc(C(F)(F)F)c1 ; "NaOH" ; O=C(Nc1ccc(Cl)cc1C(=O)O)c1ccnc(C(F)(F)F)c1]

### Step 2: 5-chloro-2-(2-(trifluoromethyl)isonicotinamido)benzoic acid

To a flask containing methyl 5-chloro-2-(2-(trifluoromethyl)isonicotinamido)benzoate (432 mg, 1.204 mmol) was added MeOH (10 mL) then Water (4 mL) and finally 5N sodium hydroxide (650 μL, 3.25 mmol). The reaction mixture was then capped and stirred at room temperature. After adding the NaOH the reaction mixture was still a suspension so added DCM (3 mL) which immediately solubilized the mixture, followed by LC/MS. After 1.75 hrs at room temperature the reaction mixture was diluted/acidified with 1N HCl, then suspended in EtOAc, washed with 1N HCl, then brine. The organic layer was then dried over Na₂SO₄, filtered and concentrated to give the title compound which was used without further purification. LRMS m/z: (M+H)⁺ calculated 345.6; found 345.0.

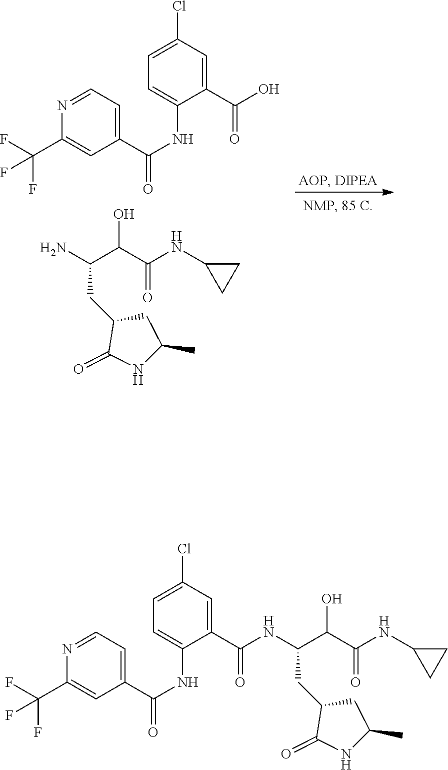

### Step 3: N-(4-chloro-2-(((2S)-4-(cyclopropylamino)-3-hydroxy-1-((3S,5R)-5-methyl-2-oxopyrrolidin-3-yl)-4-oxobutan-2-yl)carbamoyl)phenyl)-2-(trifluoromethyl)isonicotinamide

To a vial containing 5-chloro-2-(2-(trifluoromethyl)isonicotinamido)benzoic acid (112 mg, 0.325 mmol) and (3S)-3-amino-N-cyclopropyl-2-hydroxy-4-((3R,5R)-5-methyl-2-oxopyrrolidin-3-yl)butanamide (103 mg, 0.403 mmol) was added 7-azabenzotriazol-1-yloxytris(dimethylamino)phosphonium hexafluorophosphate (383 mg, 0.864 mmol) followed by NMP (1.5 mL) and finally DIPEA (145 μL, 0.830 mmol). The reaction mixture was then capped and heated immediately to 85° C. in the hood, followed by LC/MS. After 1 night at 85° C., the reaction mixture was diluted with 200 μL MeOH, then purified (without workup) by reverse phase chromatography (10-60% MeCN/H2O; 0.1% TFA modifier; 20 min gradient; Waters 30×150 mm Sunfire 5 micron C18 column; Flow=42.5 mL/min). The desired fractions were diluted with MeOH, concentrated, and then dissolved in DCM/MeOH and concentrated to give the title compound. LRMS m/z: (M+H)⁺ calculated 582.9; found 582.1.

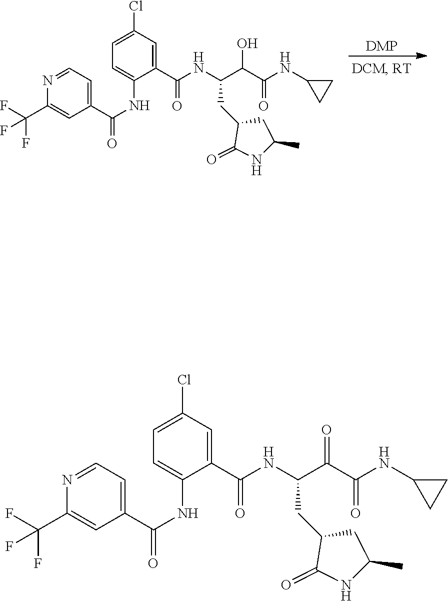

### Step 4: N-(4-chloro-2-(((S)-4-(cyclopropylamino)-1-((3 S,5R)-5-methyl-2-oxopyrrolidin-3-yl)-3,4-dioxobutan-2-yl)carbamoyl)phenyl)-2-(trifluoromethyl)isonicotinamide

To a vial containing N-(4-chloro-2-(((2S)-4-(cyclopropylamino)-3-hydroxy-1-((3S,5R)-5-methyl-2-oxopyrrolidin-3-yl)-4-oxobutan-2-yl)carbamoyl)phenyl)-2-(trifluoromethyl)isonicotinamide (140 mg, 0.241 mmol) was added Dess-Martin Periodinane (168 mg, 0.396 mmol) and sodium bicarbonate (42 mg, 0.500 mmol) and finally DCM (5 mL). The reaction mixture was then capped and stirred at room temperature, followed by LC/MS. After 60 min the reaction mixture was quenched/diluted with 4 mL of saturated sodium thiosulfate followed by 1 mL water, then diluted with 10 mL EtOAc. Stirred for 10 minutes then suspended in EtOAc, washed with saturated sodium thiosulfate, then water, then brine. The organic layer was then dried over Na2SO₄, filtered and concentrated. The resulting residue was purified by silica gel chromatography (10-100% EtOAc/Hex; 14 CV; 40 g ISCO). The desired fractions were concentrated to give the title compound. LRMS m/z: (M+H)⁺ calculated 580.9; found 580.1. ¹H NMR (500 MHz, DMSO-d6) δ 9.67 (d, J=6.6 Hz, 1H), 9.04 (d, J=5.0 Hz, 1H), 8.80 (d, J=5.0 Hz, 1H), 8.38 (d, J=8.9 Hz, 1H), 8.21 (s, 1H), 8.09 (d, J=4.9 Hz, 1H), 7.97 (d, J=2.4 Hz, 1H), 7.89 (s, 1H), 7.72 (dd, J=8.9, 2.4 Hz, 1H), 5.22-5.13 (m, 1H), 3.64-3.51 (m, 1H), 2.76-2.69 (m, 1H), 2.68-2.60 (m, 1H), 2.02-1.93 (m, 2H), 1.86-1.79 (m, 1H), 1.78-1.70 (m, 1H), 1.00 (d, J=6.3 Hz, 3H), 0.67-0.62 (m, 2H), 0.56-0.51 (m, 2H).

### Example 3

### 5-chloro-N—((S)-4-(cyclopropylamino)-1-((3S,5R)-5-methyl-2-oxopyrrolidin-3-yl)-3,4-dioxobutan-2-yl)-2-(4,4,4-trifluorobutanamido)benzamide

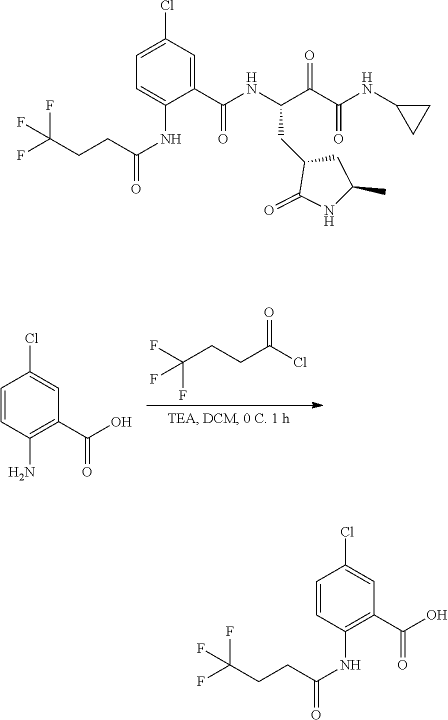 [SMILES: C[C@@H]1C[C@@H](C[C@H](NC(=O)c2cc(Cl)ccc2NC(=O)CCC(F)(F)F)C(=O)C(=O)NC2CC2)C(=O)N1 ; Nc1ccc(Cl)cc1C(=O)O ; O=C(Cl)CCC(F)(F)F ; O=C(CCC(F)(F)F)Nc1ccc(Cl)cc1C(=O)O]

### Step 1: 5-chloro-2-(4,4,4-trifluorobutanamido)benzoic acid

To a solution of 2-amino-5-chlorobenzoic acid (150 mg, 0.874 mmol) in DCM (5 mL) was added triethylamine (0.366 mL, 2.62 mmol) and 4,4,4-trifluorobutanoyl chloride (154 mg, 0.962 mmol) at 0° C. The resulting mixture was stirred for 1 h at 0° C., followed by LC/MS. Then 0.5 M HCl aq. (10 mL) was added to quench the reaction. The mixture was extracted with DCM (2×10 mL). The separated organic was dried over Na₂SO₄, filtered and concentrated. The resulting residue was purified by RP-HPLC (Column: Boston Green ODS 150*30 mm*5 m; Condition: water (TFA)-ACN Begin B 45 End B 65; Gradient Time (min): 10 100% B; Hold Time (min): 2; Flow Rate (mL/min): 25). The desired fractions were concentrated to give the title compound. LRMS m/z: (M+H)⁺ calculated 296.0; found 296.0.

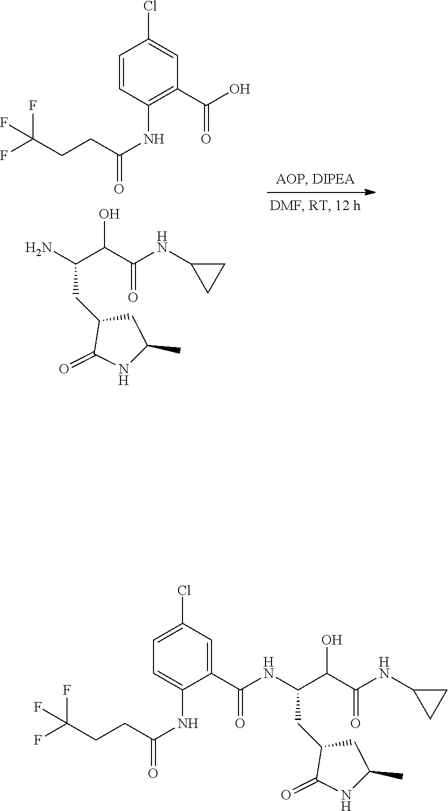

### Step 2: 5-chloro-N-((2S)-4-(cyclopropylamino)-3-hydroxy-1-((3S,5R)-5-methyl-2-oxopyrrolidin-3-yl)-4-oxobutan-2-yl)-2-(4,4,4-trifluorobutanamido)benzamide

To a mixture of 5-chloro-2-(4,4,4-trifluorobutanamido)benzoic acid (57.9 mg, 0.196 mmol) and (3S)-3-amino-N-cyclopropyl-2-hydroxy-4-((3R,5R)-5-methyl-2-oxopyrrolidin-3-yl)butanamide (50 mg, 0.196 mmol) in DMF (1.5 mL) was added AOP (104 mg, 0.235 mmol) and DIPEA (0.103 mL, 0.588 mmol) at 25° C. The resulting mixture was stirred at 25° C. 12 h, followed by LC/MS. Then the reaction mixture was directly purified by RP-HPLC (Column: Boston Green ODS 150*30 mm*5 μm; Condition: water (TFA)-ACN Begin B 33 End B 53; Gradient Time (min): 10 100% B; Hold Time (min): 2; Flow Rate (mL/min: 25). The desired fractions were concentrated to give the title compound. LRMS m/z: (M+H)+ calculated 533.1; found 533.2.

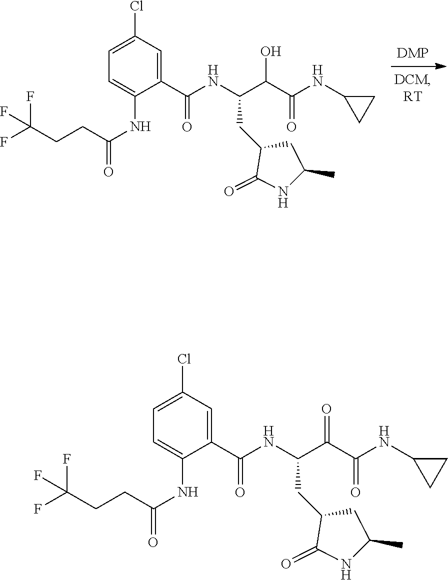 [SMILES: C[C@@H]1C[C@@H](C[C@H](NC(=O)c2cc(Cl)ccc2NC(=O)CCC(F)(F)F)C(O)C(=O)NC2CC2)C(=O)N1 ; C[C@@H]1C[C@@H](C[C@H](NC(=O)c2cc(Cl)ccc2NC(=O)CCC(F)(F)F)C(=O)C(=O)NC2CC2)C(=O)N1]

### Step 3: 5-chloro-N—((S)-4-(cyclopropylamino)-1-((3S,5R)-5-methyl-2-oxopyrrolidin-3-yl)-3,4-dioxobutan-2-yl)-2-(4,4,4-trifluorobutanamido)benzamide

To a mixture of 5-chloro-N-((2S)-4-(cyclopropylamino)-3-hydroxy-1-((3S,5R)-5-methyl-2-oxopyrrolidin-3-yl)-4-oxobutan-2-yl)-2-(4,4,4-trifluorobutanamido)benzamide (55 mg, 0.103 mmol) and NaHCO₃ (26.0 mg, 0.310 mmol) in DCM (1.5 mL) was added DMP (65.7 mg, 0.155 mmol) at 25° C. The resulting mixture was stirred at 25° C. for 2 h, followed by LC/MS. The reaction mixture was then filtered and the filtrate was concentrated. The resulting residue was purified by RP-HPLC (Column: Welch Xtimate C18 150*25 mm*5 m; Condition: water (NH4HCO3)-ACN Begin B 33 End B 63; Gradient Time (min): 11 100% B; Hold Time (min): 2; Flow Rate (mL/min): 25). The desired fractions were concentrated to give the title compound. LRMS m/z: (M+H)+ calculated 531.1; found 531.0.

¹H NMR (500 MHz, CHLOROFORM-d) δ 11.53-11.34 (m, 1H), 9.93-9.92 (m, 1H), 8.56 (d, J=9.0 Hz, 1H), 7.89 (d, J=2.4 Hz, 1H), 7.43-7.40 (m, 1H), 6.98-6.97 (m, 1H), 6.04-5.90 (m, 1H), 5.34-5.30 (m, 1H), 3.87-3.71 (m, 1H), 2.94-2.72 (m, 2H), 2.65-2.61 (m, 2H), 2.57-2.49 (m, 2H), 2.36-2.11 (m, 3H), 1.94-1.87 (m, 1H), 1.25-1.19 (m, 3H), 0.91-0.80 (m, 2H), 0.70-0.58 (m, 2H).

### Example 4

### 2-(3,5-bis(trifluoromethyl)benzamido)-5-cyano-N—((S)-1-((3S,5R)-5-methyl-2-oxopyrrolidin-3-yl)-4-(methylamino)-3,4-dioxobutan-2-yl)nicotinamide

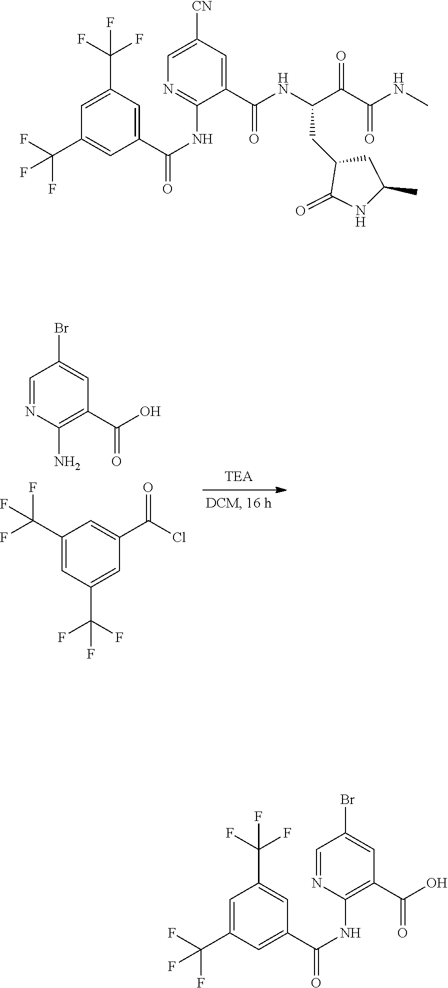

### Step 1: 2-(3,5-bis(trifluoromethyl)benzamido)-5-bromonicotinic acid

To a mixture of 2-amino-5-bromonicotinic acid (1 g, 4.61 mmol) and TEA (1.285 mL, 9.22 mmol) in DCM (25 mL) was added 3,5-bis(trifluoromethyl)benzoyl chloride (1.274 g, 4.61 mmol) slowly at 0° C. The resulting mixture was allowed to warm to room temperature and stirred a total of 16 h, followed by LC/MS. Then the reaction mixture was diluted with DCM (20 mL) and filtered. The filtrate was concentrated under reduced pressure to give the title compound which was used without further purification. LRMS m/z: (M+H)+ calculated 456.9; found 457.0.

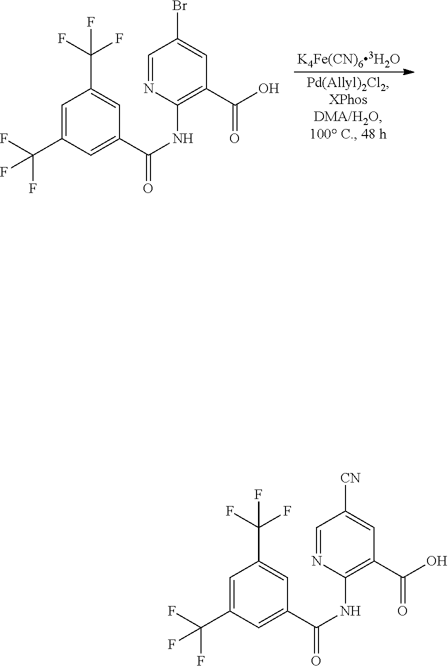 [SMILES: O=C(Nc1ncc(Br)cc1C(=O)O)c1cc(C(F)(F)F)cc(C(F)(F)F)c1 ; "K4Fe(CN)6·3H2O" ; "Pd(Allyl)2Cl2,XPhosDMA/H2O, 100° C., 48 h" ; N#Cc1cnc(NC(=O)c2cc(C(F)(F)F)cc(C(F)(F)F)c2)c(C(=O)O)c1]

### Step 2: 2-(3,5-bis(trifluoromethyl)benzamido)-5-cyanonicotinic acid

To a mixture of methyl 2-(3,5-bis(trifluoromethyl)benzamido)-5-bromonicotinate (470 mg, 0.998 mmol) and potassium hexacyanoferrate (II) trihydrate (527 mg, 1.247 mmol) in DMA (8 mL) and Water (1 mL) was added dicyclohexyl(2′,4′,6′-triisopropyl-[1,1′-biphenyl]-2-yl)phosphane (95 mg, 0.200 mmol) and allylpalladium(II) chloride (36.5 mg, 0.100 mmol) at 25° C. The reaction mixture was then degassed and backfilled with nitrogen three times. The resulting mixture was stirred at 100° C. for 48 h, followed by LC/MS. The reaction mixture was then filtered, the solid was collected via filtration, then was purified by RP-HPLC (Column: Boston Green ODS 150*30 mm*5 m, Condition: water (0.01% TFA)-ACN Begin B 42 End B 62 Gradient Time (min) 10 100% B Hold Time 2 Flow Rate (mL/min) 25). The desired fractions were concentrated to give the title compound. LRMS m/z: (M+H)+ calculated 404.0; found 404.0.

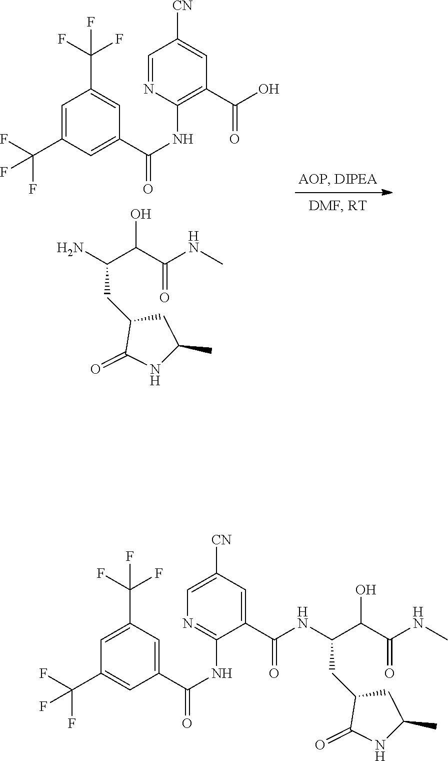

### Step 3: 2-(3,5-bis(trifluoromethyl)benzamido)-5-cyano-N-((2S)-3-hydroxy-1-((3S,5R)-5-methyl-2-oxopyrrolidin-3-yl)-4-(methylamino)-4-oxobutan-2-yl)nicotinamide

To a mixture of 2-(3,5-bis(trifluoromethyl)benzamido)-5-cyanonicotinic acid (65 mg, 0.161 mmol) and (3S)-3-amino-2-hydroxy-N-methyl-4-((3R,5R)-5-methyl-2-oxopyrrolidin-3-yl)butanamide (44.4 mg, 0.193 mmol) in DMF (2 mL) was added DIPEA (0.084 mL, 0.484 mmol), and AOP (86 mg, 0.193 mmol) at 25° C. The resulting mixture was stirred for 16 h, followed by LC/MS. Then the reaction mixture was purified by RP-HPLC (Column: Boston Green ODS 150*30 mm*5 m, Condition: water (0.01% TFA)-ACN Begin B 33 End B 53 Gradient Time (min) 5 100% B Hold Time 2 Flow Rate (mL/min) 25). The desired fractions were concentrated to give the title compound. LRMS m/z: (M+H)+ calculated 615.1; found 615.2.

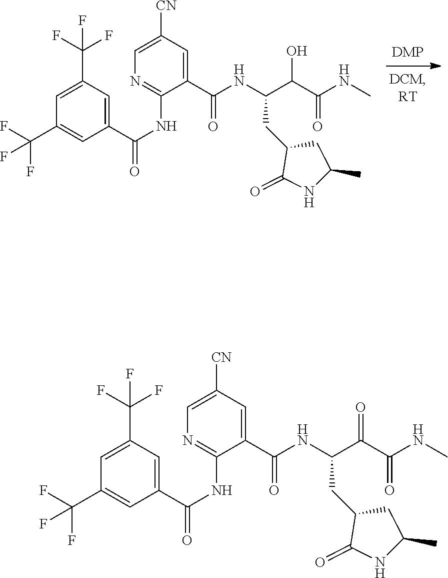 [SMILES: CNC(=O)C(O)[C@H](C[C@@H]1C[C@@H](C)NC1=O)NC(=O)c1cc(C#N)cnc1NC(=O)c1cc(C(F)(F)F)cc(C(F)(F)F)c1 ; CNC(=O)C(=O)[C@H](C[C@@H]1C[C@@H](C)NC1=O)NC(=O)c1cc(C#N)cnc1NC(=O)c1cc(C(F)(F)F)cc(C(F)(F)F)c1]

### Step 4: 2-(3,5-bis(trifluoromethyl)benzamido)-5-cyano-N—((S)-1-((3S,5R)-5-methyl-2-oxopyrrolidin-3-yl)-4-(methylamino)-3,4-dioxobutan-2-yl)nicotinamide

To a mixture of 2-(3,5-bis(trifluoromethyl)benzamido)-5-cyano-N-((2S)-3-hydroxy-1-((3S,5R)-5-methyl-2-oxopyrrolidin-3-yl)-4-(methylamino)-4-oxobutan-2-yl)nicotinamide (45 mg, 0.073 mmol) and sodium hydrogen carbonate (18.46 mg, 0.220 mmol) in DCM (2 mL) was added DMP (93 mg, 0.220 mmol) at 25° C., followed by LC/MS. After 3 hours the reaction mixture was filtered and the filtrate was concentrated under reduced pressure. The resulting residue was purified by RP-HPLC (Column: Welch Xtimate C18 150*25 mm*5 m, Condition: water (10 mM-NH4HCO3)-ACN Begin B 25 End B 55 Gradient Time (min) 11 100% B Hold Time 2 Flow Rate (mL/min) 25). The desired fractions were concentrated to give the title compound. LRMS m/z: (M+H)+ calculated 613.1; found 613.3.

¹H NMR (400 MHz, METHANOL-d4) δ 9.01-8.41 (m, 4H), 8.21 (m, J=16.0 Hz, 1H), 4.74-4.24 (m, 1H), 3.81-3.47 (m, 1H), 2.90-2.56 (m, 4H), 2.21-1.43 (m, 4H), 1.19-0.83 (m, 3H).

### Example 5

### 5-chloro-N—((S)-4-(cyclopropylamino)-1-((3S,5R)-5-methyl-2-oxopyrrolidin-3-yl)-3,4-dioxobutan-2-yl)-2-(3-(trifluoromethyl)bicyclo[1.1.1]pentane-1-carboxamido)nicotinamide

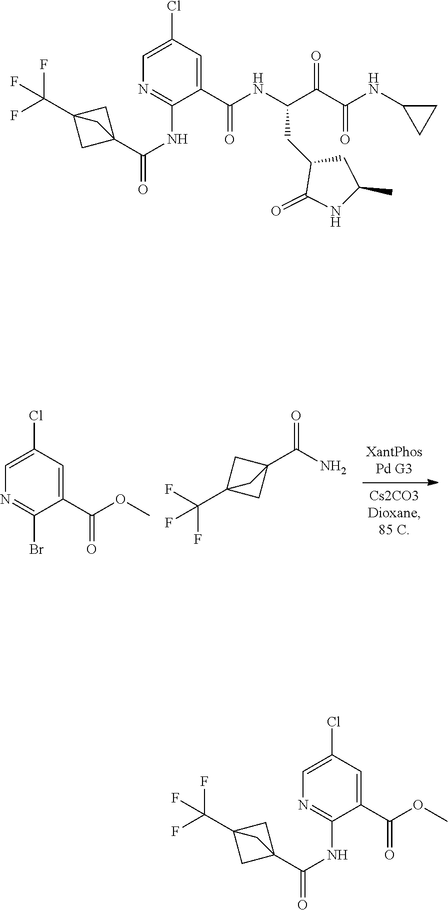

### Step 1: methyl 5-chloro-2-(3-(trifluoromethyl)bicyclo[1.1.1]pentane-1-carboxamido)nicotinate

To a vial containing methyl 2-bromo-5-chloronicotinate (630 mg, 2.52 mmol) and 3-(trifluoromethyl)bicyclo[1.1.1]pentane-1-carboxamide (570 mg, 3.18 mmol) was added cesium carbonate (2.65 g, 8.13 mmol), then XantPhos Pd G3 (405 mg, 0.427 mmol). The reaction mixture was then capped and added anhydrous Dioxane (10 mL) under an atmosphere of nitrogen. Nitrogen was bubbled through the reaction mixture for 20 seconds, then heated to 85° C. in the hood, followed by LC/MS. After 20 minutes the reaction mixture was suspended in EtOAc and diluted with water then washed with saturated NaHCO₃, then brine. The organic layer was then dried over Na₂SO₄, filtered and concentrated. The resulting residue was then purified by silica gel chromatography (0-80% EtOAc/Hex; 14 CV; 80 g ISCO). The desired fractions were concentrated to give the title compound. LRMS m/z: (M+H)+ calculated 349.0; found 349.1.

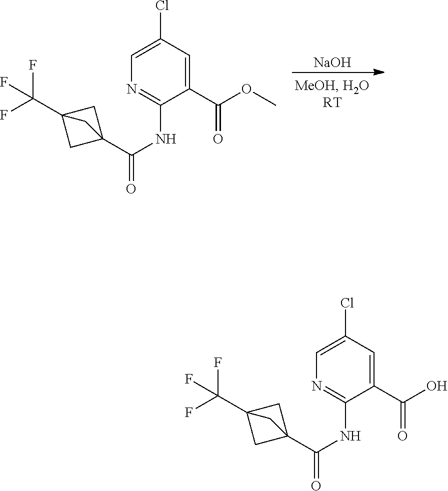 [SMILES: COC(=O)c1cc(Cl)cnc1NC(=O)C12CC(C(F)(F)F)(C1)C2 ; "NaOH" ; O=C(O)c1cc(Cl)cnc1NC(=O)C12CC(C(F)(F)F)(C1)C2]

### Step 2: 5-chloro-2-(3-(trifluoromethyl)bicyclo[1.1.1]pentane-1-carboxamido)nicotinic acid

To a flask containing methyl 5-chloro-2-(3-(trifluoromethyl)bicyclo[1.1.1]pentane-1-carboxamido)nicotinate (634 mg, 1.818 mmol) was added MeOH (6 mL) then water (3 mL) and finally 5N sodium hydroxide (750 μL, 3.75 mmol). The reaction mixture was then capped and stirred at room temperature, followed by LC/MS. After 30 min at room temperature the reaction mixture was diluted/acidified with 1N HCl, then suspended in EtOAc, washed with 1N HCl, then brine. The organic layer was then dried over Na₂SO₄, filtered and concentrated to give the title compound, which was used without further purification. LRMS m/z: (M+H)+ calculated 335.0; found 335.0.

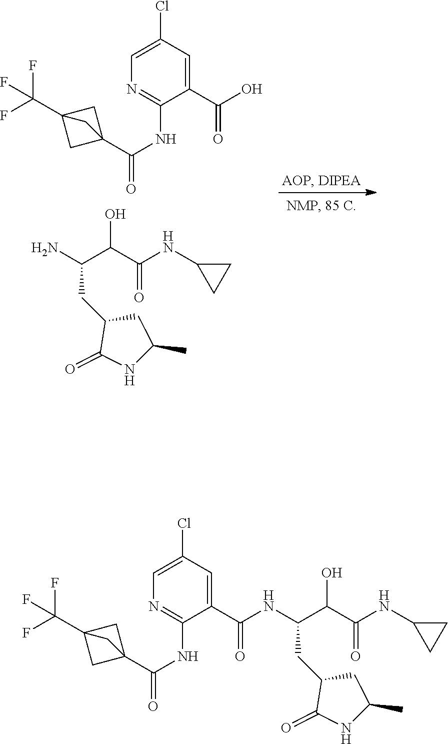

### Step 3: 5-chloro-N-((2S)-4-(cyclopropylamino)-3-hydroxy-1-((3S,5R)-5-methyl-2-oxopyrrolidin-3-yl)-4-oxobutan-2-yl)-2-(3-(trifluoromethyl)bicyclo[1.1.1]pentane-1-carboxamido)nicotinamide

To a vial containing 5-chloro-2-(3-(trifluoromethyl)bicyclo[1.1.1]pentane-1-carboxamido)nicotinic acid (116 mg, 0.347 mmol) and (3S)-3-amino-N-cyclopropyl-2-hydroxy-4-((3R,5R)-5-methyl-2-oxopyrrolidin-3-yl)butanamide (111 mg, 0.435 mmol) was added 7-azabenzotriazol-1-yloxytris(dimethylamino)phosphonium hexafluorophosphate (251 mg, 0.566 mmol) followed by NMP (1.5 mL) and finally DIPEA (150 μL, 0.859 mmol). The reaction mixture was then capped and heated immediately to 85° C. in the hood, followed by LC/MS. After 1 night at 85° C., the reaction mixture was diluted with 200 μL MeOH, then purified by reverse phase chromatography (5-50% MeCN/H2O; 0.1% TFA modifier; 20 min gradient; Waters 30×150 mm Sunfire 5 micron C18 column; Flow=42.5 mL/min). The desired fractions were diluted with MeOH and concentrated, then dissolved in DCM/MeOH and concentrated to give the title compound. LRMS m/z: (M+H)+ calculated 572.1; found 570.2.

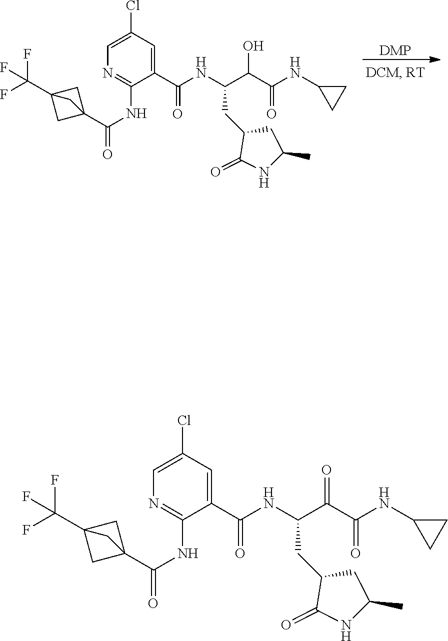

### Step 4: 5-chloro-N—((S)-4-(cyclopropylamino)-1-((3S,5R)-5-methyl-2-oxopyrrolidin-3-yl)-3,4-dioxobutan-2-yl)-2-(3-(trifluoromethyl)bicyclo[1.1.1]pentane-1-carboxamido)nicotinamide

To a vial containing 5-chloro-N-((2S)-4-(cyclopropylamino)-3-hydroxy-1-((3S,5R)-5-methyl-2-oxopyrrolidin-3-yl)-4-oxobutan-2-yl)-2-(3-(trifluoromethyl)bicyclo[1.1.1]pentane-1-carboxamido)nicotinamide (114 mg, 0.199 mmol) was added Dess-Martin Periodinane (168 mg, 0.396 mmol) followed by sodium bicarbonate (73 mg, 0.869 mmol) and finally DCM (5 mL). The reaction mixture was then capped and stirred at room temperature, followed by LC/MS. After 30 min the reaction mixture was quenched/diluted with 4 mL of saturated sodium thiosulfate followed by 1 mL water, then diluted with 10 mL EtOAc. Stirred for 10 minutes then suspended in EtOAc, washed with saturated sodium thiosulfate, then water, then brine. The organic layer was then dried over Na₂SO₄, filtered and concentrated. The resulting residue was then purified by silica gel chromatography (30-100% EtOAc/Hex; 14 CV; 40 g ISCO). The desired fractions were concentrated to give the title compound. LRMS m/z: (M+H)+ calculated 570.9; found 570.1.

¹H NMR (500 MHz, DMSO-d6) δ 10.91 (s, 1H), 9.29 (d, J=7.1 Hz, 1H), 8.80 (d, J=5.0 Hz, 1H), 8.58 (d, J=2.5 Hz, 1H), 8.10 (d, J=2.5 Hz, 1H), 7.83 (s, 1H), 5.13-5.05 (m, 1H), 3.69-3.56 (m, 1H), 2.79-2.65 (m, 2H), 2.26 (s, 6H), 2.03-1.86 (m, 3H), 1.74-1.62 (m, 1H), 1.10 (d, J=6.3 Hz, 3H), 0.69-0.63 (m, 2H), 0.60-0.52 (m, 2H).

### Example 6

### isopropyl (4-chloro-2-(((S)-4-(cyclopropylamino)-1-((3S,5R)-5-methyl-2-oxopyrrolidin-3-yl)-3,4-dioxobutan-2-yl)carbamoyl)phenyl)carbamate

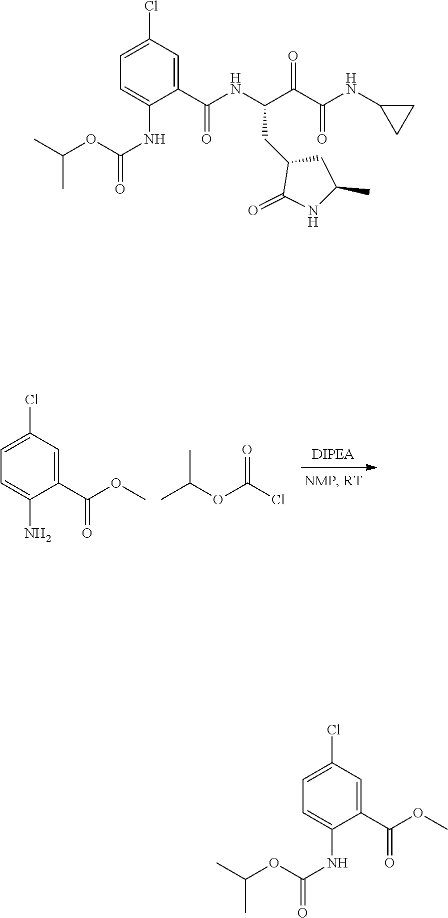

### Step 1: methyl 5-chloro-2-((isopropoxycarbonyl)amino)benzoate

To a vial containing methyl 2-amino-5-chlorobenzoate (418 mg, 2.252 mmol) was added NMP (5 mL) followed by DIPEA (0.8 mL, 4.58 mmol) and finally isopropyl chloroformate (2 mL, 4.00 mmol). The reaction mixture was then capped and stirred at room temperature, followed by LC/MS. The reaction mixture was then diluted/quenched with 400 μL MeOH then partially concentrated. The resulting residue was purified by reverse phase chromatography (25-100% MeCN/H2O; 0.1% TFA modifier; 30 min gradient; Waters 50×250 mm Sunfire 5 micron C18 column; Flow=118.1 mL/min). The desired fractions were diluted with MeOH and concentrated, then dissolved in MeOH/DCM and concentrated to give the title compound. LRMS m/z: (M+H)+ calculated 272.0; found 272.0.

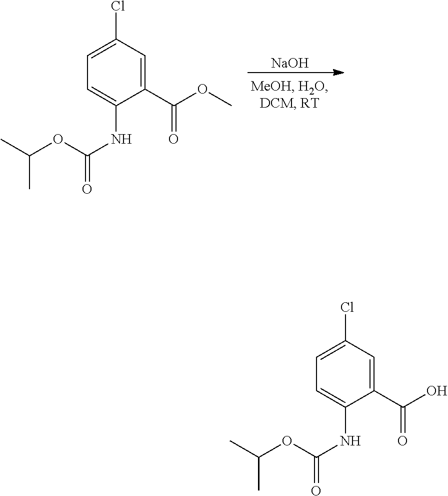 [SMILES: COC(=O)c1cc(Cl)ccc1NC(=O)OC(C)C ; "NaOH" ; CC(C)OC(=O)Nc1ccc(Cl)cc1C(=O)O]

### Step 2: 5-chloro-2-((isopropoxycarbonyl)amino)benzoic acid

To a flask containing methyl 5-chloro-2-((isopropoxycarbonyl)amino)benzoate (455 mg, 1.675 mmol) was added MeOH (8 mL) followed by water (4 mL) and finally 5N sodium hydroxide (0.7 mL, 3.50 mmol). The reaction mixture was then capped and stirred at room temperature. After added the NaOH the reaction mixture was still a suspension so added DCM (3 mL) which immediately solubilized the mixture, followed by LC/MS. After 3.5 hrs at room temperature the reaction mixture was diluted/acidified with 1N HCl, then suspended in EtOAc, washed with 1N HCl, then brine. The organic layer was then dried over Na₂SO₄, filtered and concentrated to give the title compound, which was used without further purification. LRMS m/z: (M+H)+ calculated 258.0; found 258.0.

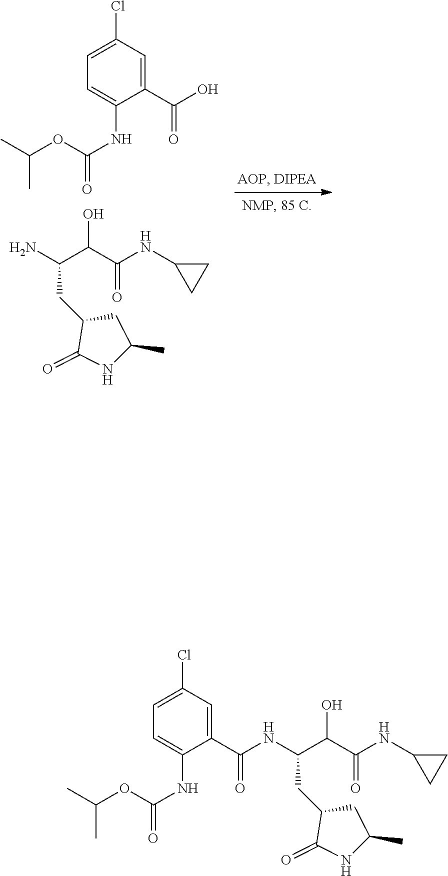

### Step 3: isopropyl (4-chloro-2-(((2S)-4-(cyclopropylamino)-3-hydroxy-1-((3S,5R)-5-methyl-2-oxopyrrolidin-3-yl)-4-oxobutan-2-yl)carbamoyl)phenyl)carbamate

To a vial containing 5-chloro-2-((isopropoxycarbonyl)amino)benzoic acid (81 mg, 0.314 mmol) and (3S)-3-amino-N-cyclopropyl-2-hydroxy-4-((3R,5R)-5-methyl-2-oxopyrrolidin-3-yl)butanamide (101 mg, 0.396 mmol) was added 7-azabenzotriazol-1-yloxytris(dimethylamino)phosphonium hexafluorophosphate (221 mg, 0.499 mmol) followed by NMP (1 mL) and finally DIPEA (145 μL, 0.830 mmol). The reaction mixture was then capped and heated immediately to 85° C. in the hood, followed by LC/MS. After 1 night at 85° C., the reaction mixture was diluted with 200 μL MeOH, then purified by reverse phase chromatography (5-50% MeCN/H2O; 0.1% TFA modifier; 20 min gradient; Waters 30×150 mm Sunfire 5 micron C18 column; Flow=42.5 mL/min). The desired fractions were concentrated, then dissolved in DCM/MeOH and concentrated to give the title compound. LRMS m/z: (M+H)+ calculated 495.1; found 495.3

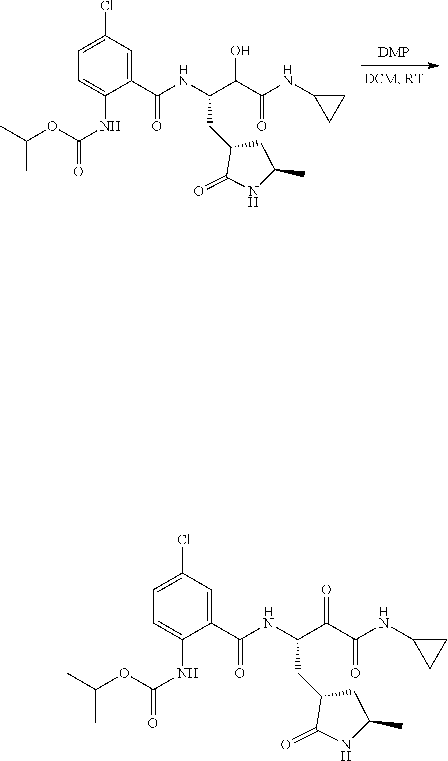 [SMILES: CC(C)OC(=O)Nc1ccc(Cl)cc1C(=O)N[C@@H](C[C@@H]1C[C@@H](C)NC1=O)C(O)C(=O)NC1CC1 ; CC(C)OC(=O)Nc1ccc(Cl)cc1C(=O)N[C@@H](C[C@@H]1C[C@@H](C)NC1=O)C(=O)C(=O)NC1CC1]

### Step 4: isopropyl (4-chloro-2-(((S)-4-(cyclopropylamino)-1-((3S,5R)-5-methyl-2-oxopyrrolidin-3-yl)-3,4-dioxobutan-2-yl)carbamoyl)phenyl)carbamate

To a vial containing isopropyl (4-chloro-2-(((2S)-4-(cyclopropylamino)-3-hydroxy-1-((3S,5R)-5-methyl-2-oxopyrrolidin-3-yl)-4-oxobutan-2-yl)carbamoyl)phenyl)carbamate (113 mg, 0.228 mmol) was added Dess-Martin Periodinane (190 mg, 0.448 mmol), then sodium bicarbonate (45 mg, 0.536 mmol) and finally DCM (5 mL). The reaction mixture was then capped and stirred at room temperature, followed by LC/MS. After 1 hr the reaction mixture was quenched/diluted with 4 mL of saturated sodium thiosulfate followed by 1 mL water, then diluted with 10 mL EtOAc. Stirred for 25 minutes then suspended in EtOAc, washed with saturated sodium thiosulfate, then water, then brine. The organic layer was then dried over Na₂SO₄, filtered and concentrated. The resulting residue was purified by silica gel chromatography (0-100% EtOAc/Hex; 14 CV; 40 g ISCO). The desired fractions were concentrated to give the title compound. LRMS m/z: (M+H)+ calculated 493.9; found 493.1.

¹H NMR (500 MHz, DMSO-d6) δ 10.56 (s, 1H), 9.66 (d, J=5.9 Hz, 1H), 8.82 (d, J=4.8 Hz, 1H), 8.25 (d, J=9.0 Hz, 1H), 8.01-7.88 (m, 2H), 7.60 (d, J=9.0 Hz, 1H), 5.16-5.05 (m, 1H), 4.92-4.83 (m, 1H), 3.68-3.57 (m, 1H), 2.81-2.72 (m, 1H), 2.71-2.61 (m, 1H), 2.05-1.94 (m, 2H), 1.93-1.85 (m, 1H), 1.81-1.73 (m, 1H), 1.24 (d, J=6.2 Hz, 6H), 1.10 (d, J=6.2 Hz, 3H), 0.69-0.63-(m, 2H), 0.60-0.54 (m, 2H).

### Example 7

### 5-chloro-N—((S)-4-(cyclopropylamino)-1-((3S,5R)-5-methyl-2-oxopyrrolidin-3-yl)-3,4-dioxobutan-2-yl)-2-(2,2-difluoro-3-phenylpropanamido)benzamide

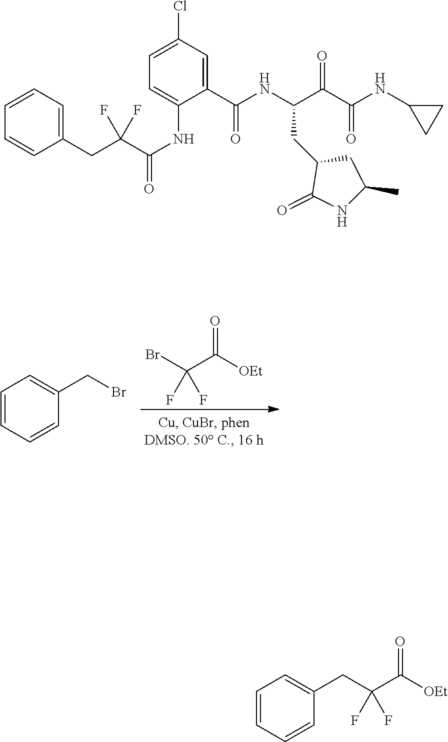 [SMILES: C[C@@H]1C[C@@H](C[C@H](NC(=O)c2cc(Cl)ccc2NC(=O)C(F)(F)Cc2ccccc2)C(=O)C(=O)NC2CC2)C(=O)N1 ; BrCc1ccccc1 ; CCOC(=O)C(F)(F)Br ; CCOC(=O)C(F)(F)Cc1ccccc1]

### Step 1: ethyl 2,2-difluoro-3-phenylpropanoate

To a solution of (bromomethyl)benzene (6.92 mL, 58.5 mmol) and ethyl 2-bromo-2,2-difluoroacetate (8.31 g, 40.9 mmol) in DMSO (10 mL) was added copper (8.55 g, 134 mmol), copper(I) bromide (0.419 g, 2.92 mmol) and 1,10-phenanthroline (2.107 g, 11.69 mmol) under an atmosphere of nitrogen. The reaction mixture was then degassed and backfilled with nitrogen (3×). The resulting mixture was stirred under nitrogen at 50° C. for 16 h, followed by LC/MS. Then the reaction mixture was filtered. The filtrate was quenched with brine (20 mL) and extracted with EtOAc (20 mL×3). The combined organic layers were dried over anhydrous Na₂SO₄, filtered and concentrated under reduced pressure. The resulting residue was purified by flash silica gel chromatography (ISCO®; 80 g SepaFlash® Silica Flash Column, eluent of 0-10% ethyl acetate/pet. ether, Gradient: @ 100 mL/min). The desired fractions were concentrated to give the title compound.

¹H NMR (400 MHz, MeOD) δ 7.32-7.27 (m, 5H), 4.22 (q, J=7.2 Hz, 2H), 3.40 (t, J=16.8 Hz, 2H), 1.22 (t, J=7.2 Hz, 3H).

¹⁹F NMR (376 MHz, MeOD) δ 106.1.

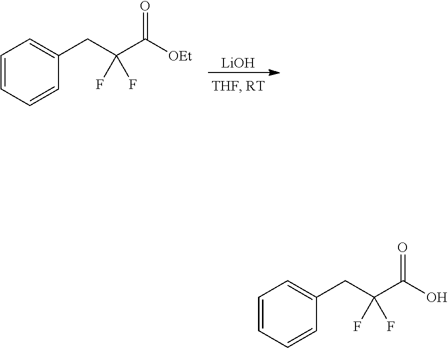

### Step 2: benzyl 2,2-difluoro-3-phenylpropanoic acid

To a solution of ethyl 2,2-difluoro-3-phenylpropanoate (4.5 g, 21.01 mmol) in THF (30 mL) and water (15 mL) was added lithium hydroxide monohydrate (5.29 g, 126 mmol). The reaction mixture was stirred at 25° C. for 24 h, followed by LC/MS. The reaction mixture was then diluted with water (30 mL) and extracted with EtOAc (30 mL×3). The aqueous phase was acidized to pH=3-4 by 1 N HCl. The resulting suspension was extracted with DCM (20 mL×2). The combined organic layers were dried over anhydrous Na₂SO₄, filtered and concentrated to give the title compound, which was used without further purification.

¹H NMR (400 MHz, MeOD) δ 7.32-7.27 (m, 5H), 3.38 (t, J=16.8 Hz, 2H).

¹⁹F NMR (376 MHz, MeOD) δ 106.1.

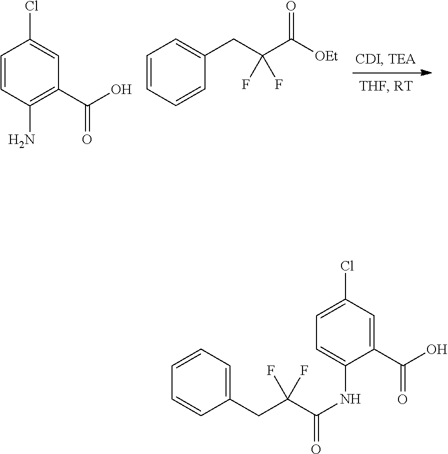

### Step 3: 5-chloro-2-(2,2-difluoro-3-phenylpropanamido)benzoic acid

To a solution of 2,2-difluoro-3-phenylpropanoic acid (150 mg, 0.806 mmol) in THF (2 mL) were added CDI (392 mg, 2.417 mmol) and TEA (0.337 mL, 2.417 mmol) at 25° C. The reaction mixture was stirred at 25° C. for 2 h then 2-amino-5-chlorobenzoic acid (152 mg, 0.886 mmol) was added to the reaction. The mixture was then stirred at 25° C. for 16 h, followed by LC/MS. Then 1 M HCl (0.5 mL) was added to the mixture which was then concentrated under reduced pressure. The resulting residue was purified by RP-HPLC (Column: Boston Green ODS 150×30 mm×5 m, Condition: water (TFA)-ACN Begin B 50 End B 70, Gradient: Time (min) 10 100% B Hold Time (min) 2 Flow Rate (mL/min) 25). The desired fractions were concentrated to give the title compound. LRMS m/z: (M+H)+ calculated 340.0; found 340.0.

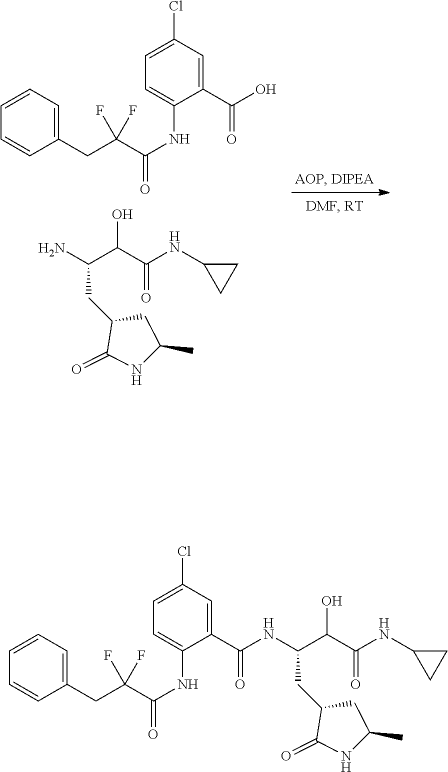

### Step 4: 5-chloro-N-((2 S)-4-(cyclopropylamino)-3-hydroxy-1-((3 S, 5R)-5-methyl-2-oxopyrrolidin-3-yl)-4-oxobutan-2-yl)-2-(2,2-difluoro-3-phenylpropanamido)benzamide

To a mixture of 5-chloro-2-(2,2-difluoro-3-phenylpropanamido)benzoic acid (35 mg, 0.103 mmol) and (3S)-3-amino-N-cyclopropyl-2-hydroxy-4-((3R,5R)-5-methyl-2-oxopyrrolidin-3-yl)butanamide (31.6 mg, 0.124 mmol) in DMF (3 mL) was added AOP (91 mg, 0.206 mmol) and DIPEA (0.054 mL, 0.309 mmol) at 25° C. The resulting reaction mixture was stirred then stirred at 25° C. for 16 h, followed by LC/MS. Water (0.5 mL) was then added to the mixture, then purified by RP-HPLC (Column: Boston Green ODS 150×30 mm×5 m, Condition: water (TFA)-ACN Begin B 38 End B 68, Gradient: Time (min) 10 100% B Hold Time (min) 2 Flow Rate (mL/min) 25). The desired fractions were concentrated to give the title compound. LRMS m/z: (M+H)+ calculated 577.2; found 577.2.

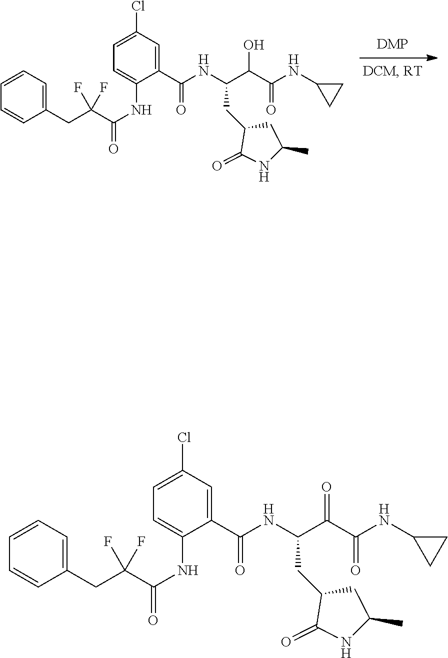 [SMILES: C[C@@H]1C[C@@H](C[C@H](NC(=O)c2cc(Cl)ccc2NC(=O)C(F)(F)Cc2ccccc2)C(O)C(=O)NC2CC2)C(=O)N1 ; C[C@@H]1C[C@@H](C[C@H](NC(=O)c2cc(Cl)ccc2NC(=O)C(F)(F)Cc2ccccc2)C(=O)C(=O)NC2CC2)C(=O)N1]

### Step 5: 5-chloro-N—((S)-4-(cyclopropylamino)-1-((3S,5R)-5-methyl-2-oxopyrrolidin-3-yl)-3,4-dioxobutan-2-yl)-2-(2,2-difluoro-3-phenylpropanamido)benzamide

To a solution of 5-chloro-N-((2S)-4-(cyclopropylamino)-3-hydroxy-1-((3S,5R)-5-methyl-2-oxopyrrolidin-3-yl)-4-oxobutan-2-yl)-2-(2,2-difluoro-3-phenylpropanamido)benzamide (45 mg, 0.078 mmol) in DCM (3 mL) was added NaHCO₃ (19.89 mg, 0.234 mmol) and DMP (99 mg, 0.234 mmol) at 25° C. The reaction mixture was stirred for 1 h at 25° C., followed by LC/MS. Then the reaction mixture was filtered and the filtrate was concentrated under reduced pressure. The resulting residue was purified by RP-HPLC (Column: Boston Green ODS 150×30 mm×5 m, Condition: water (TFA)-ACN Begin B 45 End B 75, Gradient: Time (min) 10 100% B Hold Time (min) 2 Flow Rate (mL/min) 25) to give the title compound. LRMS m/z: (M+H)+ calculated 575.1; found 575.2.

¹H NMR (400 MHz, MeOD) δ 8.47-8.37 (m, 1H), 7.92-7.71 (m, 1H), 7.57-7.50 (m, 1H), 7.31-7.22 (m, 5H), 4.48-4.31 (m, 1H), 3.87-3.68 (m, 1H), 3.47 (dt, J=9.1, 16.9 Hz, 2H), 3.00-2.69 (m, 1H), 2.69-2.48 (m, 1H), 2.41-2.18 (m, 1H), 2.14-1.89 (m, 3H), 1.24-1.11 (m, 3H), 0.83-0.65 (m, 2H), 0.64-0.40 (m, 2H).

### Example 8

### 5-chloro-N—((S)-4-(cyclopropylamino)-1-((3S,5R)-5-methyl-2-oxopyrrolidin-3-yl)-3,4-dioxobutan-2-yl)-2-((R)-2,2-difluorocyclopropane-1-carboxamido)benzamide

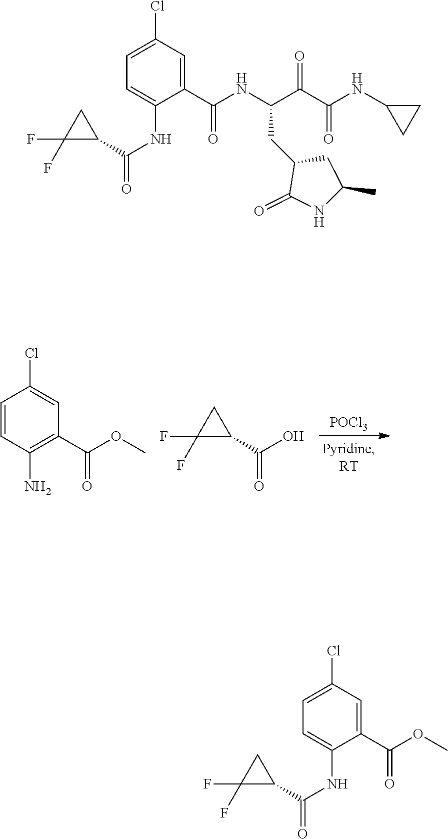

### Step 1: methyl (R)-5-chloro-2-(2,2-difluorocyclopropane-1-carboxamido)benzoate

To a vial containing methyl 2-amino-5-chlorobenzoate (367 mg, 1.977 mmol) and (1R)-2,2-difluorocyclopropane-1-carboxylic acid (313 mg, 2.56 mmol) was added Pyridine (6 mL) and finally POCl3 (0.297 mL, 3.18 mmol). The reaction mixture was then capped and stirred at room temperature, followed by LC/MS. After 20 min at room temperature, the reaction mixture was diluted with MeOH then purified by reverse phase chromatography (10-100% MeCN/H2O; 0.1% TFA modifier; 30 min gradient; Waters 50×250 mm Sunfire 5 micron C18 column; Flow=118.1 mL/min). The desired fractions were concentrated to give the title compound. LRMS m/z: (M+H)+ calculated 290.0; found 290.1.

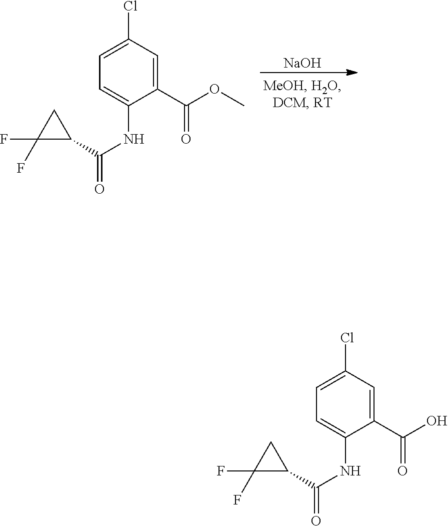 [SMILES: COC(=O)c1cc(Cl)ccc1NC(=O)[C@H]1CC1(F)F ; "NaOH" ; O=C(O)c1cc(Cl)ccc1NC(=O)[C@H]1CC1(F)F]

### Step 2: (R)-5-chloro-2-(2,2-difluorocyclopropane-1-carboxamido)benzoic acid

To a flask containing methyl (R)-5-chloro-2-(2,2-difluorocyclopropane-1-carboxamido)benzoate (450 mg, 1.554 mmol) was added MeOH (10 mL) then Water (4 mL) and finally 5N sodium hydroxide (800 μL, 4.00 mmol). The reaction mixture was then capped and stirred at room temperature. DCM (3 mL) was then added which immediately solubilized the mixture, followed by LC/MS. After 75 min at room temperature the reaction mixture was diluted with 1N HCl, then suspended in EtOAc, washed with 1N HCl, then brine. The organic layer was then dried over Na₂SO₄, filtered and concentrated to give the title compound, which was used without further purification. LRMS m/z: (M+H)+ calculated 276.0; found 276.0

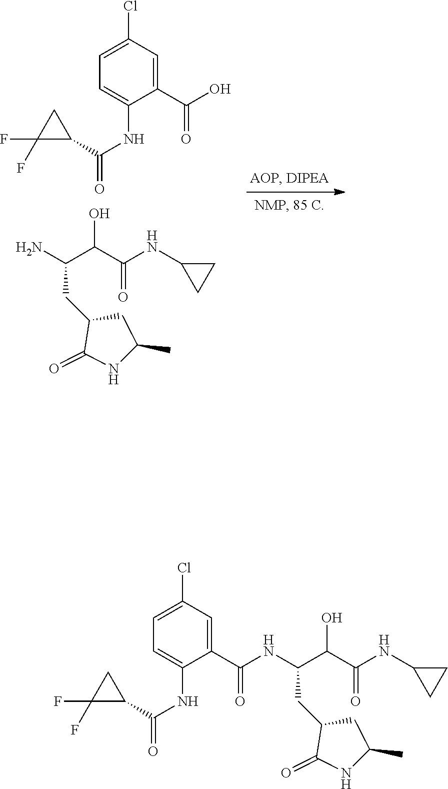

### Step 3: 5-chloro-N-((2S)-4-(cyclopropylamino)-3-hydroxy-1-((3S,5R)-5-methyl-2-oxopyrrolidin-3-yl)-4-oxobutan-2-yl)-2-((R)-2,2-difluorocyclopropane-1-carboxamido)benzamide

To a vial containing (R)-5-chloro-2-(2,2-difluorocyclopropane-1-carboxamido)benzoic acid (71.5 mg, 0.259 mmol) and (3S)-3-amino-N-cyclopropyl-2-hydroxy-4-((3R,5R)-5-methyl-2-oxopyrrolidin-3-yl)butanamide (71 mg, 0.278 mmol) was added 7-azabenzotriazol-1-yloxytris(dimethylamino)phosphonium hexafluorophosphate (190 mg, 0.429 mmol) followed by NMP (1 mL) and finally DIPEA (115 μl, 0.658 mmol). The reaction mixture was then capped and heated to 85° C. in the hood, followed by LC/MS. After 1 night at 85° C., the reaction mixture was diluted with 200 μL MeOH, then purified by reverse phase chromatography (10-70% MeCN/H2O; 0.1% TFA modifier; 20 min gradient; Waters 30×150 mm Sunfire 5 micron C18 column; Flow=42.5 mL/min). The desired fractions were diluted with MeOH and concentrated to give the title compound. LRMS m/z: (M+H)+ calculated 513.2; found 513.1.

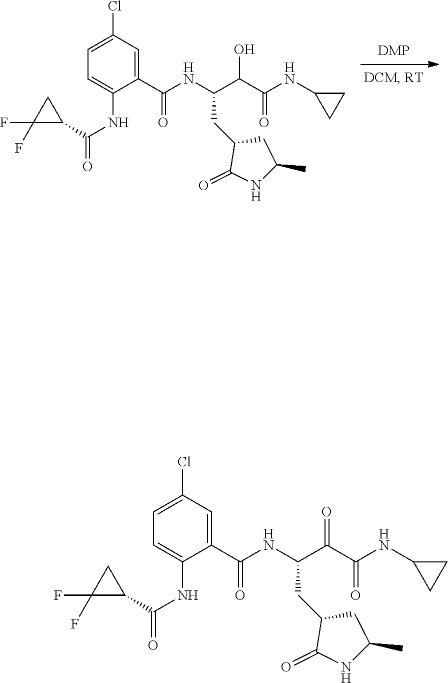 [SMILES: C[C@@H]1C[C@@H](C[C@H](NC(=O)c2cc(Cl)ccc2NC(=O)[C@H]2CC2(F)F)C(O)C(=O)NC2CC2)C(=O)N1 ; C[C@@H]1C[C@@H](C[C@H](NC(=O)c2cc(Cl)ccc2NC(=O)[C@H]2CC2(F)F)C(=O)C(=O)NC2CC2)C(=O)N1]

### Step 4: 5-chloro-N—((S)-4-(cyclopropylamino)-1-((3S,5R)-5-methyl-2-oxopyrrolidin-3-yl)-3,4-dioxobutan-2-yl)-2-((R)-2,2-difluorocyclopropane-1-carboxamido)benzamide

To a vial containing 5-chloro-N-((2S)-4-(cyclopropylamino)-3-hydroxy-1-((3S,5R)-5-methyl-2-oxopyrrolidin-3-yl)-4-oxobutan-2-yl)-2-((R)-2,2-difluorocyclopropane-1-carboxamido)benzamide (70 mg, 0.136 mmol) was added Dess-Martin Periodinane (94 mg, 0.222 mmol), sodium bicarbonate (23 mg, 0.274 mmol) and finally DCM (5 mL). The reaction mixture was then capped and stirred at room temperature, followed by LC/MS. After 60 min the reaction mixture was quenched/diluted with 4 mL of saturated sodium thiosulfate followed by 1 mL water, then diluted with 10 mL EtOAc. Stirred for 10 minutes then suspended in EtOAc, washed with saturated sodium thiosulfate, then water, then brine. The organic layer was then dried over Na₂SO₄, filtered and concentrated. The resulting residue was then purified by silica gel chromatography (0-100% EtOAc/Hex; 14 CV; 40 g ISCO). The desired fractions were concentrated to give the title compound. LRMS m/z: (M+H)+ calculated 511.2; found 511.1.

¹H NMR (500 MHz, DMSO-d6) δ 11.09 (s, 1H), 9.57 (d, J=6.4 Hz, 1H), 8.83 (d, J=5.0 Hz, 1H), 8.25 (d, J=8.9 Hz, 1H), 7.91 (s, 1H), 7.85 (d, J=2.4 Hz, 1H), 7.60 (dd, J=8.9, 2.4 Hz, 1H), 5.18-5.10 (m, 1H), 3.70-3.56 (m, 1H), 3.05-2.89 (m, 1H), 2.80-2.72 (m, 1H), 2.69-2.59 (m, 1H), 2.05-1.92 (m, 3H), 1.94-1.85 (m, 1H), 1.79-1.71 (m, 1H) 1.09 (d, J=6.3 Hz, 3H), 0.71-0.62 (m, 2H), 0.60-0.53 (m, 2H).

### Example 9

### methyl (4-chloro-2-(((S)-1-((3S,5R)-5-methyl-2-oxopyrrolidin-3-yl)-4-(methylamino)-3,4-dioxobutan-2-yl)carbamoyl)phenyl)carbamate

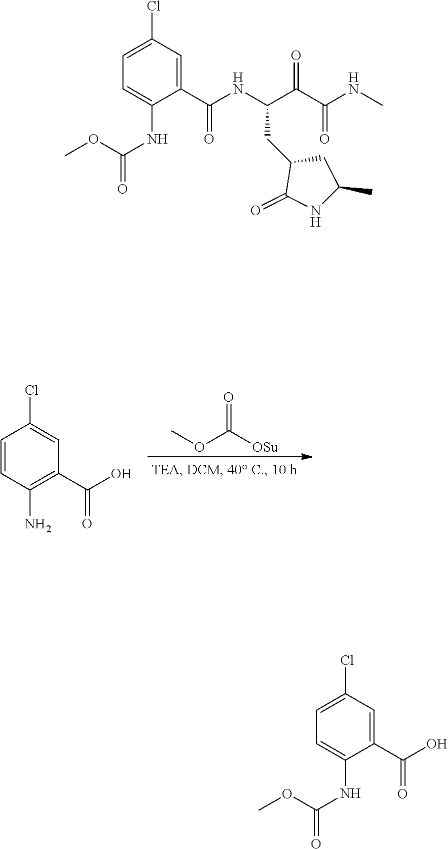

### Step 1: 5-chloro-2-((methoxycarbonyl)amino)benzoic acid

To a solution of 2,5-dioxopyrrolidin-1-yl methyl carbonate (133 mg, 0.769 mmol) and TEA (0.268 mL, 1.923 mmol) in DCM (3 mL) was added 2,5-dioxopyrrolidin-1-yl methyl carbonate (133 mg, 0.769 mmol) at 20° C. then the reaction mixture was heated to 40° C. and stirred for 10 h, followed by LC/MS. Then the reaction mixture was concentrated and the residue was purified by RP-HPLC (Column: Boston Uni C18 150*40 mm*5 m. Condition: water (0.01% TFA)-ACN Begin B 34 End B 64 Gradient Time (min) 10 100% B Hold Time 2. Flow Rate (mL/min): 60) to give the title compound. LRMS m/z: (M+H)⁺ calculated 230.0; found 230.0.

¹H NMR (400 MHz, DMSO-d6) δ 14.07 (br s, 1H), 10.70 (br s, 1H), 8.29 (d, J=9.1 Hz, 1H), 7.92 (d, J=2.6 Hz, 1H), 7.69 (dd, J=2.6, 9.1 Hz, 1H), 3.72 (s, 3H).

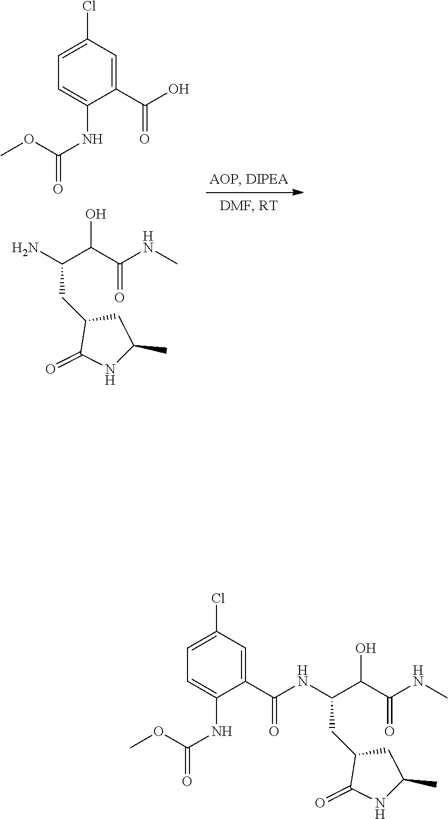

### Step 2: methyl (4-chloro-2-(((2S)-3-hydroxy-1-((3S,5R)-5-methyl-2-oxopyrrolidin-3-yl)-4-(methylamino)-4-oxobutan-2-yl)carbamoyl)phenyl)carbamate

A mixture of 5-chloro-2-((methoxycarbonyl)amino)benzoic acid (70 mg, 0.305 mmol), AOP (176 mg, 0.396 mmol), DIPEA (0.160 mL, 0.915 mmol), (3S)-3-amino-2-hydroxy-N-methyl-4-((3R,5R)-5-methyl-2-oxopyrrolidin-3-yl)butanamide (70 mg, 0.305 mmol) and DMF (3 mL) was stirred at 25° C. for 2 h, followed by LC/MS. Then water (0.2 mL) was added to the reaction mixture and it was purified directly by RP-HPLC (Column: Boston Uni C18 150*40 mm*5 m. Condition: water (0.01% TFA)-ACN Begin B 14 End B 44 Gradient Time (min) 10 100% B Hold Time 2. Flow Rate (mL/min): 60) to give the title compound. LRMS m/z: (M+H)+ calculated 441.1; found 441.2.

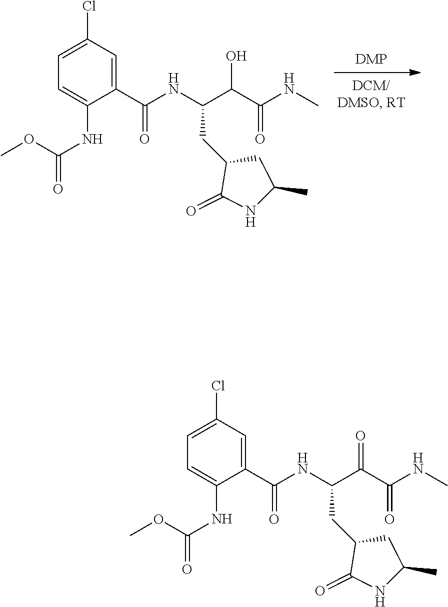

### Step 3: methyl (4-chloro-2-(((S)-1-((3S,5R)-5-methyl-2-oxopyrrolidin-3-yl)-4-(methylamino)-3,4-dioxobutan-2-yl)carbamoyl)phenyl)carbamate

A mixture of methyl (4-chloro-2-(((2S)-3-hydroxy-1-((3S,5R)-5-methyl-2-oxopyrrolidin-3-yl)-4-(methylamino)-4-oxobutan-2-yl)carbamoyl)phenyl)carbamate (60 mg, 0.136 mmol), NaHCO₃ (34.3 mg, 0.408 mmol) and DMP (115 mg, 0.272 mmol) in DCM/DMSO=3:1 (4 mL) was stirred at 20° C. for 1 h, followed by LC/MS. The reaction mixture was then filtered and the filtrate was concentrated under reduced pressure. The resulting residue was purified by RP-HPLC (Column: Waters Xbridge BEH C18 100*40 mm*10 μm. Condition: water (10 mM HCOONH4)-ACN Begin B 22 End B 52 Gradient Time (min) 11 100% B Hold Time 3. Flow Rate (mL/min): 50) to give the title compound. LRMS m/z: (M+H)⁺ calculated 439.1; found 439.2. 1H NMR (400 MHz, METHANOL-d4) δ 8.26-8.11 (m, 1H), 7.86-7.60 (m, 1H), 7.53-7.44 (m, 1H), 4.55-4.35 (m, 1H), 3.84-3.70 (m, 4H), 2.87-2.75 (m, 3H), 2.57-2.37 (m, 1H), 2.18-1.93 (m, 4H), 1.87-1.74 (m, 1H), 1.37-1.15 (m, 3H).

### Example 10

### cyclopropyl (4-chloro-2-(((S)-1-((3S,5R)-5-methyl-2-oxopyrrolidin-3-yl)-4-(methylamino)-3,4-dioxobutan-2-yl)carbamoyl)phenyl)carbamate

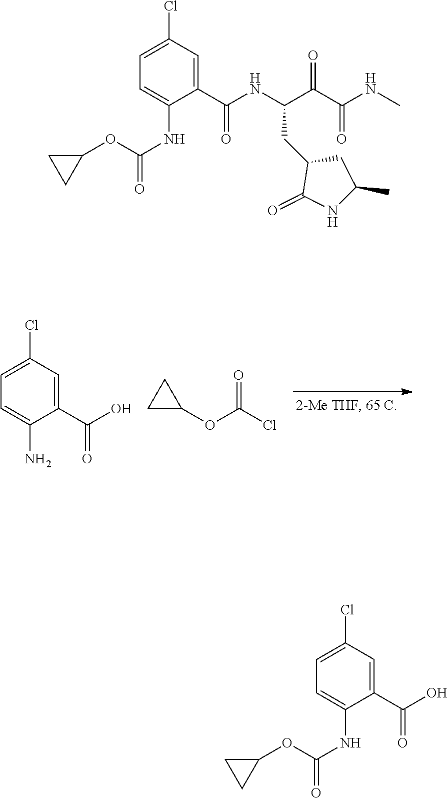

### Step 1: 5-chloro-2-((cyclopropoxycarbonyl)amino)benzoic acid

To a vial containing 2-amino-5-chlorobenzoic acid (2.21 g, 12.88 mmol) was added 2-MeTHF (40 mL) followed by cyclopropyl chloroformate (1.92 g, 15.93 mmol). The reaction mixture was then capped and stirred at 65° C., followed by LC/MS. After 1 night at 65° C. the reaction mixture was diluted/quenched with −200 mL of water, then suspended in EtOAc, and washed with 1N HCl, then brine. The organic layer was then dried over anhydrous sodium sulfate, filtered and concentrated. The resulting residue was dissolved in DCM then purified by silica gel chromatography (0-30% IPA/DCM, 14 CV, 120 g ISCO). The desired fractions were concentrated to give the title compound. LRMS m/z: (M+H)⁺ calculated 256.0; found 256.1.

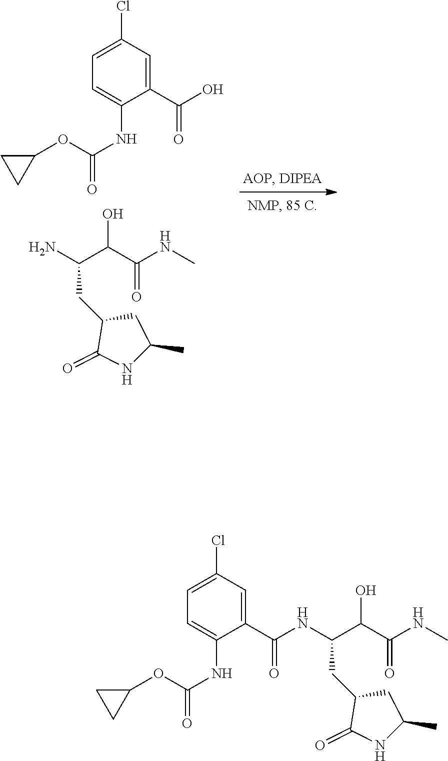

### Step 2: cyclopropyl (4-chloro-2-(((2S)-3-hydroxy-1-((3S,5R)-5-methyl-2-oxopyrrolidin-3-yl)-4-(methylamino)-4-oxobutan-2-yl)carbamoyl)phenyl)carbamate

To a vial containing 5-chloro-2-((cyclopropoxycarbonyl)amino)benzoic acid (75 mg, 0.293 mmol) and (3S)-3-amino-2-hydroxy-N-methyl-4-((3R,5R)-5-methyl-2-oxopyrrolidin-3-yl)butanamide (77 mg, 0.336 mmol) was added 7-azabenzotriazol-1-yloxytris(dimethylamino)phosphonium hexafluorophosphate (204 mg, 0.460 mmol), followed by NMP (1.5 mL) and finally DIPEA (135 μL, 0.773 mmol). The reaction mixture was then capped and heated to 85° C., followed by LC/MS. After 1 night at 85° C., the reaction mixture was diluted with 200 μL MeOH, filtered (syringe filter) and then the filtrate was purified (without workup) by reverse phase chromatography (5-60% MeCN/H2O; 0.1% TFA modifier; 20 min gradient; Waters 30×150 mm Sunfire 5 micron C18 column; Flow=42.5 mL/min). The desired fractions were concentrated, then dissolved in DCM/MeOH and concentrated to give the title compound. LRMS m/z: (M+H)⁺ calculated 467.1; found 467.4.

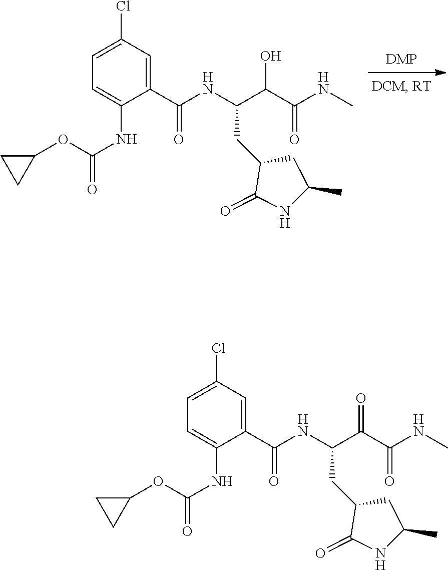 [SMILES: CNC(=O)C(O)[C@H](C[C@@H]1C[C@@H](C)NC1=O)NC(=O)c1cc(Cl)ccc1NC(=O)OC1CC1 ; CNC(=O)C(=O)[C@H](C[C@@H]1C[C@@H](C)NC1=O)NC(=O)c1cc(Cl)ccc1NC(=O)OC1CC1]

### Step 3: cyclopropyl (4-chloro-2-(((S)-1-((3S,5R)-5-methyl-2-oxopyrrolidin-3-yl)-4-(methylamino)-3,4-dioxobutan-2-yl)carbamoyl)phenyl)carbamate

To a vial containing cyclopropyl (4-chloro-2-(((2S)-3-hydroxy-1-((3S,5R)-5-methyl-2-oxopyrrolidin-3-yl)-4-(methylamino)-4-oxobutan-2-yl)carbamoyl)phenyl)carbamate (103 mg, 0.221 mmol) was added Dess-MartinPeriodinane (172 mg, 0.406 mmol) and sodium bicarbonate (41 mg, 0.488 mmol) and finally DCM (5 mL). The reaction mixture was then capped and stirred at room temperature, followed by LC/MS. After 1.5 hrs the reaction mixture was quenched/diluted with 4 mL of saturated sodium thiosulfate followed by 1 mL water, and then diluted with 10 mL EtOAc. The reaction mixture was stirred for 15 minutes at room temperature, then suspended in EtOAc, and washed with saturated sodium thiosulfate, then water, then brine. The organic layer was then dried over anhydrous sodium sulfate, filtered and concentrated. The resulting residue was dissolved in DCM & purified by silica gel chromatography (30-100% EtOAc/Hex; 14 CV; 40 g ISCO). The desired fractions were concentrated to give the title compound. LRMS m/z: (M+H)⁺ calculated 465.1; found 465.4.

¹H NMR (500 MHz, DMSO-d6) δ 10.60 (s, 1H), 9.67 (d, J=5.9 Hz, 1H), 8.74-8.69 (m, 1H), 8.23 (d, J=9.0 Hz, 1H), 8.02-7.87 (m, 2H), 7.62 (dd, J=9.0, 2.4 Hz, 1H), 5.18-5.10 (m, 1H), 4.12-4.03 (m, 1H), 3.67-3.56 (m, 1H), 2.70-2.64 (m, 4H), 2.03-1.92 (m, 2H), 1.91-1.84 (m, 1H), 1.80-1.72 (m, 1H), 1.09 (d, J=6.4 Hz, 3H), 0.73-0.64 (m, 4H).

### Example 11

### cyclopropyl (4-chloro-2-(((S)-4-(methylamino)-3,4-dioxo-1-((S)-2-oxopyrrolidin-3-yl)butan-2-yl)carbamoyl)phenyl)carbamate

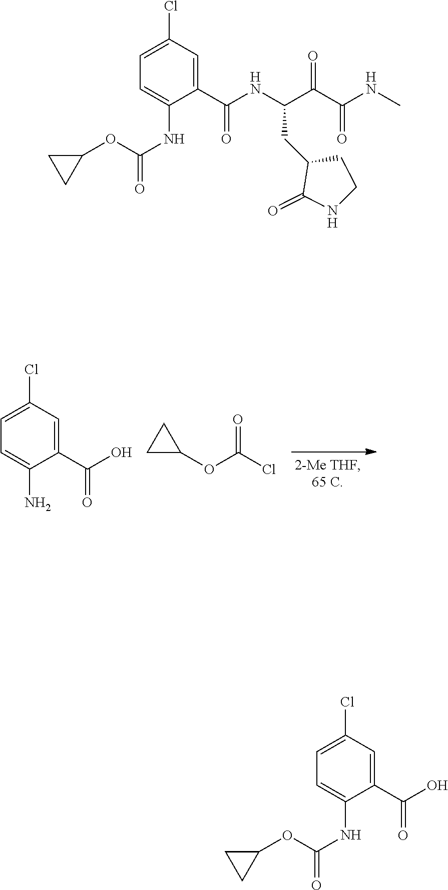

### Step 1: 5-chloro-2-((cyclopropoxycarbonyl)amino)benzoic acid

To a vial containing 2-amino-5-chlorobenzoic acid (2.21 g, 12.88 mmol) was added 2-MeTHF (40 mL) followed by cyclopropyl chloroformate (1.92 g, 15.93 mmol). The reaction mixture was then capped and stirred at 65° C., followed by LC/MS. After 1 night at 65° C. the reaction mixture was diluted/quenched with ˜200 mL of water, then suspended in EtOAc, and washed with 1N HCl, then brine. The organic layer was then dried over anhydrous sodium sulfate, filtered and concentrated. The resulting residue was dissolved in DCM then purified by silica gel chromatography (0-30% IPA/DCM, 14 CV, 120 g ISCO). The desired fractions were concentrated to give the title compound. LRMS m/z: (M+H)+ calculated 256.0; found 256.1.

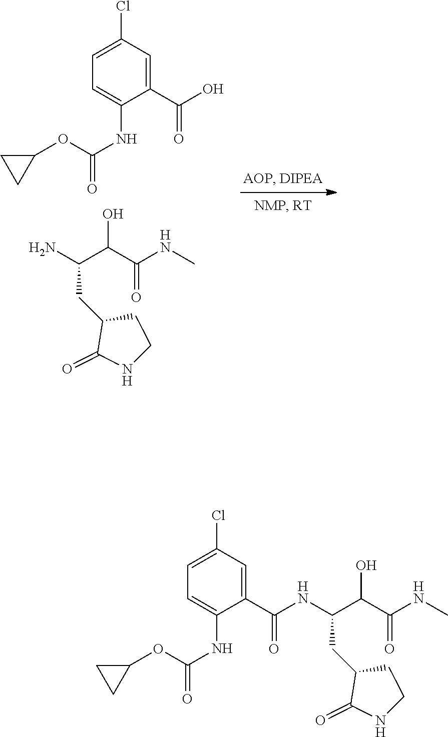

### Step 2: cyclopropyl (4-chloro-2-(((2S)-3-hydroxy-4-(methylamino)-4-oxo-1-((S)-2-oxopyrrolidin-3-yl)butan-2-yl)carbamoyl)phenyl)carbamate

To a vial containing 5-chloro-2-((cyclopropoxycarbonyl)amino)benzoic acid (75 mg, 0.293 mmol) and (3S)-3-amino-2-hydroxy-N-methyl-4-((S)-2-oxopyrrolidin-3-yl)butanamide hydrochloride (94.7 mg, 0.376 mmol) was added 7-azabenzotriazol-1-yloxytris(dimethylamino)phosphonium hexafluorophosphate (206 mg, 0.465 mmol) followed by NMP (1 mL) and finally DIPEA (135 μL, 0.773 mmol). The reaction mixture was then capped and stirred at room temperature in the hood. Followed by LC/MS. After 1 night at room temperature, the reaction mixture was diluted with 200 μL MeOH, filtered (syringe filter) then the filtrate was purified (without workup) by reverse phase chromatography (1×4.2 mL injection) (5-50% MeCN/H2O; 0.1% TFA modifier; 20 min gradient; Waters 30×150 mm Sunfire 5 micron C18 column; Flow=42.5 mL/min). The desired fractions were concentrated to give the title compound. LRMS m/z: (M+H)+ calculated 453.1; found 453.4.

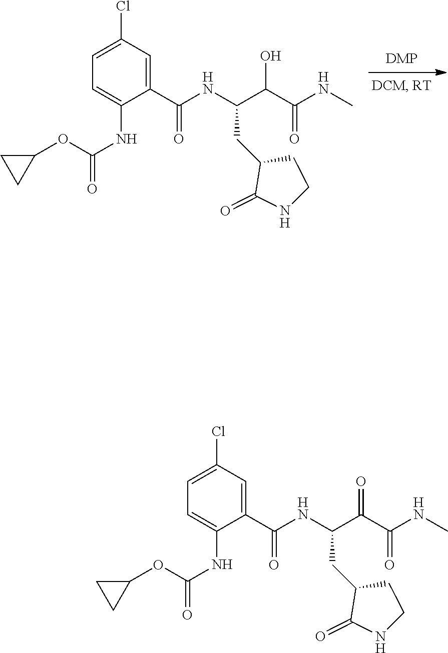 [SMILES: CNC(=O)C(O)[C@H](C[C@@H]1CCNC1=O)NC(=O)c1cc(Cl)ccc1NC(=O)OC1CC1 ; CNC(=O)C(=O)[C@H](C[C@@H]1CCNC1=O)NC(=O)c1cc(Cl)ccc1NC(=O)OC1CC1]

### Step 3: cyclopropyl (4-chloro-2-(((S)-4-(methylamino)-3,4-dioxo-1-((S)-2-oxopyrrolidin-3-yl)butan-2-yl)carbamoyl)phenyl)carbamate

To a flask containing cyclopropyl (4-chloro-2-(((2S)-3-hydroxy-4-(methylamino)-4-oxo-1-((S)-2-oxopyrrolidin-3-yl)butan-2-yl)carbamoyl)phenyl)carbamate (104 mg, 0.230 mmol) was added Dess-MartinPeriodinane (146 mg, 0.344 mmol), sodium bicarbonate (28.9 mg, 0.344 mmol) and finally DCM (5 mL) (from squirt bottle). The reaction mixture was then capped and stirred at room temperature, followed by LC/MS. After 1 hr the reaction mixture was quenched/diluted with 5 mL of saturated sodium thiosulfate followed by 5 mL water, then diluted with 10 mL EtOAc and stirred for 15 minutes at room temperature. The reaction mixture was then suspended in EtOAc, and washed with saturated sodium thiosulfate, then water, then brine. The organic layer was dried over anhydrous sodium sulfate, filtered and the filtrate was concentrated. The resulting residue was dissolved in DCM and purified by silica gel chromatography (50-100% EtOAc/Hex; 14 CV; 40 g ISCO column). The desired fractions were concentrated to give the title compound. LRMS m/z: (M+H)+ calculated 451.1; found 451.3.

¹H NMR (500 MHz, DMSO-d6) δ 10.61 (s, 1H), 9.66 (d, J=6.0 Hz, 1H), 8.76-8.69 (m, 1H), 8.23 (d, J=9.0 Hz, 1H), 7.95 (d, J=2.5 Hz, 1H), 7.85 (s, 1H), 7.62 (dd, J=9.0, 2.4 Hz, 1H), 5.18-5.11 (m, 1H), 4.11-4.04 (m, 1H), 3.24-3.12 (m, 2H), 2.67 (d, J=4.8 Hz, 3H), 2.61-2.52 (m, 1H), 2.30-2.22 (m, 1H), 2.05-1.96 (m, 1H), 1.80-1.71 (m, 2H), 0.74-0.64 (m, 4H).

### Example 105

### 5-chloro-N—((S)-4-(cyclopropylamino)-1-((3S,5R)-5-methyl-2-oxopyrrolidin-3-yl)-3,4-dioxobutan-2-yl)-2-(3-((4,4-difluorocyclohexyl)methyl)-3-methylureido)benzamide

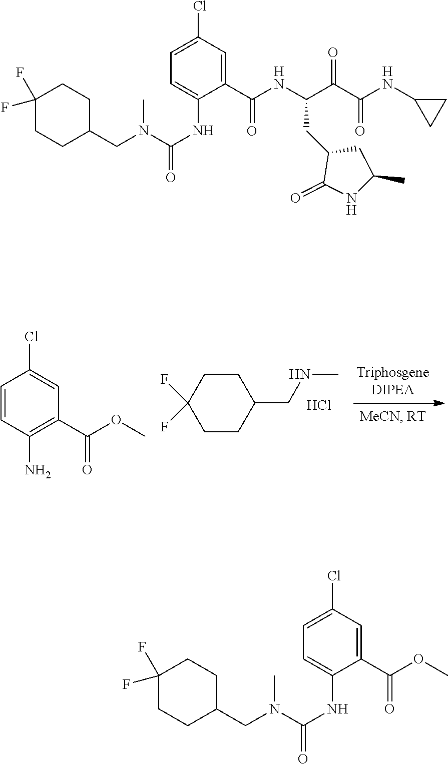

### Step 1: methyl 5-chloro-2-(3-((4,4-difluorocyclohexyl)methyl)-3-methylureido)benzoate

To a vial containing methyl 2-amino-5-chlorobenzoate (202 mg, 1.088 mmol) was added triphosgene (123 mg, 0.415 mmol), followed by MeCN (4 mL). The reaction mixture was then capped and stirred at room temperature, followed by LC/MS. After 1 night at room temperature, DIPEA (775 μL, 4.44 mmol) was added, followed by [(4,4-difluorocyclohexyl)methyl](methyl)amine hydrochloride (259 mg, 1.297 mmol). The mixture continued to stir at room temperature. After 1.5 hrs at room temperature, the reaction mixture was diluted/quenched with water, then suspended in EtOAc, and washed with saturated sodium bicarbonate, then water, and then brine. The organic layer was then dried over anhydrous sodium sulfate, then filtered and concentrated. The resulting residue was purified by silica gel chromatography (0-10% IPA/DCM; 80 g ISCO). The desired fractions were concentrated to yield the title compound. LRMS m/z: (M+H)+ calculated 375.1; found 375.2.

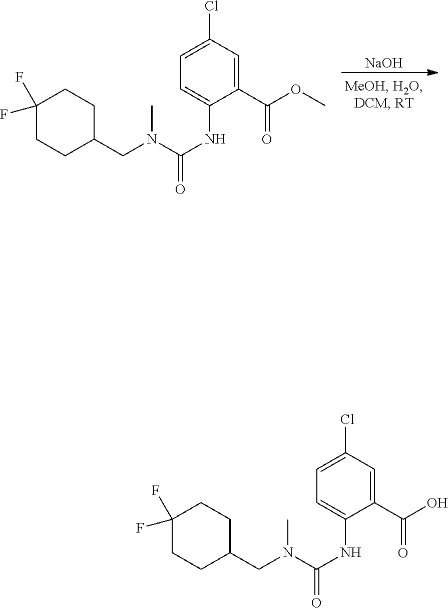 [SMILES: COC(=O)c1cc(Cl)ccc1NC(=O)N(C)CC1CCC(F)(F)CC1 ; "NaOH" ; CN(CC1CCC(F)(F)CC1)C(=O)Nc1ccc(Cl)cc1C(=O)O]

### Step 2: 5-chloro-2-(3-((4,4-difluorocyclohexyl)methyl)-3-methylureido)benzoic acid

To a flask containing methyl 5-chloro-2-(3-((4,4-difluorocyclohexyl)methyl)-3-methylureido)benzoate (308 mg, 0.822 mmol) was added MeOH (8 mL) followed by DCM (2 mL), then Water (2 mL) and finally 5N sodium hydroxide (425 μL, 2.125 mmol). The reaction mixture was then capped and stirred at room temperature, followed by LC/MS. After 1.5 hrs at room temperature, the reaction mixture was diluted/quenched with 1N HCl (10 mL, 10.00 mmol), then suspended in EtOAc, and washed with 1N HCl, followed by brine; organics were dried over anhydrous sodium sulfate, then filtered and concentrated to yield the title compound. LRMS m/z: (M+H)+ calculated 361.1; found 361.2.

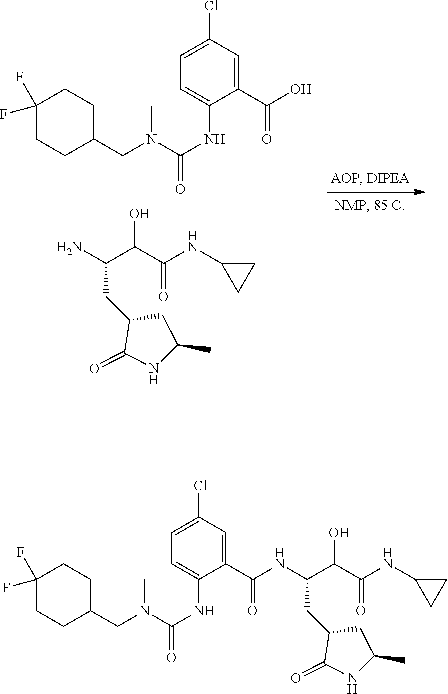 [SMILES: CN(CC1CCC(F)(F)CC1)C(=O)Nc1ccc(Cl)cc1C(=O)O ; C[C@@H]1C[C@@H](C[C@H](N)C(O)C(=O)NC2CC2)C(=O)N1 ; C[C@@H]1C[C@@H](C[C@H](NC(=O)c2cc(Cl)ccc2NC(=O)N(C)CC2CCC(F)(F)CC2)C(O)C(=O)NC2CC2)C(=O)N1]

### Step 3: 5-chloro-N-((2S)-4-(cyclopropylamino)-3-hydroxy-1-((3S,5R)-5-methyl-2-oxopyrrolidin-3-yl)-4-oxobutan-2-yl)-2-(3-((4,4-difluorocyclohexyl)methyl)-3-methylureido)benzamide

To a vial containing 5-chloro-2-(3-((4,4-difluorocyclohexyl)methyl)-3-methylureido)benzoic acid (70 mg, 0.194 mmol) and (3S)-3-amino-N-cyclopropyl-2-hydroxy-4-((3R,5R)-5-methyl-2-oxopyrrolidin-3-yl)butanamide (61 mg, 0.239 mmol) was added 7-azabenzotriazol-1-yloxytris(dimethylamino)phosphonium hexafluorophosphate (138 mg, 0.311 mmol) followed by NMP (1 mL) and finally DIPEA (90 μL, 0.515 mmol). The reaction mixture was then capped and heated immediately to 85° C. in the hood. This was followed by LC/MS. After 1 night at 85° C., the reaction mixture was diluted with 200 μL MeOH, then purified (without workup) by reverse phase chromatography (1×4.2 mL injection) (10-80% MeCN/H2O; 0.1% TFA in AQ; 20 min gradient; Waters 30×150 mm Sunfire 5 micron C18 column; Flow=42.5 mL/min). The desired fractions were concentrated then dissolved in DCM/MeOH and concentrated to yield the title compound. LRMS m/z: (M+H)+ calculated 598.2; found 598.4.

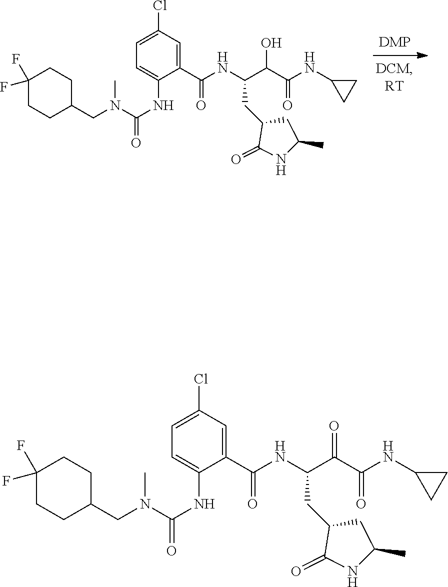 [SMILES: C[C@@H]1C[C@@H](C[C@H](NC(=O)c2cc(Cl)ccc2NC(=O)N(C)CC2CCC(F)(F)CC2)C(O)C(=O)NC2CC2)C(=O)N1 ; C[C@@H]1C[C@@H](C[C@H](NC(=O)c2cc(Cl)ccc2NC(=O)N(C)CC2CCC(F)(F)CC2)C(=O)C(=O)NC2CC2)C(=O)N1]

### Step 4: 5-chloro-N—((S)-4-(cyclopropylamino)-1-((3S,5R)-5-methyl-2-oxopyrrolidin-3-yl)-3,4-dioxobutan-2-yl)-2-(3-((4,4-difluorocyclohexyl)methyl)-3-methylureido)benzamide

To a vial containing 5-chloro-N-((2S)-4-(cyclopropylamino)-3-hydroxy-1-((3S,5R)-5-methyl-2-oxopyrrolidin-3-yl)-4-oxobutan-2-yl)-2-(3-((4,4-difluorocyclohexyl)methyl)-3-methylureido)benzamide (61 mg, 0.102 mmol) was added Dess-MartinPeriodinane (83 mg, 0.196 mmol) and sodium bicarbonate (32 mg, 0.381 mmol) followed by DCM (5 mL). The reaction mixture was then capped and stirred at room temperature, followed by LC/MS. After 1 hr the reaction mixture was quenched/diluted with 4 mL of saturated sodium thiosulfate, followed by 1 mL water, and then diluted with 10 mL EtOAc. The reaction mixture was stirred for 15 minutes, then suspended in EtOAc, and washed with saturated sodium thiosulfate, then water, and then brine. The organic layer was dried over anhydrous sodium sulfate, filtered, and concentrated. The resulting residue was then dissolved in DCM and purified by silica gel chromatography (0-100% EtOAc/Hex; 14 CV; 40 g ISCO). The desired fractions were concentrated to yield the title compound. LRMS m/z: (M+H)+ calculated 596.2; found 596.4.

¹H NMR (500 MHz, DMSO) δ 10.75 (s, 1H), 9.64 (d, J=6.1 Hz, 1H), 8.80 (d, J=5.1 Hz, 1H), 8.42 (d, J=9.1 Hz, 1H), 7.96 (s, 1H), 7.90 (d, J=2.5 Hz, 1H), 7.52 (dd, J=9.1, 2.5 Hz, 1H), 5.21-5.09 (m, 1H), 3.66-3.58 (m, 1H), 3.25-3.12 (m, 2H), 2.94 (s, 3H), 2.78-2.71 (m, 1H), 2.70-2.61 (m, 1H), 2.05-1.93 (m, 4H), 1.91-1.82 (m, 1H), 1.82-1.65 (m, 5H), 1.25-1.13 (m, 3H), 1.09 (d, J=6.3 Hz, 3H), 0.70-0.63 (m, 2H), 0.60-0.53 (m, 2H).

### Example 115

### N—((S)-4-(benzylamino)-1-((3S,5R)-5-methyl-2-oxopyrrolidin-3-yl)-3,4-dioxobutan-2-yl)-5-chloro-2-(4,4,4-trifluorobutanamido)benzamide

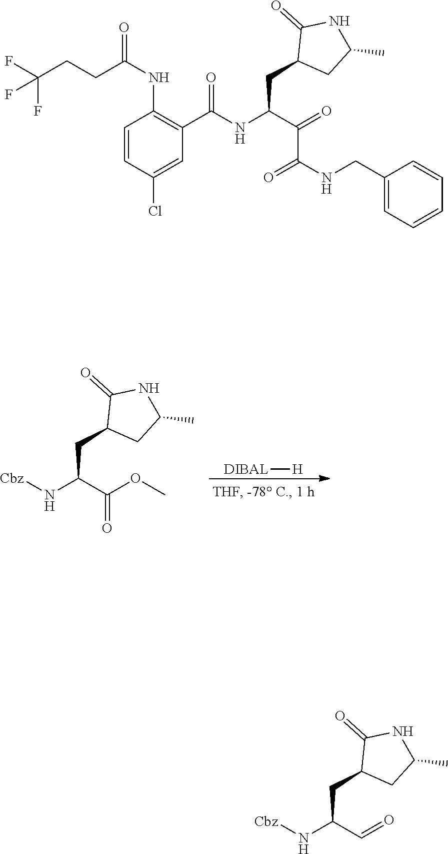

### Step 1: benzyl ((S)-1-((3S,5R)-5-methyl-2-oxopyrrolidin-3-yl)-3-oxopropan-2-yl)carbamate

To a solution of methyl (S)-2-(((benzyloxy)carbonyl)amino)-3-((3S,5R)-5-methyl-2-oxopyrrolidin-3-yl)propanoate (3 g, 8.97 mmol) in THF (40 mL) was added DIBAL-H (44.9 mL, 44.9 mmol, 1 M in toluene) at −78° C. under an atmosphere of nitrogen. The reaction mixture was stirred at −78° C. for 1 h, followed by LC/MS. The reaction mixture was quenched by anhydrous MeOH (10 mL) and saturated Rochelle's salt (30 mL) at −78° C. and the resulting mixture was warmed to room temperature. The reaction mixture was then diluted with EtOAc (50 mL) and extracted with EtOAc (3×30 mL). The combined organic phases were washed with brine (2×30 mL), then dried over anhydrous Na₂SO₄, filtered and concentrated to yield the title compound which was used directly in the next step without further purification. LRMS m/z: (M+H)+ calculated 305.1; found 305.1.

¹H NMR (400 MHz, CHLOROFORM-d) δ 9.55 (s, 1H), 7.39-7.27 (m, 5H), 7.26-7.13 (m, 1H), 6.21 (s, 1H), 5.21-5.00 (m, 2H), 4.61-4.17 (m, 1H), 3.79-3.65 (m, 1H), 2.58 (t, J=8.0 Hz, 1H), 2.05-1.83 (m, 4H), 1.20-1.06 (m, 3H).

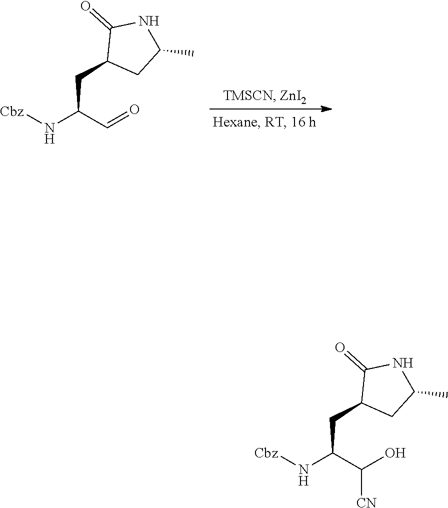 [SMILES: C[C@@H]1C[C@@H](C[C@@H](C=O)NC(=O)OCc2ccccc2)C(=O)N1 ; C[C@@H]1C[C@@H](C[C@H](NC(=O)OCc2ccccc2)C(O)C#N)C(=O)N1]

### Step 2: benzyl ((2S)-1-cyano-1-hydroxy-3-((3S,5R)-5-methyl-2-oxopyrrolidin-3-yl)propan-2-yl)carbamate

To a stirred solution of benzyl ((S)-1-((3S,5R)-5-methyl-2-oxopyrrolidin-3-yl)-3-oxopropan-2-yl)carbamate (2 g, 6.57 mmol) and ZnI₂ (1.049 g, 3.29 mmol) in n-hexane (20 mL) was added TMS-CN (1.057 mL, 7.89 mmol) and the mixture was stirred at 25° C. for 16 h. Followed by LC/MS. The reaction mixture was acidized with 1 M HCl to pH=4-5 and the mixture was stirred at 25° C. for 1 h. Then the mixture was adjusted with the saturated sodium bicarbonate to about pH=9. The mixture was extracted with EtOAc (3×20 mL). The combined organic phases were washed with brine (20 mL), dried over anhydrous Na₂SO₄, filtered and concentrated. The resulting residue was purified by flash silica gel chromatography (ISCO®; 4 g SepaFlash® Silica Flash Column, eluent of 100% ethyl acetate/pet. ether gradient @ 500 mL/min). The desired fractions were concentrated to yield the title compound. LRMS m/z: (M+H)+ calculated 332.1; found 332.1.

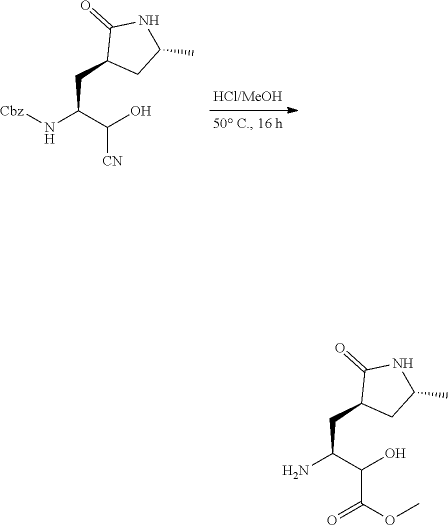 [SMILES: C[C@@H]1C[C@@H](C[C@H](NC(=O)OCc2ccccc2)C(O)C#N)C(=O)N1 ; COC(=O)C(O)[C@@H](N)C[C@@H]1C[C@@H](C)NC1=O]

### Step 3: methyl (3S)-3-amino-2-hydroxy-4-((3R,5R)-5-methyl-2-oxopyrrolidin-3-yl)butanoate hydrochloride

To a mixture of benzyl ((2S)-1-cyano-1-hydroxy-3-((3S,5R)-5-methyl-2-oxopyrrolidin-3-yl)propan-2-yl)carbamate (1.1 g, 3.32 mmol) in MeOH (5 mL) was added 4M HCl/MeOH (10 mL) at 25° C., then the resulting mixture was stirred at 50° C. for 16 h. Followed by LC/MS. The reaction mixture then concentrated. The resulting residue was purified by RP-HPLC (Column: YMC-Actus Triart C18 150*30 mm*5 μm, Condition water (0.1% TFA)-ACN Begin B 0 End B 20 Gradient Time (min) 11.5 100% B Hold Time 1 Flow Rate (mL/min) 40). The desired fractions were concentrated to yield the title compound. LRMS m/z: (M+H)+ calculated 231.1; found 231.1. ¹H NMR (400 MHz, MeOD) δ 4.54-4.23 (m, 1H), 3.83-3.75 (m, 3H), 3.75-3.57 (m, 1H), 3.54-3.36 (m, 1H), 2.92-2.60 (m, 1H), 2.03-1.93 (m, 2H), 1.92-1.62 (m, 2H), 1.38-1.30 (m, 3H).

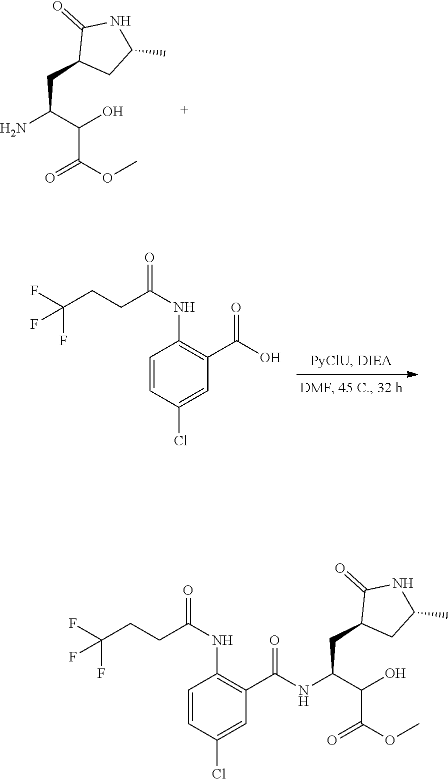 [SMILES: COC(=O)C(O)[C@@H](N)C[C@@H]1C[C@@H](C)NC1=O ; O=C(CCC(F)(F)F)Nc1ccc(Cl)cc1C(=O)O ; COC(=O)C(O)[C@H](C[C@@H]1C[C@@H](C)NC1=O)NC(=O)c1cc(Cl)ccc1NC(=O)CCC(F)(F)F]

### Step 4: methyl (3S)-3-(5-chloro-2-(4,4,4-trifluorobutanamido)benzamido)-2-hydroxy-4-((3S,5R)-5-methyl-2-oxopyrrolidin-3-yl)butanoate

To a vial was added 5-chloro-2-(4,4,4-trifluorobutanamido)benzoic acid (100 mg, 0.338 mmol) and 1-(chloro-1-pyrrolidinylmethylene)pyrrolidinium hexafluorophosphate (169 mg, 0.507 mmol) in DMF (5 mL). This mixture was stirred at 45° C. for 16 h. Then Methyl (3S)-3-amino-2-hydroxy-4-((3R,5R)-5-methyl-2-oxopyrrolidin-3-yl)butanoate (101 mg, 0.440 mmol) and DIEA (0.177 mL, 1.015 mmol) was added to the mixture, and the mixture was stirred at 45° C. for 16 h, followed by LC/MS. Water (0.5 mL) was added to the mixture and then the mixture was purified by RP-HPLC (Column: Welch Xtimate C18 150*25 mm*5 m, Condition water (0.1% TFA)-ACN Begin B 32 End B 62 Gradient Time (min) 11 100% B Hold Time 2 Flow Rate (mL/min) 25). The desired fractions were concentrated to yield the title compound. LRMS m/z: (M+H)+ calculated 508.1; found 508.1.

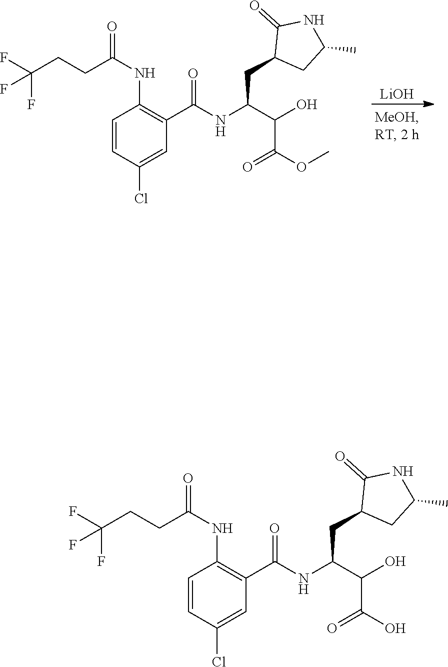 [SMILES: COC(=O)C(O)[C@H](C[C@@H]1C[C@@H](C)NC1=O)NC(=O)c1cc(Cl)ccc1NC(=O)CCC(F)(F)F ; "LiOH" ; C[C@@H]1C[C@@H](C[C@H](NC(=O)c2cc(Cl)ccc2NC(=O)CCC(F)(F)F)C(O)C(=O)O)C(=O)N1]

### Step 5: (3S)-3-(5-chloro-2-(4,4,4-trifluorobutanamido)benzamido)-2-hydroxy-4-((3S,5R)-5-methyl-2-oxopyrrolidin-3-yl)butanoic acid

To a solution of methyl (3S)-3-(5-chloro-2-(4,4,4-trifluorobutanamido)benzamido)-2-hydroxy-4-((3S,5R)-5-methyl-2-oxopyrrolidin-3-yl)butanoate (20 mg, 0.039 mmol) in THF (1 mL) and water (0.5 mL) was added lithium hydroxide monohydrate (3.30 mg, 0.079 mmol) and then the reaction mixture was stirred at 25° C. for 2 h, followed by LC/MS. Then the mixture was concentrated and the residue was acidized with 1 M HCl to pH=4-5 and then extracted with EtOAc (3×10 mL). The combined organic phases were washed with brine (10 mL), dried over anhydrous Na₂SO₄, filtered and concentrated to yield the title compound which was used without further purification. LRMS m/z: (M+H)+ calculated 494.1; found 494.1.

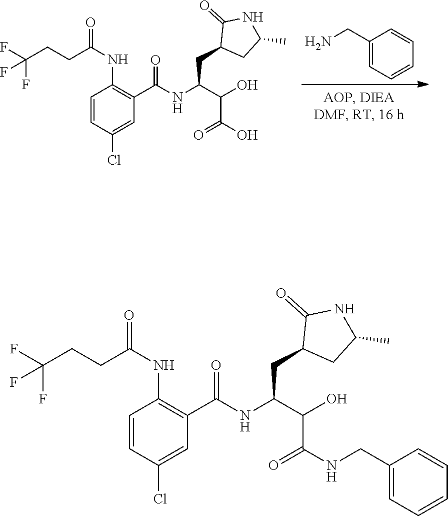 [SMILES: C[C@@H]1C[C@@H](C[C@H](NC(=O)c2cc(Cl)ccc2NC(=O)CCC(F)(F)F)C(O)C(=O)O)C(=O)N1 ; NCc1ccccc1 ; C[C@@H]1C[C@@H](C[C@H](NC(=O)c2cc(Cl)ccc2NC(=O)CCC(F)(F)F)C(O)C(=O)NCc2ccccc2)C(=O)N1]

### Step 6: N-((2S)-4-(benzylamino)-3-hydroxy-1-((3S,5R)-5-methyl-2-oxopyrrolidin-3-yl)-4-oxobutan-2-yl)-5-chloro-2-(4,4,4-trifluorobutanamido)benzamide

To a solution of (3S)-3-(5-chloro-2-(4,4,4-trifluorobutanamido)benzamido)-2-hydroxy-4-((3S,5R)-5-methyl-2-oxopyrrolidin-3-yl)butanoic acid (15 mg, 0.030 mmol) and phenylmethanamine (3.58 mg, 0.033 mmol) in DMF (1 mL) was added AOP (16.16 mg, 0.036 mmol) and DIEA (10.61 μL, 0.061 mmol) at 25° C., followed by LC/MS. After stirring 16 h at 25° C., water (0.5 mL) was added to the reaction mixture. Then the mixture was purified by RP-HPLC (Column: Welch Xtimate C18 150*25 mm*5 m, Condition water (0.1% TFA)-ACN Begin B 33 End B 63 Gradient Time (min) 11 100% B Hold Time 2 Flow Rate (mL/min) 25) to yield the title compound. LRMS m/z: (M+H)+ calculated 583.1; found 583.2.

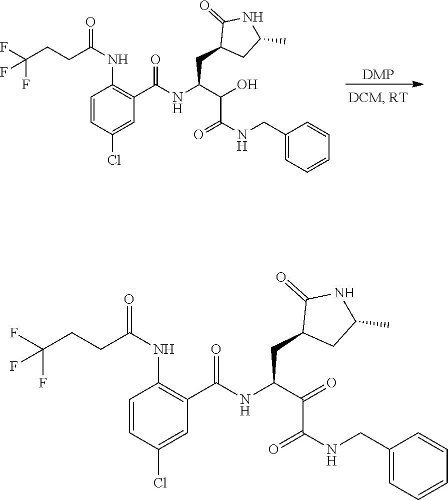

### Step 7: N—((S)-4-(benzylamino)-1-((3S,5R)-5-methyl-2-oxopyrrolidin-3-yl)-3,4-dioxobutan-2-yl)-5-chloro-2-(4,4,4-trifluorobutanamido)benzamide

A mixture of N-((2S)-4-(benzylamino)-3-hydroxy-1-((3S,5R)-5-methyl-2-oxopyrrolidin-3-yl)-4-oxobutan-2-yl)-5-chloro-2-(4,4,4-trifluorobutanamido)benzamide (10 mg, 0.017 mmol), sodium bicarbonate (4.32 mg, 0.051 mmol) and DMP (21.83 mg, 0.051 mmol) in DCM (1 mL) was stirred at 25° C. for 1 h, followed by LC/MS. The reaction mixture was then diluted with DCM, quenched with saturated Na₂SO₃ (4 mL) and saturated NaHCO₃ (4 mL). The organic phase was separated and dried over anhydrous Na₂SO₄, filtered and concentrated. The resulting residue was purified by flash silica gel chromatography (ISCO®; 4 g SepaFlash® Silica Flash Column, eluent of 50-100% ethyl acetate/pet. ether gradient @ 500 mL/min). The desired fractions were concentrated, then re-purified by RP-HPLC (Column: Welch Xtimate C18 150*25 mm*5 m, Condition water (0.10% TFA)-ACN Begin B 35 End B 65 Gradient Time (min) 11 100% B Hold Time 2 Flow Rate (mL/min) 25) to give the title compound. LRMS m/z: (M+H)+ calculated 581.1; found 581.3. ¹H NMR (400 MHz, MeOD) δ 8.45-8.13 (m, 1H), 7.83-7.65 (m, 1H), 7.54-7.44 (m, 1H), 7.38-7.00 (m, 5H), 5.52-5.02 (m, 1H), 4.53-4.36 (m, 2H), 3.77 (s, 1H), 2.71-2.63 (m, 2H), 2.56 (d, J=10.5 Hz, 2H), 2.08-1.99 (m, 2H), 1.34 (s, 3H), 1.24-1.14 (m, 3H).

The following examples were prepared according to similar methods to those described above.

| | | | MS | MS |
|---|---|---|---|---|
| Ex. | Structure | Name | Calculated | Observed |
| 12 | 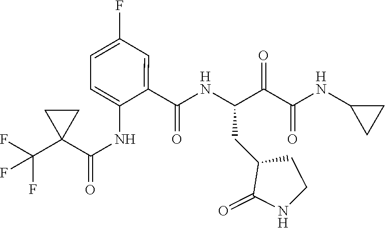 [SMILES: O=C(NC1CC1)C(=O)[C@H](C[C@@H]1CCNC1=O)NC(=O)c1cc(F)ccc1NC(=O)C1(C(F)(F)F)CC1] | N-[(1S)-3- (cyclopropylamino)-2,3- dioxo-1-[[(3S)-2- oxopyrrolidin-3- yl]methyl]propyl]-5-fluoro-2- [[1- (trifluoromethyl)cyclopropane- carbonyl]amino]benzamide | 513.5 | 513.1758 |
| 13 | 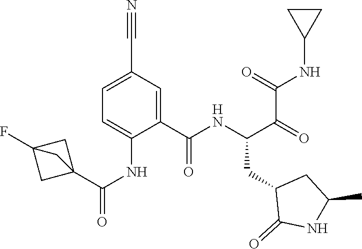 [SMILES: C[C@@H]1C[C@@H](C[C@H](NC(=O)c2cc(C#N)ccc2NC(=O)C23CC(F)(C2)C3)C(=O)C(=O)NC2CC2)C(=O)N1] | N-[4-cyano-2-[(1S)-3- (cyclopropylamino)-1- [[(3S,5R)-5-methyl-2-oxo- pyrrolidin-3-yl]methyl]-2,3- dioxo- propyl]carbamoyl]phenyl]-3- fluoro-bicyclo[1.1.1]pentane- 1-carboxamide | 510.5 | 510.2164 |
| 14 | 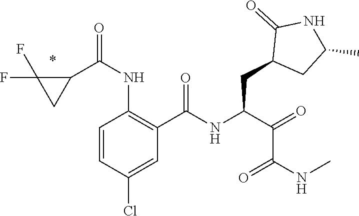 [SMILES: CNC(=O)C(=O)[C@H](C[C@@H]1C[C@@H](C)NC1=O)NC(=O)c1cc(Cl)ccc1NC(=O)C1CC1(F)F] | 5-chloro-2-[(2,2- difluorocyclopropanecarbonyl) amino]-N-[(1S)-3- (methylamino)-1-[[(3S,5R)- 5-methyl-2-oxo-pyrrolidin-3- yl]methyl]-2,3-dioxo- propyl]benzamide | 485.9 | 485.1398 |
| 15 | 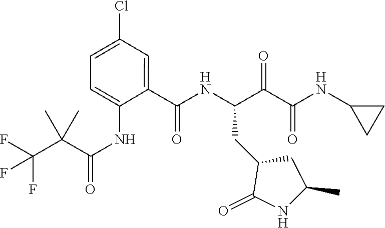 [SMILES: C[C@@H]1C[C@@H](C[C@H](NC(=O)c2cc(Cl)ccc2NC(=O)C(C)(C)C(F)(F)F)C(=O)C(=O)NC2CC2)C(=O)N1] | 5-chloro-N-[(1S)-3- (cyclopropylamino)-1- [[(3S,5R)-5-methyl-2-oxo- pyrrolidin-3-yl]methyl]-2,3- dioxo-propyl]-2-[(3,3,3- trifluoro-2,2-dimethyl- propanoyl)amino]benzamide | 545.9 | 545.1788 |
| 16 | 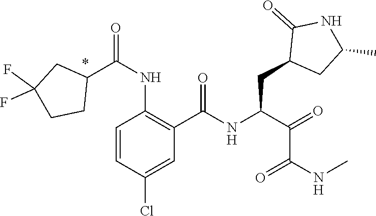 [SMILES: CNC(=O)C(=O)[C@H](C[C@@H]1C[C@@H](C)NC1=O)NC(=O)c1cc(Cl)ccc1NC(=O)C1CCC(F)(F)C1] | 5-chloro-2-[(3,3- difluorocyclopentanecarbonyl) amino]-N-[(1S)-3- (methylamino)-1-[[(3S,5R)- 5-methyl-2-oxo-pyrrolidin-3- yl]methyl]-2,3-dioxo- propyl]benzamide | 513.9 | 513.1713 |
| 17 | 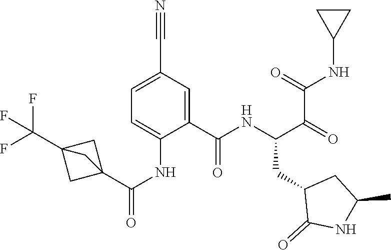 [SMILES: C[C@@H]1C[C@@H](C[C@H](NC(=O)c2cc(C#N)ccc2NC(=O)C23CC(C(F)(F)F)(C2)C3)C(=O)C(=O)NC2CC2)C(=O)N1] | N-[4-cyano-2-[(1S)-3- (cyclopropylamino)-1- [[(3S,5R)-5-methyl-2-oxo- pyrrolidin-3-yl]methyl]-2,3- dioxo- propyl]carbamoyl]phenyl]-3- (trifluoromethyl)bicyclo[1.1.1] pentane-1-carboxamide | 560.5 | 560.2131 |
| 18 | 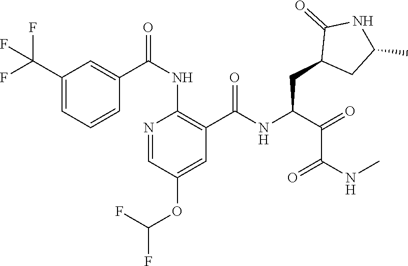 [SMILES: CNC(=O)C(=O)[C@H](C[C@@H]1C[C@@H](C)NC1=O)NC(=O)c1cc(OC(F)F)cnc1NC(=O)c1cccc(C(F)(F)F)c1] | 5-(difluoromethoxy)-N- [(1S)-3-(methylamino)-1- [[(3S,5R)-5-methyl-2-oxo- pyrrolidin-3-yl]methyl]-2,3- dioxo-propyl]-2-[[3- (trifluoromethyl)benzoyl] amino]pyridine-3-carboxamide | 586.5 | 586.1736 |
| 19 | 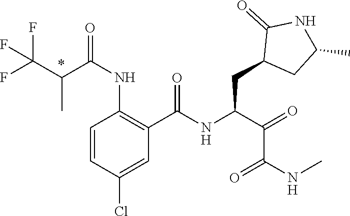 [SMILES: CNC(=O)C(=O)[C@H](C[C@@H]1C[C@@H](C)NC1=O)NC(=O)c1cc(Cl)ccc1NC(=O)C(C)C(F)(F)F] | 5-chloro-N-[(1S)-3- (methylamino)-1-[[(3S,5R)- 5-methyl-2-oxo-pyrrolidin-3- yl]methyl]-2,3-dioxo- propyl]-2-[(3,3,3-trifluoro-2- methyl- propanoyl)amino]benzamide | 505.9 | 505.1469 |
| 20 | 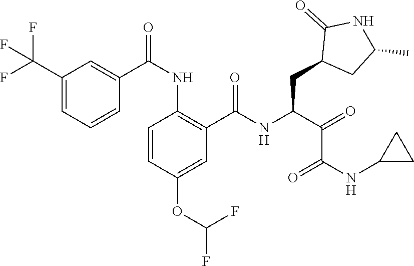 [SMILES: C[C@@H]1C[C@@H](C[C@H](NC(=O)c2cc(OC(F)F)ccc2NC(=O)c2cccc(C(F)(F)F)c2)C(=O)C(=O)NC2CC2)C(=O)N1] | N-[(1S)-3- (cyclopropylamino)-1- [[(3S,5R)-5-methyl-2-oxo- pyrrolidin-3-yl]methyl]-2,3- dioxo-propyl]-5- (difluoromethoxy)-2-[[3- (trifluoromethyl)benzoyl] amino]benzamide | 611.5 | 611.194 |
| 21 | 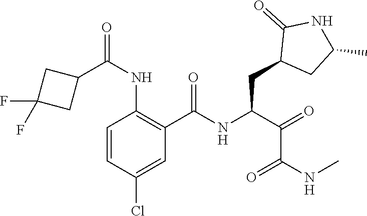 [SMILES: CNC(=O)C(=O)[C@H](C[C@@H]1C[C@@H](C)NC1=O)NC(=O)c1cc(Cl)ccc1NC(=O)C1CC(F)(F)C1] | 5-chloro-2-[(3,3- difluorocyclobutanecarbonyl) amino]-N-[(1S)-3- (methylamino)-1-[[(3S,5R)- 5-methyl-2-oxo-pyrrolidin-3- yl]methyl]-2,3-dioxo- propyl]benzamide | 499.9 | 499.1564 |
| 22 | 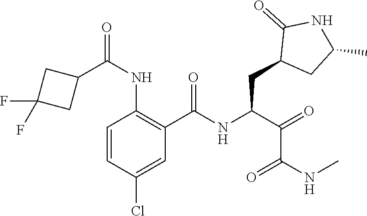 [SMILES: CNC(=O)C(=O)[C@H](C[C@@H]1C[C@@H](C)NC1=O)NC(=O)c1cc(Cl)ccc1NC(=O)C1CC(F)(F)C1] | 5-chloro-N-[(1S)-3- (cyclopropylamino)-1- [[(3S,5R)-5-methyl-2-oxo- pyrrolidin-3-yl]methyl]-2,3- dioxo-propyl]-2-[[1- (trifluoromethyl)cyclopropane- carbonyl]amino]pyridine-3- carboxamide | 544.9 | 544.1588 |
| 23 | 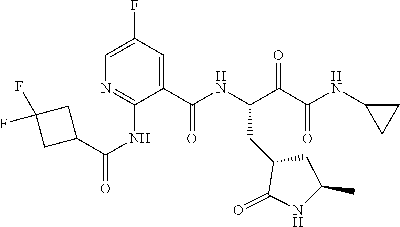 [SMILES: C[C@@H]1C[C@@H](C[C@H](NC(=O)c2cc(F)cnc2NC(=O)C2CC(F)(F)C2)C(=O)C(=O)NC2CC2)C(=O)N1] | N-[(1S)-3- (cyclopropylamino)-1- [[(3S,5R)-5-methyl-2-oxo- pyrrolidin-3-yl]methyl]-2,3- dioxo-propyl]-2-[(3,3- difluorocyclobutanecarbonyl) amino]-5-fluoro-pyridine-3- carboxamide | 510.5 | 510.1978 |
| 24 | 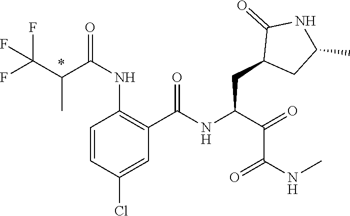 [SMILES: CNC(=O)C(=O)[C@H](C[C@@H]1C[C@@H](C)NC1=O)NC(=O)c1cc(Cl)ccc1NC(=O)C(C)C(F)(F)F] | 5-chloro-N-[(1S)-3- (methylamino)-1-[[(3S,5R)- 5-methyl-2-oxo-pyrrolidin-3- yl]methyl]-2,3-dioxo- propyl]-2-[(3,3,3-trifluoro-2- methyl- propanoyl)amino]benzamide | 505.9 | 505.1462 |
| 25 | 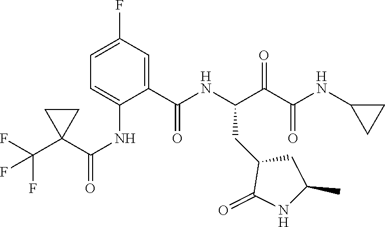 [SMILES: C[C@@H]1C[C@@H](C[C@H](NC(=O)c2cc(F)ccc2NC(=O)C2(C(F)(F)F)CC2)C(=O)C(=O)NC2CC2)C(=O)N1] | N-[(1S)-3- (cyclopropylamino)-1- [[(3S,5R)-5-methyl-2-oxo- pyrrolidin-3-yl]methyl]-2,3- dioxo-propyl]-5-fluoro-2-[[1- (trifluoromethyl)cyclopropane- carbonyl]amino]benzamide | 527.5 | 527.1922 |
| 26 | 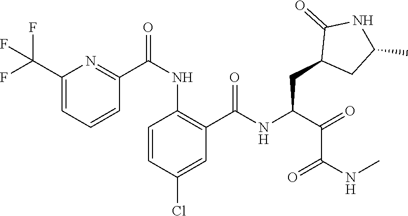 [SMILES: CNC(=O)C(=O)[C@H](C[C@@H]1C[C@@H](C)NC1=O)NC(=O)c1cc(Cl)ccc1NC(=O)c1cccc(C(F)(F)F)n1] | N-[4-chloro-2-[[(1S)-3- (methylamino)-1-[[(3S,5R)- 5-methyl-2-oxo-pyrrolidin-3- yl]methyl]-2,3-dioxo- propyl]carbamoyl]phenyl]-6- (trifluoromethyl)pyridine-2- carboxamide | 554.9 | 554.1428 |
| 27 | 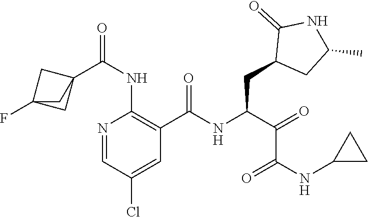 [SMILES: C[C@@H]1C[C@@H](C[C@H](NC(=O)c2cc(Cl)cnc2NC(=O)C23CC(F)(C2)C3)C(=O)C(=O)NC2CC2)C(=O)N1] | 5-chloro-N-[(1S)-3- (cyclopropylamino)-1- [[(3S,5R)-5-methyl-2-oxo- pyrrolidin-3-yl]methyl]-2,3- dioxo-propyl]-2-[(3- fluorobicyclo[1.1.1]pentane- 1-carbonyl)amino]pyridine- 3-carboxamide | 520.9 | 520.1773 |
| 28 | 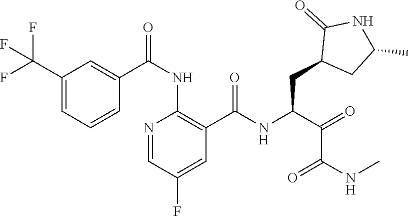 [SMILES: CNC(=O)C(=O)[C@H](C[C@@H]1C[C@@H](C)NC1=O)NC(=O)c1cc(F)cnc1NC(=O)c1cccc(C(F)(F)F)c1] | 5-fluoro-N-[(1S)-3- (methylamino)-1-[[(3S,5R)- 5-methyl-2-oxo-pyrrolidin-3- yl]methyl]-2,3-dioxo- propyl]-2-[[3- (trifluoromethyl)benzoyl] amino]pyridine-3-carboxamide | 538.5 | 538.1706 |
| 29 | 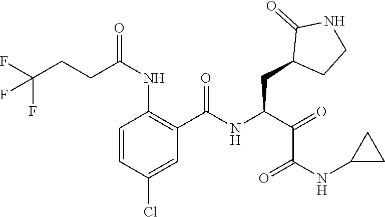 [SMILES: O=C(CCC(F)(F)F)Nc1ccc(Cl)cc1C(=O)N[C@@H](C[C@@H]1CCNC1=O)C(=O)C(=O)NC1CC1] | 5-chloro-N-[(1S)-3- (cyclopropylamino)-2,3- dioxo-1-[[(3S)-2- oxopyrrolidin-3- yl]methyl]propyl]-2-(4,4,4- trifluorobutanoylamino) benzamide | 517.9 | 517.1473 |
| 30 | 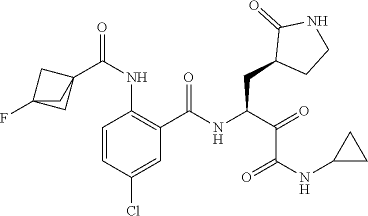 [SMILES: O=C(NC1CC1)C(=O)[C@H](C[C@@H]1CCNC1=O)NC(=O)c1cc(Cl)ccc1NC(=O)C12CC(F)(C1)C2] | N-[4-chloro-2-[[(1S)-3- (cyclopropylamino)-2,3- dioxo-1-[[(3S)-2- oxopyrrolidin-3- yl]methyl]propyl]carbamoyl] phenyl]-3-fluoro- bicyclo[1.1.1]pentane-1- carboxamide | 505.9 | 505.1664 |
| 31 | 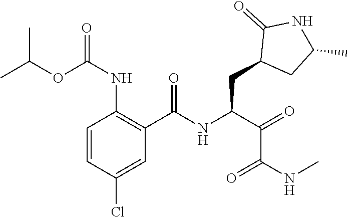 [SMILES: CNC(=O)C(=O)[C@H](C[C@@H]1C[C@@H](C)NC1=O)NC(=O)c1cc(Cl)ccc1NC(=O)OC(C)C] | isopropyl N-[4-chloro-2- [[(1S)-3-(methylamino)-1- [[(3S,5R)-5-methyl-2-oxo- pyrrolidin-3-yl]methyl]-2,3- dioxo- propyl]carbamoyl]phenyl] carbamate | 467.9 | 467.17 |
| 32 | 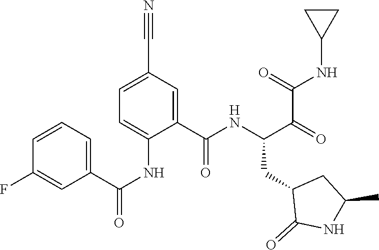 [SMILES: C[C@@H]1C[C@@H](C[C@H](NC(=O)c2cc(C#N)ccc2NC(=O)c2cccc(F)c2)C(=O)C(=O)NC2CC2)C(=O)N1] | 5-cyano-N-[(1S)-3- (cyclopropylamino)-1- [[(3S,5R)-5-methyl-2-oxo- pyrrolidin-3-yl]methyl]-2,3- dioxo-propyl]-2-[(3- fluorobenzoyl)amino] benzamide | 520.5 | 520.2002 |
| 33 | 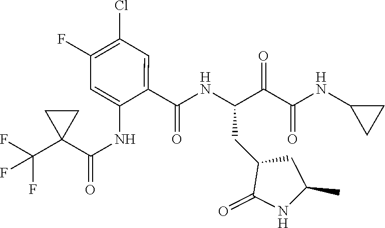 [SMILES: C[C@@H]1C[C@@H](C[C@H](NC(=O)c2cc(Cl)c(F)cc2NC(=O)C2(C(F)(F)F)CC2)C(=O)C(=O)NC2CC2)C(=O)N1] | 5-chloro-N-[(1S)-3- (cyclopropylamino)-1- [[(3S,5R)-5-methyl-2-oxo- pyrrolidin-3-yl]methyl]-2,3- dioxo-propyl]-4-fluoro-2-[[1- (trifluoromethyl)cyclopropane- carbonyl]amino]benzamide | 561.9 | 561.1538 |
| 34 | 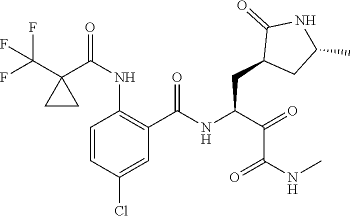 [SMILES: CNC(=O)C(=O)[C@H](C[C@@H]1C[C@@H](C)NC1=O)NC(=O)c1cc(Cl)ccc1NC(=O)C1(C(F)(F)F)CC1] | 5-chloro-N-[(1S)-3- (methylamino)-1-[[(3S,5R)- 5-methyl-2-oxo-pyrrolidin-3- yl]methyl]-2,3-dioxo- propyl]-2-[[1- (trifluoromethyl)cyclopropane- carbonyl]amino]benzamide | 517.9 | 517.2 |
| 35 | 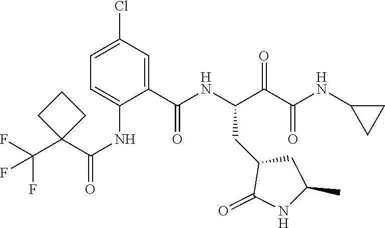 [SMILES: C[C@@H]1C[C@@H](C[C@H](NC(=O)c2cc(Cl)ccc2NC(=O)C2(C(F)(F)F)CCC2)C(=O)C(=O)NC2CC2)C(=O)N1] | 5-chloro-N-[(1S)-3- (cyclopropylamino)-1- [[(3S,5R)-5-methyl-2-oxo- pyrrolidin-3-yl]methyl]-2,3- dioxo-propyl]-2-[[1- (trifluoromethyl)cyclobutane carbonyl]amino]benzamide | 557.9 | 557.1786 |
| 36 | 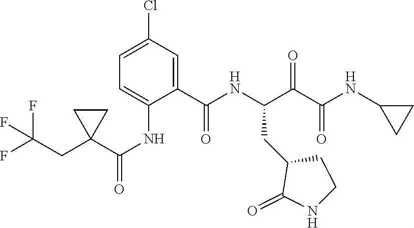 [SMILES: O=C(NC1CC1)C(=O)[C@H](C[C@@H]1CCNC1=O)NC(=O)c1cc(Cl)ccc1NC(=O)C1(CC(F)(F)F)CC1] | 5-chloro-N-[(1S)-3- (cyclopropylamino)-2,3- dioxo-1-[[(3S)-2- oxopyrrolidin-3- yl]methyl]propyl]-2-[[1- (2,2,2- trifluoroethyl)cyclopropane- carbonyl]amino]benzamide | 543.9 | 543.1631 |
| 37 | 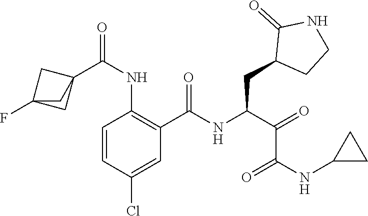 [SMILES: O=C(NC1CC1)C(=O)[C@H](C[C@@H]1CCNC1=O)NC(=O)c1cc(Cl)ccc1NC(=O)C12CC(F)(C1)C2] | N-[4-chloro-2-[[(1S)-3- (cyclopropylamino)-2,3- dioxo-1-[[(3S)-2- oxopyrrolidin-3- yl]methyl]propyl]carbamoyl] phenyl]-3-fluoro- bicyclo[1.1.1]pentane-1- carboxamide | 505.9 | 505.1657 |
| 38 | 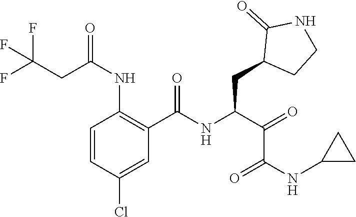 [SMILES: O=C(CC(F)(F)F)Nc1ccc(Cl)cc1C(=O)N[C@@H](C[C@@H]1CCNC1=O)C(=O)C(=O)NC1CC1] | 5-chloro-N-[(1S)-3- (cyclopropylamino)-2,3- dioxo-1-[[(3S)-2- oxopyrrolidin-3- yl]methyl]propyl]-2-(3,3,3- trifluoropropanoylamino) benzamide | 503.9 | 503.1 |
| 39 | 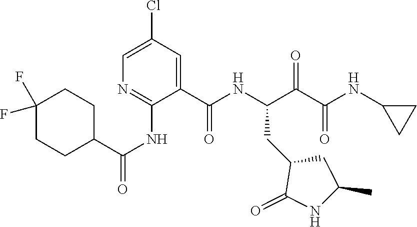 [SMILES: C[C@@H]1C[C@@H](C[C@H](NC(=O)c2cc(Cl)cnc2NC(=O)C2CCC(F)(F)CC2)C(=O)C(=O)NC2CC2)C(=O)N1] | 5-chloro-N-[(1S)-3- (cyclopropylamino)-1- [[(3S,5R)-5-methyl-2-oxo- pyrrolidin-3-yl]methyl]-2,3- dioxo-propyl]-2-[(4,4- difluorocyclohexanecarbonyl) amino]pyridine-3- carboxamide | 554.9 | 554.1993 |
| 40 | 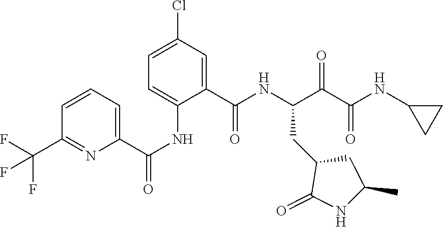 [SMILES: C[C@@H]1C[C@@H](C[C@H](NC(=O)c2cc(Cl)ccc2NC(=O)c2cccc(C(F)(F)F)n2)C(=O)C(=O)NC2CC2)C(=O)N1] | N-[4-chloro-2-[(1S)-3- (cyclopropylamino)-1- [[(3S,5R)-5-methyl-2-oxo- pyrrolidin-3-yl]methyl]-2,3- dioxo- propyl]carbamoyl]phenyl]-6- (trifluoromethyl)pyridine-2- carboxamide | 580.9 | 580.1586 |
| 41 | 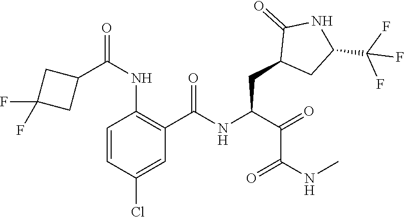 [SMILES: CNC(=O)C(=O)[C@H](C[C@@H]1C[C@@H](C(F)(F)F)NC1=O)NC(=O)c1cc(Cl)ccc1NC(=O)C1CC(F)(F)C1] | 5-chloro-2-[(3,3- difluorocyclobutanecarbonyl) amino]-N-[(1S)-3- (methylamino)-2,3-dioxo-1- [[(3R,5S)-2-oxo-5- (trifluoromethyl)pyrrolidin-3- yl]methyl]propyl]benzamide | 553.9 | 553.1281 |
| 42 | 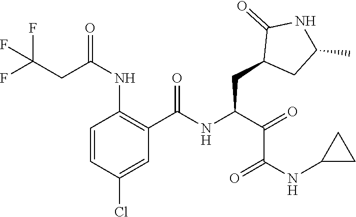 [SMILES: C[C@@H]1C[C@@H](C[C@H](NC(=O)c2cc(Cl)ccc2NC(=O)CC(F)(F)F)C(=O)C(=O)NC2CC2)C(=O)N1] | 5-chloro-N-[(1S)-3- (cyclopropylamino)-1- [[(3S,5R)-5-methyl-2-oxo- pyrrolidin-3-yl]methyl]-2,3- dioxo-propyl]-2-(3,3,3- trifluoropropanoylamino) benzamide | 517.9 | 517.147 |
| 43 | 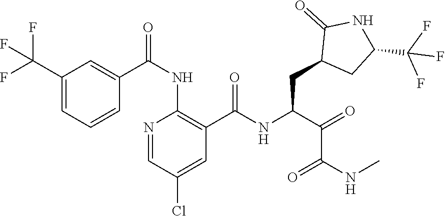 [SMILES: CNC(=O)C(=O)[C@H](C[C@@H]1C[C@@H](C(F)(F)F)NC1=O)NC(=O)c1cc(Cl)cnc1NC(=O)c1cccc(C(F)(F)F)c1] | 5-chloro-N-[(1S)-3- (methylamino)-2,3-dioxo-1- [[(3R,5S)-2-oxo-5- (trifluoromethyl)pyrrolidin-3- yl]methyl]propyl]-2-[[3- (trifluoromethyl)benzoyl] amino]pyridine-3-carboxamide | 608.9 | 608.1143 |
| 44 | 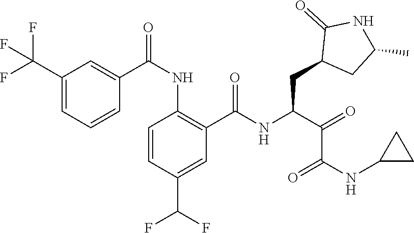 [SMILES: C[C@@H]1C[C@@H](C[C@H](NC(=O)c2cc(C(F)F)ccc2NC(=O)c2cccc(C(F)(F)F)c2)C(=O)C(=O)NC2CC2)C(=O)N1] | N-[(1S)-3- (cyclopropylamino)-1- [[(3S,5R)-5-methyl-2-oxo- pyrrolidin-3-yl]methyl]-2,3- dioxo-propyl]-5- (difluoromethyl)-2-[[3- (trifluoromethyl)benzoyl] amino]benzamide | 595.5 | 595.1984 |
| 45 | 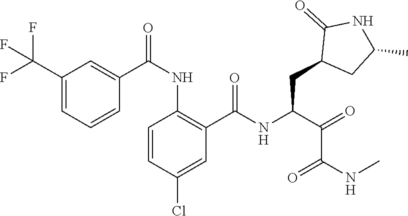 [SMILES: CNC(=O)C(=O)[C@H](C[C@@H]1C[C@@H](C)NC1=O)NC(=O)c1cc(Cl)ccc1NC(=O)c1cccc(C(F)(F)F)c1] | 5-chloro-N-[(1S)-3- (methylamino)-1-[[(3S,5R)- 5-methyl-2-oxo-pyrrolidin-3- yl]methyl]-2,3-dioxo- propyl]-2-[[3- (trifluoromethyl)benzoyl] amino]benzamide | 553.9 | 553.147 |
| 46 | 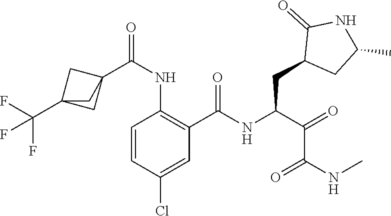 [SMILES: CNC(=O)C(=O)[C@H](C[C@@H]1C[C@@H](C)NC1=O)NC(=O)c1cc(Cl)ccc1NC(=O)C12CC(C(F)(F)F)(C1)C2] | N-[4-chloro-2-[[(1S)-3- (methylamino)-1-[[(3S,5R)- 5-methyl-2-oxo-pyrrolidin-3- yl]methyl]-2,3-dioxo- propyl]carbamoyl]phenyl]-3- (trifluoromethyl)bicyclo[1.1.1] pentane-1-carboxamide | 543.9 | 543.1628 |
| 47 | 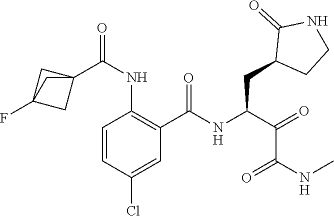 [SMILES: CNC(=O)C(=O)[C@H](C[C@@H]1CCNC1=O)NC(=O)c1cc(Cl)ccc1NC(=O)C12CC(F)(C1)C2] | N-[4-chloro-2-[(1S)-3- (methylamino)-2,3-dioxo-1- [[(3S)-2-oxopyrrolidin-3- yl]methyl]propyl]carbamoyl] phenyl]-3-fluoro- bicyclo[1.1.1]pentane-1- carboxamide | 479.9 | 479.1501 |
| 48 | 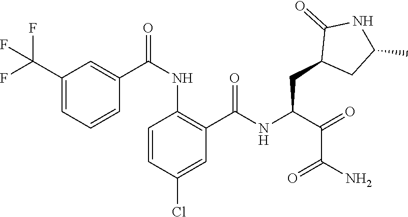 [SMILES: C[C@@H]1C[C@@H](C[C@H](NC(=O)c2cc(Cl)ccc2NC(=O)c2cccc(C(F)(F)F)c2)C(=O)C(N)=O)C(=O)N1] | N-[(1S)-3-amino-1- [[(3S,5R)-5-methyl-2-oxo- pyrrolidin-3-yl]methyl]-2,3- dioxo-propyl]-5-chloro-2-[[3- (trifluoromethyl)benzoyl] amino]benzamide | 539.9 | 539.1309 |
| 49 | 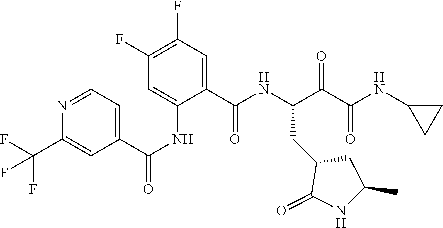 [SMILES: C[C@@H]1C[C@@H](C[C@H](NC(=O)c2cc(F)c(F)cc2NC(=O)c2ccnc(C(F)(F)F)c2)C(=O)C(=O)NC2CC2)C(=O)N1] | N-[2-[[(1S)-3- (cyclopropylamino)-1- [[(3S,5R)-5-methyl-2-oxo- pyrrolidin-3-yl]methyl]-2,3- dioxo-propyl]carbamoyl]- 4,5-difluoro-phenyl]-2- (trifluoromethyl)pyridine-4- carboxamide | 582.5 | 582.1774 |
| 50 | 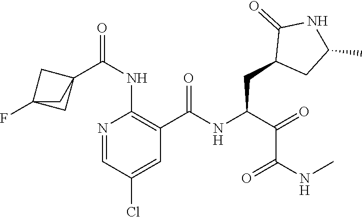 [SMILES: CNC(=O)C(=O)[C@H](C[C@@H]1C[C@@H](C)NC1=O)NC(=O)c1cc(Cl)cnc1NC(=O)C12CC(F)(C1)C2] | 5-chloro-2-[(3- fluorobicyclo[1.1.1]pentane- 1-carbonyl)amino]-N-[(1S)- 3-(methylamino)-1- [[(3S,5R)-5-methyl-2-oxo- pyrrolidin-3-yl]methyl]-2,3- dioxo-propyl]pyridine-3- carboxamide | 494.9 | 494.162 |
| 51 | 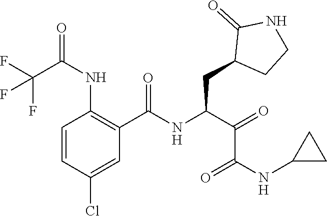 [SMILES: O=C(NC1CC1)C(=O)[C@H](C[C@@H]1CCNC1=O)NC(=O)c1cc(Cl)ccc1NC(=O)C(F)(F)F] | 5-chloro-N-[(1S)-3- (cyclopropylamino)-2,3- dioxo-1-[[(3S)-2- oxopyrrolidin-3- yl]methyl]propyl]-2-[(2,2,2- trifluoroacetyl)amino] benzamide | 489.8 | 489.1159 |
| 52 | 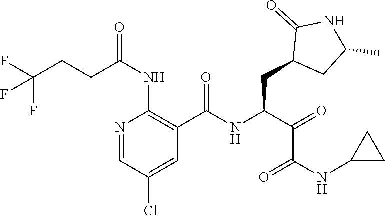 [SMILES: C[C@@H]1C[C@@H](C[C@H](NC(=O)c2cc(Cl)cnc2NC(=O)CCC(F)(F)F)C(=O)C(=O)NC2CC2)C(=O)N1] | 5-chloro-N-[(1S)-3- (cyclopropylamino)-1- [[(3S,5R)-5-methyl-2-oxo- pyrrolidin-3-yl]methyl]-2,3- dioxo-propyl]-2-(4,4,4- trifluorobutanoylamino) pyridine-3-carboxamide | 532.9 | 532.1589 |
| 53 | 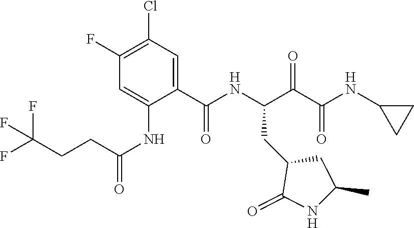 [SMILES: C[C@@H]1C[C@@H](C[C@H](NC(=O)c2cc(Cl)c(F)cc2NC(=O)CCC(F)(F)F)C(=O)C(=O)NC2CC2)C(=O)N1] | 5-chloro-N-[(1S)-3- (cyclopropylamino)-1- [[(3S,5R)-5-methyl-2-oxo- pyrrolidin-3-yl]methyl]-2,3- dioxo-propyl]-4-fluoro-2- (4,4,4- trifluorobutanoylamino) benzamide | 549.9 | 549.1537 |
| 54 | 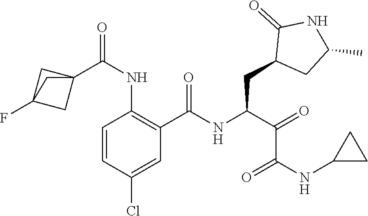 [SMILES: C[C@@H]1C[C@@H](C[C@H](NC(=O)c2cc(Cl)ccc2NC(=O)C23CC(F)(C2)C3)C(=O)C(=O)NC2CC2)C(=O)N1] | N-[4-chloro-2-[[(1S)-3- (cyclopropylamino)-1- [[(3S,5R)-5-methyl-2-oxo- pyrrolidin-3-yl]methyl]-2,3- dioxo- propyl]carbamoyl]phenyl]-3- fluoro-bicyclo[1.1.1]pentane- 1-carboxamide | 519.9 | 519.1821 |
| 55 | 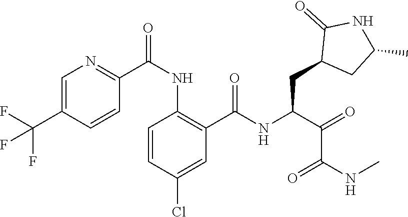 [SMILES: CNC(=O)C(=O)[C@H](C[C@@H]1C[C@@H](C)NC1=O)NC(=O)c1cc(Cl)ccc1NC(=O)c1ccc(C(F)(F)F)cn1] | N-[4-chloro-2-[[(1S)-3- (methylamino)-1-[[(3S,5R)- 5-methyl-2-oxo-pyrrolidin-3- yl]methyl]-2,3-dioxo- propyl]carbamoyl]phenyl]-5- (trifluoromethyl)pyridine-2- carboxamide | 554.9 | 554.1422 |
| 56 | 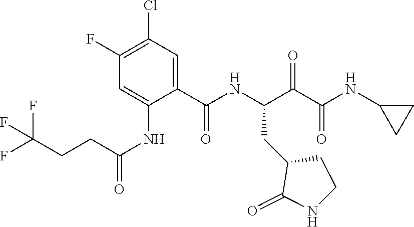 [SMILES: O=C(CCC(F)(F)F)Nc1cc(F)c(Cl)cc1C(=O)N[C@@H](C[C@@H]1CCNC1=O)C(=O)C(=O)NC1CC1] | 5-chloro-N-[(1S)-3- (cyclopropylamino)-2,3- dioxo-1-[[(3S)-2- oxopyrrolidin-3- yl]methyl]propyl]-4-fluoro-2- (4,4,4- trifluorobutanoylamino) benzamide | 535.9 | 535.1377 |
| 57 | 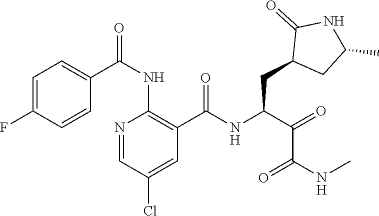 [SMILES: CNC(=O)C(=O)[C@H](C[C@@H]1C[C@@H](C)NC1=O)NC(=O)c1cc(Cl)cnc1NC(=O)c1ccc(F)cc1] | 5-chloro-2-[(4- fluorobenzoyl)amino]-N- [(1S)-3-(methylamino)-1- [[(3S,5R)-5-methyl-2-oxo- pyrrolidin-3-yl]methyl]-2,3- dioxo-propyl]pyridine-3- carboxamide | 504.9 | 504.1458 |
| 58 | 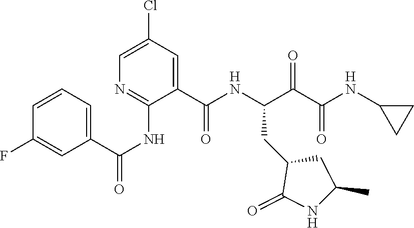 [SMILES: C[C@@H]1C[C@@H](C[C@H](NC(=O)c2cc(Cl)cnc2NC(=O)c2cccc(F)c2)C(=O)C(=O)NC2CC2)C(=O)N1] | 5-chloro-N-[(1S)-3- (cyclopropylamino)-1- [[(3S,5R)-5-methyl-2-oxo- pyrrolidin-3-yl]methyl]-2,3- dioxo-propyl]-2-[(3- fluorobenzoyl)amino] pyridine-3-carboxamide | 530.9 | 530.1619 |
| 59 | 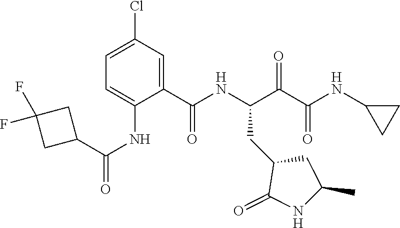 [SMILES: C[C@@H]1C[C@@H](C[C@H](NC(=O)c2cc(Cl)ccc2NC(=O)C2CC(F)(F)C2)C(=O)C(=O)NC2CC2)C(=O)N1] | 5-chloro-N-[(1S)-3- (cyclopropylamino)-1- [[(3S,5R)-5-methyl-2-oxo- pyrrolidin-3-yl]methyl]-2,3- dioxo-propyl]-2-[(3,3- difluorocyclobutanecarbonyl) amino]benzamide | 525.9 | 525.1719 |
| 60 | 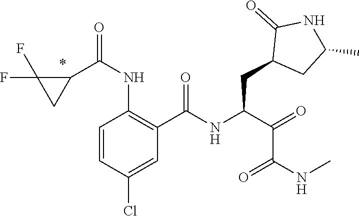 [SMILES: CNC(=O)C(=O)[C@H](C[C@@H]1C[C@@H](C)NC1=O)NC(=O)c1cc(Cl)ccc1NC(=O)C1CC1(F)F] | 5-chloro-2-[(2,2- difluorocyclopropanecarbonyl) amino]-N-[(1S)-3- (methylamino)-1-[[(3S,5R)- 5-methyl-2-oxo-pyrrolidin-3- yl]methyl]-2,3-dioxo- propyl]benzamide | 485.9 | 485.1413 |
| 61 | 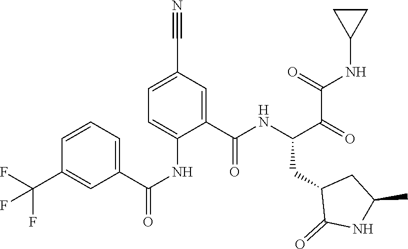 [SMILES: C[C@@H]1C[C@@H](C[C@H](NC(=O)c2cc(C#N)ccc2NC(=O)c2cccc(C(F)(F)F)c2)C(=O)C(=O)NC2CC2)C(=O)N1] | 5-cyano-N-[(1S)-3- (cyclopropylamino)-1- [[(3S,5R)-5-methyl-2-oxo- pyrrolidin-3-yl]methyl]-2,3- dioxo-propyl]-2-[[3- (trifluoromethyl)benzoyl] amino]benzamide | 570.5 | 570.1961 |
| 62 | 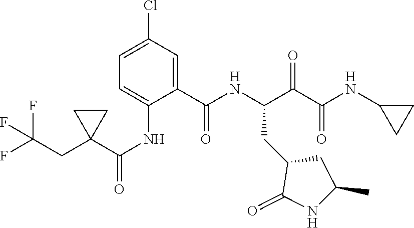 [SMILES: C[C@@H]1C[C@@H](C[C@H](NC(=O)c2cc(Cl)ccc2NC(=O)C2(CC(F)(F)F)CC2)C(=O)C(=O)NC2CC2)C(=O)N1] | 5-chloro-N-[(1S)-3- (cyclopropylamino)-1- [[(3S,5R)-5-methyl-2-oxo- pyrrolidin-3-yl]methyl]-2,3- dioxo-propyl]-2-[1-(2,2,2- trifluoroethyl)cyclopropane- carbonyl]amino]benzamide | 557.9 | 557.1785 |
| 63 | 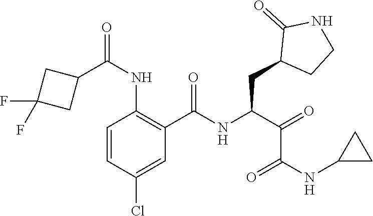 [SMILES: O=C(NC1CC1)C(=O)[C@H](C[C@@H]1CCNC1=O)NC(=O)c1cc(Cl)ccc1NC(=O)C1CC(F)(F)C1] | 5-chloro-N-[(1S)-3- (cyclopropylamino)-2,3- dioxo-1-[[(3S)-2- oxopyrrolidin-3- yl]methyl]propyl]-2-[(3,3- difluorocyclobutanecarbonyl) amino]benzamide | 511.9 | 511.156 |
| 64 | 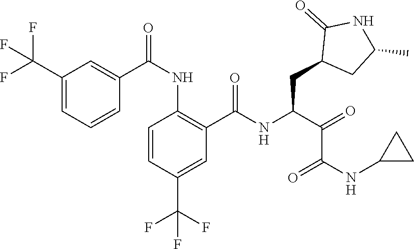 [SMILES: C[C@@H]1C[C@@H](C[C@H](NC(=O)c2cc(C(F)(F)F)ccc2NC(=O)c2cccc(C(F)(F)F)c2)C(=O)C(=O)NC2CC2)C(=O)N1] | N-[(1S)-3- (cyclopropylamino)-1- [[(3S,5R)-5-methyl-2-oxo- pyrrolidin-3-yl]methyl]-2,3- dioxo-propyl]-5- (trifluoromethyl)-2-[[3- (trifluoromethyl)benzoyl] amino]benzamide | 613.5 | 613.189 |
| 65 | 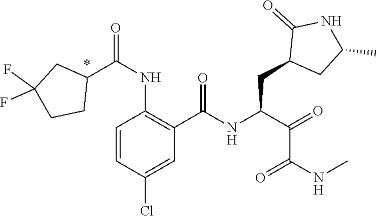 [SMILES: CNC(=O)C(=O)[C@H](C[C@@H]1C[C@@H](C)NC1=O)NC(=O)c1cc(Cl)ccc1NC(=O)C1CCC(F)(F)C1] | 5-chloro-2-[(3,3- difluorocyclopentanecarbonyl) amino]-N-[(1S)-3- (methylamino)-1-[[(3S,5R)- 5-methyl-2-oxo-pyrrolidin-3- yl]methyl]-2,3-dioxo- propyl]benzamide | 513.9 | 513.1727 |
| 66 | 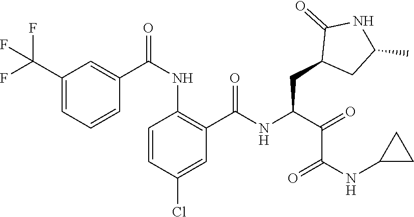 [SMILES: C[C@@H]1C[C@@H](C[C@H](NC(=O)c2cc(Cl)ccc2NC(=O)c2cccc(C(F)(F)F)c2)C(=O)C(=O)NC2CC2)C(=O)N1] | 5-chloro-N-[(1S)-3- (cyclopropylamino)-1- [[(3S,5R)-5-methyl-2-oxo- pyrrolidin-3-yl]methyl]-2,3- dioxo-propyl]-2-[[3- (trifluoromethyl)benzoyl] amino]benzamide | 579.9 | 579.1 |
| 67 | 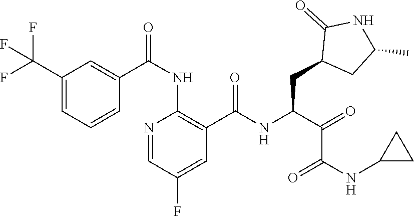 [SMILES: C[C@@H]1C[C@@H](C[C@H](NC(=O)c2cc(F)cnc2NC(=O)c2cccc(C(F)(F)F)c2)C(=O)C(=O)NC2CC2)C(=O)N1] | N-[(1S)-3- (cyclopropylamino)-1- [[(3S,5R)-5-methyl-2-oxo- pyrrolidin-3-yl]methyl]-2,3- dioxo-propyl]-5-fluoro-2-[[3- (trifluoromethyl)benzoyl] amino]pyridine-3-carboxamide | 564.5 | 564.188 |
| 68 | 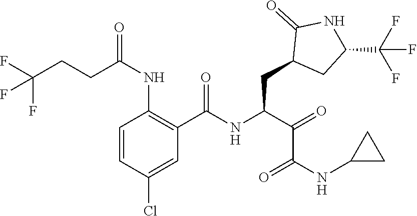 [SMILES: O=C(CCC(F)(F)F)Nc1ccc(Cl)cc1C(=O)N[C@@H](C[C@@H]1C[C@@H](C(F)(F)F)NC1=O)C(=O)C(=O)NC1CC1] | 5-chloro-N-[(1S)-3- (cyclopropylamino)-2,3- dioxo-1-[[(3R,5S)-2-oxo-5- (trifluoromethyl)pyrrolidin-3- yl]methyl]propyl]-2-(4,4,4- trifluorobutanoylamino) benzamide | 585.9 | 585.1345 |
| 69 | 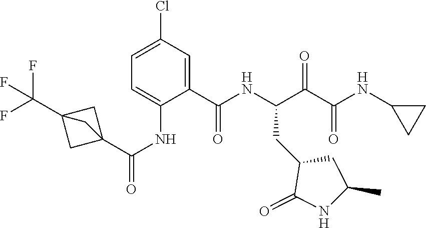 [SMILES: C[C@@H]1C[C@@H](C[C@H](NC(=O)c2cc(Cl)ccc2NC(=O)C23CC(C(F)(F)F)(C2)C3)C(=O)C(=O)NC2CC2)C(=O)N1] | N-[4-chloro-2-[[(1S)-3- (cyclopropylamino)-1- [[(3S,5R)-5-methyl-2-oxo- pyrrolidin-3-yl]methyl]-2,3- dioxo- propyl]carbamoyl]phenyl]-3- (trifluoromethyl)bicyclo[1.1.1] pentane-1-carboxamide | 569.9 | 569.1779 |
| 70 | 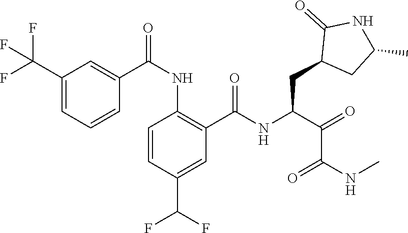 [SMILES: CNC(=O)C(=O)[C@H](C[C@@H]1C[C@@H](C)NC1=O)NC(=O)c1cc(C(F)F)ccc1NC(=O)c1cccc(C(F)(F)F)c1] | 5-(difluoromethyl)-N-[(1S)- 3-(methylamino)-1- [[(3S,5R)-5-methyl-2-oxo- pyrrolidin-3-yl]methyl]-2,3- dioxo-propyl]-2-[[3- (trifluoromethyl)benzoyl] amino]benzamide | 569.5 | 569.1829 |
| 71 | 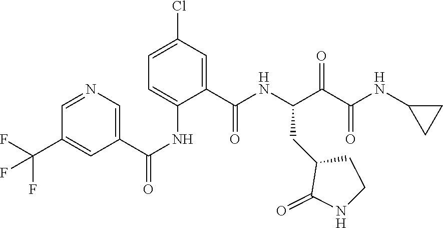 [SMILES: O=C(NC1CC1)C(=O)[C@H](C[C@@H]1CCNC1=O)NC(=O)c1cc(Cl)ccc1NC(=O)c1cncc(C(F)(F)F)c1] | N-[4-chloro-2-[[(1S)-3- (cyclopropylamino)-2,3- dioxo-1-[[(3S)-2- oxopyrrolidin-3- yl]methyl]propyl]carbamoyl] phenyl]-5- (trifluoromethyl)pyridine-3- carboxamide | 566.9 | 566.1428 |
| 72 | 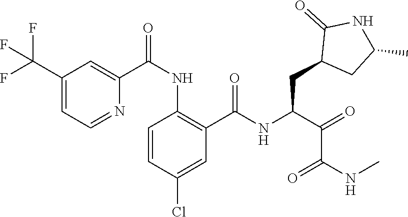 [SMILES: CNC(=O)C(=O)[C@H](C[C@@H]1C[C@@H](C)NC1=O)NC(=O)c1cc(Cl)ccc1NC(=O)c1cc(C(F)(F)F)ccn1] | N-[4-chloro-2-[[(1S)-3- (methylamino)-1-[[(3S,5R)- 5-methyl-2-oxo-pyrrolidin-3- yl]methyl]-2,3-dioxo- propyl]carbamoyl]phenyl]-4- (trifluoromethyl)pyridine-2- carboxamide | 554.9 | 554.1428 |
| 73 | 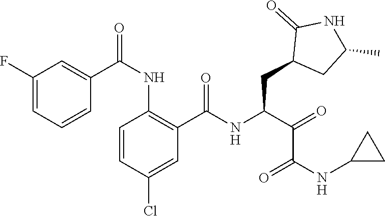 [SMILES: C[C@@H]1C[C@@H](C[C@H](NC(=O)c2cc(Cl)ccc2NC(=O)c2cccc(F)c2)C(=O)C(=O)NC2CC2)C(=O)N1] | 5-chloro-N-[(1S)-3- (cyclopropylamino)-1- [[(3S,5R)-5-methyl-2-oxo- pyrrolidin-3-yl]methyl]-2,3- dioxo-propyl]-2-[(3- fluorobenzoyl)amino] benzamide | 529.9 | 529.1656 |
| 74 | 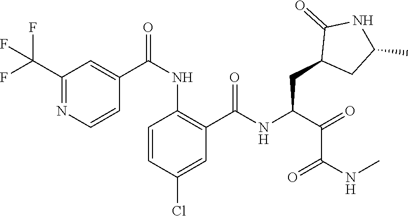 [SMILES: CNC(=O)C(=O)[C@H](C[C@@H]1C[C@@H](C)NC1=O)NC(=O)c1cc(Cl)ccc1NC(=O)c1ccnc(C(F)(F)F)c1] | N-[4-chloro-2-[(1S)-3- (methylamino)-1-[[(3S,5R)- 5-methyl-2-oxo-pyrrolidin-3- yl]methyl]-2,3-dioxo- propyl]carbamoyl]phenyl]-2- (trifluoromethyl)pyridine-4- carboxamide | 554.9 | 554.1427 |
| 75 | 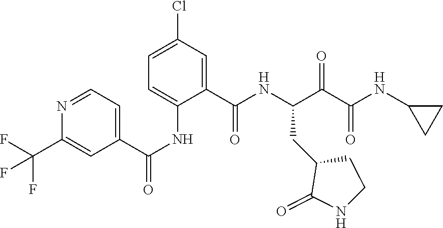 [SMILES: O=C(NC1CC1)C(=O)[C@H](C[C@@H]1CCNC1=O)NC(=O)c1cc(Cl)ccc1NC(=O)c1ccnc(C(F)(F)F)c1] | N-[4-chloro-2-[[(1S)-3- (cyclopropylamino)-2,3- dioxo-1-[[(3S)-2- oxopyrrolidin-3- yl]methyl]propyl]carbamoyl] phenyl]-2- (trifluoromethyl)pyridine-4- carboxamide | 566.9 | 566.1429 |
| 76 | 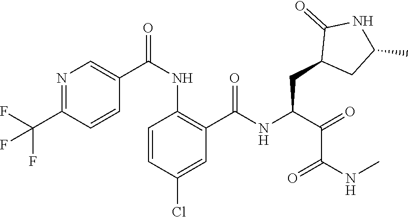 [SMILES: CNC(=O)C(=O)[C@H](C[C@@H]1C[C@@H](C)NC1=O)NC(=O)c1cc(Cl)ccc1NC(=O)c1ccc(C(F)(F)F)nc1] | N-[4-chloro-2-[[(1S)-3- (methylamino)-1-[[(3S,5R)- 5-methyl-2-oxo-pyrrolidin-3- yl]methyl]-2,3-dioxo- propyl]carbamoyl]phenyl]-6- (trifluoromethyl)pyridine-3- carboxamide | 554.9 | 554.1425 |
| 77 | 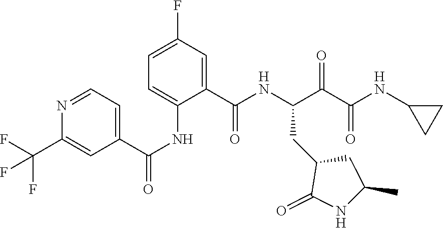 [SMILES: C[C@@H]1C[C@@H](C[C@H](NC(=O)c2cc(F)ccc2NC(=O)c2ccnc(C(F)(F)F)c2)C(=O)C(=O)NC2CC2)C(=O)N1] | N-[2-[[(1S)-3- (cyclopropylamino)-1- [[(3S,5R)-5-methyl-2-oxo- pyrrolidin-3-yl]methyl]-2,3- dioxo-propyl]carbamoyl]-4- fluoro-phenyl]-2- (trifluoromethyl)pyridine-4- carboxamide | 564.5 | 564.188 |
| 78 | 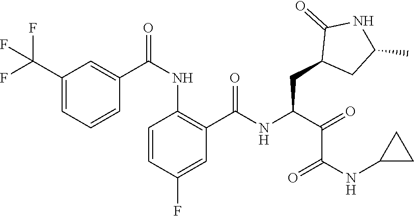 [SMILES: C[C@@H]1C[C@@H](C[C@H](NC(=O)c2cc(F)ccc2NC(=O)c2cccc(C(F)(F)F)c2)C(=O)C(=O)NC2CC2)C(=O)N1] | N-[(1S)-3- (cyclopropylamino)-1- [[(3S,5R)-5-methyl-2-oxo- pyrrolidin-3-yl]methyl]-2,3- dioxo-propyl]-5-fluoro-2-[[3- (trifluoromethyl)benzoyl] amino]benzamide | 563.5 | 563.192 |
| 79 | 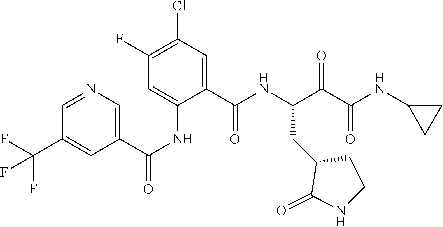 [SMILES: O=C(NC1CC1)C(=O)[C@H](C[C@@H]1CCNC1=O)NC(=O)c1cc(Cl)c(F)cc1NC(=O)c1cncc(C(F)(F)F)c1] | N-[4-chloro-2-[(1S)-3- (cyclopropylamino)-2,3- dioxo-1-[[(3S)-2- oxopyrrolidin-3- yl]methyl]propyl]carbamoyl]- 5-fluoro-phenyl]-5- (trifluoromethyl)pyridine-3- carboxamide | 584.9 | 584.1335 |
| 80 | 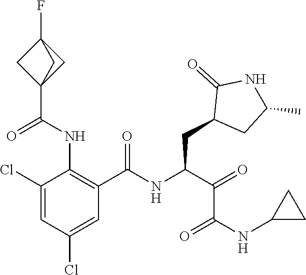 [SMILES: C[C@@H]1C[C@@H](C[C@H](NC(=O)c2cc(Cl)cc(Cl)c2NC(=O)C23CC(F)(C2)C3)C(=O)C(=O)NC2CC2)C(=O)N1] | N-[2,4-dichloro-6-[[(1S)-3- (cyclopropylamino)-1- [[(3S,5R)-5-methyl-2-oxo- pyrrolidin-3-yl]methyl]-2,3- dioxo- propyl]carbamoyl]phenyl]-3- fluoro-bicyclo[1.1.1]pentane- 1-carboxamide | 553.1 | 553.1429 |
| 81 | 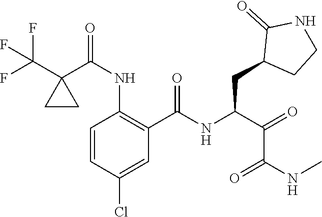 [SMILES: CNC(=O)C(=O)[C@H](C[C@@H]1CCNC1=O)NC(=O)c1cc(Cl)ccc1NC(=O)C1(C(F)(F)F)CC1] | 5-chloro-N-[(1S)-3- (methylamino)-2,3-dioxo-1- [[(3S)-2-oxopyrrolidin-3- yl]methyl]propyl]-2-[[1- (trifluoromethyl)cyclopropane- carbonyl]amino]benzamide | 503.9 | 503.1319 |
| 82 | 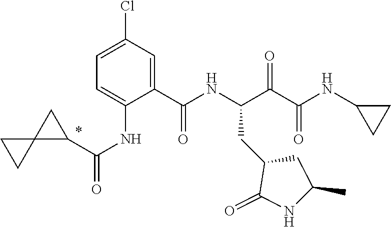 [SMILES: C[C@@H]1C[C@@H](C[C@H](NC(=O)c2cc(Cl)ccc2NC(=O)C2CC23CC3)C(=O)C(=O)NC2CC2)C(=O)N1] | N-[4-chloro-2-[[(1S)-3- (cyclopropylamino)-1- [[(3S,5R)-5-methyl-2-oxo- pyrrolidin-3-yl]methyl]-2,3- dioxo- propyl]carbamoyl]phenyl] spiro[2.2]pentane-2- carboxamide | 501.9 | 501.1901 |
| 83 | 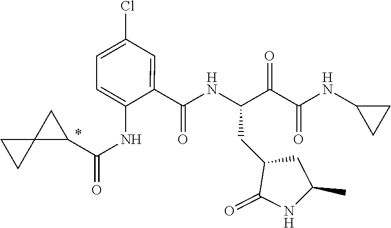 [SMILES: C[C@@H]1C[C@@H](C[C@H](NC(=O)c2cc(Cl)ccc2NC(=O)C2CC23CC3)C(=O)C(=O)NC2CC2)C(=O)N1] | N-[4-chloro-2-[[(1S)-3- (cyclopropylamino)-1- [[(3S,5R)-5-methyl-2-oxo- pyrrolidin-3-yl]methyl]-2,3- dioxo- propyl]carbamoyl]phenyl] spiro[2.2]pentane-2- carboxamide | 501.9 | 501.1901 |
| 84 | 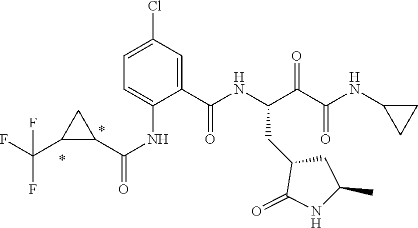 [SMILES: C[C@@H]1C[C@@H](C[C@H](NC(=O)c2cc(Cl)ccc2NC(=O)C2CC2C(F)(F)F)C(=O)C(=O)NC2CC2)C(=O)N1] | 5-chloro-N-[(1S)-3- (cyclopropylamino)-1- [[(3S,5R)-5-methyl-2-oxo- pyrrolidin-3-yl]methyl]-2,3- dioxo-propyl]-2-[[2- (trifluoromethyl)cyclopropane- carbonyl]amino]benzamide | 543.9 | 543.1629 |
| 85 | 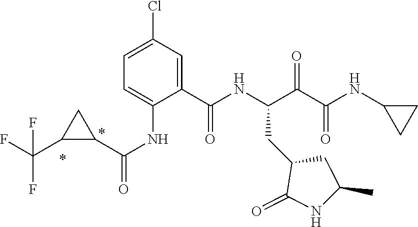 [SMILES: C[C@@H]1C[C@@H](C[C@H](NC(=O)c2cc(Cl)ccc2NC(=O)C2CC2C(F)(F)F)C(=O)C(=O)NC2CC2)C(=O)N1] | 5-chloro-N-[(1S)-3- (cyclopropylamino)-1- [[(3S,5R)-5-methyl-2-oxo- pyrrolidin-3-yl]methyl]-2,3- dioxo-propyl]-2-[[2- (trifluoromethyl)cyclopropane- carbonyl]amino]benzamide | 543.9 | 543.1625 |
| 86 | 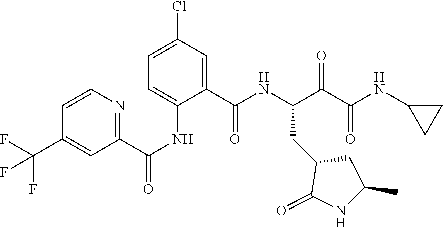 [SMILES: C[C@@H]1C[C@@H](C[C@H](NC(=O)c2cc(Cl)ccc2NC(=O)c2cc(C(F)(F)F)ccn2)C(=O)C(=O)NC2CC2)C(=O)N1] | N-[4-chloro-2-[[(1S)-3- (cyclopropylamino)-1- [[(3S,5R)-5-methyl-2-oxo- pyrrolidin-3-yl]methyl]-2,3- dioxo- propyl]carbamoyl]phenyl]-4- (trifluoromethyl)pyridine-2- carboxamide | 580.9 | 580.1582 |
| 87 | 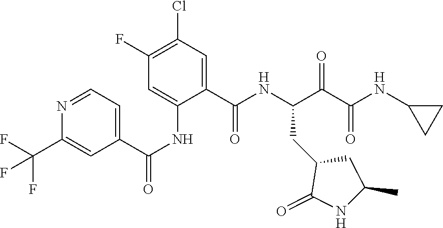 [SMILES: C[C@@H]1C[C@@H](C[C@H](NC(=O)c2cc(Cl)c(F)cc2NC(=O)c2ccnc(C(F)(F)F)c2)C(=O)C(=O)NC2CC2)C(=O)N1] | N-[4-chloro-2-[(1S)-3- (cyclopropylamino)-1- [[(3S,5R)-5-methyl-2-oxo- pyrrolidin-3-yl]methyl]-2,3- dioxo-propyl]carbamoyl]-5- fluoro-phenyl]-2- (trifluoromethyl)pyridine-4- carboxamide | 598.9 | 598.1494 |
| 88 | 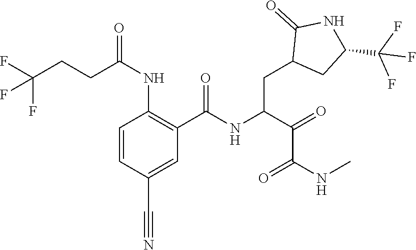 [SMILES: CNC(=O)C(=O)C(CC1C[C@@H](C(F)(F)F)NC1=O)NC(=O)c1cc(C#N)ccc1NC(=O)CCC(F)(F)F] | 5-cyano-N-[3- (methylamino)-2,3-dioxo-1- [[(5S)-2-oxo-5- (trifluoromethyl)pyrrolidin-3- yl]methyl]propyl]-2-(4,4,4- trifluorobutanoylamino) benzamide | 550.4 | 550.1526 |
| 89 | 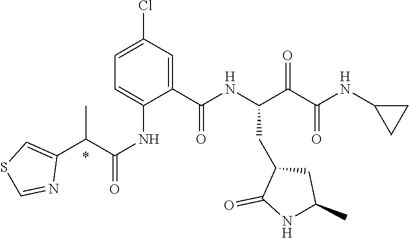 [SMILES: CC(C(=O)Nc1ccc(Cl)cc1C(=O)N[C@@H](C[C@@H]1C[C@@H](C)NC1=O)C(=O)C(=O)NC1CC1)c1cscn1] | 5-chloro-N-[(1S)-3- (cyclopropylamino)-1- [[(3S,5R)-5-methyl-2-oxo- pyrrolidin-3-yl]methyl]-2,3- dioxo-propyl]-2-(2-thiazol-4- ylpropanoylamino)benzamide | 546.2 | 546.1584 |
| 90 | 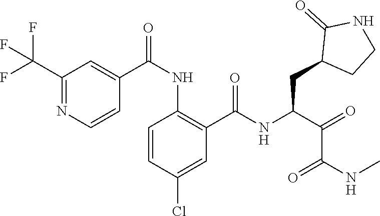 [SMILES: CNC(=O)C(=O)[C@H](C[C@@H]1CCNC1=O)NC(=O)c1cc(Cl)ccc1NC(=O)c1ccnc(C(F)(F)F)c1] | N-[4-chloro-2-[(1S)-3- (methylamino)-2,3-dioxo-1- [[(3S)-2-oxopyrrolidin-3- yl]methyl]propyl]carbamoyl] phenyl]-2- (trifluoromethyl)pyridine-4- carboxamide | 540.9 | 540.2 |
| 91 | 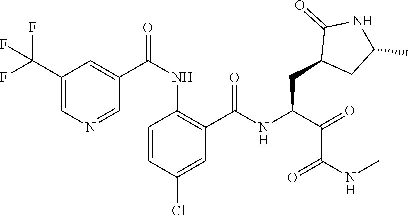 [SMILES: CNC(=O)C(=O)[C@H](C[C@@H]1C[C@@H](C)NC1=O)NC(=O)c1cc(Cl)ccc1NC(=O)c1cncc(C(F)(F)F)c1] | N-[4-chloro-2-[[(1S)-3- (methylamino)-1-[[(3S,5R)- 5-methyl-2-oxo-pyrrolidin-3- yl]methyl]-2,3-dioxo- propyl]carbamoyl]phenyl]-5- (trifluoromethyl)pyridine-3- carboxamide | 554.9 | 554.1418 |
| 92 | 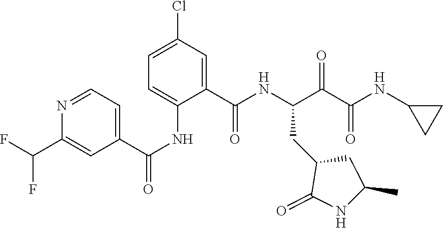 [SMILES: C[C@@H]1C[C@@H](C[C@H](NC(=O)c2cc(Cl)ccc2NC(=O)c2ccnc(C(F)F)c2)C(=O)C(=O)NC2CC2)C(=O)N1] | N-[4-chloro-2-[[(1S)-3- (cyclopropylamino)-1- [[(3S,5R)-5-methyl-2-oxo- pyrrolidin-3-yl]methyl]-2,3- dioxo- propyl]carbamoyl]phenyl]-2- (difluoromethyl)pyridine-4- carboxamide | 562.9 | 562.1678 |
| 93 | 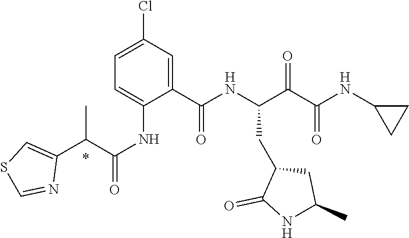 [SMILES: CC(C(=O)Nc1ccc(Cl)cc1C(=O)N[C@@H](C[C@@H]1C[C@@H](C)NC1=O)C(=O)C(=O)NC1CC1)c1cscn1] | 5-chloro-N-[(1S)-3- (cyclopropylamino)-1- [[(3S,5R)-5-methyl-2-oxo- pyrrolidin-3-yl]methyl]-2,3- dioxo-propyl]-2-(2-thiazol-4- ylpropanoylamino)benzamide | 546.2 | 546.3 |
| 94 | 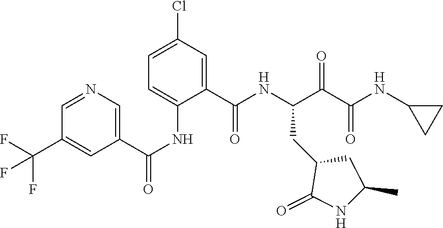 [SMILES: C[C@@H]1C[C@@H](C[C@H](NC(=O)c2cc(Cl)ccc2NC(=O)c2cncc(C(F)(F)F)c2)C(=O)C(=O)NC2CC2)C(=O)N1] | N-[4-chloro-2-[[(1S)-3- (cyclopropylamino)-1- [[(3S,5R)-5-methyl-2-oxo- pyrrolidin-3-yl]methyl]-2,3- dioxo- propyl]carbamoyl]phenyl]-5- (trifluoromethyl)pyridine-3- carboxamide | 580.9 | 580.157 |
| 95 | 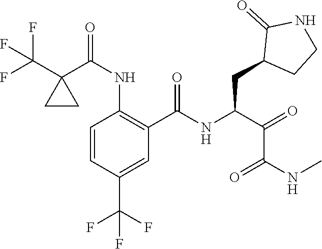 [SMILES: CNC(=O)C(=O)[C@H](C[C@@H]1CCNC1=O)NC(=O)c1cc(C(F)(F)F)ccc1NC(=O)C1(C(F)(F)F)CC1] | N-[(1S)-3-(methylamino)- 2,3-dioxo-1-[[(3S)-2- oxopyrrolidin-3- yl]methyl]propyl]-5- (trifluoromethyl)-2-[[1- (trifluoromethyl)cyclopropane- carbonyl]amino]benzamide | 537.4 | 537.1578 |
| 96 | 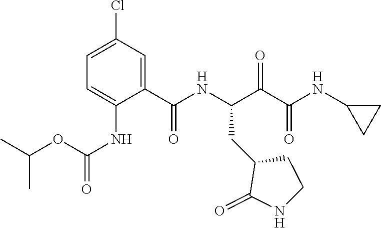 [SMILES: CC(C)OC(=O)Nc1ccc(Cl)cc1C(=O)N[C@@H](C[C@@H]1CCNC1=O)C(=O)C(=O)NC1CC1] | isopropyl N-[4-chloro-2- [[(1S)-3-(cyclopropylamino)- 2,3-dioxo-1-[[(3S)-2- oxopyrrolidin-3- yl]methyl]propyl]carbamoyl] phenyl]carbamate | 479.9 | 479.1701 |
| 97 | 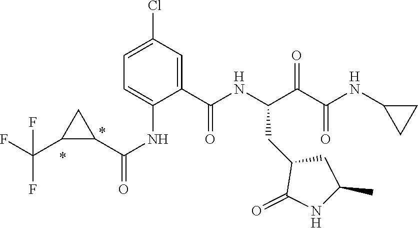 [SMILES: C[C@@H]1C[C@@H](C[C@H](NC(=O)c2cc(Cl)ccc2NC(=O)C2CC2C(F)(F)F)C(=O)C(=O)NC2CC2)C(=O)N1] | 5-chloro-N-[(1S)-3- (cyclopropylamino)-1- [[(3S,5R)-5-methyl-2-oxo- pyrrolidin-3-yl]methyl]-2,3- dioxo-propyl]-2-[[2- (trifluoromethyl)cyclopropane- carbonyl]amino]benzamide | 543.9 | 543.1614 |
| 98 | 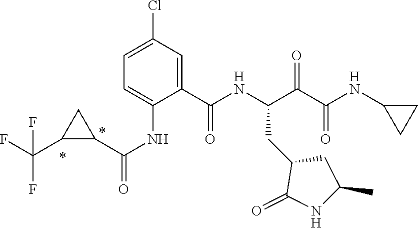 [SMILES: C[C@@H]1C[C@@H](C[C@H](NC(=O)c2cc(Cl)ccc2NC(=O)C2CC2C(F)(F)F)C(=O)C(=O)NC2CC2)C(=O)N1] | 5-chloro-N-[(1S)-3- (cyclopropylamino)-1- [[(3S,5R)-5-methyl-2-oxo- pyrrolidin-3-yl]methyl]-2,3- dioxo-propyl]-2-[[2- (trifluoromethyl)cyclopropane- carbonyl]amino]benzamide | 543.9 | 543.1627 |
| 99 | 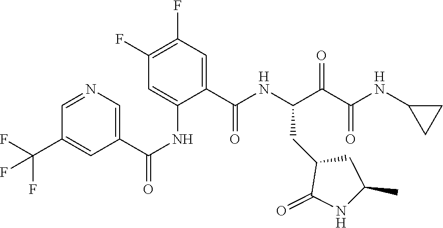 [SMILES: C[C@@H]1C[C@@H](C[C@H](NC(=O)c2cc(F)c(F)cc2NC(=O)c2cncc(C(F)(F)F)c2)C(=O)C(=O)NC2CC2)C(=O)N1] | N-[2-[[(1S)-3- (cyclopropylamino)-1- [[(3S,5R)-5-methyl-2-oxo- pyrrolidin-3-yl]methyl]-2,3- dioxo-propyl]carbamoyl]- 4,5-difluoro-phenyl]-5- (trifluoromethyl)pyridine-3- carboxamide | 582.5 | 582.1776 |
| 100 | 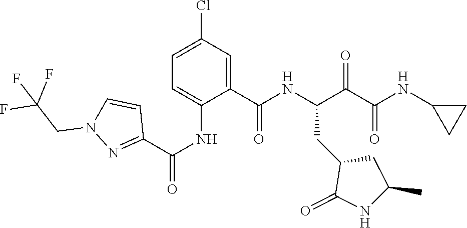 [SMILES: C[C@@H]1C[C@@H](C[C@H](NC(=O)c2cc(Cl)ccc2NC(=O)c2ccn(CC(F)(F)F)n2)C(=O)C(=O)NC2CC2)C(=O)N1] | N-[4-chloro-2-[[(1S)-3- (cyclopropylamino)-1- [[(3S,5R)-5-methyl-2-oxo- pyrrolidin-3-yl]methyl]-2,3- dioxo- propyl]carbamoyl]phenyl]-1- (2,2,2- trifluoroethyl)pyrazole-3- carboxamide | 583.9 | 583.1697 |
| 101 | 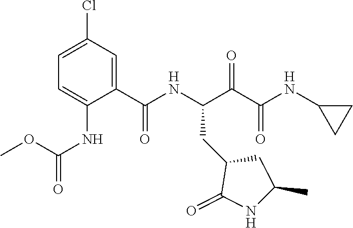 [SMILES: COC(=O)Nc1ccc(Cl)cc1C(=O)N[C@@H](C[C@@H]1C[C@@H](C)NC1=O)C(=O)C(=O)NC1CC1] | methyl N-[4-chloro-2-[(1S)- 3-(cyclopropylamino)-1- [[(3S,5R)-5-methyl-2-oxo- pyrrolidin-3-yl]methyl]-2,3- dioxo- propyl]carbamoyl]phenyl] carbamate | 465.9 | 465.1541 |
| 102 | 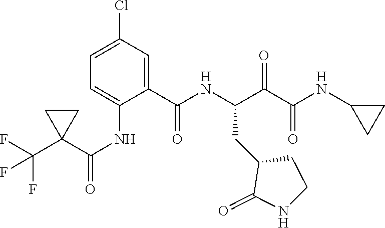 [SMILES: O=C(NC1CC1)C(=O)[C@H](C[C@@H]1CCNC1=O)NC(=O)c1cc(Cl)ccc1NC(=O)C1(C(F)(F)F)CC1] | 5-chloro-N-[(1S)-3- (cyclopropylamino)-2,3- dioxo-1-[[(3S)-2- oxopyrrolidin-3- yl]methyl]propyl]-2-[1- (trifluoromethyl)cyclopropane- carbonyl]amino]benzamide | 529.9 | 529.1466 |
| 103 | 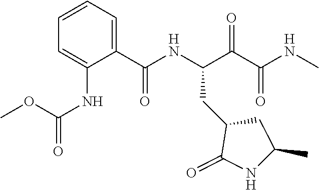 | methyl N-[2-[[(1S)-3- (methylamino)-1-[[(3S,5R)- 5-methyl-2-oxo-pyrrolidin-3- yl]methyl]-2,3-dioxo- propyl]carbamoyl]phenyl] carbamate | 405.4 | 405.1773 |
| 104 | 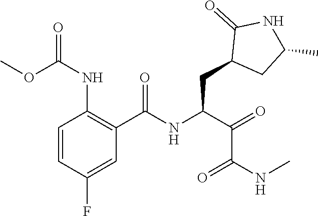 | methyl N-[4-fluoro-2-[(1S)- 3-(methylamino)-1- [[(3S,5R)-5-methyl-2-oxo- pyrrolidin-3-yl]methyl]-2,3- dioxo- propyl]carbamoyl]phenyl] carbamate | 423.4 | 423.1682 |
| 106 | 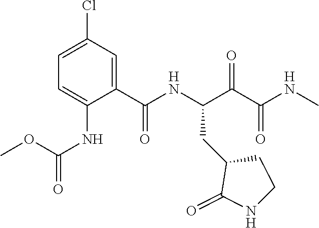 | methyl N-[4-chloro-2-[(1S)- 3-(methylamino)-2,3-dioxo- 1-[(3S)-2-oxopyrrolidin-3- yl]methyl]propyl]carbamoyl] phenyl]carbamate | 425.8 | 425.1226 |
| 107 | 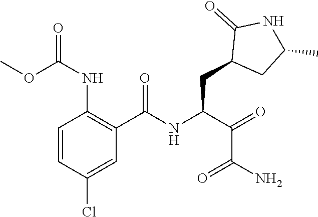 | methyl N-[2-[[(1S)-3-amino- 1-[[(3S,5R)-5-methyl-2-oxo- pyrrolidin-3-yl]methyl]-2,3- dioxo-propyl]carbamoyl]-4- chloro-phenyl]carbamate | 425.8 | 425.1224 |
| 108 | 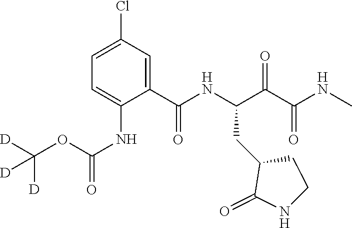 [SMILES: [2H]C([2H])([2H])OC(=O)Nc1ccc(Cl)cc1C(=O)N[C@@H](C[C@@H]1CCNC1=O)C(=O)C(=O)NC] | trideuteriomethyl N-[4- chloro-2-[[(1S)-3- (methylamino)-2,3-dioxo-1- [[(3S)-2-oxopyrrolidin-3- yl]methyl]propyl]carbamoyl] phenyl]carbamate | 428.9 | 428.1413 |
| 109 | 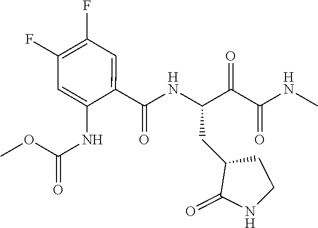 | methyl N-[4,5-difluoro-2- [[(1S)-3-(methylamino)-2,3- dioxo-1-[[(3S)-2- oxopyrrolidin-3- yl]methyl]propyl]carbamoyl] phenyl]carbamate | 427.4 | 427.1432 |
| 110 | 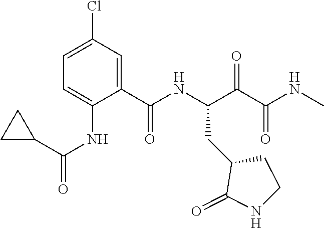 [SMILES: CNC(=O)C(=O)[C@H](C[C@@H]1CCNC1=O)NC(=O)c1cc(Cl)ccc1NC(=O)C1CC1] | 5-chloro-2- (cyclopropanecarbonylamino)- N-[(1S)-3-(methylamino)- 2,3-dioxo-1-[[(3S)-2- oxopyrrolidin-3- yl]methyl]propyl]benzamide | 435.9 | 435.1429 |
| 111 | 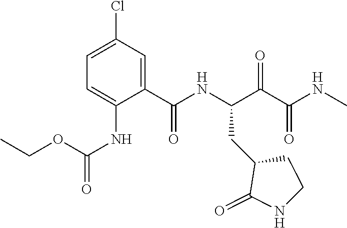 [SMILES: CCOC(=O)Nc1ccc(Cl)cc1C(=O)N[C@@H](C[C@@H]1CCNC1=O)C(=O)C(=O)NC] | ethyl N-[4-chloro-2-[[(1S)-3- (methylamino)-2,3-dioxo-1- [[(3S)-2-oxopyrrolidin-3- yl]methyl]propyl]carbamoyl] phenyl]carbamate | 439.9 | 439.1384 |
| 112 | 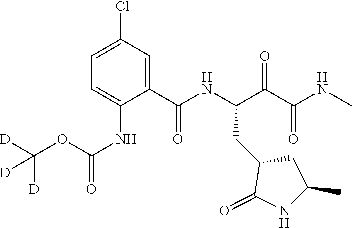 [SMILES: [2H]C([2H])([2H])OC(=O)Nc1ccc(Cl)cc1C(=O)N[C@@H](C[C@@H]1C[C@@H](C)NC1=O)C(=O)C(=O)NC] | trideuteriomethyl N-[4- chloro-2-[[(1S)-3- (methylamino)-1-[[(3S,5R)- 5-methyl-2-oxo-pyrrolidin-3- yl]methyl]-2,3-dioxo- propyl]carbamoyl]phenyl] carbamate | 442.9 | 442.157 |
| 113 | 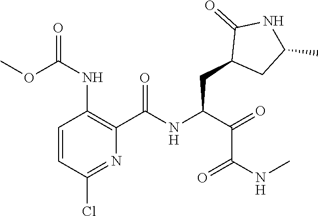 | methyl N-[6-chloro-2-[(1S)- 3-(methylamino)-1- [[(3S,5R)-5-methyl-2-oxo- pyrrolidin-3-yl]methyl]-2,3- dioxo-propyl]carbamoyl]-3- pyridyl]carbamate | 440.9 | 440.1339 |
| 114 | 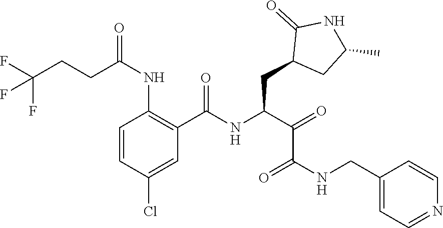 [SMILES: C[C@@H]1C[C@@H](C[C@H](NC(=O)c2cc(Cl)ccc2NC(=O)CCC(F)(F)F)C(=O)C(=O)NCc2ccncc2)C(=O)N1] | 5-chloro-N-[(1S)-1- [[(3S,5R)-5-methyl-2-oxo- pyrrolidin-3-yl]methyl]-2,3- dioxo-3-(4- pyridylmethylamino)propyl]- 2-(4,4,4- trifluorobutanoylamino) benzamide | 583.0 | 582.1733 |
| 116 | 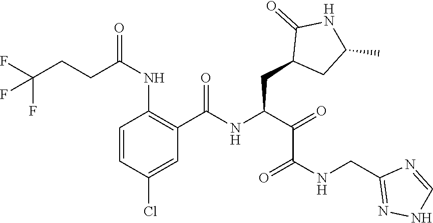 [SMILES: C[C@@H]1C[C@@H](C[C@H](NC(=O)c2cc(Cl)ccc2NC(=O)CCC(F)(F)F)C(=O)C(=O)NCc2nc[nH]n2)C(=O)N1] | 5-chloro-N-[(1S)-1- [[(3S,5R)-5-methyl-2-oxo- pyrrolidin-3-yl]methyl]-2,3- dioxo-3-(1H-1,2,4-triazol-3- ylmethylamino)propyl]-2- (4,4,4- trifluorobutanoylamino) benzamide | 572.9 | 572.1638 |
| 117 | 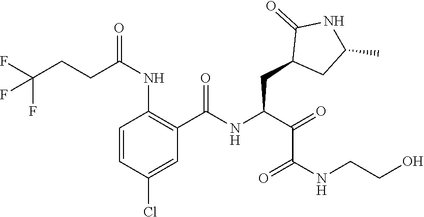 [SMILES: C[C@@H]1C[C@@H](C[C@H](NC(=O)c2cc(Cl)ccc2NC(=O)CCC(F)(F)F)C(=O)C(=O)NCCO)C(=O)N1] | 5-chloro-N-[(1S)-3-(2- hydroxyethylamino)-1- [[(3S,5R)-5-methyl-2-oxo- pyrrolidin-3-yl]methyl]-2,3- dioxo-propyl]-2-(4,4,4- trifluorobutanoylamino) benzamide | 535.9 | 535.157 |
| 118 | 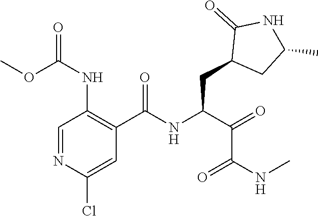 | methyl N-[6-chloro-4-[(1S)- 3-(methylamino)-1- [[(3S,5R)-5-methyl-2-oxo- pyrrolidin-3-yl]methyl]-2,3- dioxo-propyl]carbamoyl]-3- pyridyl]carbamate | 440.9 | 440.1337 |
| 119 | 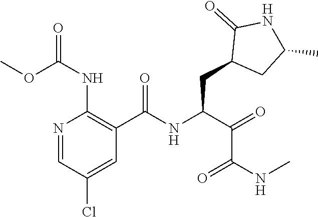 | methyl N-[5-chloro-3-[[(1S)- 3-(methylamino)-1- [[(3S,5R)-5-methyl-2-oxo- pyrrolidin-3-yl]methyl]-2,3- dioxo-propyl]carbamoyl]-2- pyridyl]carbamate | 440.9 | 440.1335 |
| 120 | 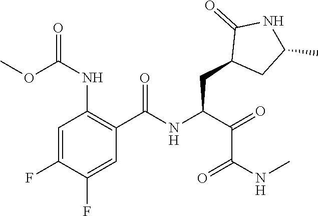 | methyl N-[4,5-difluoro-2- [[(1S)-3-(methylamino)-1- [[(3S,5R)-5-methyl-2-oxo- pyrrolidin-3-yl]methyl]-2,3- dioxo- propyl]carbamoyl]phenyl] carbamate | 441.4 | 441.1585 |
| 121 | 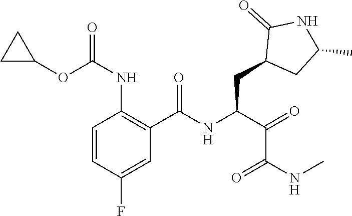 [SMILES: CNC(=O)C(=O)[C@H](C[C@@H]1C[C@@H](C)NC1=O)NC(=O)c1cc(F)ccc1NC(=O)OC1CC1] | cyclopropyl N-[4-fluoro-2- [[(1S)-3-(methylamino)-1- [[(3S,5R)-5-methyl-2-oxo- pyrrolidin-3-yl]methyl]-2,3- dioxo- propyl]carbamoyl]phenyl] carbamate | 449.4 | 449.1834 |
| 122 | 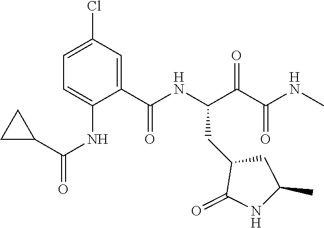 [SMILES: CNC(=O)C(=O)[C@H](C[C@@H]1C[C@@H](C)NC1=O)NC(=O)c1cc(Cl)ccc1NC(=O)C1CC1] | 5-chloro-2- (cyclopropanecarbonylamino)- N-[(1S)-3-(methylamino)- 1-[(3S,5R)-5-methyl-2-oxo- pyrrolidin-3-yl]methyl]-2,3- dioxo-propyl]benzamide | 449.9 | 449.1589 |
| 123 | 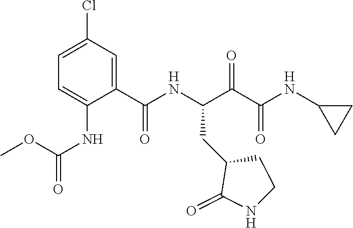 [SMILES: COC(=O)Nc1ccc(Cl)cc1C(=O)N[C@@H](C[C@@H]1CCNC1=O)C(=O)C(=O)NC1CC1] | methyl N-[4-chloro-2-[(1S)- 3-(cyclopropylamino)-2,3- dioxo-1-[[(3S)-2- oxopyrrolidin-3- yl]methyl]propyl]carbamoyl] phenyl]carbamate | 451.9 | 451.1384 |
| 124 | 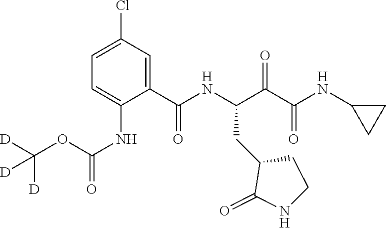 [SMILES: [2H]C([2H])([2H])OC(=O)Nc1ccc(Cl)cc1C(=O)N[C@@H](C[C@@H]1CCNC1=O)C(=O)C(=O)NC1CC1] | trideuteriomethyl N-[4- chloro-2-[[(1S)-3- (cyclopropylamino)-2,3- dioxo-1-[[(3S)-2- oxopyrrolidin-3- yl]methyl]propyl]carbamoyl] phenyl]carbamate | 454.9 | 454.1572 |
| 125 | 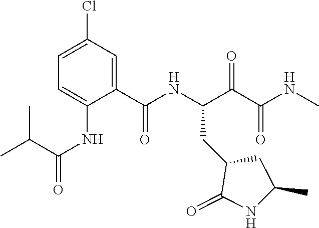 [SMILES: CNC(=O)C(=O)[C@H](C[C@@H]1C[C@@H](C)NC1=O)NC(=O)c1cc(Cl)ccc1NC(=O)C(C)C] | 5-chloro-N-[(1S)-3- (methylamino)-1-[[(3S,5R)- 5-methyl-2-oxo-pyrrolidin-3- yl]methyl]-2,3-dioxo- propyl]-2-(2- methylpropanoylamino) benzamide | 451.9 | 451.1747 |
| 126 | 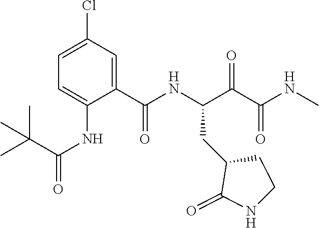 [SMILES: CNC(=O)C(=O)[C@H](C[C@@H]1CCNC1=O)NC(=O)c1cc(Cl)ccc1NC(=O)C(C)(C)C] | 5-chloro-2-(2,2- dimethylpropanoylamino)-N- [(1S)-3-(methylamino)-2,3- dioxo-1-[[(3S)-2- oxopyrrolidin-3- yl]methyl]propyl]benzamide | 451.9 | 451.1749 |
| 127 | 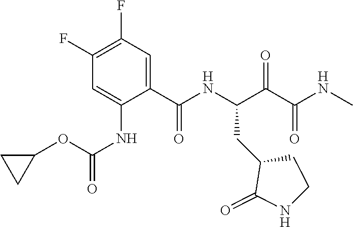 [SMILES: CNC(=O)C(=O)[C@H](C[C@@H]1CCNC1=O)NC(=O)c1cc(F)c(F)cc1NC(=O)OC1CC1] | cyclopropyl N-[4,5-difluoro- 2-[[(1S)-3-(methylamino)- 2,3-dioxo-1-[[(3S)-2- oxopyrrolidin-3- yl]methyl]propyl]carbamoyl] phenyl]carbamate | 453.4 | 453.1583 |
| 128 | 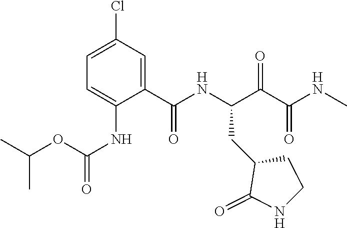 [SMILES: CNC(=O)C(=O)[C@H](C[C@@H]1CCNC1=O)NC(=O)c1cc(Cl)ccc1NC(=O)OC(C)C] | isopropyl N-[4-chloro-2- [[(1S)-3-(methylamino)-2,3- dioxo-1-[[(3S)-2- oxopyrrolidin-3- yl]methyl]propyl]carbamoyl] phenyl]carbamate | 453.9 | 453.1541 |
| 129 | 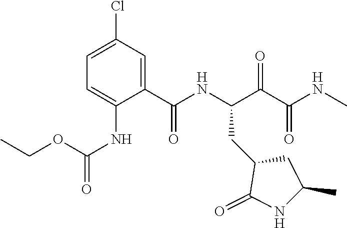 [SMILES: CCOC(=O)Nc1ccc(Cl)cc1C(=O)N[C@@H](C[C@@H]1C[C@@H](C)NC1=O)C(=O)C(=O)NC] | ethyl N-[4-chloro-2-[[(1S)-3- (methylamino)-1-[[(3S,5R)- 5-methyl-2-oxo-pyrrolidin-3- yl]methyl]-2,3-dioxo- propyl]carbamoyl]phenyl] carbamate | 453.9 | 453.1541 |
| 130 | 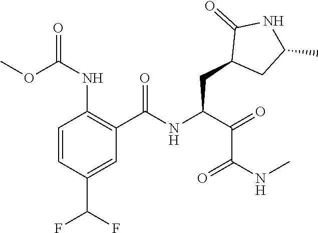 | methyl N-[4- (difluoromethyl)-2-[[(1S)-3- (methylamino)-1-[[(3S,5R)- 5-methyl-2-oxo-pyrrolidin-3- yl]methyl]-2,3-dioxo- propyl]carbamoyl]phenyl] carbamate | 455.4 | 455.174 |
| 131 | 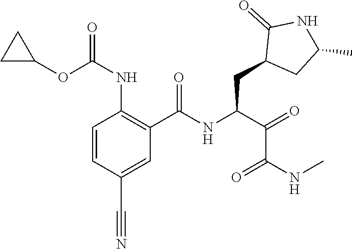 [SMILES: CNC(=O)C(=O)[C@H](C[C@@H]1C[C@@H](C)NC1=O)NC(=O)c1cc(C#N)ccc1NC(=O)OC1CC1] | cyclopropyl N-[4-cyano-2- [[(1S)-3-(methylamino)-1- [[(3S,5R)-5-methyl-2-oxo- pyrrolidin-3-yl]methyl]-2,3- dioxo- propyl]carbamoyl]phenyl] carbamate | 456.5 | 456.1884 |
| 132 | 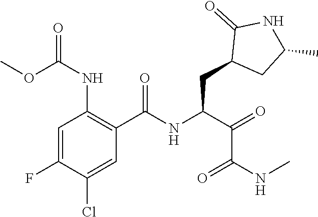 | methyl N-[4-chloro-5-fluoro- 2-[[(1S)-3-(methylamino)-1- [[(3S,5R)-5-methyl-2-oxo- pyrrolidin-3-yl]methyl]-2,3- dioxo- propyl]carbamoyl]phenyl] carbamate | 457.9 | 457.129 |
| 133 | 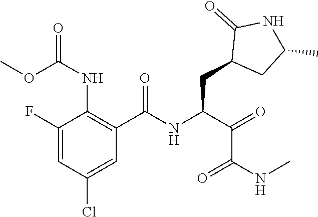 | methyl N-[4-chloro-2-fluoro- 6-[[(1S)-3-(methylamino)-1- [[(3S,5R)-5-methyl-2-oxo- pyrrolidin-3-yl]methyl]-2,3- dioxo- propyl]carbamoyl]phenyl] carbamate | 457.9 | 457.129 |
| 134 | 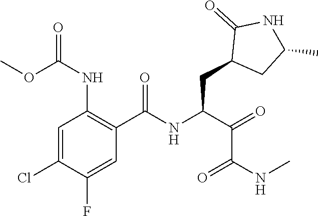 | methyl N-[5-chloro-4-fluoro- 2-[(1S)-3-(methylamino)-1- [[(3S,5R)-5-methyl-2-oxo- pyrrolidin-3-yl]methyl]-2,3- dioxo- propyl]carbamoyl]phenyl] carbamate | 457.9 | 457.129 |
| 135 | 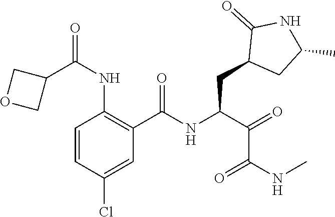 [SMILES: CNC(=O)C(=O)[C@H](C[C@@H]1C[C@@H](C)NC1=O)NC(=O)c1cc(Cl)ccc1NC(=O)C1COC1] | N-[4-chloro-2-[(1S)-3- (methylamino)-1-[[(3S,5R)- 5-methyl-2-oxo-pyrrolidin-3- yl]methyl]-2,3-dioxo- propyl]carbamoyl]phenyl] oxetane-3-carboxamide | 465.9 | 465.1542 |
| 136 | 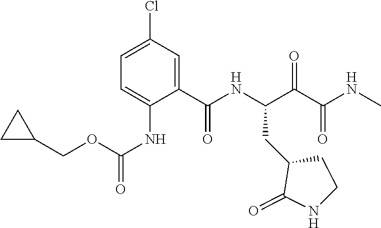 [SMILES: CNC(=O)C(=O)[C@H](C[C@@H]1CCNC1=O)NC(=O)c1cc(Cl)ccc1NC(=O)OCC1CC1] | cyclopropylmethyl N-[4- chloro-2-[[(1S)-3- (methylamino)-2,3-dioxo-1- [[(3S)-2-oxopyrrolidin-3- yl]methyl]propyl]carbamoyl] phenyl]carbamate | 465.9 | 465.1537 |
| 137 | 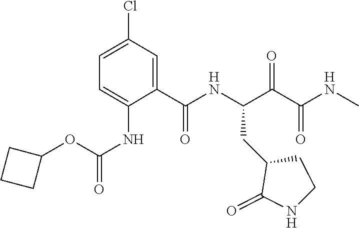 [SMILES: CNC(=O)C(=O)[C@H](C[C@@H]1CCNC1=O)NC(=O)c1cc(Cl)ccc1NC(=O)OC1CCC1] | cyclobutyl N-[4-chloro-2- [[(1S)-3-(methylamino)-2,3- dioxo-1-[[(3S)-2- oxopyrrolidin-3- yl]methyl]propyl]carbamoyl] phenyl]carbamate | 465.9 | 465.1542 |
| 138 | 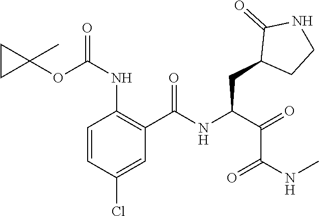 [SMILES: CNC(=O)C(=O)[C@H](C[C@@H]1CCNC1=O)NC(=O)c1cc(Cl)ccc1NC(=O)OC1(C)CC1] | (1-methylcyclopropyl) N-[4- chloro-2-[[(1S)-3- (methylamino)-2,3-dioxo-1- [[(3S)-2-oxopyrrolidin-3- yl]methyl]propyl]carbamoyl] phenyl]carbamate | 465.9 | 465.1542 |
| 139 | 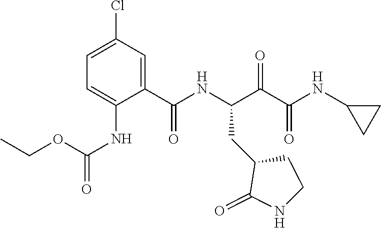 [SMILES: CCOC(=O)Nc1ccc(Cl)cc1C(=O)N[C@@H](C[C@@H]1CCNC1=O)C(=O)C(=O)NC1CC1] | ethyl N-[4-chloro-2-[(1S)-3- (cyclopropylamino)-2,3- dioxo-1-[[(3S)-2- oxopyrrolidin-3- yl]methyl]propyl]carbamoyl] phenyl]carbamate | 465.9 | 465.1542 |
| 140 | 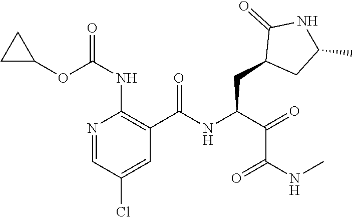 [SMILES: CNC(=O)C(=O)[C@H](C[C@@H]1C[C@@H](C)NC1=O)NC(=O)c1cc(Cl)cnc1NC(=O)OC1CC1] | cyclopropyl N-[5-chloro-3- [[(1S)-3-(methylamino)-1- [[(3S,5R)-5-methyl-2-oxo- pyrrolidin-3-yl]methyl]-2,3- dioxo-propyl]carbamoyl]-2- pyridyl]carbamate | 466.9 | 466.1491 |
| 141 | 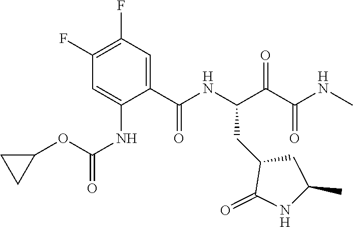 [SMILES: CNC(=O)C(=O)[C@H](C[C@@H]1C[C@@H](C)NC1=O)NC(=O)c1cc(F)c(F)cc1NC(=O)OC1CC1] | cyclopropyl N-[4,5-difluoro- 2-[[(1S)-3-(methylamino)-1- [[(3S,5R)-5-methyl-2-oxo- pyrrolidin-3-yl]methyl]-2,3- dioxo- propyl]carbamoyl]phenyl] carbamate | 467.4 | 467.1739 |
| 142 | 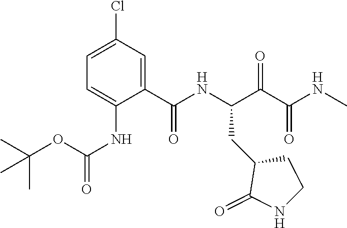 [SMILES: CNC(=O)C(=O)[C@H](C[C@@H]1CCNC1=O)NC(=O)c1cc(Cl)ccc1NC(=O)OC(C)(C)C] | tert-butyl N-[4-chloro-2- [[(1S)-3-(methylamino)-2,3- dioxo-1-[[(3S)-2- oxopyrrolidin-3- yl]methyl]propyl]carbamoyl] phenyl]carbamate | 467.9 | 467.4 |
| 143 | 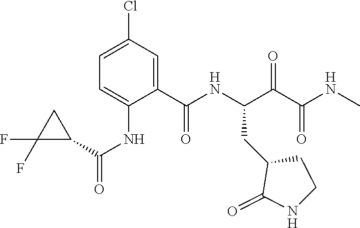 [SMILES: CNC(=O)C(=O)[C@H](C[C@@H]1CCNC1=O)NC(=O)c1cc(Cl)ccc1NC(=O)[C@H]1CC1(F)F] | 5-chloro-2-[[(1R)-2,2- difluorocyclopropanecarbonyl] amino]-N-[(1S)-3- (methylamino)-2,3-dioxo-1- [[(3S)-2-oxopyrrolidin-3- yl]methyl]propyl]benzamide | 471.9 | 471.1249 |
| 144 | 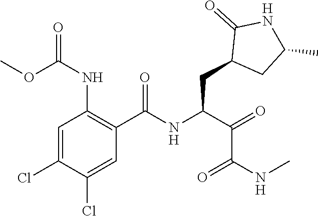 | methyl N-[4,5-dichloro-2- [[(1S)-3-(methylamino)-1- [[(3S,5R)-5-methyl-2-oxo- pyrrolidin-3-yl]methyl]-2,3- dioxo- propyl]carbamoyl]phenyl] carbamate | 474.3 | 473.0997 |
| 145 | 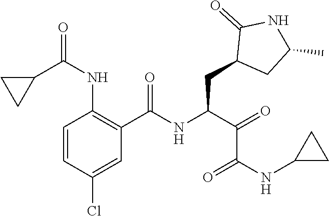 [SMILES: C[C@@H]1C[C@@H](C[C@H](NC(=O)c2cc(Cl)ccc2NC(=O)C2CC2)C(=O)C(=O)NC2CC2)C(=O)N1] | 5-chloro-2- (cyclopropanecarbonylamino)- N-[(1S)-3- (cyclopropylamino)-1- [[(3S,5R)-5-methyl-2-oxo- pyrrolidin-3-yl]methyl]-2,3- dioxo-propyl]benzamide | 475.9 | 475.1746 |
| 146 | 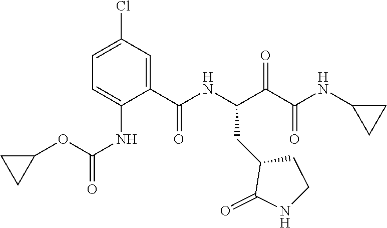 [SMILES: O=C(Nc1ccc(Cl)cc1C(=O)N[C@@H](C[C@@H]1CCNC1=O)C(=O)C(=O)NC1CC1)OC1CC1] | cyclopropyl N-[4-chloro-2- [[(1S)-3-(cyclopropylamino)- 2,3-dioxo-1-[[(3S)-2- oxopyrrolidin-3- yl]methyl]propyl]carbamoyl] phenyl]carbamate | 477.9 | 477.1541 |
| 147 | 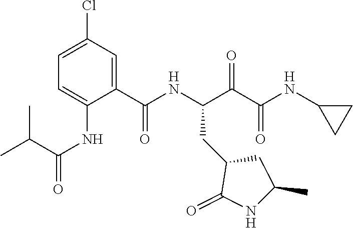 [SMILES: CC(C)C(=O)Nc1ccc(Cl)cc1C(=O)N[C@@H](C[C@@H]1C[C@@H](C)NC1=O)C(=O)C(=O)NC1CC1] | 5-chloro-N-[(1S)-3- (cyclopropylamino)-1- [[(3S,5R)-5-methyl-2-oxo- pyrrolidin-3-yl]methyl]-2,3- dioxo-propyl]-2-(2- methylpropanoylamino) benzamide | 477.9 | 477.3 |
| 148 | 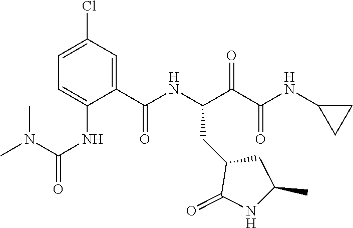 [SMILES: C[C@@H]1C[C@@H](C[C@H](NC(=O)c2cc(Cl)ccc2NC(=O)N(C)C)C(=O)C(=O)NC2CC2)C(=O)N1] | 5-chloro-N-[(1S)-3- (cyclopropylamino)-1- [[(3S,5R)-5-methyl-2-oxo- pyrrolidin-3-yl]methyl]-2,3- dioxo-propyl]-2- (dimethylcarbamoylamino) benzamide | 478.9 | 478.1856 |
| 149 | 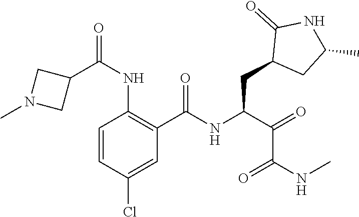 | N-[4-chloro-2-[[(1S)-3- (methylamino)-1-[[(3S,5R)- 5-methyl-2-oxo-pyrrolidin-3- yl]methyl]-2,3-dioxo- propyl]carbamoyl]phenyl]-1- methyl-azetidine-3- carboxamide | 478.9 | 478.1859 |
| 150 | 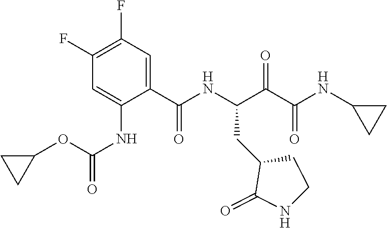 [SMILES: O=C(Nc1cc(F)c(F)cc1C(=O)N[C@@H](C[C@@H]1CCNC1=O)C(=O)C(=O)NC1CC1)OC1CC1] | cyclopropyl N-[2-[(1S)-3- (cyclopropylamino)-2,3- dioxo-1-[[(3S)-2- oxopyrrolidin-3- yl]methyl]propyl]carbamoyl]- 4,5-difluoro- phenyl]carbamate | 479.4 | 479.1742 |
| 151 | 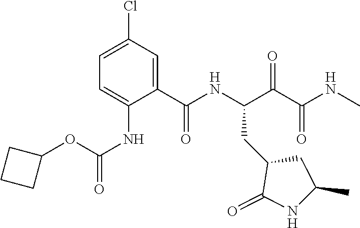 [SMILES: CNC(=O)C(=O)[C@H](C[C@@H]1C[C@@H](C)NC1=O)NC(=O)c1cc(Cl)ccc1NC(=O)OC1CCC1] | cyclobutyl N-[4-chloro-2- [[(1S)-3-(methylamino)-1- [[(3S,5R)-5-methyl-2-oxo- pyrrolidin-3-yl]methyl]-2,3- dioxo- propyl]carbamoyl]phenyl] carbamate | 479.9 | 479.1695 |
| 152 | 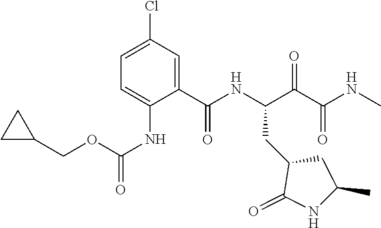 [SMILES: CNC(=O)C(=O)[C@H](C[C@@H]1C[C@@H](C)NC1=O)NC(=O)c1cc(Cl)ccc1NC(=O)OCC1CC1] | cyclopropylmethyl N-[4- chloro-2-[[(1S)-3- (methylamino)-1-[[(3S,5R)- 5-methyl-2-oxo-pyrrolidin-3- yl]methyl]-2,3-dioxo- propyl]carbamoyl]phenyl] carbamate | 479.9 | 479.1699 |
| 153 | 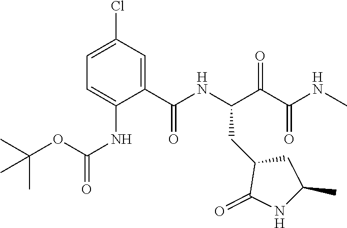 [SMILES: CNC(=O)C(=O)[C@H](C[C@@H]1C[C@@H](C)NC1=O)NC(=O)c1cc(Cl)ccc1NC(=O)OC(C)(C)C] | tert-butyl N-[4-chloro-2- [[(1S)-3-(methylamino)-1- [[(3S,5R)-5-methyl-2-oxo- pyrrolidin-3-yl]methyl]-2,3- dioxo- propyl]carbamoyl]phenyl] carbamate | 481.9 | 481.5 |
| 154 | 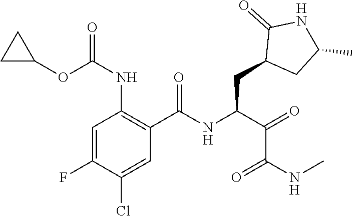 [SMILES: CNC(=O)C(=O)[C@H](C[C@@H]1C[C@@H](C)NC1=O)NC(=O)c1cc(Cl)c(F)cc1NC(=O)OC1CC1] | cyclopropyl N-[4-chloro-5- fluoro-2-[(1S)-3- (methylamino)-1-[[(3S,5R)- 5-methyl-2-oxo-pyrrolidin-3- yl]methyl]-2,3-dioxo- propyl]carbamoyl]phenyl] carbamate | 483.9 | 483.1447 |
| 155 | 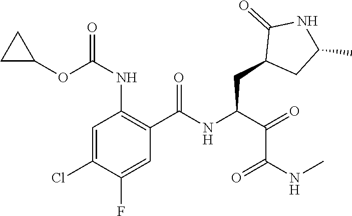 [SMILES: CNC(=O)C(=O)[C@H](C[C@@H]1C[C@@H](C)NC1=O)NC(=O)c1cc(F)c(Cl)cc1NC(=O)OC1CC1] | cyclopropyl N-[5-chloro-4- fluoro-2-[[(1S)-3- (methylamino)-1-[[(3S,5R)- 5-methyl-2-oxo-pyrrolidin-3- yl]methyl]-2,3-dioxo- propyl]carbamoyl]phenyl] carbamate | 483.9 | 483.1443 |
| 156 | 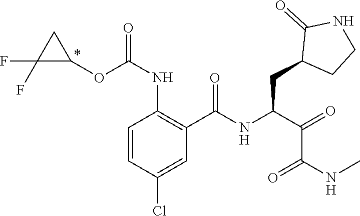 [SMILES: CNC(=O)C(=O)[C@H](C[C@@H]1CCNC1=O)NC(=O)c1cc(Cl)ccc1NC(=O)OC1CC1(F)F] | (2,2-difluorocyclopropyl) N- [4-chloro-2-[(1S)-3- (methylamino)-2,3-dioxo-1- [[(3S)-2-oxopyrrolidin-3- yl]methyl]propyl]carbamoyl] phenyl]carbamate | 487.9 | 487.1197 |
| 157 | 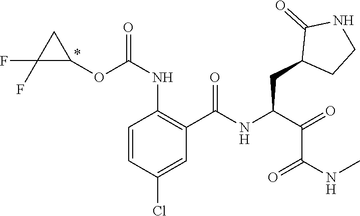 [SMILES: CNC(=O)C(=O)[C@H](C[C@@H]1CCNC1=O)NC(=O)c1cc(Cl)ccc1NC(=O)OC1CC1(F)F] | (2,2-difluorocyclopropyl) N- [4-chloro-2-[[(1S)-3- (methylamino)-2,3-dioxo-1- [[(3S)-2-oxopyrrolidin-3- yl]methyl]propyl]carbamoyl] phenyl]carbamate | 487.9 | 487.12 |
| 158 | 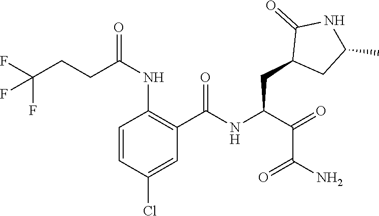 [SMILES: C[C@@H]1C[C@@H](C[C@H](NC(=O)c2cc(Cl)ccc2NC(=O)CCC(F)(F)F)C(=O)C(N)=O)C(=O)N1] | N-[(1S)-3-amino-1- [[(3S,5R)-5-methyl-2-oxo- pyrrolidin-3-yl]methyl]-2,3- dioxo-propyl]-5-chloro-2- (4,4,4- trifluorobutanoylamino) benzamide | 491.9 | 491.1308 |
| 159 | 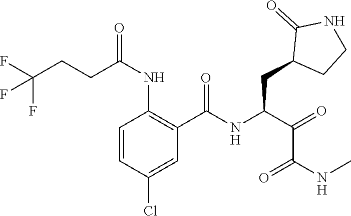 [SMILES: CNC(=O)C(=O)[C@H](C[C@@H]1CCNC1=O)NC(=O)c1cc(Cl)ccc1NC(=O)CCC(F)(F)F] | 5-chloro-N-[(1S)-3- (methylamino)-2,3-dioxo-1- [[(3S)-2-oxopyrrolidin-3- yl]methyl]propyl]-2-(4,4,4- trifluorobutanoylamino) benzamide | 491.9 | 491.1308 |
| 160 | 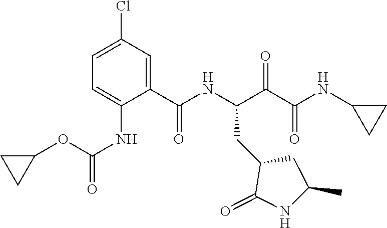 [SMILES: C[C@@H]1C[C@@H](C[C@H](NC(=O)c2cc(Cl)ccc2NC(=O)OC2CC2)C(=O)C(=O)NC2CC2)C(=O)N1] | cyclopropyl N-[4-chloro-2- [[(1S)-3-(cyclopropylamino)- 1-[[(3S,5R)-5-methyl-2-oxo- pyrrolidin-3-yl]methyl]-2,3- dioxo- propyl]carbamoyl]phenyl] carbamate | 491.9 | 491.1696 |
| 161 | 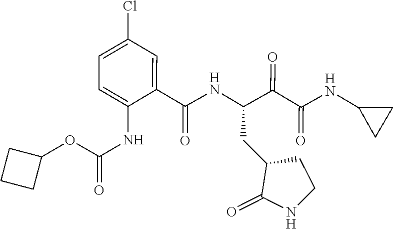 [SMILES: O=C(Nc1ccc(Cl)cc1C(=O)N[C@@H](C[C@@H]1CCNC1=O)C(=O)C(=O)NC1CC1)OC1CCC1] | cyclobutyl N-[4-chloro-2- [[(1S)-3-(cyclopropylamino)- 2,3-dioxo-1-[[(3S)-2- oxopyrrolidin-3- yl]methyl]propyl]carbamoyl] phenyl]carbamate | 491.9 | 491.1697 |
| 162 | 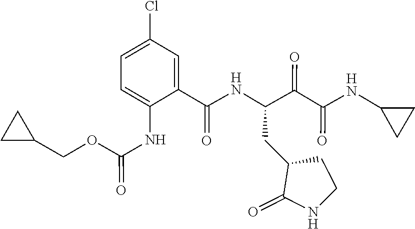 [SMILES: O=C(Nc1ccc(Cl)cc1C(=O)N[C@@H](C[C@@H]1CCNC1=O)C(=O)C(=O)NC1CC1)OCC1CC1] | cyclopropylmethyl N-[4- chloro-2-[[(1S)-3- (cyclopropylamino)-2,3- dioxo-1-[[(3S)-2- oxopyrrolidin-3- yl]methyl]propyl]carbamoyl] phenyl]carbamate | 491.9 | 491.17 |
| 163 | 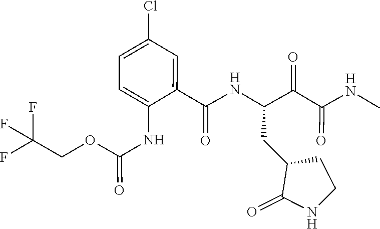 [SMILES: CNC(=O)C(=O)[C@H](C[C@@H]1CCNC1=O)NC(=O)c1cc(Cl)ccc1NC(=O)OCC(F)(F)F] | 2,2,2-trifluoroethyl N-[4- chloro-2-[[(1S)-3- (methylamino)-2,3-dioxo-1- [[(3S)-2-oxopyrrolidin-3- yl]methyl]propyl]carbamoyl] phenyl]carbamate | 493.8 | 493.1101 |
| 164 | 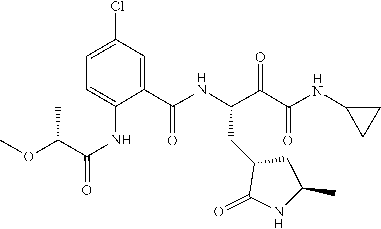 [SMILES: CO[C@H](C)C(=O)Nc1ccc(Cl)cc1C(=O)N[C@@H](C[C@@H]1C[C@@H](C)NC1=O)C(=O)C(=O)NC1CC1] | 5-chloro-N-[(1S)-3- (cyclopropylamino)-1- [[(3S,5R)-5-methyl-2-oxo- pyrrolidin-3-yl]methyl]-2,3- dioxo-propyl]-2-[[(2R)-2- methoxypropanoyl]amino] benzamide | 494.0 | 493.185 |
| 165 | 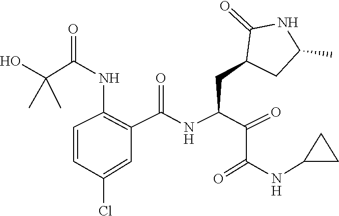 [SMILES: C[C@@H]1C[C@@H](C[C@H](NC(=O)c2cc(Cl)ccc2NC(=O)C(C)(C)O)C(=O)C(=O)NC2CC2)C(=O)N1] | 5-chloro-N-[(1S)-3- (cyclopropylamino)-1- [[(3S,5R)-5-methyl-2-oxo- pyrrolidin-3-yl]methyl]-2,3- dioxo-propyl]-2-[(2-hydroxy- 2-methyl- propanoyl)amino]benzamide | 494.0 | 493.1852 |
| 166 | 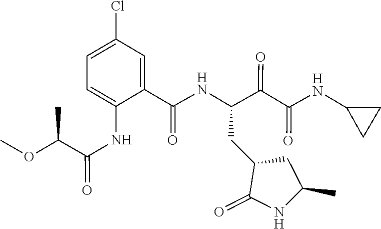 [SMILES: CO[C@@H](C)C(=O)Nc1ccc(Cl)cc1C(=O)N[C@@H](C[C@@H]1C[C@@H](C)NC1=O)C(=O)C(=O)NC1CC1] | 5-chloro-N-[(1S)-3- (cyclopropylamino)-1- [[(3S,5R)-5-methyl-2-oxo- pyrrolidin-3-yl]methyl]-2,3- dioxo-propyl]-2-[[(2S)-2- methoxypropanoyl]amino] benzamide | 494.0 | 493.1854 |
| 167 | 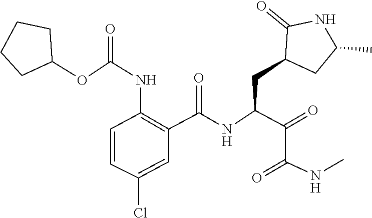 [SMILES: CNC(=O)C(=O)[C@H](C[C@@H]1C[C@@H](C)NC1=O)NC(=O)c1cc(Cl)ccc1NC(=O)OC1CCCC1] | cyclopentyl N-[4-chloro-2- [[(1S)-3-(methylamino)-1- [[(3S,5R)-5-methyl-2-oxo- pyrrolidin-3-yl]methyl]-2,3- dioxo- propyl]carbamoyl]phenyl] carbamate | 494.0 | 493.1855 |
| 168 | 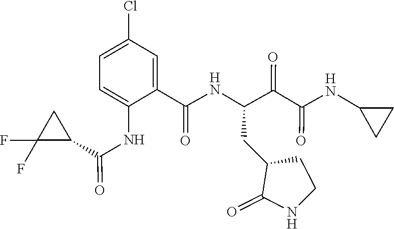 [SMILES: O=C(NC1CC1)C(=O)[C@H](C[C@@H]1CCNC1=O)NC(=O)c1cc(Cl)ccc1NC(=O)[C@H]1CC1(F)F] | 5-chloro-N-[(1S)-3- (cyclopropylamino)-2,3- dioxo-1-[[(3S)-2- oxopyrrolidin-3- yl]methyl]propyl]-2-[(IR)- 2,2- difluorocyclopropanecarbonyl] amino]benzamide | 497.9 | 497.1402 |
| 169 | 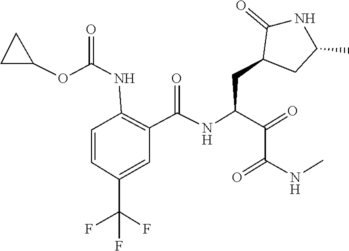 [SMILES: CNC(=O)C(=O)[C@H](C[C@@H]1C[C@@H](C)NC1=O)NC(=O)c1cc(C(F)(F)F)ccc1NC(=O)OC1CC1] | cyclopropyl N-[2-[(1S)-3- (methylamino)-1-[[(3S,5R)- 5-methyl-2-oxo-pyrrolidin-3- yl]methyl]-2,3-dioxo- propyl]carbamoyl]-4- (trifluoromethyl)phenyl] carbamate | 499.5 | 499.1802 |
| 170 | 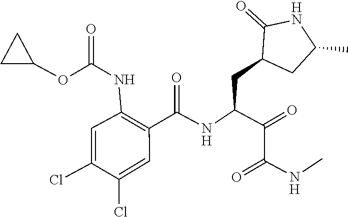 [SMILES: CNC(=O)C(=O)[C@H](C[C@@H]1C[C@@H](C)NC1=O)NC(=O)c1cc(Cl)c(Cl)cc1NC(=O)OC1CC1] | cyclopropyl N-[4,5-dichloro- 2-[[(1S)-3-(methylamino)-1- [[(3S,5R)-5-methyl-2-oxo- pyrrolidin-3-yl]methyl]-2,3- dioxo- propyl]carbamoyl]phenyl] carbamate | 500.3 | 499.1151 |
| 171 | 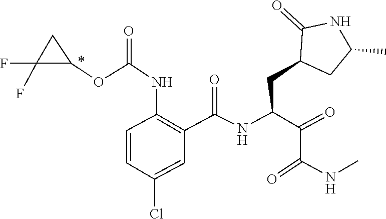 [SMILES: CNC(=O)C(=O)[C@H](C[C@@H]1C[C@@H](C)NC1=O)NC(=O)c1cc(Cl)ccc1NC(=O)OC1CC1(F)F] | (2,2-difluorocyclopropyl) N- [4-chloro-2-[(1S)-3- (methylamino)-1-[[(3S,5R)- 5-methyl-2-oxo-pyrrolidin-3- yl]methyl]-2,3-dioxo- propyl]carbamoyl]phenyl] carbamate | 501.9 | 501.1354 |
| 172 | 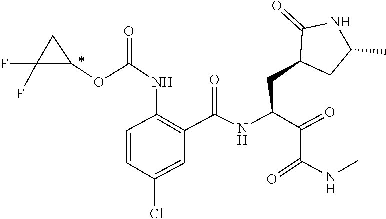 [SMILES: CNC(=O)C(=O)[C@H](C[C@@H]1C[C@@H](C)NC1=O)NC(=O)c1cc(Cl)ccc1NC(=O)OC1CC1(F)F] | (2,2-difluorocyclopropyl) N- [4-chloro-2-[(1S)-3- (methylamino)-1-[[(3S,5R)- 5-methyl-2-oxo-pyrrolidin-3- yl]methyl]-2,3-dioxo- propyl]carbamoyl]phenyl] carbamate | 501.9 | 501.1354 |
| 173 | 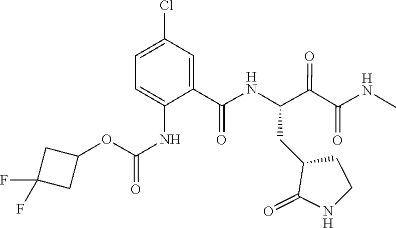 [SMILES: CNC(=O)C(=O)[C@H](C[C@@H]1CCNC1=O)NC(=O)c1cc(Cl)ccc1NC(=O)OC1CC(F)(F)C1] | (3,3-difluorocyclobutyl) N- [4-chloro-2-[(1S)-3- (methylamino)-2,3-dioxo-1- [[(3S)-2-oxopyrrolidin-3- yl]methyl]propyl]carbamoyl] phenyl]carbamate | 501.9 | 501.1353 |
| 174 | 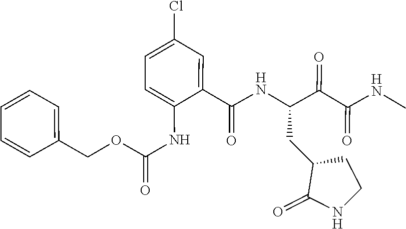 [SMILES: CNC(=O)C(=O)[C@H](C[C@@H]1CCNC1=O)NC(=O)c1cc(Cl)ccc1NC(=O)OCc1ccccc1] | benzyl N-[4-chloro-2-[(1S)- 3-(methylamino)-2,3-dioxo- 1-[[(3S)-2-oxopyrrolidin-3- yl]methyl]propyl]carbamoyl] phenyl]carbamate | 501.9 | 501.1542 |
| 175 | 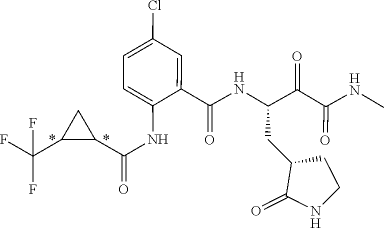 [SMILES: CNC(=O)C(=O)[C@H](C[C@@H]1CCNC1=O)NC(=O)c1cc(Cl)ccc1NC(=O)C1CC1C(F)(F)F] | 5-chloro-N-[(1S)-3- (methylamino)-2,3-dioxo-1- [[(3S)-2-oxopyrrolidin-3- yl]methyl]propyl]-2-[2- (trifluoromethyl)cyclopropane- carbonyl]amino]benzamide | 503.9 | 503.1309 |
| 176 | 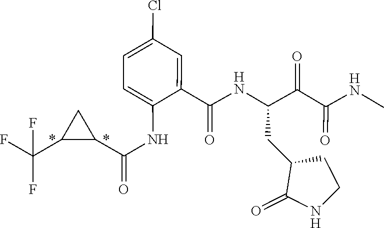 [SMILES: CNC(=O)C(=O)[C@H](C[C@@H]1CCNC1=O)NC(=O)c1cc(Cl)ccc1NC(=O)C1CC1C(F)(F)F] | 5-chloro-N-[(1S)-3- (methylamino)-2,3-dioxo-1- [[(3S)-2-oxopyrrolidin-3- yl]methyl]propyl]-2-[[2- (trifluoromethyl)cyclopropane- carbonyl]amino]benzamide | 503.9 | 503.1308 |
| 177 | 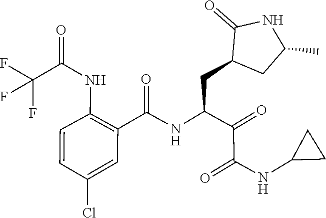 [SMILES: C[C@@H]1C[C@@H](C[C@H](NC(=O)c2cc(Cl)ccc2NC(=O)C(F)(F)F)C(=O)C(=O)NC2CC2)C(=O)N1] | 5-chloro-N-[(1S)-3- (cyclopropylamino)-1- [[(3S,5R)-5-methyl-2-oxo- pyrrolidin-3-yl]methyl]-2,3- dioxo-propyl]-2-[(2,2,2- trifluoroacetyl)amino] benzamide | 503.9 | 503.1314 |
| 178 | 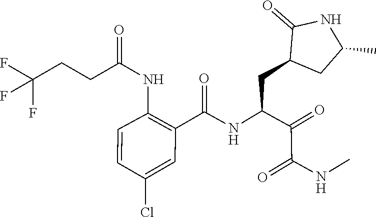 [SMILES: CNC(=O)C(=O)[C@H](C[C@@H]1C[C@@H](C)NC1=O)NC(=O)c1cc(Cl)ccc1NC(=O)CCC(F)(F)F] | 5-chloro-N-[(1S)-3- (methylamino)-1-[[(3S,5R)- 5-methyl-2-oxo-pyrrolidin-3- yl]methyl]-2,3-dioxo- propyl]-2-(4,4,4- trifluorobutanoylamino) benzamide | 505.9 | 505.1469 |
| 179 | 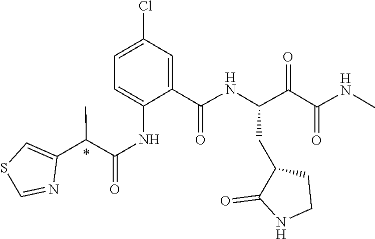 [SMILES: CNC(=O)C(=O)[C@H](C[C@@H]1CCNC1=O)NC(=O)c1cc(Cl)ccc1NC(=O)C(C)c1cscn1] | 5-chloro-N-[(1S)-3- (methylamino)-2,3-dioxo-1- [[(3S)-2-oxopyrrolidin-3- yl]methyl]propyl]-2-(2- thiazol-4- ylpropanoylamino)benzamide | 507.0 | 506.1263 |
| 180 | 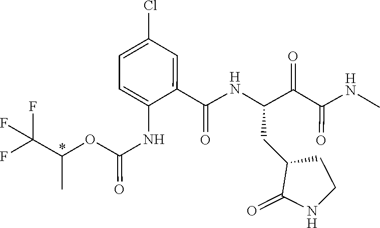 [SMILES: CNC(=O)C(=O)[C@H](C[C@@H]1CCNC1=O)NC(=O)c1cc(Cl)ccc1NC(=O)OC(C)C(F)(F)F] | (2,2,2-trifluoro-1-methyl- ethyl) N-[4-chloro-2-[(1S)- 3-(methylamino)-2,3-dioxo- 1-[[(3S)-2-oxopyrrolidin-3- yl]methyl]propyl]carbamoyl] phenyl]carbamate | 507.9 | 507.1259 |
| 181 | 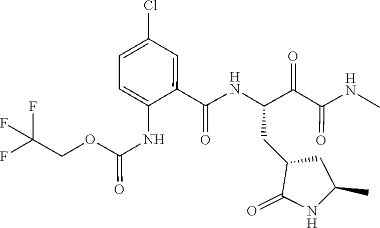 [SMILES: CNC(=O)C(=O)[C@H](C[C@@H]1C[C@@H](C)NC1=O)NC(=O)c1cc(Cl)ccc1NC(=O)OCC(F)(F)F] | 2,2,2-trifluoroethyl N-[4- chloro-2-[[(1S)-3- (methylamino)-1-[[(3S,5R)- 5-methyl-2-oxo-pyrrolidin-3- yl]methyl]-2,3-dioxo- propyl]carbamoyl]phenyl] carbamate | 507.9 | 507.1255 |
| 182 | 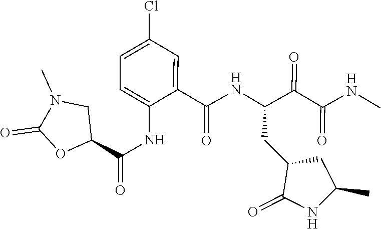 [SMILES: CNC(=O)C(=O)[C@H](C[C@@H]1C[C@@H](C)NC1=O)NC(=O)c1cc(Cl)ccc1NC(=O)[C@@H]1CN(C)C(=O)O1] | (5S)-N-[4-chloro-2-[[(1S)-3- (methylamino)-1-[[(3S,5R)- 5-methyl-2-oxo-pyrrolidin-3- yl]methyl]-2,3-dioxo- propyl]carbamoyl]phenyl]-3- methyl-2-oxo-oxazolidine-5- carboxamide | 508.9 | 508.1599 |
| 183 | 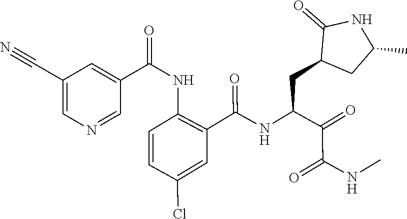 [SMILES: CNC(=O)C(=O)[C@H](C[C@@H]1C[C@@H](C)NC1=O)NC(=O)c1cc(Cl)ccc1NC(=O)c1cncc(C#N)c1] | N-[4-chloro-2-[(1S)-3- (methylamino)-1-[[(3S,5R)- 5-methyl-2-oxo-pyrrolidin-3- yl]methyl]-2,3-dioxo- propyl]carbamoyl]phenyl]-5- cyano-pyridine-3- carboxamide | 511.9 | 511.1497 |
| 184 | 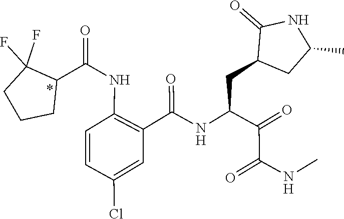 [SMILES: CNC(=O)C(=O)[C@H](C[C@@H]1C[C@@H](C)NC1=O)NC(=O)c1cc(Cl)ccc1NC(=O)C1CCCC1(F)F] | 5-chloro-2-[(2,2- difluorocyclopentanecarbonyl) amino]-N-[(1S)-3- (methylamino)-1-[[(3S,5R)- 5-methyl-2-oxo-pyrrolidin-3- yl]methyl]-2,3-dioxo- propyl]benzamide | 513.9 | 513.1718 |
| 185 | 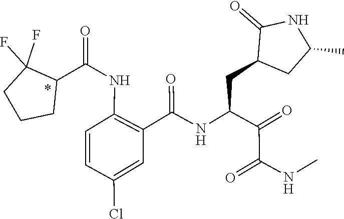 [SMILES: CNC(=O)C(=O)[C@H](C[C@@H]1C[C@@H](C)NC1=O)NC(=O)c1cc(Cl)ccc1NC(=O)C1CCCC1(F)F] | 5-chloro-2-[(2,2- difluorocyclopentanecarbonyl) amino]-N-[(1S)-3- (methylamino)-1-[[(3S,5R)- 5-methyl-2-oxo-pyrrolidin-3- yl]methyl]-2,3-dioxo- propyl]benzamide | 513.9 | 513.1717 |
| 186 | 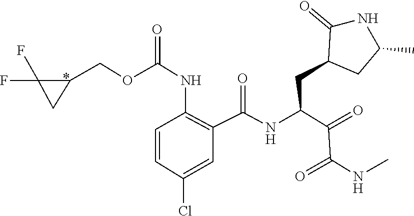 [SMILES: CNC(=O)C(=O)[C@H](C[C@@H]1C[C@@H](C)NC1=O)NC(=O)c1cc(Cl)ccc1NC(=O)OCC1CC1(F)F] | (2,2- difluorocyclopropyl)methyl N-[4-chloro-2-[[(1S)-3- (methylamino)-1-[[(3S,5R)- 5-methyl-2-oxo-pyrrolidin-3- yl]methyl]-2,3-dioxo- propyl]carbamoyl]phenyl] carbamate | 515.9 | 515.1507 |
| 187 | 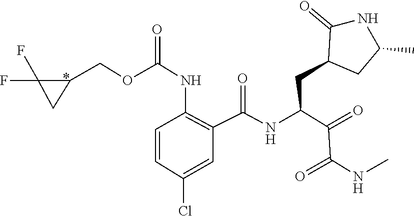 [SMILES: CNC(=O)C(=O)[C@H](C[C@@H]1C[C@@H](C)NC1=O)NC(=O)c1cc(Cl)ccc1NC(=O)OCC1CC1(F)F] | (2,2- difluorocyclopropyl)methyl N-[4-chloro-2-[(1S)-3- (methylamino)-1-[[(3S,5R)- 5-methyl-2-oxo-pyrrolidin-3- yl]methyl]-2,3-dioxo- propyl]carbamoyl]phenyl] carbamate | 515.9 | 515.1509 |
| 188 | 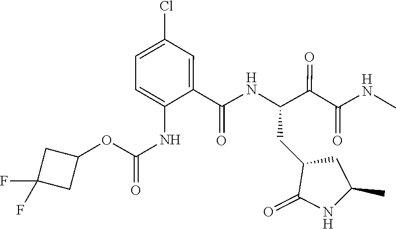 [SMILES: CNC(=O)C(=O)[C@H](C[C@@H]1C[C@@H](C)NC1=O)NC(=O)c1cc(Cl)ccc1NC(=O)OC1CC(F)(F)C1] | (3,3-difluorocyclobutyl) N- [4-chloro-2-[(1S)-3- (methylamino)-1-[[(3S,5R)- 5-methyl-2-oxo-pyrrolidin-3- yl]methyl]-2,3-dioxo- propyl]carbamoyl]phenyl] carbamate | 515.9 | 515.1512 |
| 189 | 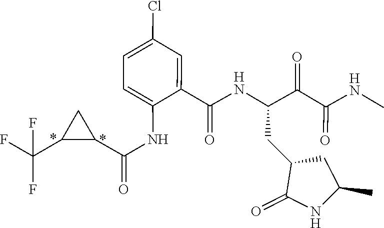 [SMILES: CNC(=O)C(=O)[C@H](C[C@@H]1C[C@@H](C)NC1=O)NC(=O)c1cc(Cl)ccc1NC(=O)C1CC1C(F)(F)F] | 5-chloro-N-[(1S)-3- (methylamino)-1-[[(3S,5R)- 5-methyl-2-oxo-pyrrolidin-3- yl]methyl]-2,3-dioxo- propyl]-2-[[2- (trifluoromethyl)cyclopropane- carbonyl]amino]benzamide | 517.9 | 517.1466 |
| 190 | 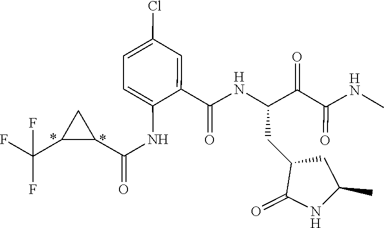 [SMILES: CNC(=O)C(=O)[C@H](C[C@@H]1C[C@@H](C)NC1=O)NC(=O)c1cc(Cl)ccc1NC(=O)C1CC1C(F)(F)F] | 5-chloro-N-[(1S)-3- (methylamino)-1-[[(3S,5R)- 5-methyl-2-oxo-pyrrolidin-3- yl]methyl]-2,3-dioxo- propyl]-2-[[2- (trifluoromethyl)cyclopropane- carbonyl]amino]benzamide | 517.9 | 517.1463 |
| 191 | 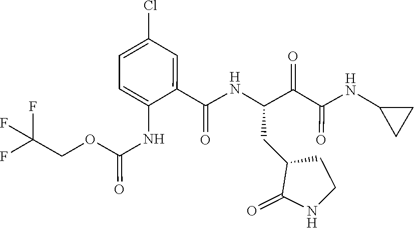 [SMILES: O=C(Nc1ccc(Cl)cc1C(=O)N[C@@H](C[C@@H]1CCNC1=O)C(=O)C(=O)NC1CC1)OCC(F)(F)F] | 2,2,2-trifluoroethyl N-[4- chloro-2-[[(1S)-3- (cyclopropylamino)-2,3- dioxo-1-[[(3S)-2- oxopyrrolidin-3- yl]methyl]propyl]carbamoyl] phenyl]carbamate | 519.9 | 519.126 |
| 192 | 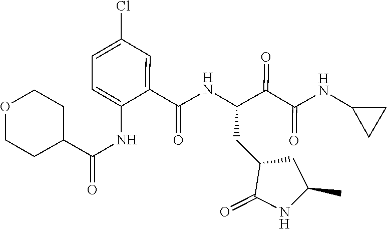 [SMILES: C[C@@H]1C[C@@H](C[C@H](NC(=O)c2cc(Cl)ccc2NC(=O)C2CCOCC2)C(=O)C(=O)NC2CC2)C(=O)N1] | N-[4-chloro-2-[[(1S)-3- (cyclopropylamino)-1- [[(3S,5R)-5-methyl-2-oxo- pyrrolidin-3-yl]methyl]-2,3- dioxo-propyl] carbamoyl]phenyl]tetra- hydropyran-4-carboxamide | 520.0 | 519.201 |
| 193 | 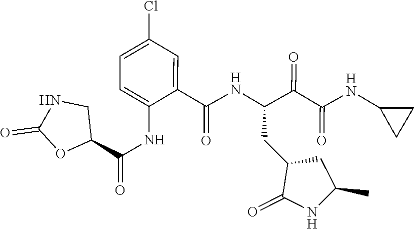 [SMILES: C[C@@H]1C[C@@H](C[C@H](NC(=O)c2cc(Cl)ccc2NC(=O)[C@@H]2CNC(=O)O2)C(=O)C(=O)NC2CC2)C(=O)N1] | (5S)-N-[4-chloro-2-[[(1S)-3- (cyclopropylamino)-1- [[(3S,5R)-5-methyl-2-oxo- pyrrolidin-3-yl]methyl]-2,3- dioxo- propyl]carbamoyl]phenyl]-2- oxo-oxazolidine-5- carboxamide | 520.9 | 520.1595 |
| 194 | 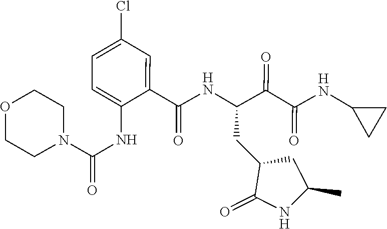 [SMILES: C[C@@H]1C[C@@H](C[C@H](NC(=O)c2cc(Cl)ccc2NC(=O)N2CCOCC2)C(=O)C(=O)NC2CC2)C(=O)N1] | N-[4-chloro-2-[[(1S)-3- (cyclopropylamino)-1- [[(3S,5R)-5-methyl-2-oxo- pyrrolidin-3-yl]methyl]-2,3- dioxo- propyl]carbamoyl]phenyl] morpholine-4-carboxamide | 521.0 | 520.196 |
| 195 | 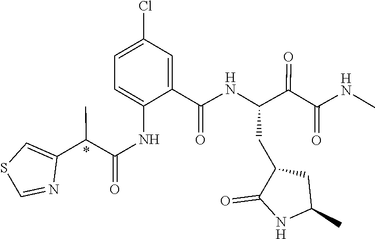 [SMILES: CNC(=O)C(=O)[C@H](C[C@@H]1C[C@@H](C)NC1=O)NC(=O)c1cc(Cl)ccc1NC(=O)C(C)c1cscn1] | 5-chloro-N-[(1S)-3- (methylamino)-1-[[(3S,5R)- 5-methyl-2-oxo-pyrrolidin-3- yl]methyl]-2,3-dioxo- propyl]-2-(2-thiazol-4- ylpropanoylamino)benzamide | 521.0 | 520.1418 |
| 196 | 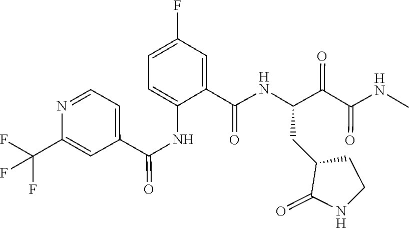 [SMILES: CNC(=O)C(=O)[C@H](C[C@@H]1CCNC1=O)NC(=O)c1cc(F)ccc1NC(=O)c1ccnc(C(F)(F)F)c1] | N-[4-fluoro-2-[[(1S)-3- (methylamino)-2,3-dioxo-1- [[(3S)-2-oxopyrrolidin-3- yl]methyl]propyl]carbamoyl] phenyl]-2- (trifluoromethyl)pyridine-4- carboxamide | 524.4 | 524.1555 |
| 197 | 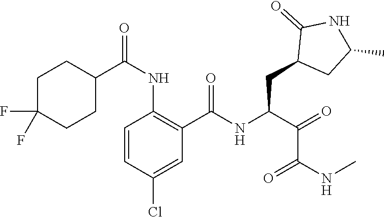 [SMILES: CNC(=O)C(=O)[C@H](C[C@@H]1C[C@@H](C)NC1=O)NC(=O)c1cc(Cl)ccc1NC(=O)C1CCC(F)(F)CC1] | 5-chloro-2-[(4,4- difluorocyclohexanecarbonyl) amino]-N-[(1S)-3- (methylamino)-1-[(3S,5R)- 5-methyl-2-oxo-pyrrolidin-3- yl]methyl]-2,3-dioxo- propyl]benzamide | 528.0 | 527.1876 |
| 198 | 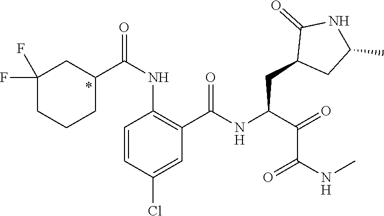 [SMILES: CNC(=O)C(=O)[C@H](C[C@@H]1C[C@@H](C)NC1=O)NC(=O)c1cc(Cl)ccc1NC(=O)C1CCCC(F)(F)C1] | 5-chloro-2-[(3,3- difluorocyclohexanecarbonyl) amino]-N-[(1S)-3- (methylamino)-1-[[(3S,5R)- 5-methyl-2-oxo-pyrrolidin-3- yl]methyl]-2,3-dioxo- propyl]benzamide | 528.0 | 527.1873 |
| 199 | 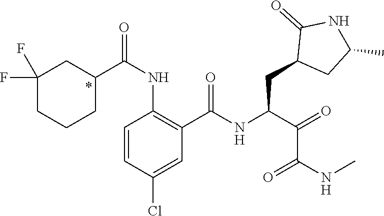 [SMILES: CNC(=O)C(=O)[C@H](C[C@@H]1C[C@@H](C)NC1=O)NC(=O)c1cc(Cl)ccc1NC(=O)C1CCCC(F)(F)C1] | 5-chloro-2-[(3,3- difluorocyclohexanecarbonyl) amino]-N-[(1S)-3- (methylamino)-1-[[(3S,5R)- 5-methyl-2-oxo-pyrrolidin-3- yl]methyl]-2,3-dioxo- propyl]benzamide | 528.0 | 527.1872 |
| 200 | 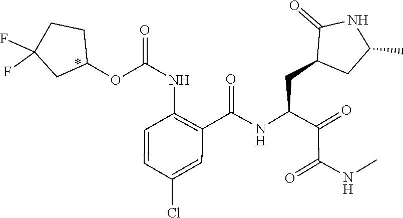 [SMILES: CNC(=O)C(=O)[C@H](C[C@@H]1C[C@@H](C)NC1=O)NC(=O)c1cc(Cl)ccc1NC(=O)OC1CCC(F)(F)C1] | (3,3-difluorocyclopentyl) N- [4-chloro-2-[[(1S)-3- (methylamino)-1-[[(3S,5R)- 5-methyl-2-oxo-pyrrolidin-3- yl]methyl]-2,3-dioxo- propyl]carbamoyl]phenyl] carbamate | 529.9 | 529.1667 |
| 201 | 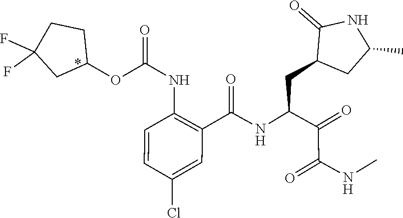 [SMILES: CNC(=O)C(=O)[C@H](C[C@@H]1C[C@@H](C)NC1=O)NC(=O)c1cc(Cl)ccc1NC(=O)OC1CCC(F)(F)C1] | (3,3-difluorocyclopentyl) N- [4-chloro-2-[(1S)-3- (methylamino)-1-[[(3S,5R)- 5-methyl-2-oxo-pyrrolidin-3- yl]methyl]-2,3-dioxo- propyl]carbamoyl]phenyl] carbamate | 529.9 | 529.1664 |
| 202 | 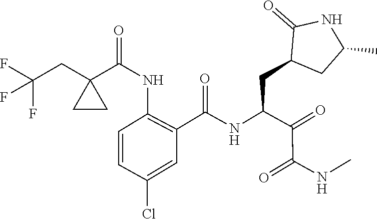 [SMILES: CNC(=O)C(=O)[C@H](C[C@@H]1C[C@@H](C)NC1=O)NC(=O)c1cc(Cl)ccc1NC(=O)C1(CC(F)(F)F)CC1] | 5-chloro-N-[(1S)-3- (methylamino)-1-[[(3S,5R)- 5-methyl-2-oxo-pyrrolidin-3- yl]methyl]-2,3-dioxo- propyl]-2-[[1-(2,2,2- trifluoroethyl)cyclopropane- carbonyl]amino]benzamide | 531.9 | 531.162 |
| 203 | 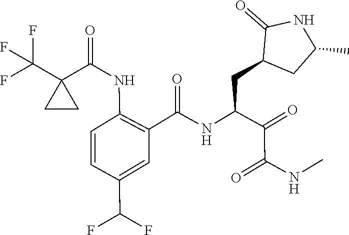 [SMILES: CNC(=O)C(=O)[C@H](C[C@@H]1C[C@@H](C)NC1=O)NC(=O)c1cc(C(F)F)ccc1NC(=O)C1(C(F)(F)F)CC1] | 5-(difluoromethyl)-N-[(1S)- 3-(methylamino)-1- [[(3S,5R)-5-methyl-2-oxo- pyrrolidin-3-yl]methyl]-2,3- dioxo-propyl]-2-[[1- (trifluoromethyl)cyclopropane- carbonyl]amino]benzamide | 533.5 | 533.182 |
| 204 | 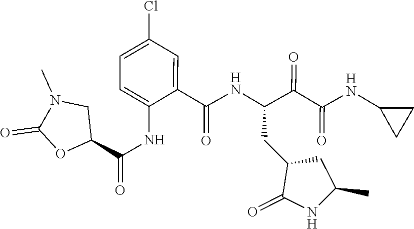 [SMILES: C[C@@H]1C[C@@H](C[C@H](NC(=O)c2cc(Cl)ccc2NC(=O)[C@@H]2CN(C)C(=O)O2)C(=O)C(=O)NC2CC2)C(=O)N1] | (5S)-N-[4-chloro-2-[[(1S)-3- (cyclopropylamino)-1- [[(3S,5R)-5-methyl-2-oxo- pyrrolidin-3-yl]methyl]-2,3- dioxo- propyl]carbamoyl]phenyl]-3- methyl-2-oxo-oxazolidine-5- carboxamide | 535.0 | 534.1757 |
| 205 | 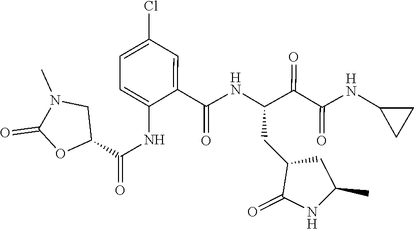 [SMILES: C[C@@H]1C[C@@H](C[C@H](NC(=O)c2cc(Cl)ccc2NC(=O)[C@H]2CN(C)C(=O)O2)C(=O)C(=O)NC2CC2)C(=O)N1] | (5R)-N-[4-chloro-2-[(1S)-3- (cyclopropylamino)-1- [[(3S,5R)-5-methyl-2-oxo- pyrrolidin-3-yl]methyl]-2,3- dioxo- propyl]carbamoyl]phenyl]-3- methyl-2-oxo-oxazolidine-5- carboxamide | 535.0 | 534.1753 |
| 206 | 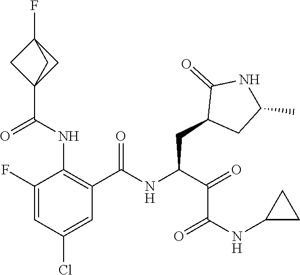 [SMILES: C[C@@H]1C[C@@H](C[C@H](NC(=O)c2cc(Cl)cc(F)c2NC(=O)C23CC(F)(C2)C3)C(=O)C(=O)NC2CC2)C(=O)N1] | N-[4-chloro-2-[(1S)-3- (cyclopropylamino)-1- [[(3S,5R)-5-methyl-2-oxo- pyrrolidin-3-yl]methyl]-2,3- dioxo-propyl]carbamoyl]-6- fluoro-phenyl]-3-fluoro- bicyclo[1.1.1]pentane-1- carboxamide | 538.0 | 537.172 |
| 207 | 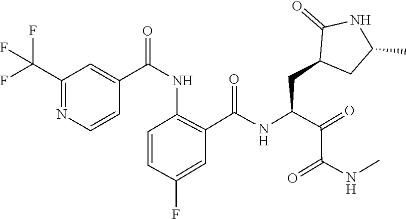 [SMILES: CNC(=O)C(=O)[C@H](C[C@@H]1C[C@@H](C)NC1=O)NC(=O)c1cc(F)ccc1NC(=O)c1ccnc(C(F)(F)F)c1] | N-[4-fluoro-2-[[(1S)-3- (methylamino)-1-[[(3S,5R)- 5-methyl-2-oxo-pyrrolidin-3- yl]methyl]-2,3-dioxo- propyl]carbamoyl]phenyl]-2- (trifluoromethyl)pyridine-4- carboxamide | 538.5 | 538.1712 |
| 208 | 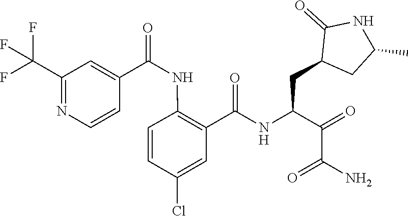 [SMILES: C[C@@H]1C[C@@H](C[C@H](NC(=O)c2cc(Cl)ccc2NC(=O)c2ccnc(C(F)(F)F)c2)C(=O)C(N)=O)C(=O)N1] | N-[2-[[(1S)-3-amino-1- [[(3S,5R)-5-methyl-2-oxo- pyrrolidin-3-yl]methyl]-2,3- dioxo-propyl]carbamoyl]-4- chloro-phenyl]-2- (trifluoromethyl)pyridine-4- carboxamide | 540.9 | 540.1261 |
| 209 | 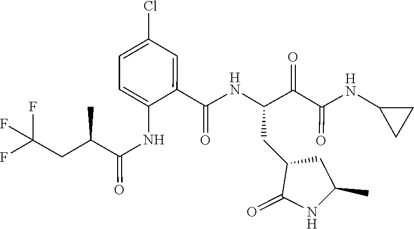 [SMILES: C[C@@H]1C[C@@H](C[C@H](NC(=O)c2cc(Cl)ccc2NC(=O)[C@H](C)CC(F)(F)F)C(=O)C(=O)NC2CC2)C(=O)N1] | 5-chloro-N-[(1S)-3- (cyclopropylamino)-1- [[(3S,5R)-5-methyl-2-oxo- pyrrolidin-3-yl]methyl]-2,3- dioxo-propyl]-2-[[(2R)-4,4,4- trifluoro-2-methyl- butanoyl]amino]benzamide | 546.0 | 545.1783 |
| 210 | 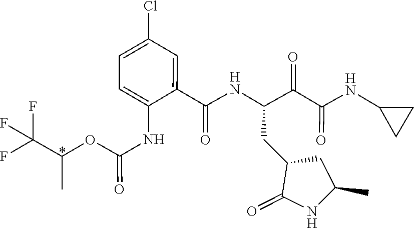 [SMILES: CC(OC(=O)Nc1ccc(Cl)cc1C(=O)N[C@@H](C[C@@H]1C[C@@H](C)NC1=O)C(=O)C(=O)NC1CC1)C(F)(F)F] | (2,2,2-trifluoro-1-methyl- ethyl) N-[4-chloro-2-[[(1S)- 3-(cyclopropylamino)-1- [[(3S,5R)-5-methyl-2-oxo- pyrrolidin-3-yl]methyl]-2,3- dioxo- propyl]carbamoyl]phenyl] carbamate | 547.9 | 547.1569 |
| 211 | 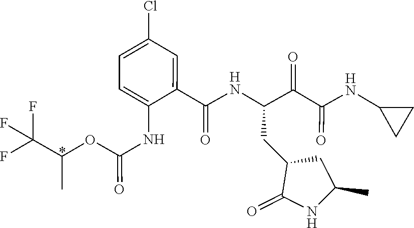 [SMILES: CC(OC(=O)Nc1ccc(Cl)cc1C(=O)N[C@@H](C[C@@H]1C[C@@H](C)NC1=O)C(=O)C(=O)NC1CC1)C(F)(F)F] | (2,2,2-trifluoro-1-methyl- ethyl) N-[4-chloro-2-[[(1S)- 3-(cyclopropylamino)-1- [[(3S,5R)-5-methyl-2-oxo- pyrrolidin-3-yl]methyl]-2,3- dioxo- propyl]carbamoyl]phenyl] carbamate | 547.9 | 547.157 |
| 212 | 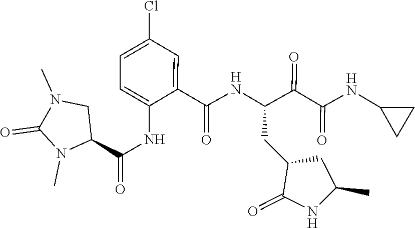 [SMILES: C[C@@H]1C[C@@H](C[C@H](NC(=O)c2cc(Cl)ccc2NC(=O)[C@@H]2CN(C)C(=O)N2C)C(=O)C(=O)NC2CC2)C(=O)N1] | (4S)-N-[4-chloro-2-[[(1S)-3- (cyclopropylamino)-1- [[(3S,5R)-5-methyl-2-oxo- pyrrolidin-3-yl]methyl]-2,3- dioxo- propyl]carbamoyl]phenyl]- 1,3-dimethyl-2-oxo- imidazolidine-4-carboxamide | 548.0 | 547.2067 |
| 213 | 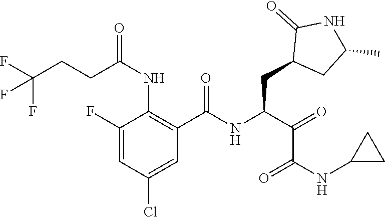 [SMILES: C[C@@H]1C[C@@H](C[C@H](NC(=O)c2cc(Cl)cc(F)c2NC(=O)CCC(F)(F)F)C(=O)C(=O)NC2CC2)C(=O)N1] | 5-chloro-N-[(1S)-3- (cyclopropylamino)-1- [[(3S,5R)-5-methyl-2-oxo- pyrrolidin-3-yl]methyl]-2,3- dioxo-propyl]-3-fluoro-2- (4,4,4- trifluorobutanoylamino) benzamide | 549.9 | 557.1976 |
| 214 | 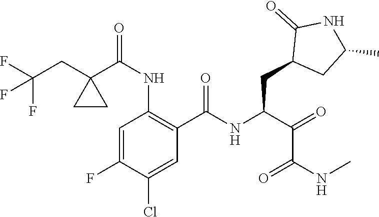 [SMILES: CNC(=O)C(=O)[C@H](C[C@@H]1C[C@@H](C)NC1=O)NC(=O)c1cc(Cl)c(F)cc1NC(=O)C1(CC(F)(F)F)CC1] | 5-chloro-4-fluoro-N-[(1S)-3- (methylamino)-1-[[(3S,5R)- 5-methyl-2-oxo-pyrrolidin-3- yl]methyl]-2,3-dioxo- propyl]-2-[[1-(2,2,2- trifluoroethyl)cyclopropane- carbonyl]amino]benzamide | 549.9 | 549.1529 |
| 215 | 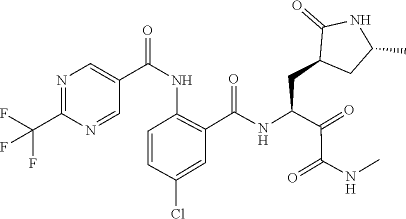 [SMILES: CNC(=O)C(=O)[C@H](C[C@@H]1C[C@@H](C)NC1=O)NC(=O)c1cc(Cl)ccc1NC(=O)c1cnc(C(F)(F)F)nc1] | N-[4-chloro-2-[[(1S)-3- (methylamino)-1-[[(3S,5R)- 5-methyl-2-oxo-pyrrolidin-3- yl]methyl]-2,3-dioxo- propyl]carbamoyl]phenyl]-2- (trifluoromethyl)pyrimidine- 5-carboxamide | 555.9 | 555.1371 |
| 216 | 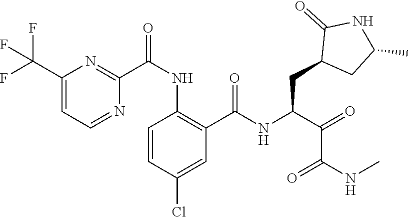 [SMILES: CNC(=O)C(=O)[C@H](C[C@@H]1C[C@@H](C)NC1=O)NC(=O)c1cc(Cl)ccc1NC(=O)c1nccc(C(F)(F)F)n1] | N-[4-chloro-2-[(1S)-3- (methylamino)-1-[[(3S,5R)- 5-methyl-2-oxo-pyrrolidin-3- yl]methyl]-2,3-dioxo- propyl]carbamoyl]phenyl]-4- (trifluoromethyl)pyrimidine- 2-carboxamide | 555.9 | 555.1367 |
| 217 | 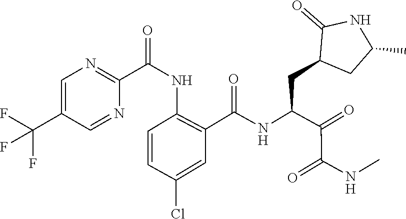 [SMILES: CNC(=O)C(=O)[C@H](C[C@@H]1C[C@@H](C)NC1=O)NC(=O)c1cc(Cl)ccc1NC(=O)c1ncc(C(F)(F)F)cn1] | N-[4-chloro-2-[[(1S)-3- (methylamino)-1-[[(3S,5R)- 5-methyl-2-oxo-pyrrolidin-3- yl]methyl]-2,3-dioxo- propyl]carbamoyl]phenyl]-5- (trifluoromethyl)pyrimidine- 2-carboxamide | 555.9 | 555.137 |
| 218 | 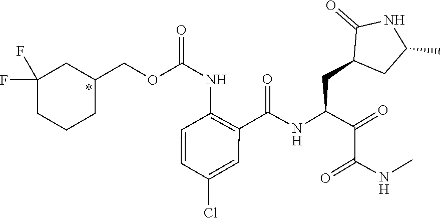 [SMILES: CNC(=O)C(=O)[C@H](C[C@@H]1C[C@@H](C)NC1=O)NC(=O)c1cc(Cl)ccc1NC(=O)OCC1CCCC(F)(F)C1] | (3,3- difluorocyclohexyl)methyl N-[4-chloro-2-[[(1S)-3- (methylamino)-1-[[(3S,5R)- 5-methyl-2-oxo-pyrrolidin-3- yl]methyl]-2,3-dioxo- propyl]carbamoyl]phenyl] carbamate | 558.0 | 557 |
| 219 | 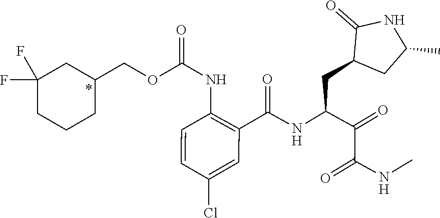 [SMILES: CNC(=O)C(=O)[C@H](C[C@@H]1C[C@@H](C)NC1=O)NC(=O)c1cc(Cl)ccc1NC(=O)OCC1CCCC(F)(F)C1] | (3,3- difluorocyclohexyl)methyl N-[4-chloro-2-[[(1S)-3- (methylamino)-1-[(3S,5R)- 5-methyl-2-oxo-pyrrolidin-3- yl]methyl]-2,3-dioxo- propyl]carbamoyl]phenyl] carbamate | 558.0 | 557.1981 |
| 220 | 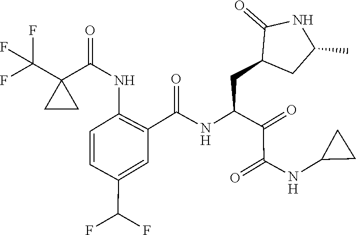 [SMILES: C[C@@H]1C[C@@H](C[C@H](NC(=O)c2cc(C(F)F)ccc2NC(=O)C2(C(F)(F)F)CC2)C(=O)C(=O)NC2CC2)C(=O)N1] | N-[(1S)-3- (cyclopropylamino)-1- [[(3S,5R)-5-methyl-2-oxo- pyrrolidin-3-yl]methyl]-2,3- dioxo-propyl]-5- (difluoromethyl)-2-[[1- (trifluoromethyl)cyclopropane- carbonyl]amino]benzamide | 559.5 | 559.1979 |
| 221 | 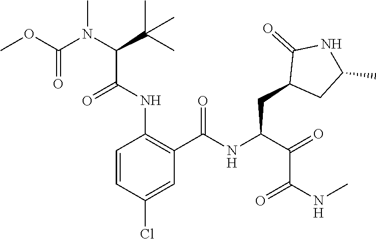 | methyl N-[(1S)-1-[[4-chloro- 2-[(1S)-3-(methylamino)-1- [[(3S,5R)-5-methyl-2-oxo- pyrrolidin-3-yl]methyl]-2,3- dioxo- propyl]carbamoyl]phenyl] carbamoyl]-2,2-dimethyl- propyl]-N-methyl-carbamate | 567.0 | 566.2381 |
| 222 | 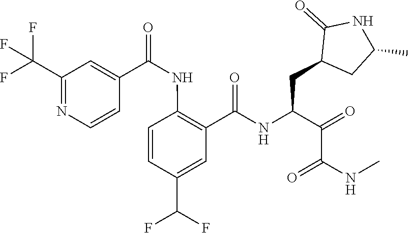 [SMILES: CNC(=O)C(=O)[C@H](C[C@@H]1C[C@@H](C)NC1=O)NC(=O)c1cc(C(F)F)ccc1NC(=O)c1ccnc(C(F)(F)F)c1] | N-[4-(difluoromethyl)-2- [[(1S)-3-(methylamino)-1- [[(3S,5R)-5-methyl-2-oxo- pyrrolidin-3-yl]methyl]-2,3- dioxo- propyl]carbamoyl]phenyl]-2- (trifluoromethyl)pyridine-4- carboxamide | 570.5 | 570.1776 |
| 223 | 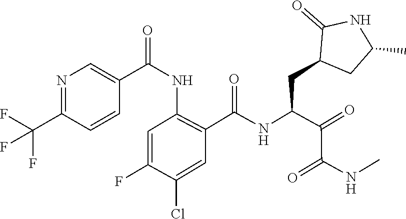 [SMILES: CNC(=O)C(=O)[C@H](C[C@@H]1C[C@@H](C)NC1=O)NC(=O)c1cc(Cl)c(F)cc1NC(=O)c1ccc(C(F)(F)F)nc1] | N-[4-chloro-5-fluoro-2- [[(1S)-3-(methylamino)-1- [[(3S,5R)-5-methyl-2-oxo- pyrrolidin-3-yl]methyl]-2,3- dioxo- propyl]carbamoyl]phenyl]-6- (trifluoromethyl)pyridine-3- carboxamide | 572.9 | 572.1324 |
| 224 | 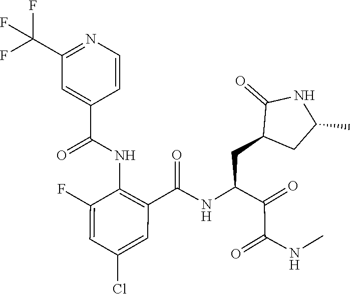 [SMILES: CNC(=O)C(=O)[C@H](C[C@@H]1C[C@@H](C)NC1=O)NC(=O)c1cc(Cl)cc(F)c1NC(=O)c1ccnc(C(F)(F)F)c1] | N-[4-chloro-2-fluoro-6- [[(1S)-3-(methylamino)-1- [[(3S,5R)-5-methyl-2-oxo- pyrrolidin-3-yl]methyl]-2,3- dioxo- propyl]carbamoyl]phenyl]-2- (trifluoromethyl)pyridine-4- carboxamide | 572.9 | 572.1323 |
| 225 | 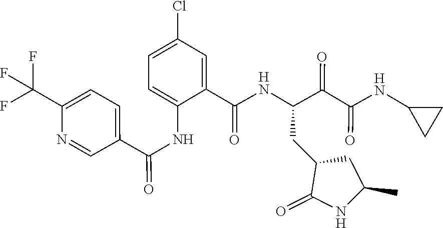 [SMILES: C[C@@H]1C[C@@H](C[C@H](NC(=O)c2cc(Cl)ccc2NC(=O)c2ccc(C(F)(F)F)nc2)C(=O)C(=O)NC2CC2)C(=O)N1] | N-[4-chloro-2-[[(1S)-3- (cyclopropylamino)-1- [[(3S,5R)-5-methyl-2-oxo- pyrrolidin-3-yl]methyl]-2,3- dioxo- propyl]carbamoyl]phenyl]-6- (trifluoromethyl)pyridine-3- carboxamide | 581.0 | 580.1571 |
| 226 | 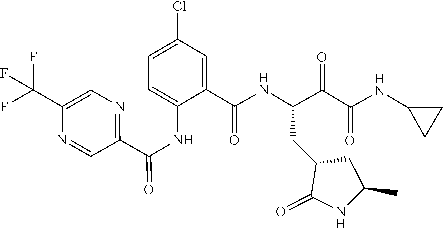 [SMILES: C[C@@H]1C[C@@H](C[C@H](NC(=O)c2cc(Cl)ccc2NC(=O)c2cnc(C(F)(F)F)cn2)C(=O)C(=O)NC2CC2)C(=O)N1] | N-[4-chloro-2-[[(1S)-3- (cyclopropylamino)-1- [[(3S,5R)-5-methyl-2-oxo- pyrrolidin-3-yl]methyl]-2,3- dioxo- propyl]carbamoyl]phenyl]-5- (trifluoromethyl)pyrazine-2- carboxamide | 581.9 | 581.1533 |
| 227 | 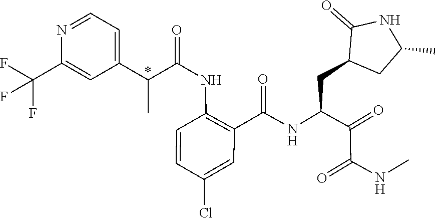 [SMILES: CNC(=O)C(=O)[C@H](C[C@@H]1C[C@@H](C)NC1=O)NC(=O)c1cc(Cl)ccc1NC(=O)C(C)c1ccnc(C(F)(F)F)c1] | 5-chloro-N-[(1S)-3- (methylamino)-1-[[(3S,5R)- 5-methyl-2-oxo-pyrrolidin-3- yl]methyl]-2,3-dioxo- propyl]-2-[2-[2- (trifluoromethyl)-4- pyridyl]propanoylamino] benzamide | 583.0 | 582.1728 |
| 228 | 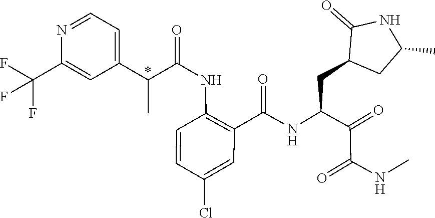 [SMILES: CNC(=O)C(=O)[C@H](C[C@@H]1C[C@@H](C)NC1=O)NC(=O)c1cc(Cl)ccc1NC(=O)C(C)c1ccnc(C(F)(F)F)c1] | 5-chloro-N-[(1S)-3- (methylamino)-1-[[(3S,5R)- 5-methyl-2-oxo-pyrrolidin-3- yl]methyl]-2,3-dioxo- propyl]-2-[2-[2- (trifluoromethyl)-4- pyridyl]propanoylamino] benzamide | 583.0 | 582.1732 |
| 229 | 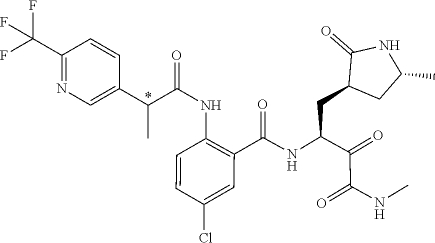 [SMILES: CNC(=O)C(=O)[C@H](C[C@@H]1C[C@@H](C)NC1=O)NC(=O)c1cc(Cl)ccc1NC(=O)C(C)c1ccc(C(F)(F)F)nc1] | 5-chloro-N-[(1S)-3- (methylamino)-1-[[(3S,5R)- 5-methyl-2-oxo-pyrrolidin-3- yl]methyl]-2,3-dioxo- propyl]-2-[2-[6- (trifluoromethyl)-3- pyridyl]propanoylamino] benzamide | 583.0 | 582.173 |
| 230 | 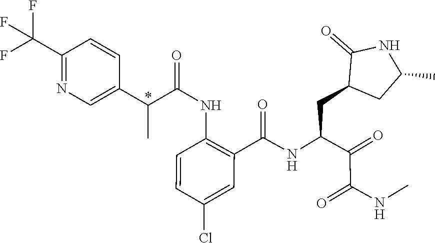 [SMILES: CNC(=O)C(=O)[C@H](C[C@@H]1C[C@@H](C)NC1=O)NC(=O)c1cc(Cl)ccc1NC(=O)C(C)c1ccc(C(F)(F)F)nc1] | 5-chloro-N-[(1S)-3- (methylamino)-1-[[(3S,5R)- 5-methyl-2-oxo-pyrrolidin-3- yl]methyl]-2,3-dioxo- propyl]-2-[2-[6- (trifluoromethyl)-3- pyridyl]propanoylamino] benzamide | 583.0 | 582.1728 |
| 231 | 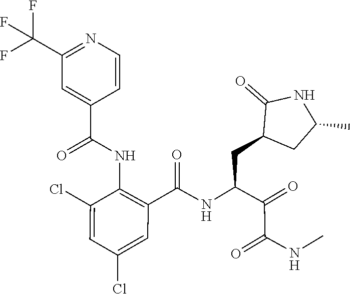 [SMILES: CNC(=O)C(=O)[C@H](C[C@@H]1C[C@@H](C)NC1=O)NC(=O)c1cc(Cl)cc(Cl)c1NC(=O)c1ccnc(C(F)(F)F)c1] | N-[2,4-dichloro-6-[[(1S)-3- (methylamino)-1-[[(3S,5R)- 5-methyl-2-oxo-pyrrolidin-3- yl]methyl]-2,3-dioxo- propyl]carbamoyl]phenyl]-2- (trifluoromethyl)pyridine-4- carboxamide | 589.4 | 588.1031 |
| 232 | 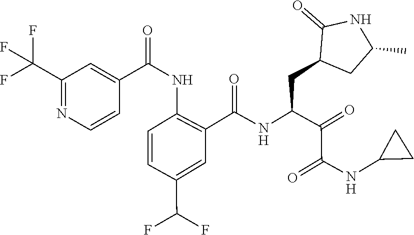 [SMILES: C[C@@H]1C[C@@H](C[C@H](NC(=O)c2cc(C(F)F)ccc2NC(=O)c2ccnc(C(F)(F)F)c2)C(=O)C(=O)NC2CC2)C(=O)N1] | N-[2-[[(1S)-3- (cyclopropylamino)-1- [[(3S,5R)-5-methyl-2-oxo- pyrrolidin-3-yl]methyl]-2,3- dioxo-propyl]carbamoyl]-4- (difluoromethyl)phenyl]-2- (trifluoromethyl)pyridine-4- carboxamide | 596.5 | 596.1929 |
| 233 | 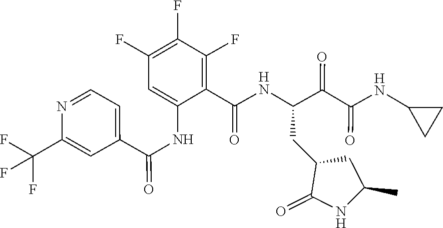 [SMILES: C[C@@H]1C[C@@H](C[C@H](NC(=O)c2c(NC(=O)c3ccnc(C(F)(F)F)c3)cc(F)c(F)c2F)C(=O)C(=O)NC2CC2)C(=O)N1] | N-[2-[[(1S)-3- (cyclopropylamino)-1- [[(3S,5R)-5-methyl-2-oxo- pyrrolidin-3-yl]methyl]-2,3- dioxo-propyl]carbamoyl]- 3,4,5-trifluoro-phenyl]-2- (trifluoromethyl)pyridine-4- carboxamide | 600.5 | 600.1678 |
| 234 | 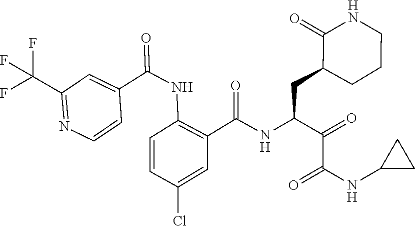 [SMILES: O=C(NC1CC1)C(=O)[C@H](C[C@@H]1CCCNC1=O)NC(=O)c1cc(Cl)ccc1NC(=O)c1ccnc(C(F)(F)F)c1] | N-[4-chloro-2-[[(1S)-3- (cyclopropylamino)-2,3- dioxo-1-[[(3S)-2-oxo-3- piperidyl]methyl]propyl] carbamoyl]phenyl]-2- (trifluoromethyl)pyridine-4- carboxamide | 580.96 | 580.1 |
| 235 | 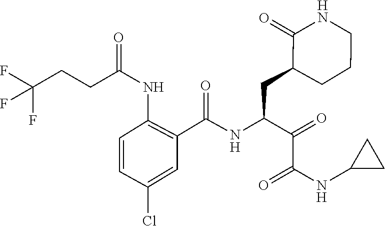 [SMILES: O=C(CCC(F)(F)F)Nc1ccc(Cl)cc1C(=O)N[C@@H](C[C@@H]1CCCNC1=O)C(=O)C(=O)NC1CC1] | 5-chloro-N-[(1S)-3- (cyclopropylamino)-2,3- dioxo-1-[[(3S)-2-oxo-3- piperidyl]methyl]propyl]-2- (4,4,4- trifluorobutanoylamino) benzamide | 531.92 | 531.1 |

### SARS2 Coronavirus 3CL Protease Assay

The enzymatic activity of SARS2 coronavirus 3CL protease was determined in a FRET (fluorescence resonance energy transfer)-based assay measuring the cleavage of a peptide substrate by recombinantly expressed and purified enzyme. Cleavage of the peptide SEQ TD NO:1 (CPC Scientific) by SARS2 3CL protease was measured in reaction buffer (50 mM Hepes pH 7.5, 0.01% Triton X-100, 0.01% BSA, 2 mM DTT). SARS2 3CL protease (5 nM final concentration) was pre-incubated with compound for 30 minutes before reaction initiation with peptide substrate (15 μM final concentration). Room temperature reactions (4 h) were quenched by addition of a high dose of inhibitor and read on an appropriate plate reader (excitation wavelength=495 nm, emission wavelength=520 nm). Data were analyzed by a standard 4 parameter fit to determine IC₅₀ values.

The compounds of the instant invention were tested in the assay described above and the results appear in the table below.

TABLE I
IC₅₀ values (nM) for Examples in the SARS2 3CLPro FRET assay

| Example | FRET Potency (nM) |
|---|---|
| 1 | 7.5 |
| 2 | 5.6 |
| 3 | 6.9 |
| 4 | 11.5 |
| 5 | 8.7 |
| 6 | 4.8 |
| 7 | 4.8 |
| 8 | 10.1 |
| 9 | 7.3 |
| 10 | 3.9 |
| 11 | 4.8 |
| 12 | 48.2 |
| 13 | 46.7 |
| 14 | 46.4 |
| 15 | 33.5 |
| 16 | 31.6 |
| 17 | 31.1 |
| 18 | 31.1 |
| 19 | 28.2 |
| 20 | 26.4 |
| 21 | 24.9 |
| 22 | 22.6 |
| 23 | 20.6 |
| 24 | 18.6 |
| 25 | 18.1 |
| 26 | 17.4 |
| 27 | 13.6 |
| 28 | 13.2 |
| 29 | 13.1 |
| 30 | 12.9 |
| 31 | 12.4 |
| 32 | 12.3 |
| 33 | 12.2 |
| 34 | 11.2 |
| 35 | 11.1 |
| 36 | 10.7 |
| 37 | 10.7 |
| 38 | 10.5 |
| 39 | 10.3 |
| 40 | 10.0 |
| 41 | 9.9 |
| 42 | 9.8 |
| 43 | 9.6 |
| 44 | 9.4 |
| 45 | 9.4 |
| 46 | 8.9 |
| 47 | 8.8 |
| 48 | 8.8 |
| 49 | 8.8 |
| 50 | 8.7 |
| 51 | 8.6 |
| 52 | 8.4 |
| 53 | 8.3 |
| 54 | 8.2 |
| 55 | 8.2 |
| 56 | 8.1 |
| 57 | 8.1 |
| 58 | 8.1 |
| 59 | 8.0 |
| 60 | 7.9 |
| 61 | 7.7 |
| 62 | 7.6 |
| 63 | 7.3 |
| 64 | 7.3 |
| 65 | 7.2 |
| 66 | 7.0 |
| 67 | 7.0 |
| 68 | 7.0 |
| 69 | 6.9 |
| 70 | 6.9 |
| 71 | 6.6 |
| 72 | 6.3 |
| 73 | 6.2 |
| 74 | 6.2 |
| 75 | 6.0 |
| 76 | 5.3 |
| 77 | 5.3 |
| 78 | 4.6 |
| 79 | 11.3 |
| 80 | 21.6 |
| 81 | 15.9 |
| 82 | 10.8 |
| 83 | 10.5 |
| 84 | 20.6 |
| 85 | 15.2 |
| 86 | 6.8 |
| 87 | 6.6 |
| 88 | 113.8 |
| 89 | 10.1 |
| 90 | 12.3 |
| 91 | 6.1 |
| 92 | 9.0 |
| 93 | 12.6 |
| 94 | 10.6 |
| 95 | 44.7 |
| 96 | 6.0 |
| 97 | 7.7 |
| 98 | 9.5 |
| 99 | 13.0 |
| 100 | 5.9 |
| 101 | 7.8 |
| 102 | 12.9 |
| 103 | 86.2 |
| 104 | 15.3 |
| 105 | 10.9 |
| 106 | 4.3 |
| 107 | 12.6 |
| 108 | 4.8 |
| 109 | 6.0 |
| 110 | 3.1 |
| 111 | 5.8 |
| 112 | 6.0 |
| 113 | 1287.0 |
| 114 | 18.9 |
| 115 | 11.5 |
| 116 | 20.9 |
| 117 | 9.3 |
| 118 | 23.9 |
| 119 | 5.6 |
| 120 | 11.1 |
| 121 | 10.9 |
| 122 | 8.1 |
| 123 | 7.3 |
| 124 | 8.2 |
| 125 | 5.6 |
| 126 | 3.8 |
| 127 | 7.9 |
| 128 | 7.2 |
| 129 | 5.9 |
| 130 | 20.7 |
| 131 | 36.5 |
| 132 | 13.8 |
| 133 | 9.9 |
| 134 | 28.2 |
| 135 | 3.0 |
| 136 | 3.6 |
| 137 | 5.4 |
| 138 | 18.6 |
| 139 | 6.3 |
| 140 | 9.2 |
| 141 | 19.8 |
| 142 | 41.1 |
| 143 | 3.0 |
| 144 | 5.3 |
| 145 | 26.8 |
| 146 | 3.9 |
| 147 | 11.6 |
| 148 | 24.7 |
| 149 | 10.8 |
| 150 | 17.9 |
| 151 | 5.1 |
| 152 | 5.0 |
| 153 | 39.8 |
| 154 | 4.5 |
| 155 | 30.4 |
| 156 | 14.6 |
| 157 | 8.9 |
| 158 | 8.3 |
| 159 | 7.9 |
| 160 | 8.8 |
| 161 | 5.9 |
| 162 | 7.5 |
| 163 | 8.6 |
| 164 | 4.8 |
| 165 | 6.3 |
| 166 | 10.1 |
| 167 | 5.5 |
| 168 | 12.0 |
| 169 | 75.3 |
| 170 | 10.5 |
| 171 | 3.2 |
| 172 | 3.7 |
| 173 | 6.7 |
| 174 | 6.5 |
| 175 | 2.8 |
| 176 | 2.5 |
| 177 | 10.9 |
| 178 | 7.0 |
| 179 | 2.5 |
| 180 | 4.5 |
| 181 | 7.2 |
| 182 | 6.7 |
| 183 | 10.1 |
| 184 | 13.5 |
| 185 | 9.0 |
| 186 | 2.5 |
| 187 | 2.5 |
| 188 | 7.2 |
| 189 | 3.5 |
| 190 | 3.1 |
| 191 | 16.1 |
| 192 | 10.3 |
| 193 | 5.2 |
| 194 | 6.8 |
| 195 | 3.1 |
| 196 | 7.6 |
| 197 | 9.4 |
| 198 | 5.5 |
| 199 | 18.7 |
| 200 | 4.6 |
| 201 | 7.0 |
| 202 | 11.9 |
| 203 | 36.6 |
| 204 | 4.7 |
| 205 | 6.6 |
| 206 | 12.8 |
| 207 | 11.4 |
| 208 | 6.9 |
| 209 | 9.8 |
| 210 | 7.8 |
| 211 | 7.3 |
| 212 | 5.6 |
| 213 | 15.4 |
| 214 | 6.8 |
| 215 | 10.1 |
| 216 | 9.6 |
| 217 | 6.7 |
| 218 | 6.8 |
| 219 | 7.1 |
| 220 | 60.3 |
| 221 | 41.9 |
| 222 | 5.2 |
| 223 | 9.0 |
| 224 | 7.2 |
| 225 | 6.8 |
| 226 | 19.1 |
| 227 | 7.4 |
| 228 | 6.1 |
| 229 | 33.5 |
| 230 | 7.0 |
| 231 | 5.6 |
| 232 | 10.4 |
| 233 | 44.0 |
| 234 | 17.1 |
| 235 | 15.1 |

What is claimed is:

1. A compound of formula I 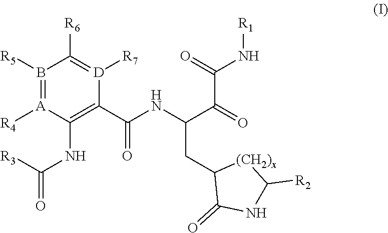
or a pharmaceutically acceptable salt thereof, wherein:
R₁ is H; (C₃-C₆)cycloalkyl; (C₁-C₆)alkyl; (C₁-C₆)alkyl-OH; phenyl; (C₁-C₆)alkyl-phenyl; (C₄-C₆)heterocycloalkyl containing 1 to 3 heteroatom(s) independently selected from N, O, or S; (C₁-C₆)alkyl-(C₄-C₆)heterocycloalkyl containing 1 to 3 heteroatom(s) independently selected from N, O, or S; (C₁-C₆)alkyl-(C₅-C₆)heteroaryl containing 1 to 3 heteroatom(s) independently selected from N, O, or S; (C₅-C₆)heteroaryl containing 1 to 3 heteroatom(s) independently selected from N, O, or S;
R₂ is H; (C₁-C₆)alkyl; (C₃-C₆)cycloalkyl; or CF₃;
R₃ is (C₁-C₆)alkyl; (C₁-C₆)alkyl-CF₃; (C₁-C₆)alkyl-OH; (C₁-C₆)alkyl-O—CH₃; (C₁-C₆)alkyl-O—CF₃; (C₁-C₆)alkyl-O—(C₃-C₁₀)cycloalkyl optionally substituted by an OH, —(C₁-C₆)alkyl, CF₃, —(C₁-C₆)alkyl-CF₃, or up to 3 halogen; (CF₂)-phenyl; (C₁-C₆)alkyl-phenyl; (C₃-C₁₀)cycloalkyl optionally substituted by an OH, —(C₁-C₆)alkyl, CF₃, —(C₁-C₆)alkyl-CF₃, or up to 3 halogen; (C₁-C₆)alkyl-(C₃-C₁₀)cycloalkyl optionally substituted by a OH, —(C₁-C₆)alkyl, CF₃, —(C₁-C₆)alkyl-CF₃, or up to 3 halogen; (C₄-C₁₀)heterocycle including up to 3 heteroatoms independently selected from N, O, and S and optionally substituted by a carbonyl, OH, —(C₁-C₆)alkyl, CF₃, —(C₁-C₆)alkyl-CF₃, or up to 3 halogen; (C₁-C₆)alkyl-(C₄-C₁₀)heterocycle including up to 3 heteroatoms independently selected from N, O, and S and optionally substituted by a carbonyl, OH, —(C₁-C₆)alkyl, CF₃, —(C₁-C₆)alkyl-CF₃, or up to 3 halogen; (C₆-C₁₀)aryl optionally substituted by an OH, —(C₁-C₆)alkyl, CF₃, —(C₁-C₆)alkyl-CF₃, or up to 3 halogen; (C₁-C₆)alkyl-(C₆-C₁₀)aryl optionally substituted by an OH, —(C₁-C₆)alkyl, CF₃, —(C₁-C₆)alkyl-CF₃, or up to 3 halogen; (C₅-C₁₀)heteroaryl including up to 4 heteroatoms independently selected from N, O, and S and optionally substituted by an OH, CN, CHF₂, —(C₁-C₆)alkyl, CF₃, —(C₁-C₆)alkyl-CF₃, —(C₃-C₆)cycloalkyl, O—(C₁-C₆)alkyl, O—CF₃, O—(C₃-C₆)cycloalkyl, or up to 3 halogen; (C₁-C₆)alkyl-(C₅-C₁₀)heteroaryl including up to 4 heteroatoms independently selected from N, O, and S and optionally substituted by an OH, CN, CHF₂, (C₃-C₆)cycloalkyl, O—(C₃-C₆)cycloalkyl, —(C₁-C₆)alkyl, O—(C₁-C₆)alkyl, CF₃, O—CF₃, —(C₁-C₆)alkyl-CF₃, or up to 3 halogen; —O—(C₁-C₆)alkyl; —O—(C₁-C₆)alkyl-(C₃-C₆)cycloalkyl optionally substituted by an OH, CF₃, —(C₁-C₆)alkyl, —(C₁-C₆)alkyl-CF₃, or up to 3 halogen; —O—(C₃-C₆)cycloalkyl optionally substituted by an OH, CF₃, —(C₁-C₆)alkyl, —(C₁-C₆)alkyl-CF₃, or up to 3 halogen; CHF₂; CF₃; (C₁-C₆)alkyl-N(CH₃)—C(O)—O—CH₃; or N(R₈)₂;
R₄ is H; F; Cl; or (C₁-C₆)alkyl;
R₅ is H; F; Cl; or (C₁-C₆)alkyl;
R₆ is H; F; Cl; CN; CF₃; O—CHF₂; O—CF₃; —(C₁-C₆)alkyl-CF₃; CHF₂; CF₃; (C₁-C₆)alkyl; (C₃-C₆)cycloalkyl; —O—(C₃-C₆)cycloalkyl; or O—(C₁-C₆)alkyl;
R₇ is H; F; Cl; or (C₁-C₆)alkyl;
R₈ is independently H; (C₁-C₆)alkyl; (C₃-C₁₀)cycloalkyl optionally substituted by an OH, —(C₁-C₆)alkyl, CF₃, —(C₁-C₆)alkyl-CF₃, or up to 3 halogen; (C₁-C₆)alkyl-(C₃-C₁₀)cycloalkyl optionally substituted by an OH, —(C₁-C₆)alkyl, CF₃, —(C₁-C₆)alkyl-CF₃, or up to 3 halogen; (C₄-C₁₀)heterocycle including up to 3 heteroatoms selected from N, O, and S and optionally substituted by an OH, —(C₁-C₆)alkyl, CF₃, —(C₁-C₆)alkyl-CF₃, or up to 3 halogen; (C₁-C₆)alkyl-(C₄-C₁₀)heterocycle including up to 3 heteroatoms selected from N, O, and S and optionally substituted by an OH, —(C₁-C₆)alkyl, CF₃, —(C₁-C₆)alkyl-CF₃, or up to 3 halogen; (C₆-C₁₀)aryl optionally substituted by an OH, —(C₁-C₆)alkyl, CF₃, —(C₁-C₆)alkyl-CF₃, or up to 3 halogen; (C₁-C₆)alkyl-(C₆-C₁₀)aryl optionally substituted by an OH, —(C₁-C₆)alkyl, CF₃, —(C₁-C₆)alkyl-CF₃, or up to 3 halogen; (C₅-C₁₀)heteroaryl including up to 3 heteroatoms selected from N, O, and S and optionally substituted by an OH, —(C₁-C₆)alkyl, CF₃, —(C₁-C₆)alkyl-CF₃, or up to 3 halogen; (C₁-C₆)alkyl-(C₅-C₁₀)heteroaryl including up to 3 heteroatoms selected from N, O, and S and optionally substituted by an OH, —(C₁-C₆)alkyl, CF₃, —(C₁-C₆)alkyl-CF₃, or up to 3 halogen;
A is C or N;
B is C or N;
D is C or N, provided that only 1 of A, B, or D can be N and that if A, B, or D is N then the respective R₄, R₅, or R₇ is absent; and
x is 1 or 2.

2. The compound of claim 1 or a pharmaceutically acceptable salt thereof, wherein R₁ is H, (C₃-C₆)cycloalkyl, or (C₁-C₆)alkyl.

3. The compound of claim 1 or a pharmaceutically acceptable salt thereof, wherein R₁ is H, CH₃, or cyclopropyl.

4. The compound of claim 1 or a pharmaceutically acceptable salt thereof, wherein R₂ is CF₃, CH₃, or H.

5. The compound of claim 1 or a pharmaceutically acceptable salt thereof, wherein R₃ is selected from the group consisting of CH₃,

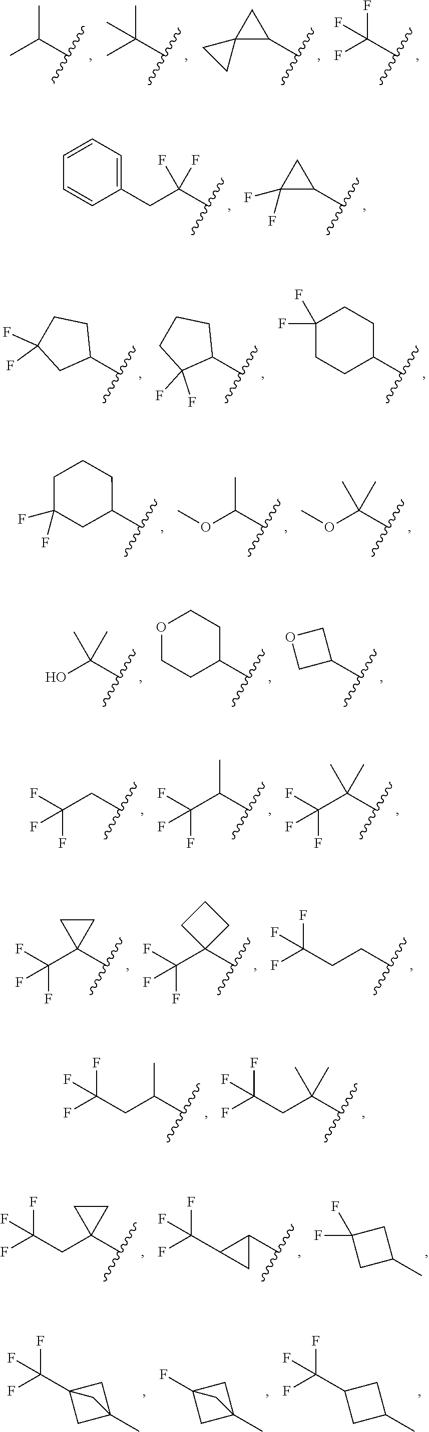

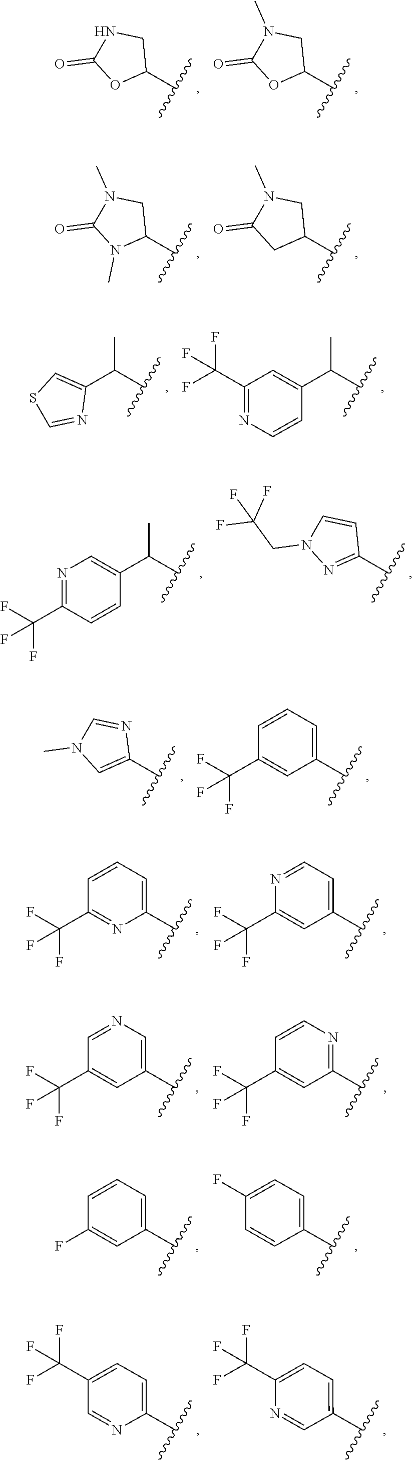

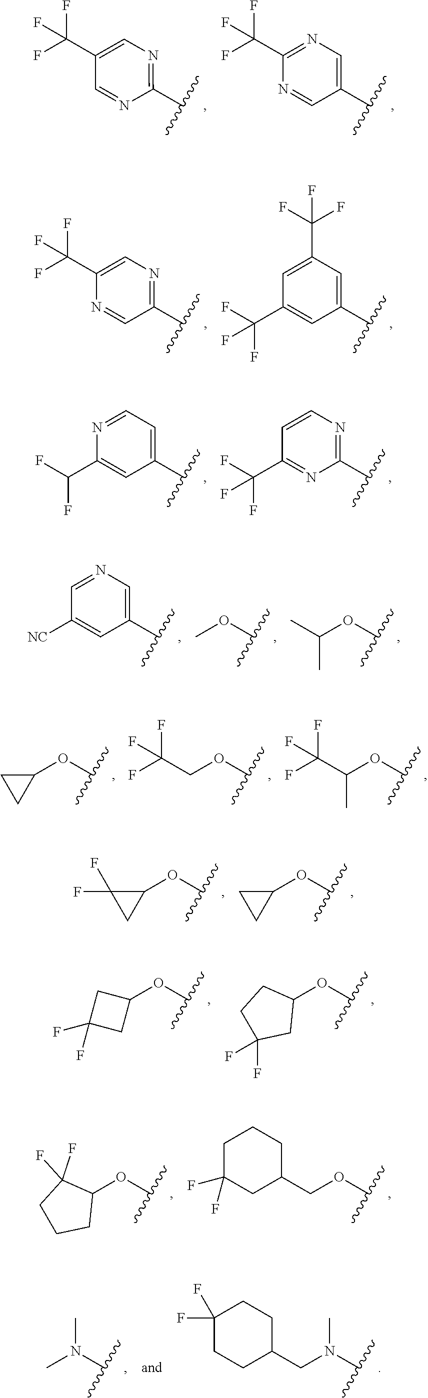

6. The compound of claim 1 or a pharmaceutically acceptable salt thereof, wherein R₄ is H, F, or Cl.

7. The compound of claim 1 or a pharmaceutically acceptable salt thereof, wherein R₅ is H, F, or Cl.

8. The compound of claim 1 or a pharmaceutically acceptable salt thereof, wherein R₆ is F, Cl, CHF₂, or CN.

9. The compound of claim 1 or a pharmaceutically acceptable salt thereof, wherein R₇ is H, F, or Cl.

10. The compound of claim 1 or a pharmaceutically acceptable salt thereof, wherein one of A, B or D is N.

11. The compound of claim 1 or a pharmaceutically acceptable salt thereof, wherein A, B, and D are all C.

12. The compound of claim 1 or a pharmaceutically acceptable salt thereof, wherein x is 1.

13. The compound of claim 1 or a pharmaceutically acceptable salt thereof, wherein the compound is selected from the group consisting of:
5-chloro-N-[(1S)-3-(cyclopropylamino)-1-[[(3S,5R)-5-methyl-2-oxo-pyrrolidin-3-yl]methyl]-2,3-dioxo-propyl]-2-[[1-(trifluoromethyl)cyclopropanecarbonyl]amino]benzamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-2-(trifluoromethyl)pyridine-4-carboxamide;
5-chloro-N-[(1S)-3-(cyclopropylamino)-1-[[(3S,5R)-5-methyl-2-oxo-pyrrolidin-3-yl]methyl]-2,3-dioxo-propyl]-2-(4,4,4-trifluorobutanoylamino)benzamide;
2-[[3,5-bis(trifluoromethyl)benzoyl]amino]-5-cyano-N-[(1S)-3-(methylamino)-1-[[(3S,5R)-5-methyl-2-oxo-pyrrolidin-3-yl]methyl]-2,3-dioxo-propyl]pyridine-3-carboxamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[[3-(trifluoromethyl)bicyclo[1.1.1]pentane-1-carbonyl]amino]pyridine-3-carboxamide;
isopropyl N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]carbamate;
5-chloro-N-[(1S)-3-(cyclopropylamino)-1-[[(3S,5R)-5-methyl-2-oxo-pyrrolidin-3-yl]methyl]-2,3-dioxo-propyl]-2-[(2,2-difluoro-3-phenyl-propanoyl)amino]benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[[(1R)-2,2-difluorocyclopropanecarbonyl]amino]benzamide;
methyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
N-[(1S)-3-(cyclopropylamino)-2,3-dioxo-1-[[(3S)-2-oxopyrrolidin-3-yl]methyl]propyl]-5-fluoro-2-[[1-(trifluoromethyl)cyclopropanecarbonyl]amino]benzamide;
N-[4-cyano-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-3-fluoro-bicyclo[1.1.1]pentane-1-carboxamide;
5-chloro-2-[(2,2-difluorocyclopropanecarbonyl)amino]-N-[(1S)-3-(methylamino)-1-[[(3S,5R)-5-methyl-2-oxo-pyrrolidin-3-yl]methyl]-2,3-dioxo-propyl]benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[(3,3,3-trifluoro-2,2-dimethyl-propanoyl)amino]benzamide;
5-chloro-2-[(3,3-difluorocyclopentanecarbonyl)amino]-N-[(1S)-3-(methylamino)-1-[[(3S,5R)-5-methyl-2-oxo-pyrrolidin-3-yl]methyl]-2,3-dioxo-propyl]benzamide;
N-[4-cyano-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-3-(trifluoromethyl)bicyclo[1.1.1]pentane-1-carboxamide;
5-(difluoromethoxy)-N-[(1S)-3-(methylamino)-1-[[(3S,5R)-5-methyl-2-oxo-pyrrolidin-3-yl]methyl]-2,3-dioxo-propyl]-2-[[3-(trifluoromethyl)benzoyl]amino]pyridine-3-carboxamide;
5-chloro-N-[(1S)-3-(methylamino)-1-[[(3S,5R)-5-methyl-2-oxo-pyrrolidin-3-yl]methyl]-2,3-dioxo-propyl]-2-[(3,3,3-trifluoro-2-methyl-propanoyl)amino]benzamide;
N-[(1S)-3-(cyclopropylamino)-1-[[(3S,5R)-5-methyl-2-oxo-pyrrolidin-3-yl]methyl]-2,3-dioxo-propyl]-5-(difluoromethoxy)-2-[[3-(trifluoromethyl)benzoyl]amino]benzamide;
5-chloro-2-[(3,3-difluorocyclobutanecarbonyl)amino]-N-[(1S)-3-(methylamino)-1-[[(3S,5R)-5-methyl-2-oxo-pyrrolidin-3-yl]methyl]-2,3-dioxo-propyl]benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[[1-(trifluoromethyl)cyclopropanecarbonyl]amino]pyridine-3-carboxamide;
N-[(1S)-3-(cyclopropylamino)-1-[[(3S,5R)-5-methyl-2-oxo-pyrrolidin-3-yl]methyl]-2,3-dioxo-propyl]-2-[(3,3-difluorocyclobutanecarbonyl)amino]-5-fluoro-pyridine-3-carboxamide;
5-chloro-N-[(1S)-3-(methylamino)-1-[[(3S,5R)-5-methyl-2-oxo-pyrrolidin-3-yl]methyl]-2,3-dioxo-propyl]-2-[(3,3,3-trifluoro-2-methyl-propanoyl)amino]benzamide;
N-[(1S)-3-(cyclopropylamino)-1-[[(3S,5R)-5-methyl-2-oxo-pyrrolidin-3-yl]methyl]-2,3-dioxo-propyl]-5-fluoro-2-[[1-(trifluoromethyl)cyclopropanecarbonyl]amino]benzamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-6-(trifluoromethyl)pyridine-2-carboxamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[(3-fluorobicyclo[1.1.1]pentane-1-carbonyl)amino]pyridine-3-carboxamide;
5-fluoro-N-[(1S)-3-(methylamino)-1-[[(3S,5R)-5-methyl-2-oxo-pyrrolidin-3-yl]methyl]-2,3-dioxo-propyl]-2-[[3-(trifluoromethyl)benzoyl]amino]pyridine-3-carboxamide;
5-chloro-N-[(1S)-3-(cyclopropylamino)-2,3-dioxo-1-[[(3S)-2-oxopyrrolidin-3-yl]methyl]propyl]-2-(4,4,4-trifluorobutanoylamino)benzamide;
N-[4-chloro-2-[[(1S)-3-(cyclopropylamino)-2,3-dioxo-1-[[(3S)-2-oxopyrrolidin-3-yl]methyl]propyl]carbamoyl]phenyl]-3-fluoro-bicyclo[1.1.1]pentane-1-carboxamide;
isopropyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
5-cyano-N-[(1S)-3-(cyclopropylamino)-1-[[(3S,5R)-5-methyl-2-oxo-pyrrolidin-3-yl]methyl]-2,3-dioxo-propyl]-2-[(3-fluorobenzoyl)amino]benzamide;
5-chloro-N-[(1S)-3-(cyclopropylamino)-1-[[(3S,5R)-5-methyl-2-oxo-pyrrolidin-3-yl]methyl]-2,3-dioxo-propyl]-4-fluoro-2-[[1-(trifluoromethyl)cyclopropanecarbonyl]amino]benzamide;
5-chloro-N-[(1S)-3-(methylamino)-1-[[(3S,5R)-5-methyl-2-oxo-pyrrolidin-3-yl]methyl]-2,3-dioxo-propyl]-2-[[1-(trifluoromethyl)cyclopropanecarbonyl]amino]benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[[1-(trifluoromethyl)cyclobutanecarbonyl]amino]benzamide;
5-chloro-N-[(1S)-3-(cyclopropylamino)-2,3-dioxo-1-[[(3S)-2-oxopyrrolidin-3-yl]methyl]propyl]-2-[[1-(2,2,2-trifluoroethyl)cyclopropanecarbonyl]amino]benzamide;
N-[4-chloro-2-[[(1S)-3-(cyclopropylamino)-2,3-dioxo-1-[[(3S)-2-oxopyrrolidin-3-yl]methyl]propyl]carbamoyl]phenyl]-3-fluoro-bicyclo[1.1.1]pentane-1-carboxamide;
5-chloro-N-[(1S)-3-(cyclopropylamino)-2,3-dioxo-1-[[(3S)-2-oxopyrrolidin-3-yl]methyl]propyl]-2-(3,3,3-trifluoropropanoylamino)benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[(4,4-difluorocyclohexanecarbonyl)amino]pyridine-3-carboxamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-6-(trifluoromethyl)pyridine-2-carboxamide;
5-chloro-2-[(3,3-difluorocyclobutanecarbonyl)amino]-N-[(1S)-3-(methylamino)-2,3-dioxo-1-[[(3R,5S)-2-oxo-5-(trifluoromethyl)pyrrolidin-3-yl]methyl]propyl]benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-(3,3,3-trifluoropropanoylamino)benzamide;
5-chloro-N-[(1S)-3-(methylamino)-2,3-dioxo-1-[[(3R,5S)-2-oxo-5-(trifluoromethyl)pyrrolidin-3-yl]methyl]propyl]-2-[[3-(trifluoromethyl)benzoyl]amino]pyridine-3-carboxamide;
N-[(1S)-3-(cyclopropylamino)-1-[[(3S,5R)-5-methyl-2-oxo-pyrrolidin-3-yl]methyl]-2,3-dioxo-propyl]-5-(difluoromethyl)-2-[[3-(trifluoromethyl)benzoyl]amino]benzamide;
5-chloro-N-[(1S)-3-(methylamino)-1-[[(3S,5R)-5-methyl-2-oxo-pyrrolidin-3-yl]methyl]-2,3-dioxo-propyl]-2-[[3-(trifluoromethyl)benzoyl]amino]benzamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-3-(trifluoromethyl)bicyclo[1.1.1]pentane-1-carboxamide;
N-[4-chloro-2-[[(1S)-3-(methylamino)-2,3-dioxo-1-[[(3S)-2-oxopyrrolidin-3-yl]methyl]propyl]carbamoyl]phenyl]-3-fluoro-bicyclo[1.1.1]pentane-1-carboxamide;
N-[(1S)-3-amino-1-[[(3S,5R)-5-methyl-2-oxo-pyrrolidin-3-yl]methyl]-2,3-dioxo-propyl]-5-chloro-2-[[3-(trifluoromethyl)benzoyl]amino]benzamide;
N-[2-[[(1S)-3-(cyclopropylamino)-1-[[(3S,5R)-5-methyl-2-oxo-pyrrolidin-3-yl]methyl]-2,3-dioxo-propyl]carbamoyl]-4,5-difluoro-phenyl]-2-(trifluoromethyl)pyridine-4-carboxamide;
5-chloro-2-[(3-fluorobicyclo[1.1.1]pentane-1-carbonyl)amino]-N-[(1S)-3-(methylamino)-1-[[(3S,5R)-5-methyl-2-oxo-pyrrolidin-3-yl]methyl]-2,3-dioxo-propyl]pyridine-3-carboxamide;
5-chloro-N-[(1S)-3-(cyclopropylamino)-2,3-dioxo-1-[[(3S)-2-oxopyrrolidin-3-yl]methyl]propyl]-2-[(2,2,2-trifluoroacetyl)amino]benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-(4,4,4-trifluorobutanoylamino)pyridine-3-carboxamide;
5-chloro-N-[(1S)-3-(cyclopropylamino)-1-[[(3S,5R)-5-methyl-2-oxo-pyrrolidin-3-yl]methyl]-2,3-dioxo-propyl]-4-fluoro-2-(4,4,4-trifluorobutanoylamino)benzamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-3-fluoro-bicyclo[1.1.1]pentane-1-carboxamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-5-(trifluoromethyl)pyridine-2-carboxamide;
5-chloro-N-[(1S)-3-(cyclopropylamino)-2,3-dioxo-1-[[(3S)-2-oxopyrrolidin-3-yl]methyl]propyl]-4-fluoro-2-(4,4,4-trifluorobutanoylamino)benzamide;
5-chloro-2-[(4-fluorobenzoyl)amino]-N-[(1S)-3-(methylamino)-1-[[(3S,5R)-5-methyl-2-oxo-pyrrolidin-3-yl]methyl]-2,3-dioxo-propyl]pyridine-3-carboxamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[(3-fluorobenzoyl)amino]pyridine-3-carboxamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[(3,3-difluorocyclobutanecarbonyl)amino]benzamide;
5-chloro-2-[(2,2-difluorocyclopropanecarbonyl)amino]-N-[(1S)-3-(methylamino)-1-[[(3S,5R)-5-methyl-2-oxo-pyrrolidin-3-yl]methyl]-2,3-dioxo-propyl]benzamide;
5-cyano-N-[(1S)-3-(cyclopropylamino)-1-[[(3S,5R)-5-methyl-2-oxo-pyrrolidin-3-yl]methyl]-2,3-dioxo-propyl]-2-[[3-(trifluoromethyl)benzoyl]amino]benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[[1-(2,2,2-trifluoroethyl)cyclopropanecarbonyl]amino]benzamide;
5-chloro-N-[(1S)-3-(cyclopropylamino)-2,3-dioxo-1-[[(3S)-2-oxopyrrolidin-3-yl]methyl]propyl]-2-[(3,3-difluorocyclobutanecarbonyl)amino]benzamide;
N-[(1S)-3-(cyclopropylamino)-1-[[(3S,5R)-5-methyl-2-oxo-pyrrolidin-3-yl]methyl]-2,3-dioxo-propyl]-5-(trifluoromethyl)-2-[[3-(trifluoromethyl)benzoyl]amino]benzamide;
5-chloro-2-[(3,3-difluorocyclopentanecarbonyl)amino]-N-[(1S)-3-(methylamino)-1-[[(3S,5R)-5-methyl-2-oxo-pyrrolidin-3-yl]methyl]-2,3-dioxo-propyl]benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[[3-(trifluoromethyl)benzoyl]amino]benzamide;
N-[(1S)-3-(cyclopropylamino)-1-[[(3S,5R)-5-methyl-2-oxo-pyrrolidin-3-yl]methyl]-2,3-dioxo-propyl]-5-fluoro-2-[[3-(trifluoromethyl)benzoyl]amino]pyridine-3-carboxamide;
5-chloro-N-[(1S)-3-(cyclopropylamino)-2,3-dioxo-1-[[(3R,5S)-2-oxo-5-(trifluoromethyl)pyrrolidin-3-yl]methyl]propyl]-2-(4,4,4-trifluorobutanoylamino)benzamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-3-(trifluoromethyl)bicyclo[1.1.1]pentane-1-carboxamide;
5-(difluoromethyl)-N-[(1S)-3-(methylamino)-1-[[(3S,5R)-5-methyl-2-oxo-pyrrolidin-3-yl]methyl]-2,3-dioxo-propyl]-2-[[3-(trifluoromethyl)benzoyl]amino]benzamide;
N-[4-chloro-2-[[(1S)-3-(cyclopropylamino)-2,3-dioxo-1-[[(3S)-2-oxopyrrolidin-3-yl]methyl]propyl]carbamoyl]phenyl]-5-(trifluoromethyl)pyridine-3-carboxamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-4-(trifluoromethyl)pyridine-2-carboxamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[(3-fluorobenzoyl)amino]benzamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-2-(trifluoromethyl)pyridine-4-carboxamide;
N-[4-chloro-2-[[(1S)-3-(cyclopropylamino)-2,3-dioxo-1-[[(3S)-2-oxopyrrolidin-3-yl]methyl]propyl]carbamoyl]phenyl]-2-(trifluoromethyl)pyridine-4-carboxamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-6-(trifluoromethyl)pyridine-3-carboxamide;
N-[2-[[(1S)-3-(cyclopropylamino)-1-[[(3S,5R)-5-methyl-2-oxo-pyrrolidin-3-yl]methyl]-2,3-dioxo-propyl]carbamoyl]-4-fluoro-phenyl]-2-(trifluoromethyl)pyridine-4-carboxamide;
N-[(1S)-3-(cyclopropylamino)-1-[[(3S,5R)-5-methyl-2-oxo-pyrrolidin-3-yl]methyl]-2,3-dioxo-propyl]-5-fluoro-2-[[3-(trifluoromethyl)benzoyl]amino]benzamide;
N-[4-chloro-2-[[(1S)-3-(cyclopropylamino)-2,3-dioxo-1-[[(3S)-2-oxopyrrolidin-3-yl]methyl]propyl]carbamoyl]-5-fluoro-phenyl]-5-(trifluoromethyl)pyridine-3-carboxamide;
N-[2,4-dichloro-6-[[(1S)-3-(cyclopropylamino)-1-[[(3S,5R)-5-methyl-2-oxo-pyrrolidin-3-yl]methyl]-2,3-dioxo-propyl]carbamoyl]phenyl]-3-fluoro-bicyclo[1.1.1]pentane-1-carboxamide;
5-chloro-N-[(1S)-3-(methylamino)-2,3-dioxo-1-[[(3S)-2-oxopyrrolidin-3-yl]methyl]propyl]-2-[[1-(trifluoromethyl)cyclopropanecarbonyl]amino]benzamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]spiro[2.2]pentane-2-carboxamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]spiro[2.2]pentane-2-carboxamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[[2-(trifluoromethyl)cyclopropanecarbonyl]amino]benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[[2-(trifluoromethyl)cyclopropanecarbonyl]amino]benzamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-4-(trifluoromethyl)pyridine-2-carboxamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]-5-fluoro-phenyl]-2-(trifluoromethyl)pyridine-4-carboxamide;
5-cyano-N-[3-(methylamino)-2,3-dioxo-1-[[(5S)-2-oxo-5-(trifluoromethyl)pyrrolidin-3-yl]methyl]propyl]-2-(4,4,4-trifluorobutanoylamino)benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-(2-thiazol-4-ylpropanoylamino)benzamide;
N-[4-chloro-2-[[(1S)-3-(methylamino)-2,3-dioxo-1-[[(3S)-2-oxopyrrolidin-3-yl]methyl]propyl]carbamoyl]phenyl]-2-(trifluoromethyl)pyridine-4-carboxamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-5-(trifluoromethyl)pyridine-3-carboxamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-2-(difluoromethyl)pyridine-4-carboxamide;
5-chloro-N-[(1S)-3-(cyclopropylamino)-1-[[(3S,5R)-5-methyl-2-oxo-pyrrolidin-3-yl]methyl]-2,3-dioxo-propyl]-2-(2-thiazol-4-ylpropanoylamino)benzamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-5-(trifluoromethyl)pyridine-3-carboxamide;
N-[(1S)-3-(methylamino)-2,3-dioxo-1-[[(3S)-2-oxopyrrolidin-3-yl]methyl]propyl]-5-(trifluoromethyl)-2-[[1-(trifluoromethyl)cyclopropanecarbonyl]amino]benzamide;
isopropyl N-[4-chloro-2-[[(1S)-3-(cyclopropylamino)-2,3-dioxo-1-[[(3S)-2-oxopyrrolidin-3-yl]methyl]propyl]carbamoyl]phenyl]carbamate;
5-chloro-N-[(1S)-3-(cyclopropylamino)-1-[[(3S,5R)-5-methyl-2-oxo-pyrrolidin-3-yl]methyl]-2,3-dioxo-propyl]-2-[[2-(trifluoromethyl)cyclopropanecarbonyl]amino]benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[[2-(trifluoromethyl)cyclopropanecarbonyl]amino]benzamide;
N-[2-[[(1S)-3-(cyclopropylamino)-1-[[(3S,5R)-5-methyl-2-oxo-pyrrolidin-3-yl]methyl]-2,3-dioxo-propyl]carbamoyl]-4,5-difluoro-phenyl]-5-(trifluoromethyl)pyridine-3-carboxamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-1-(2,2,2-trifluoroethyl)pyrazole-3-carboxamide;
methyl N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]carbamate;
cyclopropyl (4-chloro-2-(((S)-4-(methylamino)-3,4-dioxo-1-((S)-2-oxopyrrolidin-3-yl)butan-2-yl)carbamoyl)phenyl)carbamate;
cyclopropyl (4-chloro-2-(((S)-1-((3S,5R)-5-methyl-2-oxopyrrolidin-3-yl)-4-(methylamino)-3,4-dioxobutan-2-yl)carbamoyl)phenyl)carbamate;
5-chloro-N-[(1S)-3-(cyclopropylamino)-2,3-dioxo-1-[[(3S)-2-oxopyrrolidin-3-yl]methyl]propyl]-2-[[1-(trifluoromethyl)cyclopropanecarbonyl]amino]benzamide;
methyl N-[2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
methyl N-[4-fluoro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
methyl N-[4-chloro-2-[[(1S)-3-(methylamino)-2,3-dioxo-1-[[(3S)-2-oxopyrrolidin-3-yl]methyl]propyl]carbamoyl]phenyl]carbamate;
methyl N-[2-[[(1S)-3-amino-1-[[(3S,5R)-5-methyl-2-oxo-pyrrolidin-3-yl]methyl]-2,3-dioxo-propyl]carbamoyl]-4-chloro-phenyl]carbamate;
trideuteriomethyl N-[4-chloro-2-[[(1S)-3-(methylamino)-2,3-dioxo-1-[[(3S)-2-oxopyrrolidin-3-yl]methyl]propyl]carbamoyl]phenyl]carbamate;
methyl N-[4,5-difluoro-2-[[(1S)-3-(methylamino)-2,3-dioxo-1-[[(3S)-2-oxopyrrolidin-3-yl]methyl]propyl]carbamoyl]phenyl]carbamate;
5-chloro-2-(cyclopropanecarbonylamino)-N-[(1S)-3-(methylamino)-2,3-dioxo-1-[[(3S)-2-oxopyrrolidin-3-yl]methyl]propyl]benzamide;
ethyl N-[4-chloro-2-[[(1S)-3-(methylamino)-2,3-dioxo-1-[[(3S)-2-oxopyrrolidin-3-yl]methyl]propyl]carbamoyl]phenyl]carbamate;
trideuteriomethyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
methyl N-[6-chloro-2-[[(1S)-3-(methylamino)-1-[[(3S,5R)-5-methyl-2-oxo-pyrrolidin-3-yl]methyl]-2,3-dioxo-propyl]carbamoyl]-3-pyridyl]carbamate;
5-chloro-N-[(1S)-1-[[(3S,5R)-5-methyl-2-oxo-pyrrolidin-3-yl]methyl]-2,3-dioxo-3-(4-pyridylmethylamino)propyl]-2-(4,4,4-trifluorobutanoylamino)benzamide;
5-chloro-N-[(1S)-1-[[(3S,5R)-5-methyl-2-oxo-pyrrolidin-3-yl]methyl]-2,3-dioxo-3-(1H-1,2,4-triazol-3-ylmethylamino)propyl]-2-(4,4,4-trifluorobutanoylamino)benzamide;
5-chloro-N-[(1S)-3-(2-hydroxyethylamino)-1-[[(3S,5R)-5-methyl-2-oxo-pyrrolidin-3-yl]methyl]-2,3-dioxo-propyl]-2-(4,4,4-trifluorobutanoylamino)benzamide;
methyl N-[6-chloro-4-[[(1S)-3-(methylamino)-1-[[(3S,5R)-5-methyl-2-oxo-pyrrolidin-3-yl]methyl]-2,3-dioxo-propyl]carbamoyl]-3-pyridyl]carbamate;
methyl N-[5-chloro-3-[[(1S)-3-(methylamino)-1-[[(3S,5R)-5-methyl-2-oxo-pyrrolidin-3-yl]methyl]-2,3-dioxo-propyl]carbamoyl]-2-pyridyl]carbamate;
methyl N-[4,5-difluoro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
cyclopropyl N-[4-fluoro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
5-chloro-2-(cyclopropanecarbonylamino)-N-[(1S)-3-(methylamino)-1-[[(3S,5R)-5-methyl-2-oxo-pyrrolidin-3-yl]methyl]-2,3-dioxo-propyl]benzamide;
methyl N-[4-chloro-2-[[(1S)-3-(cyclopropylamino)-2,3-dioxo-1-[[(3S)-2-oxopyrrolidin-3-yl]methyl]propyl]carbamoyl]phenyl]carbamate;
trideuteriomethyl N-[4-chloro-2-[[(1S)-3-(cyclopropylamino)-2,3-dioxo-1-[[(3S)-2-oxopyrrolidin-3-yl]methyl]propyl]carbamoyl]phenyl]carbamate;
5-chloro-N-[(1S)-3-(methylamino)-1-[[(3S,5R)-5-methyl-2-oxo-pyrrolidin-3-yl]methyl]-2,3-dioxo-propyl]-2-(2-methylpropanoylamino)benzamide;
5-chloro-2-(2,2-dimethylpropanoylamino)-N-[(1S)-3-(methylamino)-2,3-dioxo-1-[[(3S)-2-oxopyrrolidin-3-yl]methyl]propyl]benzamide;
cyclopropyl N-[4,5-difluoro-2-[[(1S)-3-(methylamino)-2,3-dioxo-1-[[(3S)-2-oxopyrrolidin-3-yl]methyl]propyl]carbamoyl]phenyl]carbamate;
isopropyl N-[4-chloro-2-[[(1S)-3-(methylamino)-2,3-dioxo-1-[[(3S)-2-oxopyrrolidin-3-yl]methyl]propyl]carbamoyl]phenyl]carbamate;
ethyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
methyl N-[4-(difluoromethyl)-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
cyclopropyl N-[4-cyano-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
methyl N-[4-chloro-5-fluoro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
methyl N-[4-chloro-2-fluoro-6-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
methyl N-[5-chloro-4-fluoro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]oxetane-3-carboxamide;
cyclopropylmethyl N-[4-chloro-2-[[(1S)-3-(methylamino)-2,3-dioxo-1-[[(3S)-2-oxopyrrolidin-3-yl]methyl]propyl]carbamoyl]phenyl]carbamate;
cyclobutyl N-[4-chloro-2-[[(1S)-3-(methylamino)-2,3-dioxo-1-[[(3S)-2-oxopyrrolidin-3-yl]methyl]propyl]carbamoyl]phenyl]carbamate;
(1-methylcyclopropyl)N-[4-chloro-2-[[(1S)-3-(methylamino)-2,3-dioxo-1-[[(3S)-2-oxopyrrolidin-3-yl]methyl]propyl]carbamoyl]phenyl]carbamate;
ethyl N-[4-chloro-2-[[(1S)-3-(cyclopropylamino)-2,3-dioxo-1-[[(3S)-2-oxopyrrolidin-3-yl]methyl]propyl]carbamoyl]phenyl]carbamate;
cyclopropyl N-[5-chloro-3-[[(1S)-3-(methylamino)-1-[[(3S,5R)-5-methyl-2-oxo-pyrrolidin-3-yl]methyl]-2,3-dioxo-propyl]carbamoyl]-2-pyridyl]carbamate;
cyclopropyl N-[4,5-difluoro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
tert-butyl N-[4-chloro-2-[[(1S)-3-(methylamino)-2,3-dioxo-1-[[(3S)-2-oxopyrrolidin-3-yl]methyl]propyl]carbamoyl]phenyl]carbamate;
5-chloro-2-[[(1R)-2,2-difluorocyclopropanecarbonyl]amino]-N-[(1 S)-3-(methylamino)-2,3-dioxo-1-[[(3S)-2-oxopyrrolidin-3-yl]methyl]propyl]benzamide;
methyl N-[4,5-dichloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
5-chloro-2-(cyclopropanecarbonylamino)-N-[(1S)-3-(cyclopropylamino)-1-[[(3S,5R)-5-methyl-2-oxo-pyrrolidin-3-yl]methyl]-2,3-dioxo-propyl]benzamide;
cyclopropyl N-[4-chloro-2-[[(1S)-3-(cyclopropylamino)-2,3-dioxo-1-[[(3S)-2-oxopyrrolidin-3-yl]methyl]propyl]carbamoyl]phenyl]carbamate;
5-chloro-N-[(1S)-3-(cyclopropylamino)-1-[[(3S,5R)-5-methyl-2-oxo-pyrrolidin-3-yl]methyl]-2,3-dioxo-propyl]-2-(2-methylpropanoylamino)benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-(dimethylcarbamoylamino)benzamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-1-methyl-azetidine-3-carboxamide;
cyclopropyl N-[2-[[(1S)-3-(cyclopropylamino)-2,3-dioxo-1-[[(3S)-2-oxopyrrolidin-3-yl]methyl]propyl]carbamoyl]-4,5-difluoro-phenyl]carbamate;
cyclobutyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
cyclopropylmethyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
tert-butyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
cyclopropyl N-[4-chloro-5-fluoro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
cyclopropyl N-[5-chloro-4-fluoro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
(2,2-difluorocyclopropyl)N-[4-chloro-2-[[(1S)-3-(methylamino)-2,3-dioxo-1-[[(3S)-2-oxopyrrolidin-3-yl]methyl]propyl]carbamoyl]phenyl]carbamate;
(2,2-difluorocyclopropyl)N-[4-chloro-2-[[(1S)-3-(methylamino)-2,3-dioxo-1-[[(3S)-2-oxopyrrolidin-3-yl]methyl]propyl]carbamoyl]phenyl]carbamate;
N-[(1S)-3-amino-1-[[(3S,5R)-5-methyl-2-oxo-pyrrolidin-3-yl]methyl]-2,3-dioxo-propyl]-5-chloro-2-(4,4,4-trifluorobutanoylamino)benzamide;
5-chloro-N-[(1S)-3-(methylamino)-2,3-dioxo-1-[[(3S)-2-oxopyrrolidin-3-yl]methyl]propyl]-2-(4,4,4-trifluorobutanoylamino)benzamide;
cyclopropyl N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]carbamate;
cyclobutyl N-[4-chloro-2-[[(1S)-3-(cyclopropylamino)-2,3-dioxo-1-[[(3S)-2-oxopyrrolidin-3-yl]methyl]propyl]carbamoyl]phenyl]carbamate;
cyclopropylmethyl N-[4-chloro-2-[[(1S)-3-(cyclopropylamino)-2,3-dioxo-1-[[(3S)-2-oxopyrrolidin-3-yl]methyl]propyl]carbamoyl]phenyl]carbamate;
2,2,2-trifluoroethyl N-[4-chloro-2-[[(1S)-3-(methylamino)-2,3-dioxo-1-[[(3S)-2-oxopyrrolidin-3-yl]methyl]propyl]carbamoyl]phenyl]carbamate;
5-chloro-N-[(1S)-3-(cyclopropylamino)-1-[[(3S,5R)-5-methyl-2-oxo-pyrrolidin-3-yl]methyl]-2,3-dioxo-propyl]-2-[[(2R)-2-methoxypropanoyl]amino]benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[(2-hydroxy-2-methyl-propanoyl)amino]benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[[(2S)-2-methoxypropanoyl]amino]benzamide;
cyclopentyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
5-chloro-N-[(1S)-3-(cyclopropylamino)-2,3-dioxo-1-[[(3S)-2-oxopyrrolidin-3-yl]methyl]propyl]-2-[[(1R)-2,2-difluorocyclopropanecarbonyl]amino]benzamide;
cyclopropyl N-[2-[[(1S)-3-(methylamino)-1-[[(3S,5R)-5-methyl-2-oxo-pyrrolidin-3-yl]methyl]-2,3-dioxo-propyl]carbamoyl]-4-(trifluoromethyl)phenyl]carbamate;
cyclopropyl N-[4,5-dichloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
(2,2-difluorocyclopropyl)N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
(2,2-difluorocyclopropyl)N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
(3,3-difluorocyclobutyl)N-[4-chloro-2-[[(1S)-3-(methylamino)-2,3-dioxo-1-[[(3S)-2-oxopyrrolidin-3-yl]methyl]propyl]carbamoyl]phenyl]carbamate;
benzyl N-[4-chloro-2-[[(1S)-3-(methylamino)-2,3-dioxo-1-[[(3S)-2-oxopyrrolidin-3-yl]methyl]propyl]carbamoyl]phenyl]carbamate;
5-chloro-N-[(1S)-3-(methylamino)-2,3-dioxo-1-[[(3S)-2-oxopyrrolidin-3-yl]methyl]propyl]-2-[[2-(trifluoromethyl)cyclopropanecarbonyl]amino]benzamide;
5-chloro-N-[(1S)-3-(methylamino)-2,3-dioxo-1-[[(3S)-2-oxopyrrolidin-3-yl]methyl]propyl]-2-[[2-(trifluoromethyl)cyclopropanecarbonyl]amino]benzamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[(2,2,2-trifluoroacetyl)amino]benzamide;
5-chloro-N-[(1S)-3-(methylamino)-1-[[(3S,5R)-5-methyl-2-oxo-pyrrolidin-3-yl]methyl]-2,3-dioxo-propyl]-2-(4,4,4-trifluorobutanoylamino)benzamide;
5-chloro-N-[(1S)-3-(methylamino)-2,3-dioxo-1-[[(3S)-2-oxopyrrolidin-3-yl]methyl]propyl]-2-(2-thiazol-4-ylpropanoylamino)benzamide;
(2,2,2-trifluoro-1-methyl-ethyl)N-[4-chloro-2-[[(1S)-3-(methylamino)-2,3-dioxo-1-[[(3S)-2-oxopyrrolidin-3-yl]methyl]propyl]carbamoyl]phenyl]carbamate;
2,2,2-trifluoroethyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
(5S)—N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-3-methyl-2-oxo-oxazolidine-5-carboxamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-5-cyano-pyridine-3-carboxamide;
5-chloro-2-[(2,2-difluorocyclopentanecarbonyl)amino]-N-[(1S)-3-(methylamino)-1-[[(3S,5R)-5-methyl-2-oxo-pyrrolidin-3-yl]methyl]-2,3-dioxo-propyl]benzamide;
5-chloro-2-[(2,2-difluorocyclopentanecarbonyl)amino]-N-[(1S)-3-(methylamino)-1-[[(3S,5R)-5-methyl-2-oxo-pyrrolidin-3-yl]methyl]-2,3-dioxo-propyl]benzamide;
(2,2-difluorocyclopropyl)methyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
(2,2-difluorocyclopropyl)methyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
(3,3-difluorocyclobutyl)N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
5-chloro-N-[(1S)-3-(methylamino)-1-[[(3S,5R)-5-methyl-2-oxo-pyrrolidin-3-yl]methyl]-2,3-dioxo-propyl]-2-[[2-(trifluoromethyl)cyclopropanecarbonyl]amino]benzamide;
5-chloro-N-[(1S)-3-(methylamino)-1-[[(3S,5R)-5-methyl-2-oxo-pyrrolidin-3-yl]methyl]-2,3-dioxo-propyl]-2-[[2-(trifluoromethyl)cyclopropanecarbonyl]amino]benzamide;
2,2,2-trifluoroethyl N-[4-chloro-2-[[(1S)-3-(cyclopropylamino)-2,3-dioxo-1-[[(3S)-2-oxopyrrolidin-3-yl]methyl]propyl]carbamoyl]phenyl]carbamate;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]tetrahydropyran-4-carboxamide;
(5S)—N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-2-oxo-oxazolidine-5-carboxamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]morpholine-4-carboxamide;
5-chloro-N-[(1S)-3-(methylamino)-1-[[(3S,5R)-5-methyl-2-oxo-pyrrolidin-3-yl]methyl]-2,3-dioxo-propyl]-2-(2-thiazol-4-ylpropanoylamino)benzamide;
N-[4-fluoro-2-[[(1S)-3-(methylamino)-2,3-dioxo-1-[[(3S)-2-oxopyrrolidin-3-yl]methyl]propyl]carbamoyl]phenyl]-2-(trifluoromethyl)pyridine-4-carboxamide;
5-chloro-2-[(4,4-difluorocyclohexanecarbonyl)amino]-N-[(1S)-3-(methylamino)-1-[[(3S,5R)-5-methyl-2-oxo-pyrrolidin-3-yl]methyl]-2,3-dioxo-propyl]benzamide;
5-chloro-2-[(3,3-difluorocyclohexanecarbonyl)amino]-N-[(1S)-3-(methylamino)-1-[[(3S,5R)-5-methyl-2-oxo-pyrrolidin-3-yl]methyl]-2,3-dioxo-propyl]benzamide;
5-chloro-2-[(3,3-difluorocyclohexanecarbonyl)amino]-N-[(1S)-3-(methylamino)-1-[[(3S,5R)-5-methyl-2-oxo-pyrrolidin-3-yl]methyl]-2,3-dioxo-propyl]benzamide;
(3,3-difluorocyclopentyl)N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
(3,3-difluorocyclopentyl)N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
5-chloro-N-[(1S)-3-(methylamino)-1-[[(3S,5R)-5-methyl-2-oxo-pyrrolidin-3-yl]methyl]-2,3-dioxo-propyl]-2-[[1-(2,2,2-trifluoroethyl)cyclopropanecarbonyl]amino]benzamide;
5-(difluoromethyl)-N-[(1S)-3-(methylamino)-1-[[(3S,5R)-5-methyl-2-oxo-pyrrolidin-3-yl]methyl]-2,3-dioxo-propyl]-2-[[1-(trifluoromethyl)cyclopropanecarbonyl]amino]benzamide;
(5S)—N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-3-methyl-2-oxo-oxazolidine-5-carboxamide;
(5R)—N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-3-methyl-2-oxo-oxazolidine-5-carboxamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]-6-fluoro-phenyl]-3-fluoro-bicyclo[1.1.1]pentane-1-carboxamide;
N-[4-fluoro-2-[[(1S)-3-(methylamino)-1-[[(3S,5R)-5-methyl-2-oxo-pyrrolidin-3-yl]methyl]-2,3-dioxo-propyl]carbamoyl]phenyl]-2-(trifluoromethyl)pyridine-4-carboxamide;
N-[2-[[(1S)-3-amino-1-[[(3S,5R)-5-methyl-2-oxo-pyrrolidin-3-yl]methyl]-2,3-dioxo-propyl]carbamoyl]-4-chloro-phenyl]-2-(trifluoromethyl)pyridine-4-carboxamide;
5-chloro-N-[(1S)-3-(cyclopropylamino)-1-[[(3S,5R)-5-methyl-2-oxo-pyrrolidin-3-yl]methyl]-2,3-dioxo-propyl]-2-[[(2R)-4,4,4-trifluoro-2-methyl-butanoyl]amino]benzamide;
(2,2,2-trifluoro-1-methyl-ethyl)N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]carbamate;
(2,2,2-trifluoro-1-methyl-ethyl)N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]carbamate;
(4S)—N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-1,3-dimethyl-2-oxo-imidazolidine-4-carboxamide;
5-chloro-N-[(1S)-3-(cyclopropylamino)-1-[[(3S,5R)-5-methyl-2-oxo-pyrrolidin-3-yl]methyl]-2,3-dioxo-propyl]-3-fluoro-2-(4,4,4-trifluorobutanoylamino)benzamide;
5-chloro-4-fluoro-N-[(1S)-3-(methylamino)-1-[[(3S,5R)-5-methyl-2-oxo-pyrrolidin-3-yl]methyl]-2,3-dioxo-propyl]-2-[[1-(2,2,2-trifluoroethyl)cyclopropanecarbonyl]amino]benzamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-2-(trifluoromethyl)pyrimidine-5-carboxamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-4-(trifluoromethyl)pyrimidine-2-carboxamide;
N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]-5-(trifluoromethyl)pyrimidine-2-carboxamide;
(3,3-difluorocyclohexyl)methyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
(3,3-difluorocyclohexyl)methyl N-[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamate;
N-[(1S)-3-(cyclopropylamino)-1-[[(3S,5R)-5-methyl-2-oxo-pyrrolidin-3-yl]methyl]-2,3-dioxo-propyl]-5-(difluoromethyl)-2-[[1-(trifluoromethyl)cyclopropanecarbonyl]amino]benzamide;
methyl N-[(1S)-1-[[4-chloro-2-[[(1S)-3-(methylamino)-1-[[(3S,5R)-5-methyl-2-oxo-pyrrolidin-3-yl]methyl]-2,3-dioxo-propyl]carbamoyl]phenyl]carbamoyl]-2,2-dimethyl-propyl]-N-methyl-carbamate;
N-[4-(difluoromethyl)-2-[[(1S)-3-(methylamino)-1-[[(3S,5R)-5-methyl-2-oxo-pyrrolidin-3-yl]methyl]-2,3-dioxo-propyl]carbamoyl]phenyl]-2-(trifluoromethyl)pyridine-4-carboxamide;
N-[4-chloro-5-fluoro-2-[[(1S)-3-(methylamino)-1-[[(3S,5R)-5-methyl-2-oxo-pyrrolidin-3-yl]methyl]-2,3-dioxo-propyl]carbamoyl]phenyl]-6-(trifluoromethyl)pyridine-3-carboxamide;
N-[4-chloro-2-fluoro-6-[[(1S)-3-(methylamino)-1-[[(3S,5R)-5-methyl-2-oxo-pyrrolidin-3-yl]methyl]-2,3-dioxo-propyl]carbamoyl]phenyl]-2-(trifluoromethyl)pyridine-4-carboxamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-6-(trifluoromethyl)pyridine-3-carboxamide;
N-[4-chloro-2-[[(1S)-3-(cyclopropylamino)-1-[[(3S,5R)-5-methyl-2-oxo-pyrrolidin-3-yl]methyl]-2,3-dioxo-propyl]carbamoyl]phenyl]-5-(trifluoromethyl)pyrazine-2-carboxamide;
5-chloro-N-[(1S)-3-(methylamino)-1-[[(3S,5R)-5-methyl-2-oxo-pyrrolidin-3-yl]methyl]-2,3-dioxo-propyl]-2-[2-[2-(trifluoromethyl)-4-pyridyl]propanoylamino]benzamide;
5-chloro-N-[(1S)-3-(methylamino)-1-[[(3S,5R)-5-methyl-2-oxo-pyrrolidin-3-yl]methyl]-2,3-dioxo-propyl]-2-[2-[2-(trifluoromethyl)-4-pyridyl]propanoylamino]benzamide;
5-chloro-N-[(1S)-3-(methylamino)-1-[[(3S,5R)-5-methyl-2-oxo-pyrrolidin-3-yl]methyl]-2,3-dioxo-propyl]-2-[2-[6-(trifluoromethyl)-3-pyridyl]propanoylamino]benzamide;
5-chloro-N-[(1S)-3-(methylamino)-1-[[(3S,5R)-5-methyl-2-oxo-pyrrolidin-3-yl]methyl]-2,3-dioxo-propyl]-2-[2-[6-(trifluoromethyl)-3-pyridyl]propanoylamino]benzamide;
N-[2,4-dichloro-6-[[(1S)-3-(methylamino)-1-[[(3S,5R)-5-methyl-2-oxo-pyrrolidin-3-yl]methyl]-2,3-dioxo-propyl]carbamoyl]phenyl]-2-(trifluoromethyl)pyridine-4-carboxamide;
N-[2-[[(1S)-3-(cyclopropylamino)-1-[[(3S,5R)-5-methyl-2-oxo-pyrrolidin-3-yl]methyl]-2,3-dioxo-propyl]carbamoyl]-4-(difluoromethyl)phenyl]-2-(trifluoromethyl)pyridine-4-carboxamide;
N-[2-[[(1S)-3-(cyclopropylamino)-1-[[(3S,5R)-5-methyl-2-oxo-pyrrolidin-3-yl]methyl]-2,3-dioxo-propyl]carbamoyl]-3,4,5-trifluoro-phenyl]-2-(trifluoromethyl)pyridine-4-carboxamide;
N-[4-chloro-2-[[(1S)-3-(cyclopropylamino)-2,3-dioxo-1-[[(3S)-2-oxo-3-piperidyl]methyl]propyl]carbamoyl]phenyl]-2-(trifluoromethyl)pyridine-4-carboxamide; and
5-chloro-N-[(1S)-3-(cyclopropylamino)-2,3-dioxo-1-[[(3S)-2-oxo-3-piperidyl]methyl]propyl]-2-(4,4,4-trifluorobutanoylamino)benzamide.

14. The compound of claim 1 or a pharmaceutically acceptable salt thereof, wherein the compound is selected from the group consisting of:

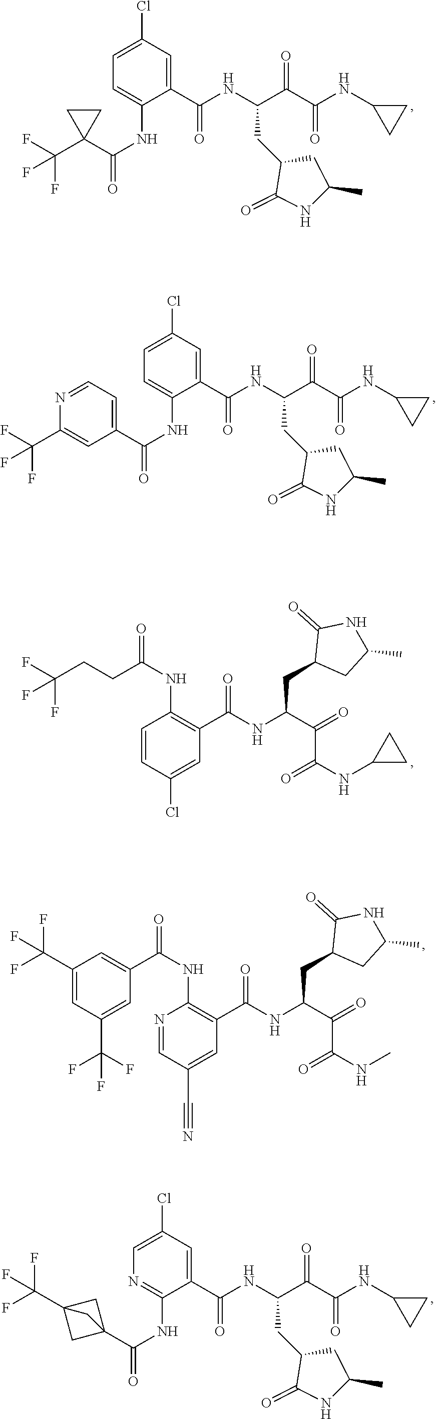 [SMILES: C[C@@H]1C[C@@H](C[C@H](NC(=O)c2cc(Cl)ccc2NC(=O)C2(C(F)(F)F)CC2)C(=O)C(=O)NC2CC2)C(=O)N1 ; C[C@@H]1C[C@@H](C[C@H](NC(=O)c2cc(Cl)ccc2NC(=O)c2ccnc(C(F)(F)F)c2)C(=O)C(=O)NC2CC2)C(=O)N1 ; C[C@@H]1C[C@@H](C[C@H](NC(=O)c2cc(Cl)ccc2NC(=O)CCC(F)(F)F)C(=O)C(=O)NC2CC2)C(=O)N1 ; CNC(=O)C(=O)[C@H](C[C@@H]1C[C@@H](C)NC1=O)NC(=O)c1cc(C#N)cnc1NC(=O)c1cc(C(F)(F)F)cc(C(F)(F)F)c1 ; C[C@@H]1C[C@@H](C[C@H](NC(=O)c2cc(Cl)cnc2NC(=O)C23CC(C(F)(F)F)(C2)C3)C(=O)C(=O)NC2CC2)C(=O)N1]

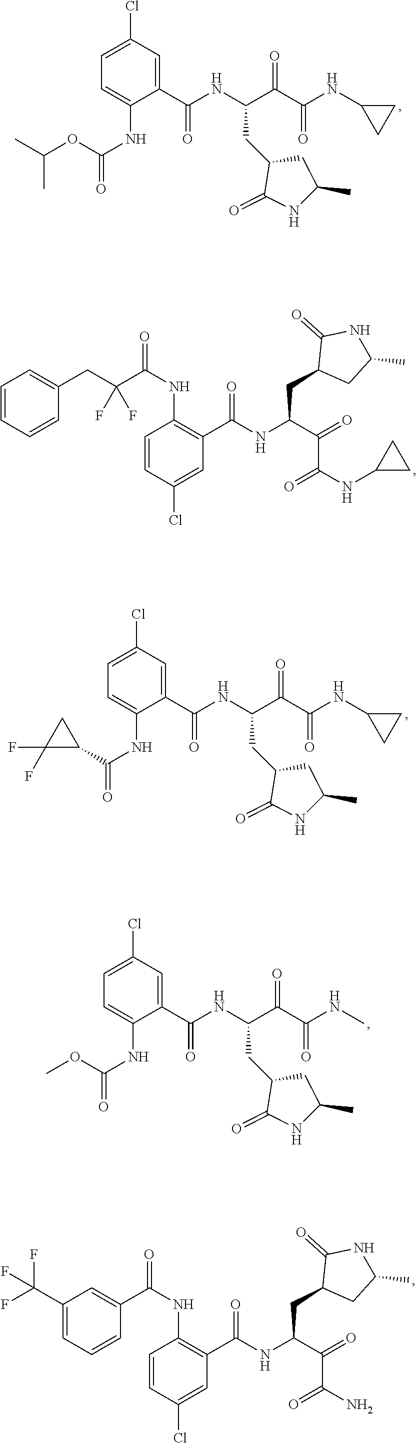

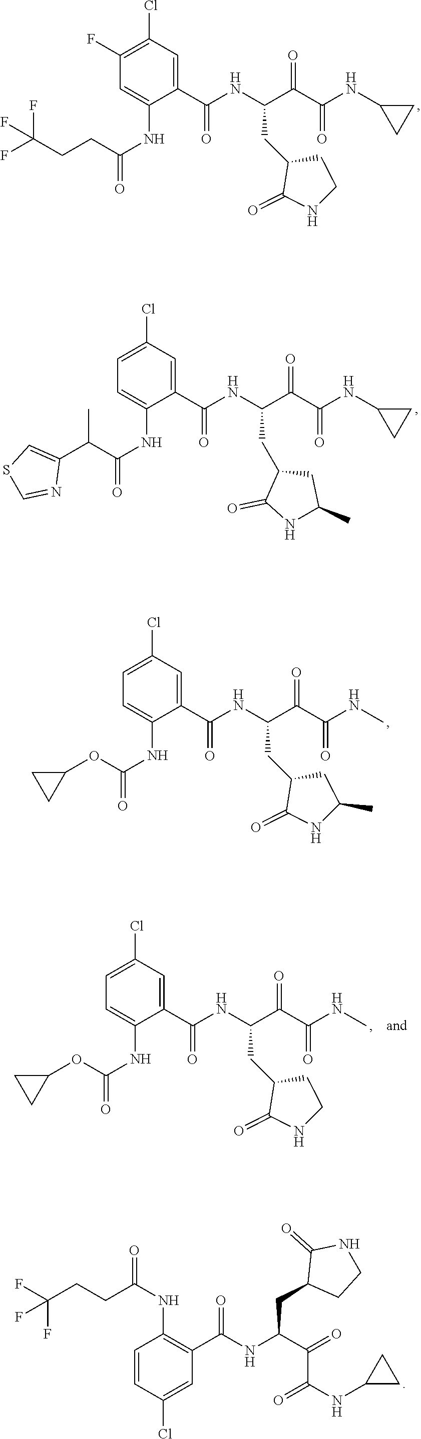 [SMILES: O=C(CCC(F)(F)F)Nc1cc(F)c(Cl)cc1C(=O)N[C@@H](C[C@@H]1CCNC1=O)C(=O)C(=O)NC1CC1 ; CC(C(=O)Nc1ccc(Cl)cc1C(=O)N[C@@H](C[C@@H]1C[C@@H](C)NC1=O)C(=O)C(=O)NC1CC1)c1cscn1 ; CNC(=O)C(=O)[C@H](C[C@@H]1C[C@@H](C)NC1=O)NC(=O)c1cc(Cl)ccc1NC(=O)OC1CC1 ; CNC(=O)C(=O)[C@H](C[C@@H]1CCNC1=O)NC(=O)c1cc(Cl)ccc1NC(=O)OC1CC1 ; O=C(CCC(F)(F)F)Nc1ccc(Cl)cc1C(=O)N[C@@H](C[C@@H]1CCNC1=O)C(=O)C(=O)NC1CC1]

15. A pharmaceutical composition comprising the compound of claim 1 or a pharmaceutically acceptable salt thereof and a pharmaceutically acceptable carrier.

16. The pharmaceutical composition of claim 15 in the form of an orally administered tablet or capsule.

17. The pharmaceutical composition of claim 15, further comprising one or more additional therapeutic agent(s).

18. The pharmaceutical composition of claim 17, wherein the one or more additional therapeutic agent(s) are selected from molnupiravir, pomotrelvir, ensitrelvir, nirmatrelvir, and ritonavir.

19. A method for prophylaxis or treatment of a coronavirus infection, comprising administering an effective amount of the compound of claim 1 or a pharmaceutically acceptable salt thereof to a patient in need thereof.

20. The method of claim 19, wherein the coronavirus infection is a SARS-CoV, SARS-CoV-2 or MERS-CoV infection.

21. The method of claim 20, wherein the coronavirus infection is a SARS-CoV-2 infection.

22. The method of claim 19, further comprising one or more additional therapeutic agent(s) to the patient.

23. The method of claim 22, wherein the one or more additional therapeutic agent(s) are selected from molnupiravir, pomotrelvir, ensitrelvir, nirmatrelvir, and ritonavir.

24. The compound of claim 1 or a pharmaceutically acceptable salt thereof, for use as a medical treatment.

25. The use of claim 24, wherein the medical treatment is for prophylaxis or treatment of a coronavirus infection.

26. The use of claim 25, wherein the coronavirus infection is a SARS-CoV, SARS-CoV-2 or MERS-CoV infection.
